(19) United States
(12) Patent Application Publication
(10) Pub. No.: US 2023/0257351 A1
(43) Pub. Date: Aug. 17, 2023

(54) SUBSTITUTED N-PHENYLACETAMIDES HAVING P2X4 RECEPTOR ANTAGONISTIC ACTIVITY

(71) Applicant: Bayer Aktiengesellschaft, Leverkusen (DE)

(72) Inventors: Stefan BÄURLE, Berlin (DE); Sven RING, Jena (DE); Jens NAGEL, Daxweiler (DE); Alexis LAUX-BIEHLMANN, Lörrach (DE); Reinhard NUBBEMEYER, Berlin (DE); Elisabeth POOK, Wuppertal (DE); Sven WITTROCK, Berlin (DE); Daryl Simon WALTER, Oxfordshire (GB); Robert John SCOTT, Oxfordshire (GB)

(73) Assignee: Bayer Aktiengesellschaft, Leverkusen (DE)

(21) Appl. No.: 18/012,570

(22) PCT Filed: Jun. 28, 2021
(86) PCT No.: PCT/EP2021/067713

§ 371 Date: Dec. 22, 2022

(30) Foreign Application Priority Data
Jun. 30, 2020 (EP) 20183306.8
Jan. 15, 2021 (EP) 21151884.0

(51) Int. Cl. C07D 213/75; C07D 237/20; A61P 29/00

(52) U.S. Cl. CPC C07D 213/75; C07D 237/20; A61P 29/00

(57) ABSTRACT

Substituted N-phenylacetamide compounds of general formula (I), methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases.

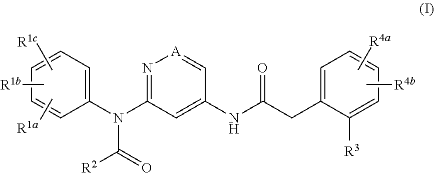

The present invention covers substituted N-phenylacetamide compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular for the treatment or prophylaxis of diseases associated with pain, pain syndromes (acute and chronic), inflammatory-induced pain, neuropathic pain, diabetic neuropathic pain, diabetic neuropathy, cancer-associated pain, chemotherapy or intoxication induced pain, pelvic pain, endometriosis-associated pain as well as endometriosis as such, bladder pain syndrome; asthma, bronchiolitis obliterans syndrome, chronic obstructive pulmonary disease (COPD), chronic cough, diseases related to goblet cells and lung fibrosis, liver fibrosis, fatty liver disorders, NASH (Non-Alcoholic Steato-Hepatitis); brain ischemia, ischemic brain injury, ischemic stroke, haemorrhagic stroke, traumatic brain injury, spinal cord injury, aneurysm; chronic itch, pruritus; osteoarthritis, burning mouth syndrome, migraine disorders, irritable bowel disease; urology related syndromes like, overactive urinary bladder, interstitial cystitis, bladder pain syndrome. The present invention, as described and defined herein, covers pharmaceutical compositions and combinations comprising an active ingredient which is an antagonist or a negative allosteric modulator of P2X4.

### BACKGROUND

The present invention covers substituted N-phenylacetamides of general formula (I) which are antagonists or negative allosteric modulators of P2X4. Adenosine triphosphate ATP is widely recognized as an important neurotransmitter implicated in various physiological and pathophysiological roles by acting through different subtypes of purinergic receptors (Burnstock 1993, Drug Dev Res 28:196-206; Burnstock 2011, Prog Neurobiol 95:229-274). To date, seven members of the P2X family have been cloned, comprising P2X1-7 (Burnstock 2013, Front Cell Neurosci 7:227). The P2X4 receptor is a ligand-gated ion channel that is expressed on a variety of cell types largely known to be involved in inflammatory/immune processes specifically including monocytes, macrophages, mast cells and microglia cells (Wang et al., 2004, BMC Immunol 5:16; Brone et al., 2007 Immunol Lett 113:83-89). Activation of P2X4 by extracellular ATP is known, amongst other things, to lead to release of pro-inflammatory cytokines and prostaglandins (PGE2) (Bo et al., 2003 Cell Tissue Res 313:159-165; Ulmann et al., 2010, EMBO Journal 29:2290-2300; de Ribero Vaccari et al., 2012, J Neurosci 32:3058-3066). Numerous lines of evidence in the literature using animal models implicate P2X4 receptor in nociception and pain. Mice lacking the P2X4 receptor do not develop pain hypersensitivity in response to numerous inflammatory challenges such as complete Freunds Adjuvant (CFA), carrageenan or formalin (Ulmann et al., 2010, EMBO Journal 29:2290-2300). In addition, mice lacking the P2X4R do not develop mechanical allodynia after peripheral nerve injury, indicating very prominent role of P2X4 in neuropathic pain conditions (Tsuda et al., 2009, Mol Pain 5:28; Ulmann et al., 2008, J Neurocsci 28:11263-11268). Moehring et al. (Elife. 2018 Jan. 16; 7 “Keratinocytes mediate innocuous and noxious touch via ATP-P2X4 signaling”) reported experiments identifying P2X4 signalling as a critical component of baseline mammalian tactile sensation. These experiments lay a vital foundation for subsequent studies into the dysfunctional signalling that occurs in cutaneous pain and itch disorders.

Besides the extensively described role of P2X4 in acute and chronic pain-related diseases (Trang and Salter, 2012, Purinergic Signalling 8:621-628; Burnstock, 2013 Eur J Pharmacol 716:24-40), P2X4 is considered as a critically important mediator of inflammatory diseases such as, respiratory diseases (e.g. asthma, COPD), lung diseases including fibrosis, cancer and atherosclerosis (Burnstock et al., 2012 Pharmacol Rev. 64:834-868).

EP 2 597 088 A1 describes P2X4 receptor antagonists and in particular a diazepine derivative of formula (III) or a pharmacologically acceptable salt thereof. Said document further disclosed the use of P2X4 receptor antagonist diazepine derivatives represented by the formula (I), (II), (Ill), or its pharmacologically acceptable salt, which shows P2X4 receptor antagonism, being effective as an agent for prevention or treatment of nociceptive, inflammatory, and neuropathic pain. In more detail, EP 2 597 088 A1 describes P2X4 receptor antagonists being effective as a preventive or therapeutic agent for pain caused by various cancers, diabetic neuritis, viral diseases such as herpes, and osteoarthritis. The preventive or therapeutic agent according to EP 2 597 088 A1 can also be used in combination with other agents such as opioid analgesic (e.g., morphine, fentanyl), sodium channel inhibitor (e.g., novocaine, lidocaine), or NSAIDs (e.g., aspirin, ibuprofen). The P2X4 receptor antagonist used for pain caused by cancers can be also used in combination with a carcinostatic such as a chemotherapic. Further P2X4 receptor antagonists and their use are disclosed in WO2013105608, WO2015005467 and WO2015005468, WO2016198374, WO2017191000, WO2018/104305, WO2018/104307.

“Discovery and characterization of novel, potent and selective P2X4 receptor antagonists for the treatment of pain” was presented at the Society for Neuroscience Annual Meeting 2014 (Carrie A Bowen et al.; poster N. 241.1) Said poster describes the methods to identify novel, potent and selective small-molecule antagonists that inhibit P2X4 across species, and how to evaluate selected compounds in experimental models of neuropathic and inflammatory pain. In particular a method for human, rat, mouse P2X4R FLIPR-based screening, a human P2X4R electrophysiology assay, a suitable mouse neuropathy model and a mouse inflammation model were described.

WO2015088564 and WO2015088565 provide P2X4 receptor modulating compounds, methods of their synthesis, pharmaceutical compositions comprising the compounds, and methods of their use. Said P2X4 receptor modulating compounds are useful for the treatment, prevention, and/or management of various disorders, including but not limited to, chronic pain, neuropathy, inflammatory diseases and central nervous system disorders.

US2018/0280409 describes methods for the treatment of a human subject who has had a stroke by administering to the subject a pharmaceutical composition including an antagonist of the P2X4 receptor. The antagonist of the P2X4 receptor can be administered in the acute phase of stroke, optionally in combination with a thrombolytic therapeutic or a procedure on the subject involving a clot-removal device.

WO2019081573A1 describes as well pharmaceutical compositions and combinations comprising an active ingredient which is an antagonist or a negative allosteric modulator of P2X4 for the treatment or prophylaxis of brain ischemia, ischemic brain injury, Ischemic Stroke (IS), haemorrhagic stroke, traumatic brain injury, spinal cord injury.

WO2019177117A1 describes a drug for preventing or treating cough, the drug containing as an active ingredient a compound having a P2X4 receptor antagonizing action, a tautomer, stereoisomer, or pharmacologically acceptable salt of said compound, or a hydrate or solvate thereof.

There is no reference in the state of the art about substituted N-phenylacetamides of general formula (I) as described and defined herein and to the use of said compounds for manufacturing a pharmaceutical composition for the treatment or prophylaxis of a disease, particularly to the use of substituted aromatic sulfonamides of general formula (I) for the treatment or prophylaxis of diseases associated with pain, or for the treatment or prophylaxis of pain syndromes (acute and chronic), inflammatory-induced pain, neuropathic pain including diabetic neuropathic pain, pelvic pain, cancer or chemotherapy-associated pain, endometriosis-associated pain as well as endometriosis as such, bladder pain syndrome, cancer as such, and proliferative diseases as such like endometriosis, as a sole agent or in combination with other active ingredients.

Therefore, the inhibitors of P2X4 of the current invention represent valuable compounds that should complement therapeutic options either as single agents or in combination with other drugs.

### DESCRIPTION OF THE INVENTION

In accordance with a first aspect, the present invention covers compounds of general formula (I):

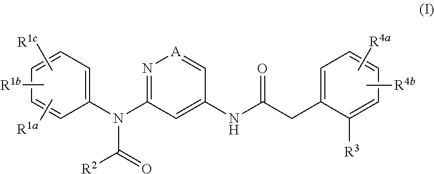

in which A is CH or N

R^{1a}, R^{1b}, and R^{1c} mean independently from each other a hydrogen atom, a halogen atom, cyano, (C₁-C₃)-alkyl, (C₁-C₃)-haloalkyl, (C₁-C₃)-alkoxy,

R² is (C₁-C₃)-alkyl;

R³ means a halogen atom, cyano, (C₁-C₃)-alkyl, (C₁-C₃)-haloalkyl, (C₁-C₃)-alkoxy,

R^{4a} and R^{4b} mean independently from each other a hydrogen atom, a halogen atom, cyano, (C₁-C₃)-alkyl, (C₁-C₃)-haloalkyl, (C₁-C₃)-alkoxy,

and stereoisomers, tautomers, N-oxides, hydrates, solvates, and salts thereof, and mixtures of same.

In a second aspect, the present invention covers compounds of general formula (Ia):

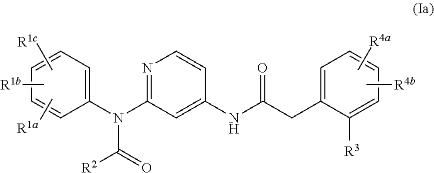

in which

R^{1a}, R^{1b}, and R^{1c} mean independently from each other a hydrogen atom, a halogen atom, cyano, (C₁-C₃)-alkyl, C₁-C₃-haloalkyl, (C₁-C₃)-alkoxy;

R² is (C₁-C₃)-alkyl;

R³ means a halogen atom, cyano, (C₁-C₃)-alkyl, (C₁-C₃)-haloalkyl, (C₁-C₃)-alkoxy;

R^{4a} and R^{4b} mean independently from each other a hydrogen atom, a halogen atom, cyano, (C₁-C₃)-alkyl, (C₁-C₃)-haloalkyl, (C₁-C₃)-alkoxy;

and stereoisomers, tautomers, N-oxides, hydrates, solvates, and salts thereof, and mixtures of same.

In a further aspect, the present invention covers compounds of general formula (Ib):

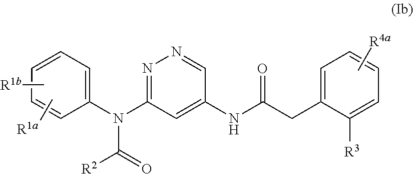

R^{1a}, R^{1b}, and R^{1c} mean independently from each other a hydrogen atom, a halogen atom, cyano, (C₁-C₃)-alkyl, (C₁-C₃)-haloalkyl, (C₁-C₃)-alkoxy,

R² is (C₁-C₃)-alkyl;

R³ means a halogen atom, cyano, (C₁-C₃)-alkyl, (C₁-C₃)-haloalkyl, (C₁-C₃)-alkoxy;

R^{4a} and R^{4b} mean independently from each other a hydrogen atom, a halogen atom, cyano, (C₁-C₃)-alkyl, (C₁-C₃)-haloalkyl, (C₁-C₃)-alkoxy;

and stereoisomers, tautomers, N-oxides, hydrates, solvates, and salts thereof, and mixtures of same.

### Definitions

The term “comprising” when used in the specification includes “consisting of”.

If within the present text any item is referred to as “as mentioned herein”, it means that it may be mentioned anywhere in the present text.

The terms as mentioned in the present text have the following meanings: The term “halogen atom” means a fluorine, chlorine, bromine or iodine atom, particularly a fluorine, chlorine or bromine atom, more particularly fluorine or chlorine atom.

In the context of the present invention, the substituents and residues have the following meanings, unless specified otherwise:

(C₁-C₃)-Alkyl in the context of the invention means a straight-chain or branched alkyl group having 1, 2, or 3 carbon atoms, such as: methyl, ethyl, n-propyl, isopropyl, and isobutyl, for example. (C₁-C₃)-Alkoxy in the context of the invention means a straight-chain or branched alkoxy group having 1, 2, or 3 carbon atoms, such as: methoxy, ethoxy, n-propoxy, and isopropoxy, for example.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centers, depending upon the location and nature of the various substituents desired. It is possible that one or more asymmetric carbon atoms are present in the (R) or (S) configuration, which can result in racemic mixtures In certain instances, it is possible that asymmetry also be present due to restricted rotation about a given bond, for example, the central bond adjoining two substituted aromatic rings of the specified compounds.

The purification and the separation of such materials can be accomplished by standard techniques known in the art.

The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive but are converted (for example metabolically or hydrolytically) into compounds according to the invention during their residence time in the body.

In a further embodiment of the first aspect, the present invention covers compounds of formula (I), supra, in which R^{1a}, and R^{1b} mean independently from each other a halogen atom, cyano, (C₁-C₃)-alkyl, (C₁-C₃)-haloalkyl, (C₁-C₃)-alkoxy; and R^{1c} is a hydrogen atom.

According to a further embodiment of the invention R^{1a} is in position 4 of the phenyl ring and means a halogen atom, cyano, (C₁-C₃)-alkyl, (C₁-C₃)-haloalkyl, (C₁-C₃)-alkoxy; R^{1b} means a hydrogen atom a halogen atom, cyano, (C₁-C₃)-alkyl, (C₁-C₃)-haloalkyl, (C₁-C₃)-alkoxy; and R^{1c} is a hydrogen atom.

Furthermore, in relation to a further form of the invention, R^{1a} is in position 4 of the phenyl ring and means a halogen atom, cyano, (C₁-C₃)-alkyl, (C₁-C₃)-haloalkyl, (C₁-C₃)-alkoxy; R^{1b} is in position 3 of the phenyl ring and a hydrogen atom a halogen atom, cyano, (C₁-C₃)-alkyl, (C₁-C₃)-haloalkyl, (C₁-C₃)-alkoxy; and R^{1c} is a hydrogen atom.

In a further specific embodiment of the invention, R^{1a} means a halogen atom, cyano, (C₁-C₃)-alkyl, (C₁-C₃)-haloalkyl, (C₁-C₃)-alkoxy; R^{1b} and R^{1c} are a hydrogen atom.

A specific embodiment of the invention is that in which R² means methyl, ethyl or n-propyl; more particularly R² means a methyl.

According to a further embodiment of the invention, R³ means a chlorine, fluorine, cyano, or a hydrogen atom In a further specific embodiment of the invention, R^{4a} is a halogen atom, cyano, (C₁-C₃)-alkyl, (C₁-C₃)-haloalkyl, (C₁-C₃)-alkoxy; and R^{4b} is a hydrogen atom.

Furthermore, in relation to a further form of the invention, R^{4a} is a halogen atom, cyano, (C₁-C₃)-alkyl, (C₁-C₃)-haloalkyl, (C₁-C₃)-alkoxy; and R^{4b} is a halogen atom, cyano, (C₁-C₃)-alkyl, (C₁-C₃)-haloalkyl, (C₁-C₃)-alkoxy.

The invention further comprises particular embodiments in which R³ means a chlorine, fluorine, cyano, R^{4a} is a halogen atom, cyano, (C₁-C₃)-alkyl, (C₁-C₃)-haloalkyl, (C₁-C₃)-alkoxy in position 3 or 6 of the phenyl group; and R^{4b} is a hydrogen atom.

In a further specific embodiment of the invention R³ means a chlorine, fluorine, cyano, R^{4a} is a halogen atom, cyano, (C₁-C₃)-alkyl, (C₁-C₃)-haloalkyl, (C₁-C₃)-alkoxy in position 6 of the phenyl group; and R^{4b} is a halogen atom, cyano, (C₁-C₃)-alkyl, (C₁-C₃)-haloalkyl, (C₁-C₃)-alkoxy in position 4 of the phenyl group.

In a further embodiment of the first aspect, the present invention covers combinations of two or more of the above-mentioned embodiments under the heading “further embodiments of the first aspect of the present invention”.

The present invention covers any sub-combination within any embodiment or aspect of the present invention of compounds of general formula (I), supra. The present invention covers any sub-combination within any embodiment or aspect of the present invention of intermediate compounds of general formula (VII), (VIII). (XIII). (XIV).

The present invention covers the compounds of general formula (I) which are disclosed in the Example Section of this text, infra, namely:
1. N-{4-[2-(2-chlorophenyl)acetamido]pyridin-2-yl}-N-(3,4-difluorophenyl)acetamide
2. N-{4-[2-(2-chloro-3-fluorophenyl)acetamido]pyridin-2-yl}-N-(4-fluorophenyl)acetamide
3. N-{4-[2-(2-chloro-4-fluorophenyl)acetamido]pyridin-2-yl}-N-[3-(difluoromethyl)phenyl]acetamide
4. N-{4-[2-(2-chloro-3-fluorophenyl)acetamido]pyridin-2-yl}-N-[3-(difluoromethyl)phenyl]acetamide
5. N-{4-[2-(2,6-dichlorophenyl)acetamido]pyridin-2-yl}-N-[3-(difluoromethyl)phenyl]acetamide
6. N-{4-[2-(2-chlorophenyl)acetamido]pyridin-2-yl}-N-[3-(trifluoromethyl)phenyl]acetamide
7. N-{4-[2-(2-chloro-4-fluorophenyl)acetamido]pyridin-2-yl}-N-[3-(trifluoromethyl)phenyl]acetamide
8. N-{4-[2-(2-chloro-3-fluorophenyl)acetamido]pyridin-2-yl}-N-[3-(trifluoromethyl)phenyl]acetamide
9. N-{4-[2-(2,6-dichlorophenyl)acetamido]pyridin-2-yl}-N-[3-(trifluoromethyl)phenyl]acetamide
10. N-{4-[2-(2-chlorophenyl)acetamido]pyridin-2-yl}-N-[4-cyano-3-(trifluoromethyl)phenyl]acetamide
11. N-{4-[2-(2-chloro-3-fluorophenyl)acetamido]pyridin-2-yl}-N-[4-cyano-3-(trifluoromethyl)phenyl]acetamide
12. N-[4-cyano-3-(trifluoromethyl)phenyl]-N-{4-[2-(2,6-dichlorophenyl)acetamido]pyridin-2-yl}acetamide
13. N-{4-[2-(2-chlorophenyl)acetamido]pyridin-2-yl}-N-(3-cyanophenyl)acetamide
14. N-(3-cyanophenyl)-N-{4-[2-(2,6-dichlorophenyl)acetamido]pyridin-2-yl}acetamide
15. N-{4-[2-(2-chloro-6-fluorophenyl)acetamido]pyridin-2-yl}-N-(3-cyanophenyl)acetamide
16. N-(4-chloro-3-cyanophenyl)-N-{4-[2-(2-chlorophenyl)acetamido]pyridin-2-yl}acetamide
17. N-(4-chloro-3-cyanophenyl)-N-{4-[2-(2-chloro-4-fluorophenyl)acetamido]pyridin-2-yl}acetamide
18. N-(4-chloro-3-cyanophenyl)-N-{4-[2-(2-chloro-3-fluorophenyl)acetamido]pyridin-2-yl}acetamide
19. N-(4-chloro-3-cyanophenyl)-N-{4-[2-(2,6-dichlorophenyl)acetamido]pyridin-2-yl}acetamide
20. N-(4-{2-[2-chloro-3-(trifluoromethyl)phenyl]acetamido}pyridin-2-yl)-N-(3-cyano-4-fluorophenyl)acetamide
21. N-{4-[2-(2-chlorophenyl)acetamido]pyridin-2-yl}-N-(3-cyano-4-fluorophenyl)acetamide
22. N-{4-[2-(2-chloro-4-fluorophenyl)acetamido]pyridin-2-yl}-N-(3-cyano-4-fluorophenyl)acetamide
23. N-{4-[2-(2-chloro-3-fluorophenyl)acetamido]pyridin-2-yl}-N-(3-cyano-4-fluorophenyl)acetamide
24. N-(3-cyano-4-fluorophenyl)-N-{4-[2-(2,6-dichlorophenyl)acetamido]pyridin-2-yl}acetamide
25. N-{4-[2-(2-chlorophenyl)acetamido]pyridin-2-yl}-N-phenylacetamide
26. N-{4-[2-(2-chloro-4-fluorophenyl)acetamido]pyridin-2-yl}-N-phenylacetamide
27. N-{4-[2-(2,6-dichlorophenyl)acetamido]pyridin-2-yl}-N-phenylacetamide
28. N-{4-[2-(2-chloro-6-fluorophenyl)acetamido]pyridin-2-yl}-N-phenylacetamide
29. N-{4-[2-(2,3-dimethylphenyl)acetamido]pyridin-2-yl}-N-(4-fluorophenyl)acetamide
30. N-(4-fluorophenyl)-N-(4-{2-[2-(trifluoromethyl)phenyl]acetamido}pyridin-2-yl)acetamide
31. N-(4-{2-[4-chloro-2-(trifluoromethyl)phenyl]acetamido}pyridin-2-yl)-N-(4-fluorophenyl)acetamide
32. N-(4-fluorophenyl)-N-(4-{2-[3-fluoro-2-(trifluoromethyl)phenyl]acetamido}pyridin-2-yl)acetamide
33. N-{4-[2-(2-chloro-6-cyanophenyl)acetamido]pyridin-2-yl}-N-(4-fluorophenyl)acetamide
34. N-{4-[2-(2,6-dimethylphenyl)acetamido]pyridin-2-yl}-N-(4-fluorophenyl)acetamide
35. N-(4-{2-[2-chloro-3-(trifluoromethyl)phenyl]acetamido}pyridin-2-yl)-N-(4-fluorophenyl)acetamide
36. N-{4-[2-(2-chlorophenyl)acetamido]pyridin-2-yl}-N-(4-fluorophenyl)acetamide
37. N-{4-[2-(2-chloro-4-fluorophenyl)acetamido]pyridin-2-yl}-N-(4-fluorophenyl)acetamide
38. N-{4-[2-(2,6-dichlorophenyl)acetamido]pyridin-2-yl}-N-(4-fluorophenyl)acetamide
39. N-{4-[2-(2,6-dichloro-4-fluorophenyl)acetamido]pyridin-2-yl}-N-(4-fluorophenyl)acetamide
40. N-{4-[2-(2-chloro-6-fluorophenyl)acetamido]pyridin-2-yl}-N-(4-fluorophenyl)acetamide
41. N-{4-[2-(2-chloro-4,6-difluorophenyl)acetamido]pyridin-2-yl}-N-(4-fluorophenyl)acetamide
42. N-(4-fluorophenyl)-N-(4-{2-[2-(trifluoromethoxy)phenyl]acetamido}pyridin-2-yl)acetamide
43. N-{4-[2-(2-chlorophenyl)acetamido]pyridin-2-yl}-N-(4-methylphenyl)acetamide
44. N-{4-[2-(2-chloro-4-fluorophenyl)acetamido]pyridin-2-yl}-N-(4-methylphenyl)acetamide
45. N-{4-[2-(2-chloro-3-fluorophenyl)acetamido]pyridin-2-yl}-N-(4-methylphenyl)acetamide
46. N-{4-[2-(2,6-dichlorophenyl)acetamido]pyridin-2-yl}-N-(4-methylphenyl)acetamide
47. N-{4-[2-(2-chlorophenyl)acetamido]pyridin-2-yl}-N-[4-(difluoromethoxy)phenyl]acetamide
48. N-{4-[2-(2-chloro-3-fluorophenyl)acetamido]pyridin-2-yl}-N-[4-(difluoromethoxy)phenyl]acetamide
49. N-{4-[2-(2,6-dichlorophenyl)acetamido]pyridin-2-yl}-N-[4-(difluoromethoxy)phenyl]acetamide
50. N-(3-chloro-4-fluorophenyl)-N-{4-[2-(2-chlorophenyl)acetamido]pyridin-2-yl}acetamide
51. N-(3-chloro-4-fluorophenyl)-N-{4-[2-(2-chloro-4-fluorophenyl)acetamido]pyridin-2-yl}acetamide
52. N-(3-chloro-4-fluorophenyl)-N-{4-[2-(2-chloro-3-fluorophenyl)acetamido]pyridin-2-yl}acetamide
53. N-(3-chloro-4-fluorophenyl)-N-{4-[2-(2,6-dichlorophenyl)acetamido]pyridin-2-yl}acetamide
54. N-{4-[2-(2-chlorophenyl)acetamido]pyridin-2-yl}-N-(3-fluorophenyl)acetamide
55. N-{4-[2-(2-chloro-4-fluorophenyl)acetamido]pyridin-2-yl}-N-(3-fluorophenyl)acetamide
56. N-{4-[2-(2,6-dichlorophenyl)acetamido]pyridin-2-yl}-N-(3-fluorophenyl)acetamide
57. N-{4-[2-(2,6-dichloro-4-fluorophenyl)acetamido]pyridin-2-yl}-N-(3-fluorophenyl)acetamide
58. N-(4-chloro-3-fluorophenyl)-N-{4-[2-(2-chlorophenyl)acetamido]pyridin-2-yl}acetamide
59. N-(4-chloro-3-fluorophenyl)-N-{4-[2-(2-chloro-4-fluorophenyl)acetamido]pyridin-2-yl}acetamide
60. N-(4-chloro-3-fluorophenyl)-N-{4-[2-(2-chloro-3-fluorophenyl)acetamido]pyridin-2-yl}acetamide
61. N-(4-chloro-3-fluorophenyl)-N-{4-[2-(2,6-dichlorophenyl)acetamido]pyridin-2-yl}acetamide
62. N-(3,4-difluorophenyl)-N-{4-[2-(2,3-dimethylphenyl)acetamido]pyridin-2-yl}acetamide
63. N-(3,4-difluorophenyl)-N-(4-{2-[2-(trifluoromethyl)phenyl]acetamido}pyridin-2-yl)acetamide
64. N-{4-[2-(2,4-dichloro-6-methylphenyl)acetamido]pyridin-2-yl}-N-(3,4-difluorophenyl)acetamide
65. N-{4-[2-(2-chloro-4,6-dimethylphenyl)acetamido]pyridin-2-yl}-N-(3,4-difluorophenyl)acetamide
66. N-(3,4-difluorophenyl)-N-{4-[2-(2,6-dimethylphenyl)acetamido]pyridin-2-yl}acetamide
67. N-{4-[2-(2,4-dichlorophenyl)acetamido]pyridin-2-yl}-N-(3,4-difluorophenyl)acetamide
68. N-{4-[2-(2-chloro-4-fluorophenyl)acetamido]pyridin-2-yl}-N-(3,4-difluorophenyl)acetamide
69. N-{4-[2-(2-chloro-4-nitrophenyl)acetamido]pyridin-2-yl}-N-(3,4-difluorophenyl)acetamide
70. N-{4-[2-(2-chloro-4-methoxyphenyl)acetamido]pyridin-2-yl}-N-(3,4-difluorophenyl)acetamide
71. N-{4-[2-(2-chloro-3-fluorophenyl)acetamido]pyridin-2-yl}-N-(3,4-difluorophenyl)acetamide
72. N-{4-[2-(2,6-dichlorophenyl)acetamido]pyridin-2-yl}-N-(3,4-difluorophenyl)acetamide
73. N-{4-[2-(2,6-dichloro-4-fluorophenyl)acetamido]pyridin-2-yl}-N-(3,4-difluorophenyl)acetamide
74. N-{4-[2-(2,6-dichloro-4-methylphenyl)acetamido]pyridin-2-yl}-N-(3,4-difluorophenyl)acetamide
75. N-{4-[2-(2,6-dichloro-4-ethylphenyl)acetamido]pyridin-2-yl}-N-(3,4-difluorophenyl)acetamide
76. N-(3,4-difluorophenyl)-N-(4-{2-[2-(trifluoromethoxy)phenyl]acetamido}pyridin-2-yl)acetamide
77. N-{4-[2-(2-chlorophenyl)acetamido]pyridin-2-yl}-N-(3-fluoro-4-methoxyphenyl)acetamide
78. N-{4-[2-(2-chloro-4-fluorophenyl)acetamido]pyridin-2-yl}-N-(3-fluoro-4-methoxyphenyl)acetamide
79. N-{4-[2-(2-chloro-3-fluorophenyl)acetamido]pyridin-2-yl}-N-(3-fluoro-4-methoxyphenyl)acetamide
80. N-{4-[2-(2,6-dichlorophenyl)acetamido]pyridin-2-yl}-N-(3-fluoro-4-methoxyphenyl)acetamide
81. N-{4-[2-(2-chloro-3-fluorophenyl)acetamido]pyridin-2-yl}-N-[3-fluoro-4-(methanesulfonyl)phenyl]acetamide
82. N-{4-[2-(2,6-dichlorophenyl)acetamido]pyridin-2-yl}-N-[3-fluoro-4-(methanesulfonyl)phenyl]acetamide
83. N-(3,5-difluorophenyl)-N-{4-[2-(2,3-dimethylphenyl)acetamido]pyridin-2-yl}acetamide
84. N-(3,5-difluorophenyl)-N-(4-{2-[2-(trifluoromethyl)phenyl]acetamido}pyridin-2-yl)acetamide
85. N-(3,5-difluorophenyl)-N-{4-[2-(2,6-dimethylphenyl)acetamido]pyridin-2-yl}acetamide
86. N-{4-[2-(2-chlorophenyl)acetamido]pyridin-2-yl}-N-(3,5-difluorophenyl)acetamide
87. N-{4-[2-(2-chloro-4-fluorophenyl)acetamido]pyridin-2-yl}-N-(3,5-difluorophenyl)acetamide
88. N-{4-[2-(2-chloro-3-fluorophenyl)acetamido]pyridin-2-yl}-N-(3,5-difluorophenyl)acetamide
89. N-{4-[2-(2,6-dichlorophenyl)acetamido]pyridin-2-yl}-N-(3,5-difluorophenyl)acetamide
90. N-{4-[2-(2,6-dichloro-4-fluorophenyl)acetamido]pyridin-2-yl}-N-(3,5-difluorophenyl)acetamide
91. N-(3,5-difluorophenyl)-N-(4-{2-[2-(trifluoromethoxy)phenyl]acetamido}pyridin-2-yl)acetamide
92. N-{4-[2-(2-chlorophenyl)acetamido]pyridin-2-yl}-N-(3,5-difluoro-4-methylphenyl)acetamide
93. N-{4-[2-(2-chloro-4-fluorophenyl)acetamido]pyridin-2-yl}-N-(3,5-difluoro-4-methylphenyl)acetamide
94. N-{4-[2-(2-chloro-3-fluorophenyl)acetamido]pyridin-2-yl}-N-(3,5-difluoro-4-methylphenyl)acetamide
95. N-{4-[2-(2,6-dichlorophenyl)acetamido]pyridin-2-yl}-N-(3,5-difluoro-4-methylphenyl)acetamide
96. N-{4-[2-(2-chlorophenyl)acetamido]pyridin-2-yl}-N-(3,5-difluoro-4-methoxyphenyl)acetamide
97. N-{4-[2-(2-chloro-4-fluorophenyl)acetamido]pyridin-2-yl}-N-(3,5-difluoro-4-methoxyphenyl)acetamide
98. N-{4-[2-(2-chloro-3-fluorophenyl)acetamido]pyridin-2-yl}-N-(3,5-difluoro-4-methoxyphenyl)acetamide
99. N-{4-[2-(2,6-dichlorophenyl)acetamido]pyridin-2-yl}-N-(3,5-difluoro-4-methoxyphenyl)acetamide
100. N-{4-[2-(2-chlorophenyl)acetamido]pyridin-2-yl}-N-(3-methoxyphenyl)acetamide
101. N-{4-[2-(2-chloro-3-fluorophenyl)acetamido]pyridin-2-yl}-N-(3-methoxyphenyl)acetamide
102. N-{4-[2-(2-chlorophenyl)acetamido]pyridin-2-yl}-N-[3-(trifluoromethoxy)phenyl]acetamide
103. N-{4-[2-(2-chloro-3-fluorophenyl)acetamido]pyridin-2-yl}-N-(4-fluoro-3-methoxyphenyl)acetamide
104. N-{4-[2-(2,6-dichlorophenyl)acetamido]pyridin-2-yl}-N-(4-fluoro-3-methoxyphenyl)acetamide
105. N-(2-chlorophenyl)-N-(4-{2-[2-chloro-3-(trifluoromethyl)phenyl]acetamido}pyridin-2-yl)acetamide
106. N-(2-chlorophenyl)-N-{4-[2-(2-chlorophenyl)acetamido]pyridin-2-yl}acetamide
107. N-{4-[2-(2-chloro-4-fluorophenyl)acetamido]pyridin-2-yl}-N-(2-chlorophenyl)acetamide
108. N-{4-[2-(2-chloro-3-fluorophenyl)acetamido]pyridin-2-yl}-N-(2-chlorophenyl)acetamide
109. N-(2-chlorophenyl)-N-{4-[2-(2,6-dichlorophenyl)acetamido]pyridin-2-yl}acetamide
110. N-(2-chloro-5-fluorophenyl)-N-(4-{2-[2-chloro-3-(trifluoromethyl)phenyl]acetamido}pyridin-2-yl)acetamide
111. N-(2-chloro-5-fluorophenyl)-N-{4-[2-(2-chlorophenyl)acetamido]pyridin-2-yl}acetamide
112. N-(2-chloro-5-fluorophenyl)-N-{4-[2-(2-chloro-4-fluorophenyl)acetamido]pyridin-2-yl}acetamide
113. N-(2-chloro-5-fluorophenyl)-N-{4-[2-(2-chloro-3-fluorophenyl)acetamido]pyridin-2-yl}acetamide
114. N-(2-chloro-5-fluorophenyl)-N-{4-[2-(2,6-dichlorophenyl)acetamido]pyridin-2-yl}acetamide
115. N-{4-[2-(2-chlorophenyl)acetamido]pyridin-2-yl}-N-(2-fluorophenyl)acetamide
116. N-{4-[2-(2-chloro-4-fluorophenyl)acetamido]pyridin-2-yl}-N-(2-fluorophenyl)acetamide
117. N-{4-[2-(2-chloro-3-fluorophenyl)acetamido]pyridin-2-yl}-N-(2-fluorophenyl)acetamide
118. N-{4-[2-(2,6-dichlorophenyl)acetamido]pyridin-2-yl}-N-(2-fluorophenyl)acetamide
119. N-{4-[2-(2-chloro-3-fluorophenyl)acetamido]pyridin-2-yl}-N-[2-fluoro-4-(trifluoromethyl)phenyl]acetamide
120. N-{4-[2-(2,6-dichlorophenyl)acetamido]pyridin-2-yl}-N-[2-fluoro-4-(trifluoromethyl)phenyl]acetamide
121. N-(4-{2-[2-chloro-3-(trifluoromethyl)phenyl]acetamido}pyridin-2-yl)-N-(2,3-difluorophenyl)acetamide.
122. N-{4-[2-(2-chlorophenyl)acetamido]pyridin-2-yl}-N-(2,3-difluorophenyl)acetamide
123. N-{4-[2-(2-chloro-4-fluorophenyl)acetamido]pyridin-2-yl}-N-(2,3-difluorophenyl)acetamide
124. N-{4-[2-(2-chloro-3-fluorophenyl)acetamido]pyridin-2-yl}-N-(2,3-difluorophenyl)acetamide
125. N-{4-[2-(2,6-dichlorophenyl)acetamido]pyridin-2-yl}-N-(2,3-difluorophenyl)acetamide
126. N-{4-[2-(2-chlorophenyl)acetamido]pyridin-2-yl}-N-(2,4-difluorophenyl)acetamide
127. N-{4-[2-(2-chloro-4-fluorophenyl)acetamido]pyridin-2-yl}-N-(2,4-difluorophenyl)acetamide
128. N-{4-[2-(2-chloro-3-fluorophenyl)acetamido]pyridin-2-yl}-N-(2,4-difluorophenyl)acetamide
129. N-{4-[2-(2,6-dichlorophenyl)acetamido]pyridin-2-yl}-N-(2,4-difluorophenyl)acetamide
130. N-{4-[2-(2-chloro-6-fluorophenyl)acetamido]pyridin-2-yl}-N-(2,4-difluorophenyl)acetamide
131. N-(3-chlorophenyl)-N-{4-[2-(2-chlorophenyl)acetamido]pyridin-2-yl}acetamide
132. N-{4-[2-(2-chloro-4-fluorophenyl)acetamido]pyridin-2-yl}-N-(3-chlorophenyl)acetamide
133. N-{4-[2-(2-chloro-3-fluorophenyl)acetamido]pyridin-2-yl}-N-(3-chlorophenyl)acetamide
134. N-(3-chlorophenyl)-N-{4-[2-(2,6-dichlorophenyl)acetamido]pyridin-2-yl}acetamide
135. N-{4-[2-(2-chloro-6-fluorophenyl)acetamido]pyridin-2-yl}-N-(3-chlorophenyl)acetamide
136. N-(3-chloro-5-fluorophenyl)-N-{4-[2-(2-chlorophenyl)acetamido]pyridin-2-yl}acetamide
137. N-(3-chloro-5-fluorophenyl)-N-{4-[2-(2-chloro-4-fluorophenyl)acetamido]pyridin-2-yl}acetamide
138. N-(3-chloro-5-fluorophenyl)-N-{4-[2-(2-chloro-3-fluorophenyl)acetamido]pyridin-2-yl}acetamide
139. N-(3-chloro-5-fluorophenyl)-N-{4-[2-(2,6-dichlorophenyl)acetamido]pyridin-2-yl}acetamide
140. N-(3-chloro-5-fluorophenyl)-N-{4-[2-(2-chloro-6-fluorophenyl)acetamido]pyridin-2-yl}acetamide
141. N-{4-[2-(2-chloro-4-fluorophenyl)acetamido]pyridin-2-yl}-N-(4-fluorophenyl)butanamide
142. N-{4-[2-(2-chloro-6-fluorophenyl)acetamido]pyridin-2-yl}-N-(4-fluorophenyl)butanamide
143. N-{4-[2-(2-chlorophenyl)acetamido]pyridin-2-yl}-N-(4-fluorophenyl)butanamide
144. N-{4-[2-(2,6-dichlorophenyl)acetamido]pyridin-2-yl}-N-(4-fluorophenyl)butanamide
145. N-{4-[2-(2-Chlorphenyl)acetamido]pyridin-2-yl}-N-(3-cyan-5-fluorphenyl)acetamide
146. N-(3-Cyan-5-fluorphenyl)-N-{4-[2-(2,6-dichlorphenyl)acetamido]pyridin-2-yl}acetamide
147. N-{4-[2-(2-Chlor-3-fluorphenyl)acetamido]pyridin-2-yl}-N-(3-cyan-5-fluorphenyl)acetamide
148. N-{4-[2-(2-Chlor-6-fluorphenyl)acetamido]pyridin-2-yl}-N-(3-cyan-5-fluorphenyl)acetamide
149. N-{4-[2-(2-Chlor-4-fluorphenyl)acetamido]pyridin-2-yl}-N-(3-cyan-5-fluorphenyl)acetamide
150. N-(2-Chlor-4-fluorphenyl)-N-{4-[2-(2-chlorphenyl)acetamido]pyridin-2-yl}acetamide
151. N-(2-Chlor-4-fluorphenyl)-N-{4-[2-(2-chlor-4-fluorphenyl)acetamido]pyridin-2-yl}acetamide
152. N-(2-Chlor-4-fluorphenyl)-N-{4-[2-(2,6-dichlorphenyl)acetamido]pyridin-2-yl}acetamide
153. N-(2-Chlor-4-fluorphenyl)-N-{4-[2-(2-chlor-6-fluorphenyl)acetamido]pyridin-2-yl}acetamide
154. N-(2-Chlor-4-fluorphenyl)-N-{4-[2-(2-chlor-3-fluorphenyl)acetamido]pyridin-2-yl}acetamide
155. N-[3-Chlor-4-(methylsulfonyl)phenyl]-N-{4-[2-(2,6-dichlorphenyl)acetamido]pyridin-2-yl}acetamide
156. N-{4-[2-(2-Chlor-3-fluorphenyl)acetamido]pyridin-2-yl}-N-[3-chlor-4-(methylsulfonyl)phenyl]acetamide
157. N-[3-Chlor-4-(methylsulfonyl)phenyl]-N-{4-[2-(2-chlorphenyl)acetamido]pyridin-2-yl}acetamide
158. N-{4-[2-(2-Chlor-6-fluorphenyl)acetamido]pyridin-2-yl}-N-[3-chlor-4-(methylsulfonyl)phenyl]acetamide
159. N-{4-[2-(2-Chlor-4-fluorphenyl)acetamido]pyridin-2-yl}-N-[3-chlor-4-(methylsulfonyl)phenyl]acetamide
160. N-{4-[2-(2-Chlor-3-fluorphenyl)acetamido]pyridin-2-yl}-N-(3-fluor-5-methoxyphenyl)acetamide
161. N-{4-[2-(2-Chlorphenyl)acetamido]pyridin-2-yl}-N-(3-fluor-5-methoxyphenyl)acetamide
162. N-{4-[2-(2,6-Dichlorphenyl)acetamido]pyridin-2-yl}-N-(3-fluor-5-methoxyphenyl)acetamide
163. N-{4-[2-(2-Chlor-4-fluorphenyl)acetamido]pyridin-2-yl}-N-(3-fluor-5-methoxyphenyl)acetamide
164. N-{4-[2-(2-Chlor-6-fluorphenyl)acetamido]pyridin-2-yl}-N-(3-fluor-5-methoxyphenyl)acetamide
165. N-{4-[2-(2,6-Dichlorphenyl)acetamido]pyridin-2-yl}-N-[2-(difluormethyl)phenyl]acetamide
166. N-{4-[2-(2-Chlorphenyl)acetamido]pyridin-2-yl}-N-[2-(difluormethyl)phenyl]acetamide
167. N-{4-[2-(2-Chlor-3-fluorphenyl)acetamido]pyridin-2-yl}-N-[2-(difluormethyl)phenyl]acetamide
168. N-{4-[2-(2-Chlor-6-fluorphenyl)acetamido]pyridin-2-yl}-N-[2-(difluormethyl)phenyl]acetamide
169. N-{4-[2-(2-Chlor-4-fluorphenyl)acetamido]pyridin-2-yl}-N-[2-(difluormethyl)phenyl]acetamide
170. N-{4-[2-(2-Chlorphenyl)acetamido]pyridin-2-yl}-N-(2,4-dimethylphenyl)acetamide
171. N-{4-[2-(2-Chlor-3-fluorphenyl)acetamido]pyridin-2-yl}-N-(2,4-dimethylphenyl)acetamide
172. N-{4-[2-(2-Chlor-6-fluorphenyl)acetamido]pyridin-2-yl}-N-(2,4-dimethylphenyl)acetamide
173. N-{4-[2-(2,6-Dichlorphenyl)acetamido]pyridin-2-yl}-N-(2,4-dimethylphenyl)acetamide
174. N-{4-[2-(2-Chlor-4-fluorphenyl)acetamido]pyridin-2-yl}-N-(2,4-dimethylphenyl)acetamide
175. N-{4-[2-(2-Chlorphenyl)acetamido]pyridin-2-yl}-N-(3-cyan-5-methylphenyl)acetamide
176. N-(3-Cyan-5-methylphenyl)-N-{4-[2-(2,6-dichlorphenyl)acetamido]pyridin-2-yl}acetamide
177. N-{4-[2-(2-Chlor-3-fluorphenyl)acetamido]pyridin-2-yl}-N-(3-cyan-5-methylphenyl)acetamide
178. N-{4-[2-(2-Chlor-4-fluorphenyl)acetamido]pyridin-2-yl}-N-(3-cyan-5-methylphenyl)acetamide
179. N-{4-[2-(2-Chlor-6-fluorphenyl)acetamido]pyridin-2-yl}-N-(3-cyan-5-methylphenyl)acetamide
180. N-(3-Chlor-4-methylphenyl)-N-{4-[2-(2-chlorphenyl)acetamido]pyridin-2-yl}acetamide
181. N-{4-[2-(2-Chlor-3-fluorphenyl)acetamido]pyridin-2-yl}-N-(3-chlor-4-methylphenyl)acetamide
182. N-(3-Chlor-4-methylphenyl)-N-{4-[2-(2,6-dichlorphenyl)acetamido]pyridin-2-yl}acetamide
183. N-{4-[2-(2-Chlor-6-fluorphenyl)acetamido]pyridin-2-yl}-N-(3-chlor-4-methylphenyl)acetamide
184. N-{4-[2-(2-Chlor-6-fluorphenyl)acetamido]pyridin-2-yl}-N-(4-fluor-2,3-dimethylphenyl)acetamide
185. N-{5-[2-(2,6-dichlorophenyl)acetamido]pyridazin-3-yl}-N-phenylacetamide
186. N-{5-[2-(2-chloro-3-fluorophenyl)acetamido]pyridazin-3-yl}-N-phenylacetamide
187. N-{5-[2-(2-chlorophenyl)acetamido]pyridazin-3-yl}-N-phenylacetamide
188. N-{5-[2-(2-chloro-4-fluorophenyl)acetamido]pyridazin-3-yl}-N-phenylacetamide
189. N-{5-[2-(2-chloro-6-fluorophenyl)acetamido]pyridazin-3-yl}-N-(4-fluorophenyl)acetamide
190. N-{5-[2-(2-chloro-4-fluorophenyl)acetamido]pyridazin-3-yl}-N-(4-fluorophenyl)acetamide
191. N-{5-[2-(2-chloro-3-fluorophenyl)acetamido]pyridazin-3-yl}-N-(4-fluorophenyl)acetamide
192. N-{5-[2-(2-chlorophenyl)acetamido]pyridazin-3-yl}-N-(4-fluorophenyl)acetamide
193. N-{5-[2-(2,6-dichlorophenyl)acetamido]pyridazin-3-yl}-N-(4-fluorophenyl)acetamide
194. N-{5-[2-(2,6-dichlorophenyl)acetamido]pyridazin-3-yl}-N-(3-fluorophenyl)acetamide
195. N-{5-[2-(2-chloro-3-fluorophenyl)acetamido]pyridazin-3-yl}-N-(3-fluorophenyl)acetamide
196. N-{5-[2-(2-chlorophenyl)acetamido]pyridazin-3-yl}-N-(3-fluorophenyl)acetamide
197. N-{5-[2-(2-chloro-6-fluorophenyl)acetamido]pyridazin-3-yl}-N-(3-fluorophenyl)acetamide
198. N-{5-[2-(2-chloro-4-fluorophenyl)acetamido]pyridazin-3-yl}-N-(3-fluorophenyl)acetamide
199. N-{5-[2-(2-chlorophenyl)acetamido]pyridazin-3-yl}-N-(3,4-difluorophenyl)acetamide
200. N-{5-[2-(2,6-dichlorophenyl)acetamido]pyridazin-3-yl}-N-(3,4-difluorophenyl)acetamide
201. N-{5-[2-(2-chloro-3-fluorophenyl)acetamido]pyridazin-3-yl}-N-(3,4-difluorophenyl)acetamide
202. N-{5-[2-(2-chloro-4-fluorophenyl)acetamido]pyridazin-3-yl}-N-(3,4-difluorophenyl)acetamide
203. N-{5-[2-(2-chloro-6-fluorophenyl)acetamido]pyridazin-3-yl}-N-(3,4-difluorophenyl)acetamide
204. N-{5-[2-(2,6-dichlorophenyl)acetamido]pyridazin-3-yl}-N-(3,5-difluorophenyl)acetamide
205. N-{5-[2-(2-chlorophenyl)acetamido]pyridazin-3-yl}-N-(3,5-difluorophenyl)acetamide
206. N-{5-[2-(2-chloro-3-fluorophenyl)acetamido]pyridazin-3-yl}-N-(3,5-difluorophenyl)acetamide
207. N-{5-[2-(2-chloro-4-fluorophenyl)acetamido]pyridazin-3-yl}-N-(3,5-difluorophenyl)acetamide
208. N-{5-[2-(2-chloro-6-fluorophenyl)acetamido]pyridazin-3-yl}-N-(3,5-difluorophenyl)acetamide

The compounds according to the invention of general formula (I) can be prepared according to the following schemes 1, 2 and 3. The schemes and procedures described below illustrate synthetic routes to the compounds of general formula (I) of the invention and are not intended to be limiting. It is clear to the person skilled in the art that the order of transformations as exemplified in schemes 1, 2 and 3 can be modified in various ways. The order of transformations exemplified in these schemes is therefore not intended to be limiting. In addition, interconversion of any of the substituents, R^{1a}, R^{1b}, R^{1c}, R², R³, R^{4a}, or R^{4b} can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Scheme 1 depicts the synthesis starting from aromatic amines of the formula (II), and synthons of formula (III), wherein Hal stands for Cl, Br, I or a triflate, Br being preferred; and wherein A stands for CH. The two starting materials can be cross-coupled by Pd-mediated reactions (Buchwald-Hartwig-coupling) known to those skilled in the art. A suitable solvent like for example N,N-dimethylformamide, 1,4-dioxane or toluene is used and a base such as potassium carbonate, potassium phosphate, caesium carbonate or potassium tert-butanolate is added. Appropriate palladium catalysts in combination with suitable phosphine ligands are utilized as catalyst catalyst-ligand system, for example bis(dibenzylidenaceton) palladium(0) and 4,5-bis-(diphenylphosphino)-9,9-dimethyl xanthene (Xantphos). The reaction is performed at temperatures between 80° C. and 120° C., preferred at 100° C. until complete conversion, typically for 18 h. Aromatic amines of general formula (IV) may react according to standard procedures with carboxylic acid anhydrides (V) or the corresponding acetyl chlorides (VI) to yield amides of general formula (VII). In case of the use of anhydrides (V) like e.g. acetanhyride, it may also serve as solvent. N,N-dimethylaminopyridine may be used as catalyst (0.1 eq). The reaction usually takes place between 100 and 130° C. until complete conversion (2-18 h). In case of the use of carboxylic acid chloride, e.g. acetyl chloride, dichloromethane may be used as solvent and a base, e.g. triethyl amine, is added. The nitro group in compounds of the general formula (VII) are reduced to the corresponding amino group of compounds of general formula (VIII) via procedures known to those skilled in the art, e.g. via hydrogenation in presence of a suitable catalyst like palladium or platinum, e.g. 10% Pd on activated charcoal. Preferably, atmospheric hydrogen pressure is utilized. Suitable solvents like ethanol, methanol or ethyl acetate (which is preferred) are used. Alternatively, other reduction methods are used, most notably the reduction with iron powder (5 eq.) in acetic acid. The mixture is stirred vigorously until complete conversion (2-18 h). Aromatic amines of general formula (VIII) may react with carboxylic acids of general formula (IX) by methods known to those skilled in the art to give the amide compounds of general formula (I). The reaction is mediated by activating a carboxylic acid of general formula (IX) with reagents such as dicyclohexylcarbodiimide (DCC), 1-(3-dimethylaminopropyl)-3-ethylcarbodiimide (EDCl), N-hydroxybenzotriazole (HOBT), N-[(dimethylamino)-(3H-[1,2,3]triazolo[4,5-b]pyridin-3-yloxy)methyliden]-N-methylmethanaminium hexafluorophosphate (HATU) or propylphosphonic anhydride (T3P). For example, the reaction with HATU or T3P takes place in an inert solvent, such as N,N-dimethylformamide, dichloromethane or dimethyl sulfoxide in the presence of the appropriate aromatic amine of general formula (VIII) and a tertiary amine (such as triethylamine or diisopropylethylamine) at temperatures between −30° C. and +80° C.

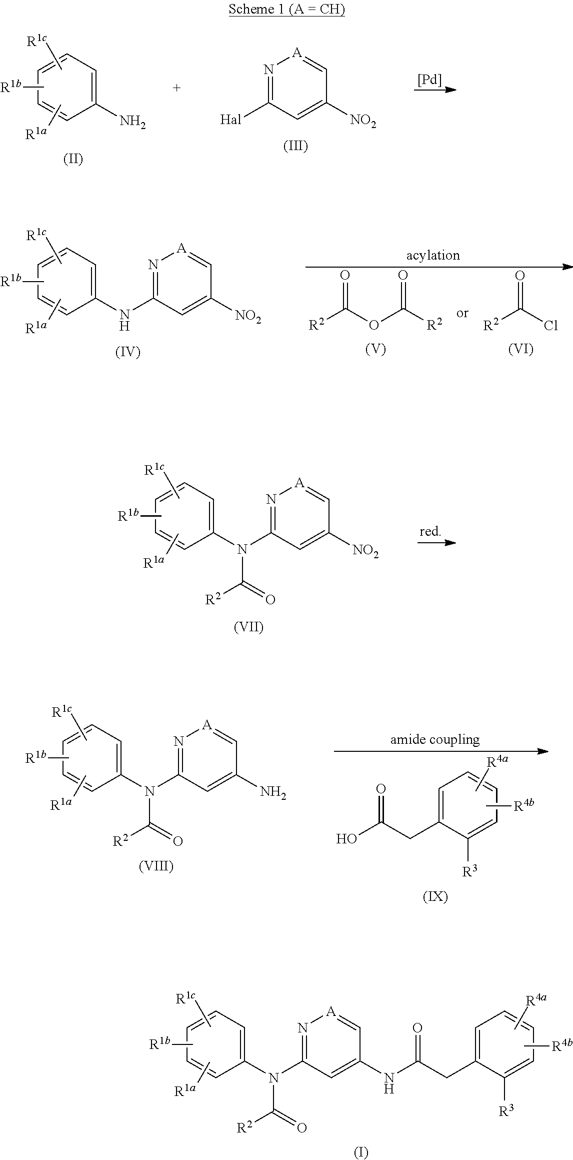

in which A is CH and R^{1a}, R^{1b}, R^{1c}, R² and R³, R^{4a}, R^{4b} are as defined for the compound of general formula (I) supra.

As an alternative, the first step described in scheme 1 may also be performed using an aromatic halide of general formula (X) and a synthon of general formula (XI) (scheme 2).

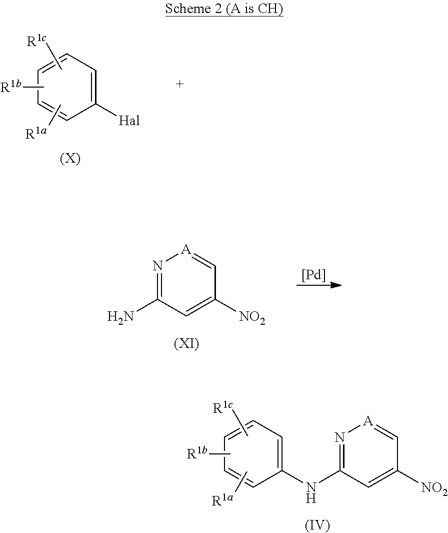

in which A is CH and R^{1a}, R^{1b}, and R^{1c} are as defined for the compound of general formula (I) supra.

The sequence of the synthesis steps may be changed as appropriate.

For example, in case A=N, the steps were performed as outlined in scheme 3. Compound (XII) was used as starting materials. First, the amide coupling using carboxylic acids of type (IX) were carried out, followed by Pd-catalyzed cross coupling with aromatic amines of general formula (II) and acylation with acid chlorides of type (VI).

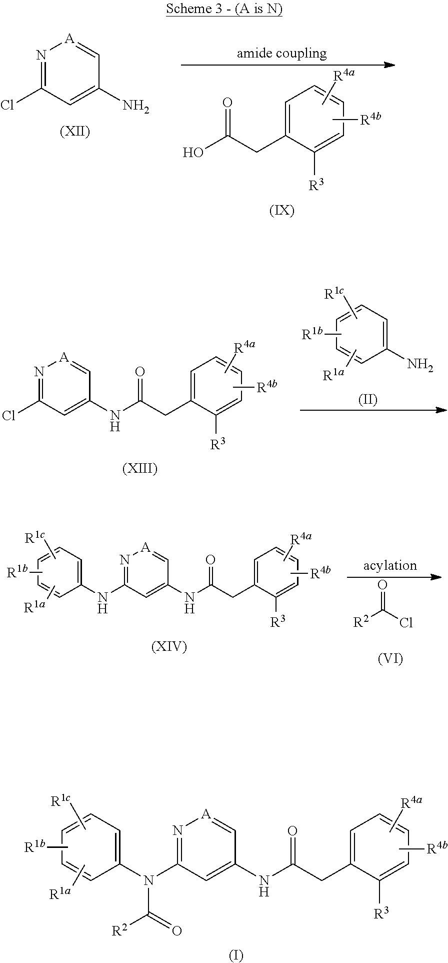

in which A is N and R^{1a}, R^{1b}, R^{1c}, R² and R³, R^{4a}, R^{4b} are as defined for the compound of general formula (I) supra.

Compounds (II), (Ill), (V), (VI), (IX), (X) and (XI) are either commercially available or can be prepared according to procedures available from the public domain, as understandable to the person skilled in the art. Specific examples are described in the Experimental Section.

An alternative approach to synthesize compounds of general formula (I) is depicted in scheme 3A.

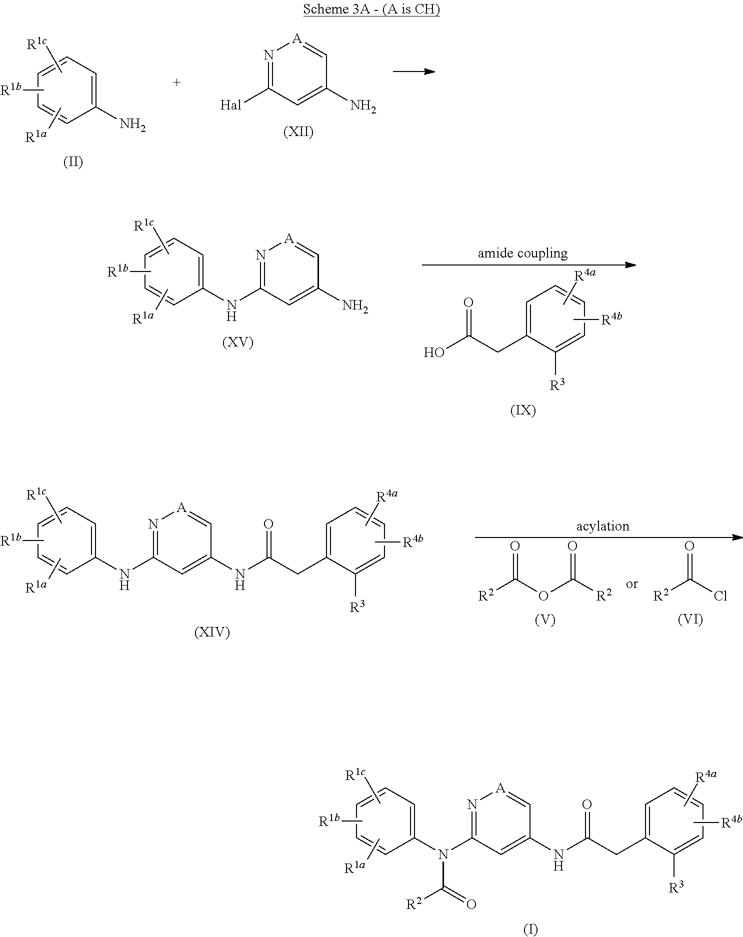

This synthesis starts from aromatic amines of the formula (II), and synthons of formula (XII), wherein Hal stands for Cl, Br, I or a triflate, Cl being preferred; and wherein A stands for CH. The two starting materials can be coupled by heating in higher boiling solvents, preferably in sulfolan (60°-130° C., 10-20 h, typically 130° C., for 18 h) in the presence of hydrochloric acid (1 eq). Alternatively, a cross-coupling by Pd-mediated reactions (Buchwald-Hartwig-coupling) known to those skilled in the art is also possible.

Aromatic amines of general formula (XV) may react with carboxylic acids of general formula (IX) by methods known to those skilled in the art to give the amide compounds of general formula (XIV). In particular, the coupling can be performed by activation with 1,1′-carbonyldiimidazole (1.0-1.5 eq.) in preferably N,N-dimethylacetamide as solvent. The reaction mixture is typically stirred at temperatures between r.t. and 80° C. (typically 40° C.) for 10 h to 24 h (typically 18 h).

Aromatic amines of general formula (XIV) may react according to standard procedures with carboxylic acid anhydrides (V) or the corresponding acyl chlorides (VI) to yield amides of general formula (I). In case of the use of anhydrides (V) like e.g. acetanhydride, it may also serve as solvent. N,N-dimethylaminopyridine may be used as catalyst (0.1 eq). The reaction usually takes place between 100 and 130° C. until complete conversion (2-18 h). In case of the use of carboxylic acid chloride, e.g. acetyl chloride, dichloromethane or, more preferred, rac-2-methyltetrahydrofuran, may be used as solvent. A base, e.g. triethyl amine or N,N-diisopropylethylamine (1-2 eq., typically 1.4 eq.), is added. Conversion takes place typically at room temperature in 1 to 24 h, typically in 18 h).

Particularly, the invention covers the intermediate compounds of general formula (VII) and (VIII):

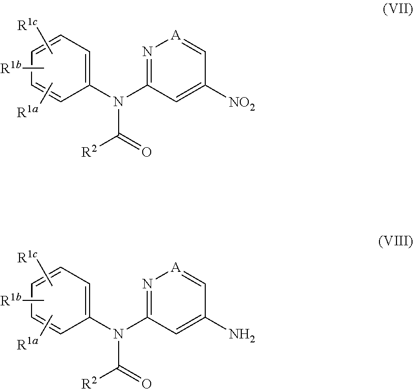

in which A, R^{1a}, R^{1b}, R^{1c}, and R² are as defined for the compound of general formula (I) supra.

Intermediate compounds of general formula (VII) according to the invention are in particular:
39. N-(3,4-difluorophenyl)-N-(4-nitropyridin-2-yl)acetamide
40. N-[3-(difluoromethyl)phenyl]-N-(4-nitropyridin-2-yl)acetamide
41. N-(4-nitropyridin-2-yl)-N-[3-(trifluoromethyl)phenyl]acetamide
42. N-[4-cyano-3-(trifluoromethyl)phenyl]-N-(4-nitropyridin-2-yl)acetamide
43. N-(3-cyanophenyl)-N-(4-nitropyridin-2-yl)acetamide
44. N-(4-chloro-3-cyanophenyl)-N-(4-nitropyridin-2-yl)acetamide
45. N-(3-cyano-4-fluorophenyl)-N-(4-nitropyridin-2-yl)acetamide
46. N-(4-nitropyridin-2-yl)-N-phenylacetamide
47. N-(4-fluorophenyl)-N-(4-nitropyridin-2-yl)acetamide
48. N-(4-methylphenyl)-N-(4-nitropyridin-2-yl)acetamide
49. N-[4-(difluoromethoxy)phenyl]-N-(4-nitropyridin-2-yl)acetamide
50. N-(3-chloro-4-fluorophenyl)-N-(4-nitropyridin-2-yl)acetamide
51. N-(3-fluorophenyl)-N-(4-nitropyridin-2-yl)acetamide
52. N-(4-chloro-3-fluorophenyl)-N-(4-nitropyridin-2-yl)acetamide
53. N-(3-fluoro-4-methoxyphenyl)-N-(4-nitropyridin-2-yl)acetamide
54. N-[3-fluoro-4-(methanesulfonyl)phenyl]-N-(4-nitropyridin-2-yl)acetamide
55. N-(3,5-difluorophenyl)-N-(4-nitropyridin-2-yl)acetamide
56. N-(3,5-difluoro-4-methylphenyl)-N-(4-nitropyridin-2-yl)acetamide
57. N-(3,5-difluoro-4-methoxyphenyl)-N-(4-nitropyridin-2-yl)acetamide
58. N-(3-methoxyphenyl)-N-(4-nitropyridin-2-yl)acetamide
59. N-(4-nitropyridin-2-yl)-N-[3-(trifluoromethoxy)phenyl]acetamide
60. N-(4-fluoro-3-methoxyphenyl)-N-(4-nitropyridin-2-yl)acetamide
61. N-(2-chlorophenyl)-N-(4-nitropyridin-2-yl)acetamide
62. N-(2-chloro-5-fluorophenyl)-N-(4-nitropyridin-2-yl)acetamide
63. N-(2-fluorophenyl)-N-(4-nitropyridin-2-yl)acetamide
64. N-[2-fluoro-4-(trifluoromethyl)phenyl]-N-(4-nitropyridin-2-yl)acetamide
65. N-(2,3-difluorophenyl)-N-(4-nitropyridin-2-yl)acetamide
66. N-(2,4-difluorophenyl)-N-(4-nitropyridin-2-yl)acetamide
67. N-(3-chlorophenyl)-N-(4-nitropyridin-2-yl)acetamide
68. N-(3-chloro-5-fluorophenyl)-N-(4-nitropyridin-2-yl)acetamide
69. N-(3-Cyan-5-fluorphenyl)-N-(4-nitropyridin-2-yl)acetamide
70. N-(2-Chlor-4-fluorphenyl)-N-(4-nitropyridin-2-yl)acetamide
71. N-[3-Chlor-4-(methylsulfonyl)phenyl]-N-(4-nitropyridin-2-yl)acetamid
72. N-(3-Fluor-5-methoxyphenyl)-N-(4-nitropyridin-2-yl)acetamide
73. N-[2-(Difluormethyl)phenyl]-N-(4-nitropyridin-2-yl)acetamide
74. N-(2,4-Dimethylphenyl)-N-(4-nitropyridin-2-yl)acetamide
75. N-(3-Cyan-5-methylphenyl)-N-(4-nitropyridin-2-yl)acetamide
76. N-(4-Fluor-2,3-dimethylphenyl)-N-(4-nitropyridin-2-yl)acetamide
77. N-(3-Chlor-4-methylphenyl)-N-(4-nitropyridin-2-yl)acetamide
78. N-(4-fluorophenyl)-N-(4-nitropyridin-2-yl)butanamide
Furthermore, intermediate compounds of general formula (VIII) are:
79. N-(4-aminopyridin-2-yl)-N-(3,4-difluorophenyl)acetamide
80. N-(4-aminopyridin-2-yl)-N-(4-fluorophenyl)acetamide
81. N-(4-aminopyridin-2-yl)-N-[3-(difluoromethyl)phenyl]acetamide
82. N-(4-aminopyridin-2-yl)-N-[3-(trifluoromethyl)phenyl]acetamide
83. N-(4-aminopyridin-2-yl)-N-[4-cyano-3-(trifluoromethyl)phenyl]acetamide
84. N-(4-aminopyridin-2-yl)-N-(3-cyanophenyl)acetamide
85. N-(4-aminopyridin-2-yl)-N-(4-chloro-3-cyanophenyl)acetamide
86. N-(4-aminopyridin-2-yl)-N-(3-cyano-4-fluorophenyl)acetamide
87. N-(4-aminopyridin-2-yl)-N-phenylacetamide
88. N-(4-aminopyridin-2-yl)-N-(4-methylphenyl)acetamide
89. N-(4-aminopyridin-2-yl)-N-[4-(difluoromethoxy)phenyl]acetamide
90. N-(4-aminopyridin-2-yl)-N-(3-chloro-4-fluorophenyl)acetamide
91. N-(4-aminopyridin-2-yl)-N-(3-fluorophenyl)acetamide
92. N-(4-aminopyridin-2-yl)-N-(4-chloro-3-fluorophenyl)acetamide
93. N-(4-aminopyridin-2-yl)-N-(3-fluoro-4-methoxyphenyl)acetamide
94. N-(4-aminopyridin-2-yl)-N-[3-fluoro-4-(methanesulfonyl)phenyl]acetamide
95. N-(4-aminopyridin-2-yl)-N-(3,5-difluorophenyl)acetamide
96. N-(4-aminopyridin-2-yl)-N-(3,5-difluoro-4-methylphenyl)acetamide
97. N-(4-aminopyridin-2-yl)-N-(3,5-difluoro-4-methoxyphenyl)acetamide
98. N-(4-aminopyridin-2-yl)-N-(3-methoxyphenyl)acetamide
99. N-(4-aminopyridin-2-yl)-N-[3-(trifluoromethoxy)phenyl]acetamide
100. N-(4-aminopyridin-2-yl)-N-(4-fluoro-3-methoxyphenyl)acetamide
101. N-(4-aminopyridin-2-yl)-N-(2-chlorophenyl)acetamide
102. N-(4-aminopyridin-2-yl)-N-(2-chloro-5-fluorophenyl)acetamide
103. N-(4-aminopyridin-2-yl)-N-(2-fluorophenyl)acetamide
104. N-(4-aminopyridin-2-yl)-N-[2-fluoro-4-(trifluoromethyl)phenyl]acetamide
105. N-(4-aminopyridin-2-yl)-N-(2,3-difluorophenyl)acetamide
106. N-(4-aminopyridin-2-yl)-N-(2,4-difluorophenyl)acetamide
107. N-(4-aminopyridin-2-yl)-N-(3-chlorophenyl)acetamide
108. N-(4-aminopyridin-2-yl)-N-(3-chloro-5-fluorophenyl)acetamide
109. N-(4-Aminopyridin-2-yl)-N-(3-cyan-5-fluorphenyl)acetamide
110. N-(4-Aminopyridin-2-yl)-N-(2-chlor-4-fluorphenyl)acetamide
111. N-(4-Aminopyridin-2-yl)-N-[3-chlor-4-(methylsulfonyl)phenyl]acetamide
112. N-(4-Aminopyridin-2-yl)-N-(3-fluor-5-methoxyphenyl)acetamide
113. N-(4-Aminopyridin-2-yl)-N-[2-(difluormethyl)phenyl]acetamide
114. N-(4-Aminopyridin-2-yl)-N-(2,4-dimethylphenyl)acetamide
115. N-(4-Aminopyridin-2-yl)-N-(3-cyan-5-methylphenyl)acetamide
116. N-(4-Aminopyridin-2-yl)-N-(3-chlor-4-methylphenyl)acetamide
117. N-(4-aminopyridin-2-yl)-N-(4-fluorophenyl)butanamide

Particularly, the invention covers the intermediate compounds of general formula (XIII):

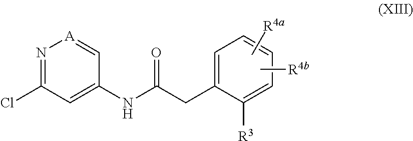

in which A, R^{4a}, R^{4b}, and R³ are as defined for the compound of general formula (I) supra.

Intermediate compounds of general formula (XIII) according to the invention are in particular:
118. N-(6-chloropyridazin-4-yl)-2-(2,6-dichlorophenyl)acetamide
119. 2-(2-chloro-3-fluorophenyl)-N-(6-chloropyridazin-4-yl)acetamide
120. 2-(2-chlorophenyl)-N-(6-chloropyridazin-4-yl)acetamide
121. 2-(2-chloro-4-fluorophenyl)-N-(6-chloropyridazin-4-yl)acetamide
122. 2-(2-chloro-6-fluorophenyl)-N-(6-chloropyridazin-4-yl)acetamide

Particularly, the invention covers the intermediate compounds of general formula (XIV):

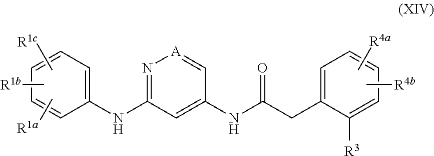

in which A, R^{1a}, R^{1b}, R^{1c}, and R³, R^{4a}, R^{4b} are as defined for the compound of general formula (I) supra.

Intermediate compounds of general formula (XIV) according to the invention are in particular:
123. N-(6-anilinopyridazin-4-yl)-2-(2,6-dichlorophenyl)acetamide
124. N-(6-anilinopyridazin-4-yl)-2-(2-chloro-3-fluorophenyl)acetamide
125. N-(6-anilinopyridazin-4-yl)-2-(2-chlorophenyl)acetamide
126. N-(6-anilinopyridazin-4-yl)-2-(2-chloro-4-fluorophenyl)acetamide
127. 2-(2-chloro-6-fluorophenyl)-N-[6-(4-fluoroanilino)pyridazin-4-yl]acetamide
128. 2-(2-chloro-4-fluorophenyl)-N-[6-(4-fluoroanilino)pyridazin-4-yl]acetamide
129. 2-(2-chloro-3-fluorophenyl)-N-[6-(4-fluoroanilino)pyridazin-4-yl]acetamide
130. 2-(2-chlorophenyl)-N-[6-(4-fluoroanilino)pyridazin-4-yl]acetamide
131. 2-(2,6-dichlorophenyl)-N-[6-(4-fluoroanilino)pyridazin-4-yl]acetamide
132. 2-(2,6-dichlorophenyl)-N-[6-(3-fluoroanilino)pyridazin-4-yl]acetamide
133. 2-(2-chloro-3-fluorophenyl)-N-[6-(3-fluoroanilino)pyridazin-4-yl]acetamide
134. 2-(2-chlorophenyl)-N-[6-(3-fluoroanilino)pyridazin-4-yl]acetamide
135. 2-(2-chloro-6-fluorophenyl)-N-[6-(3-fluoroanilino)pyridazin-4-yl]acetamide
136. 2-(2-chloro-4-fluorophenyl)-N-[6-(3-fluoroanilino)pyridazin-4-yl]acetamide
137. 2-(2-chlorophenyl)-N-[6-(3,4-difluoroanilino)pyridazin-4-yl]acetamide
138. 2-(2,6-dichlorophenyl)-N-[6-(3,4-difluoroanilino)pyridazin-4-yl]acetamide
139. 2-(2-chloro-3-fluorophenyl)-N-[6-(3,4-difluoroanilino)pyridazin-4-yl]acetamide
140. 2-(2-chloro-4-fluorophenyl)-N-[6-(3,4-difluoroanilino)pyridazin-4-yl]acetamide
141. 2-(2-chloro-6-fluorophenyl)-N-[6-(3,4-difluoroanilino)pyridazin-4-yl]acetamide
142. 2-(2,6-dichlorophenyl)-N-[6-(3,5-difluoroanilino)pyridazin-4-yl]acetamide
143. 2-(2-chlorophenyl)-N-[6-(3,5-difluoroanilino)pyridazin-4-yl]acetamide
144. 2-(2-chloro-3-fluorophenyl)-N-[6-(3,5-difluoroanilino)pyridazin-4-yl]acetamide
145. 2-(2-chloro-4-fluorophenyl)-N-[6-(3,5-difluoroanilino)pyridazin-4-yl]acetamide
146. 2-(2-chloro-6-fluorophenyl)-N-[6-(3,5-difluoroanilino)pyridazin-4-yl]acetamide
148. 2-(2-chloro-3-fluorophenyl)-N-[2-(4-fluoroanilino)pyridin-4-yl]acetamide

In accordance with a further aspect, the present invention covers the use of said intermediate compounds for the preparation of a compound of general formula (I) as defined supra.

The compounds of general formula (I) of the present invention can be converted to any salt, more particularly pharmaceutically acceptable salts, as described herein, by any method which is known to the person skilled in the art. Similarly, any salt of a compound of general formula (I) of the present invention can be converted into the free compound, by any method which is known to the person skilled in the art.

A compound according to the invention is used for the manufacture of a medicament.

Compounds of general formula (I) of the present invention demonstrate a valuable pharmacological spectrum of action which could not have been predicted. Compounds of the present invention have surprisingly been found to effectively inhibit P2X4, as antagonists or negative allosteric modulators, and it is possible therefore that said compounds be used for the treatment or prophylaxis of diseases.

Compounds of the present invention can be utilized to inhibit, antagonize, negative allosteric modulate, etc., the P2X4 receptor. This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

The present invention also provides methods of treating the following syndromes, diseases or disorders:
Genitourinary, gastrointestinal, respiratory, proliferative and pain-related diseases, conditions and disorders;
Gynecological diseases including primary and secondary dysmenorrhea, dyspareunia, vulvudynia, endometriosis and adenomyosis; endometriosis-associated pain; endometriosis-associated symptoms, wherein said symptoms are in particular abdominal pain, dysmenorrhea, dyspareunia, dysuria, dyschezia or pelvic hypersensitivity;
Urinary tract disease states including those associated with bladder outlet obstruction; overactive bladder or cystitis, interstitial cystitis, bladder pain syndrome, urinary incontinence conditions such as reduced bladder capacity, increased frequency of micturition, urge incontinence, stress incontinence, or bladder hyperreactivity; benign prostatic hypertrophy; prostatic hyperplasia; prostatitis; detrusor hyperreflexia; overactive urinary bladder and symptoms related to overactive urinary bladder wherein said symptoms are in particular increased urinary frequency, nocturia, urinary urgency or urge incontinence; pelvic hypersensitivity; urethritis; prostatitis; prostatodynia; cystitis, in particular interstitial cystitis; idiopathic bladder hypersensitivity; kidney disease as hyperprostaglandin E syndrome, classic Bartter syndrome;
Epilepsy, partial and generalized seizures;
Respiratory disorders including asthma, chronic obstructive pulmonary disease, pulmonary fibrosis, interstitial pulmonary fibrosis, bronchospasm, chronic chough, refractory chronic cough, idiopathic chronic cough;
Gastrointestinal disorders including irritable bowel syndrome (IBS), inflammatory bowel disease (IBD), biliary colic and other biliary disorders, renal colic, diarrhea-dominant IBS; gastroesophageal reflux, gastrointestinal distension, Crohn's disease and the like;
Fatty liver disorders, in particular NASH (Non-Alcoholic Steato-Hepatitis); fibrotic diseases including lung fibrosis, heart fibrosis, kidney fibrosis and fibrosis of other organs; metabolic syndrome including, for example, insulin resistance, hypertension, refractory hypertension, dyslipoproteinaemia and obesity, diabetes mellitus, in particular Diabetes type II, myocardial infarction; atherosclerosis; lipid disorders;
Neurodegenerative disorders such as Alzheimer's disease, Parkinson's disease, Stroke, brain ischemia, ischemic brain injury, Ischemic Stroke (IS), haemorrhagic stroke, traumatic brain injury, spinal cord injury;
Chronic itch, pruritus;
Wound healing, impaired wound healing;
Heart disorders including ischemia reperfusion injury, cardiac ischemia;
Arthritis, chronic arthritis, juvenile arthritis, osteoarthritis, rheumatoid arthritis, juvenile rheumatoid arthritis, ankylosing spondylitis and related neuralgias, and disease of the skeleton like degeneration of the joints, gout and acute gout, hemophilic arthropathy (Burnstock et al., 2012 Pharmacol Rev. 64:834-868).

The present invention also provides methods of treating the following pain syndromes, diseases or disorders:
Pain-associated syndromes, disease or disorders, including hyperalgesia, allodynia, acute and chronic inflammatory and neuropathic pain, abdominal pain such as functional bowel disorders, irritable bowel syndrome, inflammatory bowel disease, bladder pain syndrome, inflammatory pain, low back pain, surgical pain, visceral pain, dental pain, periodontitis, premenstrual pain, endometriosis-associated pain, pain associated with fibrotic diseases, central pain;
Pain due to burning mouth syndrome, pain due to burns;
Pain due to migraine (acute and prophylactic treatment), cluster headaches, pain due to nerve injury, traumatic nerve injury, post-traumatic injuries (including fractures and sport injuries);
Pain due to neuritis, neuralgias;
Pain due to poisoning;
Pain due to ischemic injury;
Pain due to interstitial cystitis;
Cancer pain, cancer cachexia, pain due to viral, parasitic or bacterial infections;
Pain due to traumatic nerve-injury, pain due to post-traumatic injuries (including fractures and sport injuries), pain due to trigeminal neuralgia, pain associated with small fiber neuropathy, pain associated with diabetic neuropathy, postherpetic neuralgia, chronic lower back pain, neck pain, phantom limb pain, pelvic pain syndrome, chronic pelvic pain, neuroma pain, complex regional pain syndrome, fibromyalgia, myofascial disorders, pain associated with gastrointestinal distension, chronic arthritic pain and related neuralgias;
Pain associated with cancer, morphine-resistant pain, pain associated with chemotherapy, HIV and HIV treatment-induced neuropathy.

The pain may be mild pain, moderate pain, severe pain, musculoskeletal pain, particularly acute, subacute and chronic musculoskeletal pain syndromes such as bursitis, burns, injuries, and pain following surgical (post-operative pain) and dental procedures as well as the preemptive treatment of surgical pain, complex regional pain syndrome, neuropathic pain, back pain such as acute visceral pain, neuropathies, acute trauma, chemotherapy-induced mononeuropathy pain states, polyneuropathy pain states (such as diabetic peripheral neuropathy and/or chemotherapy induced neuropathy), autonomic neuropathy pain states, peripheral nervous system (PNS) lesion or central nervous system (CNS) lesion or disease related pain states, polyradiculopathies of cervical, lumbar or sciatica type, cauda equina syndrome, piriformis syndrome, paraplegia, quadriplegia, pain states related to various Polyneuritis conditions underlying various infections, chemical injuries, radiation exposure, underlying disease or deficiency conditions (such as beriberi, vitamin deficiencies, hypothyroidism, porphyria, cancer, autoimmune disease such as multiple sclerosis and spinal-cord injury, ischemia, neurodegeneration, stroke, post stroke pain, inflammatory disorders, oesophagitis, gastroeosophagal reflux disorder (GERD), pelvic hypersensitivity, cystitis, stomach, duodenal ulcer, muscle pain, pain due to colicky and referred pain.

Compounds of the invention are thus expected to be useful in the treatment of inflammation. The term “inflammation” is also understood to include any inflammatory disease, disorder or condition per se, any condition that has an inflammatory component associated with it, and/or any condition characterized by inflammation as a symptom, including, inter alia, acute, chronic, ulcerative, fibrotic, allergic and autoimmune diseases, infection by pathogens, immune reactions due to hypersensitivity, entering foreign bodies, physical injury, necrosis, endometriosis and other forms of inflammation known to those skilled in the art.

The compounds according to the invention are useful for the relief of pain, fever and inflammation of a variety of conditions including rheumatic fever, symptoms associated with influenza or other viral infections, common cold, toothache, sprains and strains, myositis, synovitis.

The compounds of the present invention may also be useful in the treatment, viral infections (e.g. influenza, common cold, herpes zoster, hepatitis C and HIV), bacterial infections, fungal infections, surgical or dental procedures, malignancies (e.g. melanoma, breast cancer, colon cancer, lung cancer and prostate cancer), rheumatic fever, Hodgkin's disease, systemic lupus erythematosus, vasculitis, pancreatitis, nephritis, bursitis, wound healing, impaired wound healing, dermatitis, eczema, diabetes mellitus, autoimmune diseases, allergic disorders, rhinitis, ulcers, mild to moderately active ulcerative colitis, familial adenomatous polyposis, coronary heart disease, sarcoidosis and any other disease with an inflammatory component.

Compounds of the invention are also expected to be useful in the treatment of conditions associated or causing bone loss in a subject. Conditions that may be mentioned in this regard include osteoporosis, Paget's disease and/or periodontal diseases.

These disorders have been well characterized in humans, but also exist with a similar etiology in other mammals and can be treated by administering pharmaceutical compositions of the present invention.

The term “treating” or “treatment” as used in the present text is used conventionally, e.g., the management or care of a subject for the purpose of combating, alleviating, reducing, relieving, improving the condition of a disease or disorder, such as those reported above.

The compounds of the present invention can be used in particular in therapy and prevention, i.e. prophylaxis, of the following syndromes, diseases or disorders:
pain, pain syndromes (acute and chronic), inflammatory-induced pain, neuropathic pain including diabetic neuropathic pain and diabetic neuropathy, cancer-associated pain, chemotherapy or intoxication induced pain, pelvic pain, endometriosis-associated pain as well as endometriosis as such, bladder pain syndrome;
lung diseases and respiratory disorder in particular asthma, bronchiolitis obliterans syndrome, COPD, chronic cough, diseases related to goblet cells and lung fibrosis, liver diseases, in particular liver fibrosis, fatty liver disorders, NASH (Non-Alcoholic Steato-Hepatitis);
brain ischemia, ischemic brain injury, Ischemic Stroke (IS), haemorrhagic stroke, traumatic brain injury, spinal cord injury, aneurysm;
Chronic itch, pruritus;
osteoarthritis, burning mouth syndrome, migraine disorders, irritable bowel disease;
urology related syndromes like, overactive urinary bladder, interstitial cystitis, bladder pain syndrome.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, particularly of the diseases reported above.

The pharmaceutical activity of the compounds according to the invention can be explained by their activity as inhibitors, antagonizing and/or negative allosteric modulating, the P2X4 receptor.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular of the diseases reported above.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular of the diseases reported above.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular of the diseases reported above.

In accordance with a further aspect, the present invention covers a method of treatment or prophylaxis of diseases, in particular of the diseases reported above, using an effective amount of a compound of general formula (I), as described supra, or stereoisomers, tautomers, N-oxides, hydrates, solvates, and salts thereof, particularly pharmaceutically acceptable salts thereof, or mixtures of same. In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

The compounds of the present invention can be administered as the sole pharmaceutical agent or in combination with one or more other pharmaceutically active ingredients where the combination causes no unacceptable adverse effects. The present invention also covers such pharmaceutical combinations.

Based upon standard laboratory techniques known to evaluate compounds useful for the treatment and prevention, i.e. prophylaxis, of the syndromes, diseases or disorders reported above, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

NMR peak forms are stated as they appear in the spectra, possible higher order effects have not been considered. Chemical shifts are given in ppm; all spectra were calibrated to solvent residual peak. Integrals are given in integers.

Alternatively, the ¹H-NMR data of selected compounds are listed in the form of ¹H-NMR peaklists. Therein, for each signal peak the 5 value in ppm is given, followed by the signal intensity, reported in round brackets. The 5 value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations
The following table lists the abbreviations used herein.

| Abbreviation | Meaning |
|---|---|
| ACN | acetonitrile |
| Ac₂O | acetic anhydride |
| ACOH | acetic acid (ethanoic acid) |
| aq. | aqueous |
| Boc | tert-butoxycarbonyl |
| Br | broad (1H-NMR signal) |
| cat. | catalytic |
| conc. | concentrated |
| Cl | chemical ionisation |
| d | doublet |
| DAD | diode array detector |
| DCM | dichloromethane |
| dd | double-doublet |
| DIPEA | diisopropylethylamine |
| DMA | N,N-dimethylacetamide |
| DMF | N,N-dimethylformamide |
| DMSO | dimethylsulfoxide |
| dt | double-triplet |
| EDC | 1-ethyl-3-(3-dimethylaminopropyl)carbodiimide |
| ELSD | Evaporative Light Scattering Detector |
| EtOAc | ethyl acetate |
| EtOH | ethanol |
| eq. | equivalent |
| ESI | electrospray (ES) ionisation |
| h or hrs | hour(s) |
| HATU | 1-[bis(dimethylamino)methylene]-1H-1,2,3-triazolo[4,5- |
| | b]pyridinium 3-oxid hexafluorophosphate |
| HCl | hydrochloric acid |
| HPLC | high performance liquid chromatography |
| LC-MS | liquid chromatography mass spectrometry |
| m | multiplet |
| min | minute(s) |
| MeCN | acetonitrile |
| MeOH | methanol |
| MS | mass spectrometry |
| NMR | nuclear magnetic resonance spectroscopy: chemical |
| | shifts (δ) are given in ppm. The chemical shifts were |
| | corrected by setting the DMSO signal to 2.50 ppm unless |
| | otherwise stated. |
| Pd/C | palladium on activated charcoal |
| PdCl₂(dppf) | [1,1′-bis(diphenylphosphino)ferrocene] |
| | dichloropalladium(II) |
| Pd(dba)₂ | bis(dibenzylideneacetone)palladium |
| q | quartet |
| r.t. or rt or RT | room temperature |
| rac | racemic |
| Rt | retention time (as measured either with HPLC or UPLC) |
| | in minutes |
| S | singlet |
| sat. | saturated |
| SM | starting material |
| SQD | Single-Quadrupole-Detector |
| t | triplet |
| T3P | propylphosphonic anhydride |
| td | triple-doublet |
| TEA | triethylamine |
| TFA | trifluoroacetic acid |
| THF | tetrahydrofuran |
| UPLC | ultra performance liquid chromatography |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g.

Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### UPLC-MS Standard Procedures

Analytical UPLC-MS was performed as described below. The masses (m/z) are reported from the positive mode electrospray ionisation unless the negative mode is indicated (ESI−). In most of the cases method 1 is used. If not, it is indicated.

### Method 1:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### Method 2:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

### Method 3:

Instrument: Waters Acquity Platform ZQ4000; column: Waters BEHC 18, 50 mm×2.1 mm, 1.7μ; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.

### Experimental Section—General Procedures

### General Procedure A:

Formation of bisarylamines from aromatic amide and 2-bromo-4-nitropyridine (GP A): Aromatic amine (1.0 eq.) and 2-bromo-4-nitropyridine (1.4 eq) were dissolved in toluene or 1,4-dioxane or DMF (ca. 70 eq.). Under inert atmosphere (Argon), bis(dibenzylidenaceton) palladium(0) (CAS [32005-36-0], 0.03 eq.), 4,5-bis-(diphenylphosphino)-9,9-dimethyl xanthene (Xantphos, CAS [161265-03-8], 0.07 eq.), and caesium carbonate (1.6 eq.) were added and the mixture stirred at 100° C. for ca. 18 h. After cooling to rt, the catalyst was filtered off via celite and rinsed with ethyl acetate. The filtrate was partitioned between water and ethyl acetate and extracted with ethyl acetate. The combined arganic layers were washed with brine, dried with sodium sulfate and the solvents removed in vacuo. The crude product was purified via chromatography.

### General Procedure B:

Formation of bisarylamines from arylhalogenide and 2-amino-4-nitro-pyridine (GP B): Aromatic bromide (1.0-1.4 eq., alternatively, the corresponding iodide may be used), 4-nitropyridine-2-amine (1.0 eq.) and caesium carbonate (1.6 eq.) were dissolved in 1,4-dioxane or toluene. The mixture was degassed, and under argon atmosphere, bis(dibenzylidenaceton) palladium(0) (CAS [32005-36-0], 0.03 eq.) and 4,5-bis-(diphenylphosphino)-9,9-dimethyl xanthene (Xantphos, CAS [161265-03-8], 0.07 eq.) were added. The mixture was stirred at 100° C. for 18 h. After cooling to rt, the solids were filtered off and rinsed with ethyl acetate. The filtrate was partitioned between water and ethyl acetate and extracted with ethyl acetate. The combined arganic layers were washed with brine, dried with sodium sulfate and the solvents removed in vacuo. The crude product was purified via chromatography.

### General Procedure C:

Acylation of bisarylamines (GP C):

The bisarylamines were dissolved in acetic anhydride (or the respective corresponding homologue) as reagent and solvent (ca. 50 eq.), 4-N,N-dimethylaminopyridine (0.1 eq.) was added and the mixture stirred at 110-130° C. until complete conversion (2-18 h).

After cooling to rt, the mixture was either concentrated to dryness in vacuo and directly purified via chromatography or an aqueous workup was done. In this case, the mixture was partitioned between ethyl acetate and water, extracted with ethyl acetate, washed with brine, dried with sodium sulfate and the solvents removed in vacuo. The crude product was purified by chromatography.

### General Procedure D:

Reduction of nitro compounds by catalytic hydrogenation (GP D):

The nitro compound was dissolved in ethyl acetate and the palladium catalyst (10% Pd on activated carbon, 0.1 eq. Pd) was added. The mixture was degassed and charged with hydrogen and hydrogenated at 1 atm hydrogen pressure until complete conversion. Then the catalyst was filtered off and the filtrated concentrated to dryness. The product could be obtained without further purification.

### General Procedure E:

Reduction of nitro compounds with iron (GP E): The nitro compound was dissolved in acetic acid and iron powder (5 eq.) was added.

The mixture was vigorously stirred for 2-18 h, until complete conversion. Solids were filtered off via a celite pad and rinsed with ethyl acetate. The organic phase evaporated to dryness. Optionally, the residue was either codestilled several times with toluene until all acetic acid was removed or it was partitioned between ethyl acetate and water and sat. aqueous sodium bicarbonate solution added until pH >7. The phases were separated, the aqueous layer extracted with ethyl acetate and the combined organic layers were washed with sat. aqueous sodium bicarbonate solution and brine and dried with sodium sulfate. The solvents were removed in vacuo and the product was taken to the next step without further purification.

### General Procedure F:

Acylation of amino-pyridazines (GP F):

6-Chloro-4-pyridazinamine and carboxylic acid (1-2 eq.) were dissolved in DMF and T3P (1-propanephosphinic anhydride, 50% in DMF, CAS [68957-94-8], 4.8 eq.) and N,N-Diisopropylethylamin (6 eq.) were added and the mixture stirred at 80° C. until complete conversion. Then the mixture was evaporated to a small volume, poured into water and filtered off. Then the solid was used in the following step as it was or it was purified by HPLC if necessary.

### General Procedure G:

Aromatic nucleophilic substitution of chloropyridazine (GP G): The N-acylated (6-chloropyridazin-4-yl)acetamide was dissolved in ethanol and an aniline derivative (1 eq.) was added. Optionally, 4-methylbenzenesulfonic acid hydrate (1 eq.) could be added to enhance the turnover. Then the mixture was stirred at 80° C. for 48 hrs and evaporated. The residue was purified by HPLC.

### General Procedure H:

Amide formation with HATU (GP H):

Amine and carboxylic acid (1.2 eq.) were dissolved in DMF and HATU (2-(7-aza-1H-benzotriazole-1-yl)-1,1,3,3-tetramethyluronium hexafluorophosphate, CAS [148893-10-1], 1.2 eq.) and triethylamine (5 eq.) were added and the mixture stirred at rt until complete conversion. Then the mixture was poured into water, extracted with ethyl acetate, the combined organic layers washed with brine, dried with sodium sulfate and the solvents evaporated. The crude product was purified by chromatography.

### General Procedure I:

Amide formation with T3P (GP I):

Amine and carboxylic acid (1-2 eq.) were dissolved in DMF and T3P (1-propanephosphinic anhydride, 50% in DMF, CAS [68957-94-8], 3 eq.) and triethylamine (6 eq.) were added and the mixture stirred at rt until complete conversion. Then the mixture was poured into water, extracted with ethyl acetate, the combined organic layers washed with brine, dried with sodium sulfate and the solvents evaporated. The crude product was purified by chromatography.

### General Procedure J:

Acetylation amino-pyrazines (GP J):

The aminopyrazines were dissolved in dichloromethane and acetal chloride (1.5 eq.) and triethylamine (1.8 eq.) were added and the mixture stirred at rt for 18 h. The mixture was concentrated in vacuo and directly purified via chromatography.

### Experimental Section—Intermediates

### Intermediate 1

### N-(3,4-difluorophenyl)-4-nitropyridin-2-amine

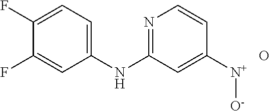

According to GP A, 3,4-difluoroaniline (454 mg, 3.52 mmol) and 2-bromo-4-nitropyridine (1.00 g, 4.93 mmol, 1.4 eq.) in toluene (25 mL) were converted to 658 mg of the title compound (74% of theory) as a yellow solid.

¹H NMR (400 MHz, DMSO-d6) δ[ppm] 7.29-7.34 (m, 1H), 7.35-7.42 (m, 1H), 6.92 (dd, 1H), 7.43-7.45 (m, 1H), 7.52 (dd, 1H), 8.00 (ddd, 1H), 8.49 (d, 1H), 9.92 (s, 1H).

LCMS (Method 1): Rt=1.24 min, MS (ESIpos) m/z=252 [M+H]⁺

### Intermediate 2

### N-(3-fluorophenyl)-4-nitropyridin-2-amine

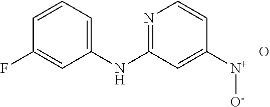

According to GP B, 4-nitropyridine-2-amine (2.00 g, 14.4 mmol) and 1-bromo-3-fluorobenzene (3.52 g, 20.1 mmol, 1.4 eq.) in toluene (75 mL) were converted to 810 mg of the title compound (20% of theory) as a reddish solid.

¹H NMR (400 MHz, DMSO-d6) δ[ppm] 6.77-6.81 (m, 1H), 7.32-7.35 (m, 2H), 7.45 (dd, 1H), 7.56 (d, 1H), 7.80-7.85 (m, 1H), 8.51 (d, 1H), 9.92 (s, 1H).

LCMS (Method 3): Rt=1.13 min, MS (ESIpos) m/z=234 [M+H]+

TABLE 1
Intermediates 3-38: the following intermediates were synthesized accordingly

| | | LC-MS method | |
|---|---|---|---|
| | Structure | Retention time | Synthesis |
| Intermediate | IUPAC-Name | Mass found | Method |
| Int. 3 | 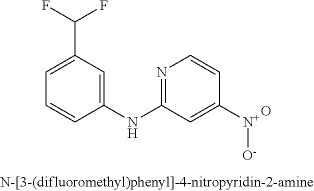 | Method 3 Rₜ = 1.13 min m/z = 266 [M + H]⁺ | GP A |
| Int. 4 | 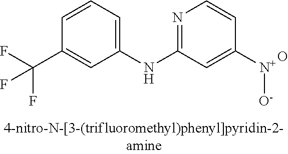 | Method 3 Rₜ = 1.27 min m/z = 284 [M + H]⁺ | GP A |
| Int. 5 | 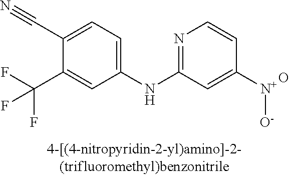 | Method 3 Rₜ = 1.19 min m/z = 309 [M + H]⁺ | GP A |
| Int. 6 | 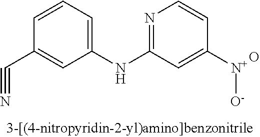 | Method 1 Rₜ = 1.13 min m/z = 239 [M − H]⁻ | GP B |
| Int. 7 | 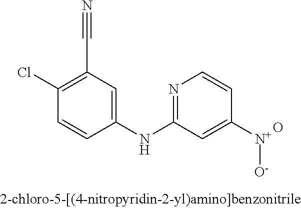 | Method 3 Rₜ = 1.16 min m/z = 275 [M + H]⁺ | GP A |
| Int. 8 | 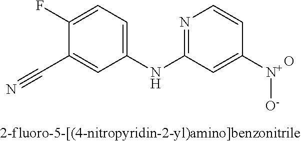 | Method 3 Rₜ = 1.07 min m/z = 259 [M + H]⁺ | GP A |
| Int. 9 | 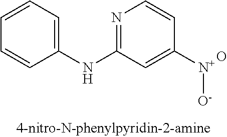 | Method 1 Rₜ = 1.15 min m/z = 216 [M + H]⁺ | GP B |
| Int. 10 | 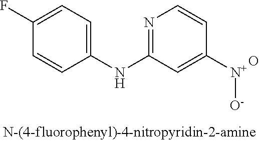 | Method 1 Rₜ = 1.16 min m/z = 234 [M + H]⁺ | GP A |
| Int. 11 | 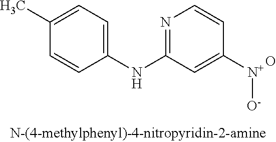 | Method 3 Rₜ = 1.15 min m/z = 230 [M + H]⁺ | GP A |
| Int. 12 | 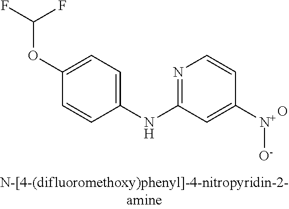 | Method 3 Rₜ = 1.14 min m/z = 282 [M + H]⁺ | GP A |
| Int. 13 | 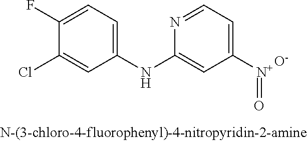 | Method 1 Rₜ = 1.30 min m/z = 268 [M + H]⁺ | GP A |
| Int. 14 | 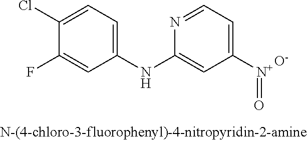 | Method 1 Rₜ = 1.29 min m/z = 266 [M − H]⁻ | GP A |
| Int. 15 | 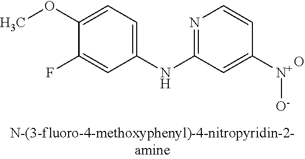 | Method 3 Rₜ = 1.07 min m/z = 264 [M + H]⁺ | GP A |
| Int. 16 | 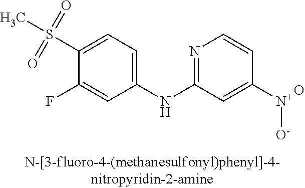 | Method 3 Rₜ = 0.91 min m/z = 312 [M + H]⁺ | GP A |
| Int. 17 | 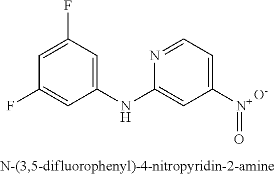 | Method 3 Rₜ = 1.22 min m/z = 252 [M + H]⁺ | GP B |
| Int. 18 | 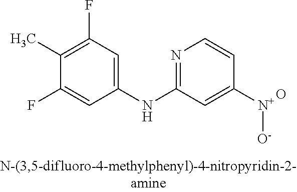 | Method 3 Rₜ = 1.29 min m/z = 266 [M + H]⁺ | GP A |
| Int. 19 | 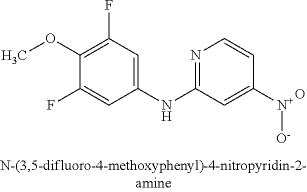 | Method 3 Rₜ = 1.18 min m/z = 282 [M + H]⁺ | GP A |
| Int. 20 | 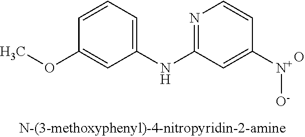 | Method 3 Rₜ = 1.06 min m/z = 246 [M + H]⁺ | GP A |
| Int. 21 | 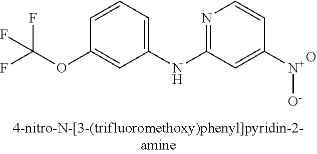 | Method 3 Rₜ = 1.30 min m/z = 300 [M + H]⁺ | GP A |
| Int. 22 | 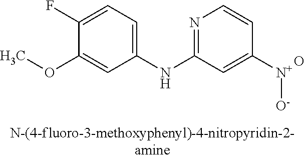 | Method 3 Rₜ = 1.07 min m/z = 264 [M + H]⁺ | GP A |
| Int. 23 | 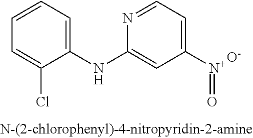 | Method 1 Rₜ = 1.21 min m/z = 250 [M + H]⁺ | GP A |
| Int. 24 | 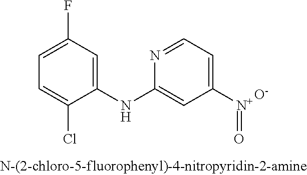 | Method 1 Rₜ = 1.26 min m/z = 266 [M − H]⁻ | GP A |
| Int. 25 | 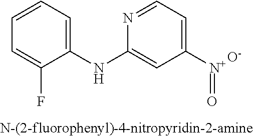 | Method 1 Rₜ = 1.13 min m/z = 234 [M + H]⁺ | GP A |
| Int. 26 | 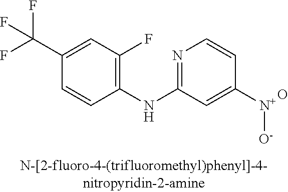 | Method 3 Rₜ = 1.29 min m/z = 302 [M + H]⁺ | GP A |
| Int. 27 | 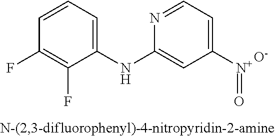 | Method 1 Rₜ = 1.16 min m/z = 250 [M − H]⁻ | GP A |
| Int. 28 | 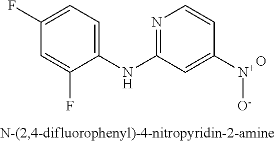 | Method 1 Rₜ = 1.18 min m/z = 252 [M]⁺ | GP A |
| Int. 29 | 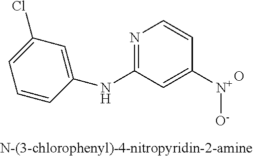 | Method 1 Rₜ = 1.29 min m/z = 250 [M + H]⁺ | GP A |
| Int. 30 | 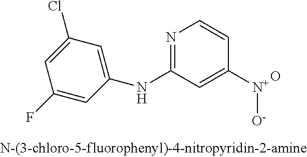 | Method 1 Rₜ = 1.35 min m/z = 268 [M + H]⁺ | GP A |
| Int. 31 | 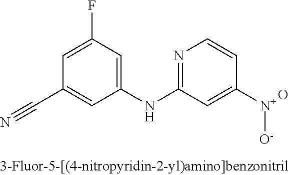 | Method 1 Rₜ = 1.20 min m/z = 257 [M − H]⁻ | GP A |
| Int. 32 | 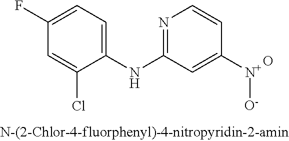 | Method 1 Rₜ = 1.25 min m/z = 268 [M + H]⁺ | GP A |
| Int. 33 | 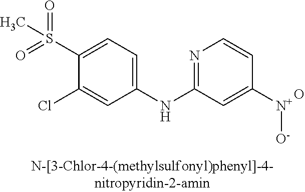 | Method 1 Rₜ = 1.07 min m/z = 326 [M − H]⁻ | GP A |
| Int. 34 | 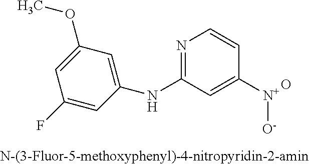 | Method 1 Rₜ = 1.25 min m/z = 264 [M + H]⁺ | GP A |
| Int. 35 | 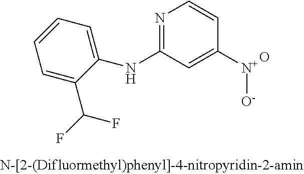 | Method 1 Rₜ = 1.17 min m/z = 266 [M + H]⁺ | GP A |
| Int. 36 | 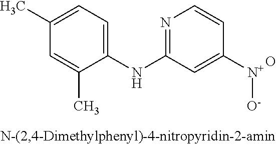 | Method 1 Rₜ = 1.27 min m/z = 244 [M + H]⁺ | GP A |
| Int. 37 | 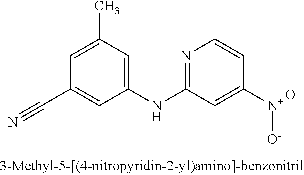 | Method 1 Rₜ = 1.21 min m/z = 255 [M + H]⁺ | GP A |
| Int. 38 | 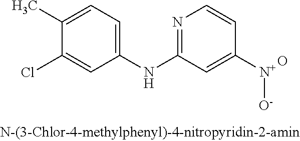 | Method 1 Rₜ = 1.37 min m/z = 264 [M + H]⁺ | GP A |

### Intermediate 39

### N-(3,4-difluorophenyl)-N-(4-nitropyridin-2-yl)acetamide

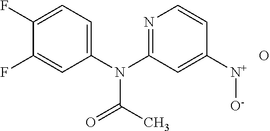

According to GP C, 655 mg (2.61 mmol) N-(3,4-difluorophenyl)-4-nitropyridin-2-amine (Int. 1) was dissolved in 13 mL acetic anhydride, DMAP (0.1 eq., 32 mg, 0.26 mmol) was added and the mixture stirred at 100° C. for 18 h. After cooling to rt, the reaction mixture was partitioned between ethyl acetate and water, extracted with ethyl acetate, washed with brine, dried with sodium sulfate and the solvents removed in vacuo. The crude product was purified by chromatography to yield 765 mg (93% of theory) of the title compound.

¹H NMR (400 MHz, DMSO-d6) δ[ppm] 2.03 (s, 3H), 7.30-7.34 (m, 1H), 7.52-7.58 (m, 1H), 7.66-7.72 (m, 1H), 7.95 (dd, 1H), 8.57 (d, 1H), 8.66 (d, 1H).

LCMS (method 1): Rt=1.08 min, MS (ESIpos) m/z=294 [M+H]+

TABLE 2
Intermediates 40-77: the following
intermediates were synthesized accordingly

| | | LC-MS |
|---|---|---|
| | | method |
| | | Retention |
| Inter- | | time |
| medi- | Structure | Mass |
| ate | IUPAC-Name | found |
| Int. 40 | 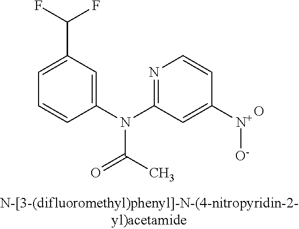 | Method 3 Rₜ = 0.96 min m/z = 308 [M + H]⁺ |
| Int. 41 | 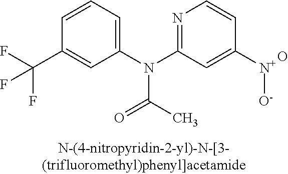 | Method 3 Rₜ = 1.09 min m/z = 326 [M + H]⁺ |
| Int. 42 | 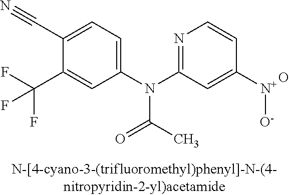 | Method 3 Rₜ = 1.04 min m/z = 351 [M + H]⁺ |
| Int. 43 | 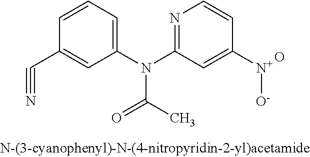 | Method 1 Rₜ = 0.95 min m/z = 283 [M + H]⁺ |
| Int. 44 | 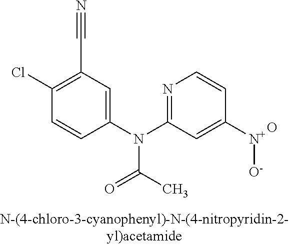 | Method 3 Rₜ = 0.97 min m/z = 317 [M + H]⁺ |
| Int. 45 | 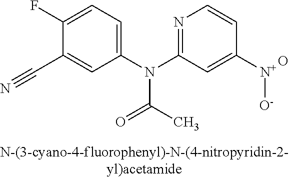 | Method 1 Rₜ = 0.98 min m/z = 300 [M]+ |
| Int. 46 | 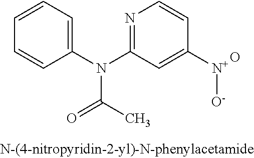 | Method 2 Rₜ = 1.00 min m/z = 258 [M + H]⁺ |
| Int. 47 | 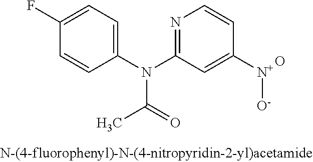 | Method 2 Rₜ = 1.03 min m/z = 276 [M + H]⁺ |
| Int. 48 | 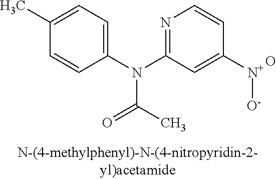 | Method 3 Rₜ = 1.00 min m/z = 272 [M + H]⁺ |
| Int. 49 | 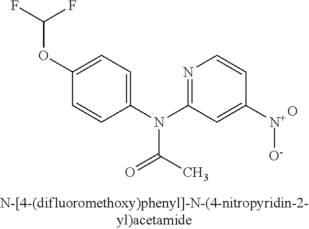 | Method 3 Rₜ = 0.99 min m/z = 324 [M + H]⁺ |
| Int. 50 | 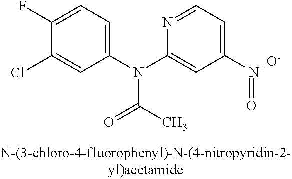 | Method 1 Rₜ = 1.13 min m/z = 310 [M + H]⁺ |
| Int. 51 | 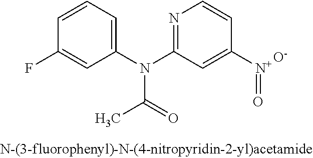 | Method 3 Rₜ = 0.94 min m/z = 276 [M + H]⁺ |
| Int. 52 | 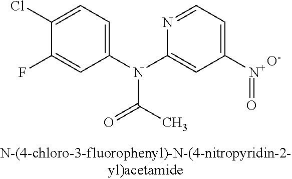 | Method 1 Rₜ = 1.14 min m/z = 310 [M + H]⁺ |
| Int. 53 | 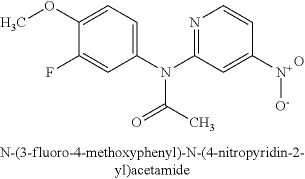 | Method 3 Rₜ = 0.93 min m/z = 306 [M + H]⁺ |
| Int. 54 | 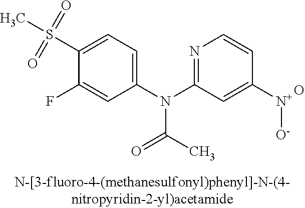 | Method 3 Rₜ = 0.74 min m/z = 354 [M + H]⁺ |
| Int. 55 | 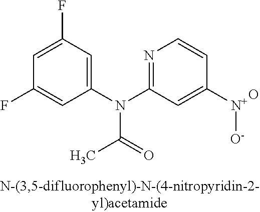 | Method 1 Rₜ = 1.09 min m/z = 294 [M + H]⁺ |
| Int. 56 | 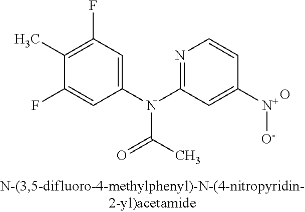 | Method 3 Rₜ = 1.10 min m/z = 308 [M + H]⁺ |
| Int. 57 | 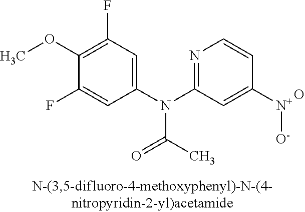 | Method 3 Rₜ = 1.01 min m/z = 324 [M + H]⁺ |
| Int. 58 | 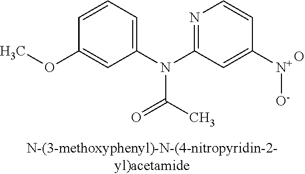 | Method 1 Rₜ = 1.01 min m/z = 288 [M + H]⁺ |
| Int. 59 | 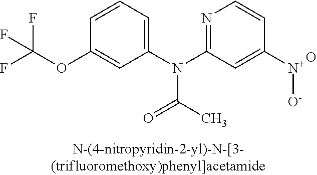 | Method 3 Rₜ = 1.14 min m/z = 342 [M + H]⁺ |
| Int. 60 | 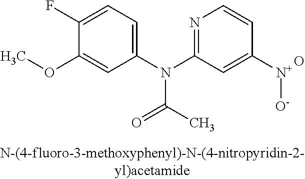 | Method 3 Rₜ = 0.94 min m/z = 306 [M + H]⁺ |
| Int. 61 | 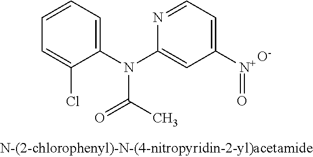 | Method 1 Rₜ = 1.11 min m/z = 292 [M + H]⁺ |
| Int. 62 | 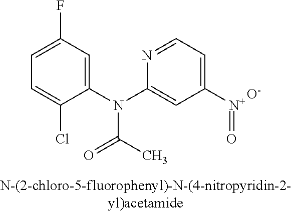 | Method 1 Rₜ = 1.13 min m/z = 310 [M + H]⁺ |
| Int. 63 | 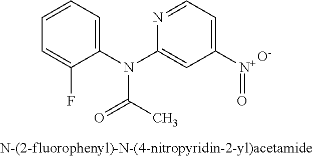 | Method 1 Rₜ = 1.03 min m/z = 276 [M + H]⁺ |
| Int. 64 | 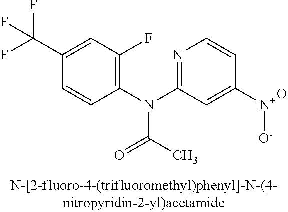 | Method 3 Rₜ = 1.16 min m/z = 344 [M + H]⁺ |
| Int. 65 | 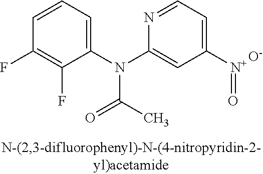 | Method 1 Rₜ = 1.07 min m/z = 294 [M + H]⁺ |
| Int. 66 | 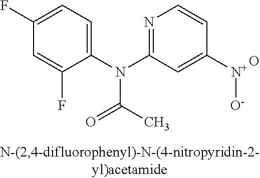 | Method 1 Rₜ = 1.10 min m/z = 294 [M + H]⁺ |
| Int. 67 | 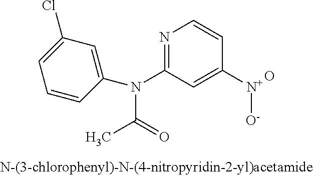 | Method 1 Rₜ = 1.13 min m/z = 292 [M + H]⁺ |
| Int. 68 | 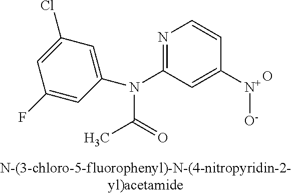 | Method 1 Rₜ = 1.17 min m/z = 310 [M + H]⁺ |
| Int. 69 | 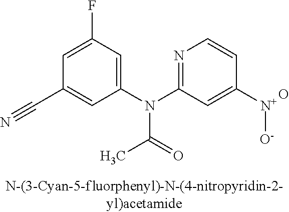 | Method 1 Rₜ = 1.03 min m/z = 301 [M + H]⁺ |
| Int. 70 | 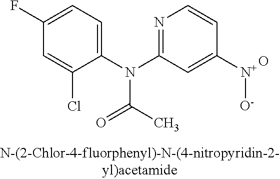 | Method 1 Rₜ = 1.18 min m/z = 310 [M + H]⁺ |
| Int. 71 | 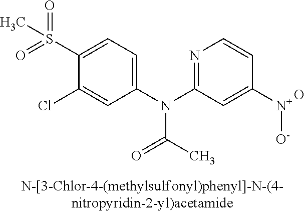 | Method 1 Rₜ = 0.93 min m/z = 370 [M + H]⁺ |
| Int. 72 | 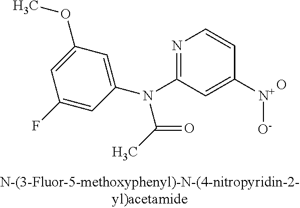 | Method 1 Rₜ = 1.09 min m/z = 306 [M + H]⁺ |
| Int. 73 | 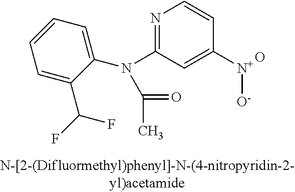 | Method 1 Rₜ = 1.13 min m/z = 308 [M + H]⁺ |
| Int. 74 | 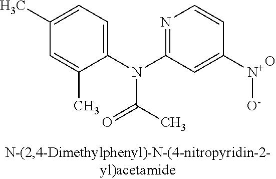 | Method 1 Rₜ = 1.22 min m/z = 286 [M + H]⁺ |
| Int. 75 | 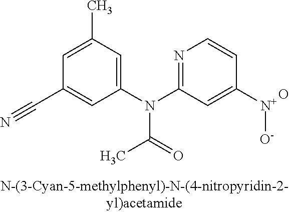 | Method 1 Rₜ = 1.05 min m/z = 297 [M + H]⁺ |
| Int. 76 | 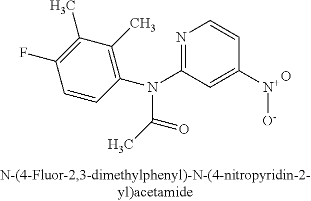 | Method 1 Rₜ = 1.22 min m/z = 304 [M + H]⁺ |
| Int. 77 | 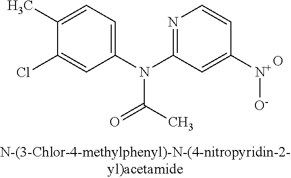 | Method 1 Rₜ = 1.23 min m/z = 306 [M + H]⁺ |

TABLE 3
Intermediate 78: the following intermediate was synthesized according GP
C, using butanoic anhydride

| | | LC-MS method |
|---|---|---|
| | Structure | Retention time |
| Intermediate | IUPAC-Name | Mass found |
| Int. 78 | 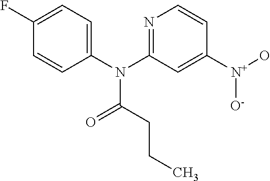 [SMILES: CCCC(=O)N(c1ccc(F)cc1)c1cc([NH+]([O-])O)ccn1] N-(4-fluorophenyl)-N-(4-nitropyridin-2- yl)butanamide | Method 1 Rₜ = 1.24 min m/z = 304 [M + H]⁺ |

### Intermediate 79

### N-(4-aminopyridin-2-yl)-N-(3,4-difluorophenyl)acetamide

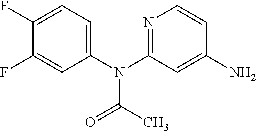 [SMILES: CC(=O)N(c1ccc(F)c(F)c1)c1cc(N)ccn1]

According to GP D, N-(3,4-difluorophenyl)-N-(4-nitropyridin-2-yl)acetamide (Int. 39, 745 mg, 2.54 mmol) were dissolved in ethyl acetate (15 mL), the palladium catalyst was added (10% Pd on activated charcoal, 270 mg, 0.1 eq.) and the mixture hydrogenated (1 atm hydrogen) for 3 h at rt. The catalyst was filtered off and the solvent evaporated to dryness, to yield 669 mg (94% of theory) of the title compound.

¹H NMR (400 MHz, DMSO-d6) δ[ppm] 1.97 (s, 3H), 6.27 (s, br, 2H), 6.43 (dd, 1H), 6.46 (s, br, 1H), 7.02-7.06 (m, 1H), 7.39-7.46 (m, 2H), 7.89 (d, 1H).

LCMS (method 1): Rt=0.78 min, MS (ESIpos) m/z=264 [M+H]⁺

### Intermediate 80

### N-(4-aminopyridin-2-yl)-N-(4-fluorophenyl)acetamide

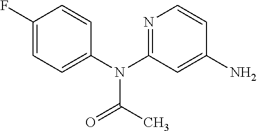 [SMILES: CC(=O)N(c1ccc(F)cc1)c1cc(N)ccn1]

According to GP E, N-(4-fluorophenyl)-N-(4-nitropyridin-2-yl)acetamide (Int. 47, 1.70 g, 6.18 mmol) were dissolved in acetic acid (70 mL) and iron powder (5 eq., 1.72 g, 30.9 mmol) was added portion wise. The mixture was vigorously stirred for 2 h at rt. Then the solids were filtered off via a pad of celite, rinsed with ethyl acetate, and the filtrate was concentrated in vacuo. The product was taken to the next step without further purification.

¹H NMR (400 MHz, DMSO-d6) δ[ppm] 1.96 (s, 3H), 6.23 (s, br, 2H), 6.41 (dd, 1H), 6.43 (s, br, 1H), 7.17-7.22 (m, 2H), 7.25-7.30 (m, 2H), 7.87 (d, 1H).

LCMS (Method 2): Rt=0.58 min, MS (ESIpos) m/z=246 [M+H]⁺

TABLE 4
Intermediates 81-116: the following aromatic amines were generated by
reduction of the corresponding nitro compounds, using an appropriate general
procedure (GP D, GP E)

| | | LC-MS method |
|---|---|---|
| | Structure | Retention time |
| Intermediate | IUPAC-Name | Mass found |
| Int. 81 | 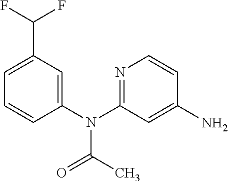 [SMILES: CC(=O)N(c1cccc(C(F)F)c1)c1cc(N)ccn1] N-(4-aminopyridin-2-yl)-N-[3- (difluoromethyl)phenyl]acetamide | Method 1 Rₜ = 0.75 min m/z = 278 [M + H]⁺ |
| Int. 82 | 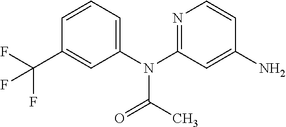 [SMILES: CC(=O)N(c1cccc(C(F)(F)F)c1)c1cc(N)ccn1] N-(4-aminopyridin-2-yl)-N-[3- (trifluoromethyl)phenyl]acetamide | Method 1 Rₜ = 0.86 min m/z = 296 [M + H]⁺ |
| Int. 83 | 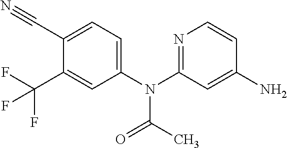 [SMILES: CC(=O)N(c1ccc(C#N)c(C(F)(F)F)c1)c1cc(N)ccn1] N-(4-aminopyridin-2-yl)-N-[4-cyano-3- (trifluoromethyl)phenyl]acetamide | Method 1 Rₜ = 0.87 min m/z = 321 [M + H]⁺ |
| Int. 84 | 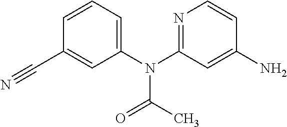 [SMILES: CC(=O)N(c1cccc(C#N)c1)c1cc(N)ccn1] N-(4-aminopyridin-2-yl)-N-(3- cyanophenyl)acetamide | Method 1 Rₜ = 0.67 min m/z = 253 [M + H]⁺ |
| Int. 85 | 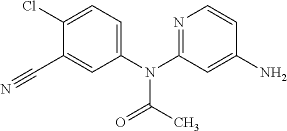 [SMILES: CC(=O)N(c1ccc(Cl)c(C#N)c1)c1cc(N)ccn1] N-(4-aminopyridin-2-yl)-N-(4-chloro-3- cyanophenyl)acetamide | Method 1 Rₜ = 0.79 min m/z = 287 [M + H]⁺ |
| Int. 86 | 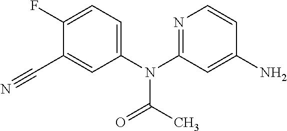 [SMILES: CC(=O)N(c1ccc(F)c(C#N)c1)c1cc(N)ccn1] N-(4-aminopyridin-2-yl)-N-(3-cyano-4- fluorophenyl)acetamide | Method 1 Rₜ = 0.71 min m/z = 271 [M + H]⁺ |
| Int. 87 | 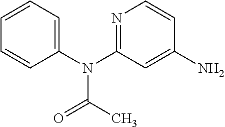 [SMILES: CC(=O)N(c1ccccc1)c1cc(N)ccn1] N-(4-aminopyridin-2-yl)-N- phenylacetamide | Method 2 Rₜ = 0.57 min m/z = 227 [M]⁺ |
| Int. 88 | 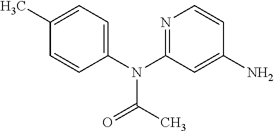 [SMILES: CC(=O)N(c1ccc(C)cc1)c1cc(N)ccn1] N-(4-aminopyridin-2-yl)-N-(4- methylphenyl)acetamide | Method 1 Rₜ = 0.75 min m/z = 242 [M + H]⁺ |
| Int. 89 | 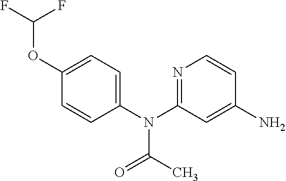 [SMILES: CC(=O)N(c1ccc(OC(F)F)cc1)c1cc(N)ccn1] N-(4-aminopyridin-2-yl)-N-[4- (difluoromethoxy)phenyl]acetamide | Method 1 Rₜ = 0.79 min m/z = 294 [M + H]⁺ |
| Int. 90 | 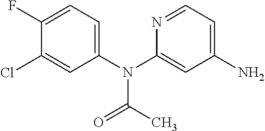 [SMILES: CC(=O)N(c1ccc(F)c(Cl)c1)c1cc(N)ccn1] N-(4-aminopyridin-2-yl)-N-(3-chloro-4- fluorophenyl)acetamide | Method 1 Rₜ = 0.81 min m/z = 280 [M + H]⁺ |
| Int. 91 | 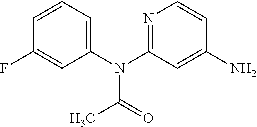 [SMILES: CC(=O)N(c1cccc(F)c1)c1cc(N)ccn1] N-(4-aminopyridin-2-yl)-N-(3- fluorophenyl)acetamide | Method 1 Rₜ = 0.72 min m/z = 246 [M + H]⁺ |
| Int. 92 | 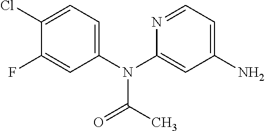 [SMILES: CC(=O)N(c1ccc(Cl)c(F)c1)c1cc(N)ccn1] N-(4-aminopyridin-2-yl)-N-(4-chloro-3- fluorophenyl)acetamide | Method 1 Rₜ = 0.83 min m/z = 280 [M + H]⁺ |
| Int. 93 | 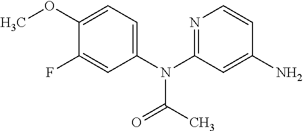 [SMILES: COc1ccc(N(C(C)=O)c2cc(N)ccn2)cc1F] N-(4-aminopyridin-2-yl)-N-(3-fluoro-4- methoxyphenyl)acetamide | Method 1 Rₜ = 0.70 min m/z = 276 [M + H]⁺ |
| Int. 94 | 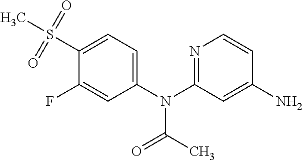 [SMILES: CC(=O)N(c1ccc(S(C)(=O)=O)c(F)c1)c1cc(N)ccn1] N-(4-aminopyridin-2-yl)-N-[3-fluoro-4- (methanesulfonyl)phenyl]acetamide | Method 1 Rₜ = 0.63 min m/z = 324 [M + H]⁺ |
| Int. 95 | 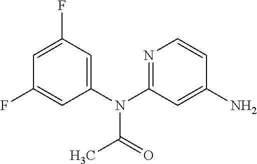 [SMILES: CC(=O)N(c1cc(F)cc(F)c1)c1cc(N)ccn1] N-(4-aminopyridin-2-yl)-N-(3,5- difluorophenyl)acetamide | Method 1 Rₜ = 0.78 min m/z = 264 [M + H]⁺ |
| Int. 96 | 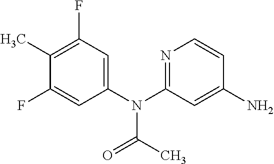 [SMILES: CC(=O)N(c1cc(F)c(C)c(F)c1)c1cc(N)ccn1] N-(4-aminopyridin-2-yl)-N-(3,5-difluoro-4- methylphenyl)acetamide | Method 1 Rₜ = 0.87 min m/z = 278 [M + H]⁺ |
| Int. 97 | 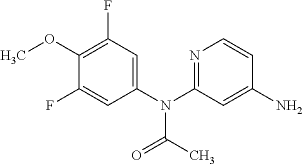 [SMILES: COc1c(F)cc(N(C(C)=O)c2cc(N)ccn2)cc1F] N-(4-aminopyridin-2-yl)-N-(3,5-difluoro-4- methoxyphenyl)acetamide | Method 1 Rₜ = 0.78 min m/z = 294 [M + H]⁺ |
| Int. 98 | 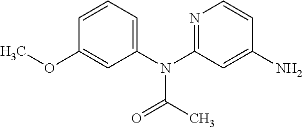 [SMILES: COc1cccc(N(C(C)=O)c2cc(N)ccn2)c1] N-(4-aminopyridin-2-yl)-N-(3- methoxyphenyl)acetamide | Method 1 Rₜ = 0.69 min m/z = 258 [M + H]⁺ |
| Int. 99 | 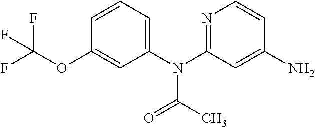 [SMILES: CC(=O)N(c1cccc(OC(F)(F)F)c1)c1cc(N)ccn1] N-(4-aminopyridin-2-yl)-N-[3- (trifluoromethoxy)phenyl]acetamide | Method 1 Rₜ = 0.90 min m/z = 312 [M + H]⁺ |
| Int. 100 | 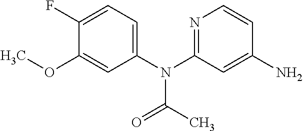 [SMILES: COc1cc(N(C(C)=O)c2cc(N)ccn2)ccc1F] N-(4-aminopyridin-2-yl)-N-(4-fluoro-3- methoxyphenyl)acetamide | Method 1 Rₜ = 0.72 min m/z = 276 [M + H]⁺ |
| Int. 101 | 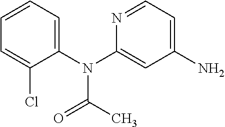 [SMILES: CC(=O)N(c1cc(N)ccn1)c1ccccc1Cl] N-(4-aminopyridin-2-yl)-N-(2- chlorophenyl)acetamide | Method 1 Rₜ = 0.76 min m/z = 262 [M + H]⁺ |
| Int. 102 | 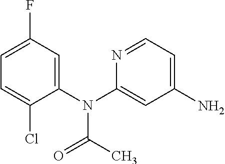 [SMILES: CC(=O)N(c1cc(N)ccn1)c1cc(F)ccc1Cl] N-(4-aminopyridin-2-yl)-N-(2-chloro-5- fluorophenyl)acetamide | Method 1 Rₜ = 0.78 min m/z = 280 [M + H]⁺ |
| Int. 103 | 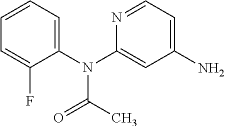 [SMILES: CC(=O)N(c1cc(N)ccn1)c1ccccc1F] N-(4-aminopyridin-2-yl)-N-(2- fluorophenyl)acetamide | Method 1 Rₜ = 0.69 min m/z = 246 [M + H]⁺ |
| Int. 104 | 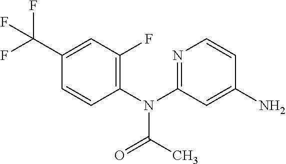 [SMILES: CC(=O)N(c1cc(N)ccn1)c1ccc(C(F)(F)F)cc1F] N-(4-aminopyridin-2-yl)-N-[2-fluoro-4- (trifluoromethyl)phenyl]acetamide | Method 1 Rₜ = 0.90 min m/z = 314 [M + H]⁺ |
| Int. 105 | 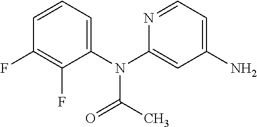 [SMILES: CC(=O)N(c1cc(N)ccn1)c1cccc(F)c1F] N-(4-aminopyridin-2-yl)-N-(2,3- difluorophenyl)acetamide | Method 1 Rₜ = 0.73 min m/z = 264 [M + H]⁺ |
| Int. 106 | 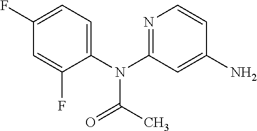 [SMILES: CC(=O)N(c1cc(N)ccn1)c1ccc(F)cc1F] N-(4-aminopyridin-2-yl)-N-(2,4- difluorophenyl)acetamide | Method 1 Rₜ = 0.74 min m/z = 264 [M + H]⁺ |
| Int. 107 | 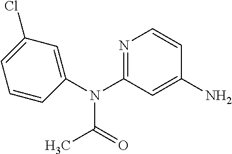 [SMILES: CC(=O)N(c1cccc(Cl)c1)c1cc(N)ccn1] N-(4-aminopyridin-2-yl)-N-(3- chlorophenyl)acetamide | Method 1 Rₜ = 0.79 min m/z = 262 [M + H]⁺ |
| Int. 108 | 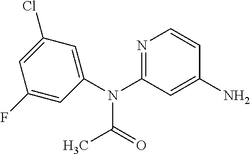 [SMILES: CC(=O)N(c1cc(F)cc(Cl)c1)c1cc(N)ccn1] N-(4-aminopyridin-2-yl)-N-(3-chloro-5- fluorophenyl)acetamide | Method 1 Rₜ = 0.85 min m/z = 280 [M + H]⁺ |
| Int. 109 | 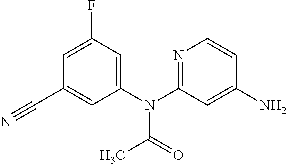 [SMILES: CC(=O)N(c1cc(F)cc(C#N)c1)c1cc(N)ccn1] N-(4-Aminopyridin-2-yl)-N-(3-cyan-5- fluorphenyl)acetamide | Method 1 Rₜ = 0.73 min m/z = 271 [M + H]⁺ |
| Int. 110 | 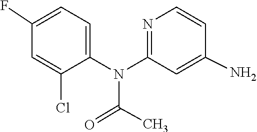 [SMILES: CC(=O)N(c1cc(N)ccn1)c1ccc(F)cc1Cl] N-(4-Aminopyridin-2-yl)-N-(2-chlor-4- fluorphenyl)acetamide | Method 1 Rₜ = 0.81 min m/z = 280 [M + H]⁺ |
| Int. 111 | 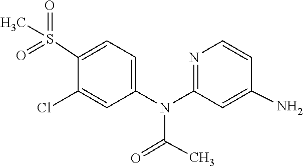 [SMILES: CC(=O)N(c1ccc(S(C)(=O)=O)c(Cl)c1)c1cc(N)ccn1] N-(4-Aminopyridin-2-yl)-N-[3-chlor-4- (methylsulfonyl)phenyl]acetamide | Method 1 Rₜ = 0.69 min m/z = 340 [M + H]⁺ |
| Int. 112 | 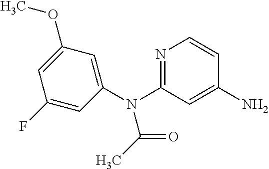 [SMILES: COc1cc(F)cc(N(C(C)=O)c2cc(N)ccn2)c1] N-(4-Aminopyridin-2-yl)-N-(3-fluor-5- methoxyphenyl)acetamide | Method 1 Rₜ = 0.77 min m/z = 276 [M + H]⁺ |
| Int. 113 | 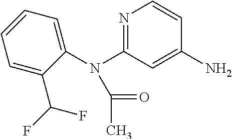 [SMILES: CC(=O)N(c1cc(N)ccn1)c1ccccc1C(F)F] N-(4-Aminopyridin-2-yl)-N-[2- (difluormethyl)phenyl]acetamide | Method 1 Rₜ = 0.82 min m/z = 278 [M + H]⁺ |
| Int. 114 | 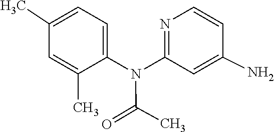 [SMILES: CC(=O)N(c1cc(N)ccn1)c1ccc(C)cc1C] N-(4-Aminopyridin-2-yl)-N-(2,4- dimethylphenyl)acetamide | Method 1 Rₜ = 0.85 min m/z = 256 [M + H]⁺ |
| Int. 115 | 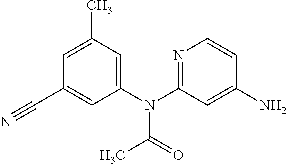 [SMILES: CC(=O)N(c1cc(C)cc(C#N)c1)c1cc(N)ccn1] N-(4-Aminopyridin-2-yl)-N-(3-cyan-5- methylphenyl)acetamide | Method 1 Rₜ = 0.75 min m/z = 267 [M + H]⁺ |
| Int. 116 | 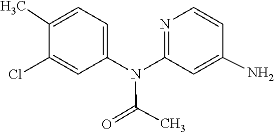 [SMILES: CC(=O)N(c1ccc(C)c(Cl)c1)c1cc(N)ccn1] N-(4-Aminopyridin-2-yl)-N-(3-chlor-4- methylphenyl)acetamide | Method 1 Rₜ = 0.88 min m/z = 276 [M + H]⁺ |

TABLE 5
Intermediate 117: The following aromatic amine was generated by reduction
of the corresponding nitro compound Int. 78, using an appropriate GP E

| | | LC-MS method |
|---|---|---|
| | Structure | Retention time |
| Intermediate | IUPAC-Name | Mass found |
| Int. 117 | 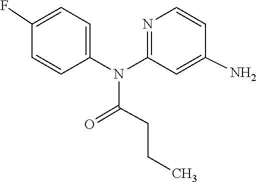 [SMILES: CCCC(=O)N(c1ccc(F)cc1)c1cc(N)ccn1] N-(4-aminopyridin-2-yl)-N-(4- fluorophenyl)butanamide | Method 1 Rₜ = 0.88 min m/z = 272 [M − H] |

### Intermediate 118—Intermediates with Pyridazine Core

### N-(6-chloropyridazin-4-yl)-2-(2,6-dichlorophenyl)acetamide

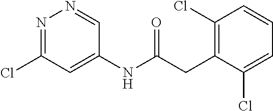 [SMILES: O=C(Cc1c(Cl)cccc1Cl)Nc1cnnc(Cl)c1]

According to GP F, 6-Chloro-4-pyridazinamine (500 mg, 3.86 mmol) and 2,6-dichlorophenylacetic acid (1.8 g, 1.5 eq.) were dissolved in DMF (10 mL) and T3P (11 ml, 18.5 mmol, 4.8 eq.) and diisopropylethylamin (4 ml, 23 mmol, 6 eq.) were added. The mixture was stirred at 80° C. for 18 h, then it was evaporated to a small volume, poured into water and filtered off to give the title compound as a solid.

¹H NMR (400 MHz, DMSO-d6) δ[ppm]: 2.518 (1.54), 2.523 (1.07), 2.888 (0.43), 4.155 (16.00), 5.758 (0.48), 7.356 (2.72), 7.375 (3.84), 7.378 (3.97), 7.397 (4.93), 7.507 (12.48), 7.527 (7.63), 8.050 (7.79), 8.056 (7.25), 9.184 (8.25), 9.190 (8.33), 11.326 (3.56).

LCMS (Method 1): Rt=1.01 min, MS (ESIpos) m/z=314 [M−H]⁻

TABLE 6
Intermediates 119-122: the following aminopyridazine amides were
generated using GP F

| | | LC-MS method |
|---|---|---|
| | Structure | Retention time |
| Intermediate | IUPAC-Name | Mass found |
| Int. 119 | 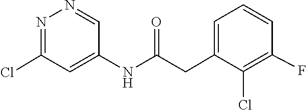 [SMILES: O=C(Cc1cccc(F)c1Cl)Nc1cnnc(Cl)c1] 2-(2-chloro-3-fluorophenyl)-N-(6- chloropyridazin-4-yl)acetamide | Method 1 Rₜ = 0.94 min m/z = 300 [M + H]⁺ |
| Int. 120 | 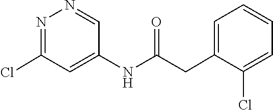 [SMILES: O=C(Cc1ccccc1Cl)Nc1cnnc(Cl)c1] 2-(2-chlorophenyl)-N-(6-chloropyridazin-4- yl)acetamide | Method 1 Rₜ = 1.00 min m/z = 282 [M + H]⁺ |
| Int. 121 | 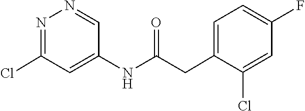 [SMILES: O=C(Cc1ccc(F)cc1Cl)Nc1cnnc(Cl)c1] 2-(2-chloro-4-fluorophenyl)-N-(6- chloropyridazin-4-yl)acetamide | Method 1 Rₜ = 0.96 min m/z = 300 [M + H]⁺ |
| Int. 122 | 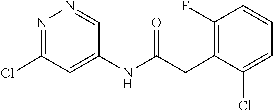 [SMILES: O=C(Cc1c(F)cccc1Cl)Nc1cnnc(Cl)c1] 2-(2-chloro-6-fluorophenyl)-N-(6- chloropyridazin-4-yl)acetamide | Method 1 Rₜ = 1.00 min m/z = 300 [M + H]⁺ |

### Intermediate 123

### N-(6-anilinopyridazin-4-yl)-2-(2,6-dichlorophenyl)acetamide

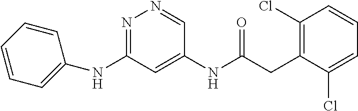 [SMILES: O=C(Cc1c(Cl)cccc1Cl)Nc1cnnc(Nc2ccccc2)c1]

According to GP G, N-(6-chloropyridazin-4-yl)-2-(2,6-dichlorophenyl)acetamide (100 mg, 0.31 mmol) was dissolved in 3 ml ethanol, aniline (29 μl, 0.31 mmol) was added and the mixture was stirred at 80° C. for 48 hrs. Then the mixture was evaporated and purified by HPLC. Yield 75 mg (63%) of the title compound.

LCMS (Method 1): Rt=1.10 min, MS (ESIpos) m/z=373 [M+H]⁺

TABLE 7
Intermediates 124-146: the following intermediates were generated
accordingly, using GP G

| | | LC-MS method |
|---|---|---|
| | Structure | Retention time |
| Intermediate | IUPAC-Name | Mass found |
| Int. 124 | 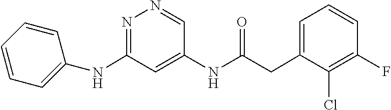 [SMILES: O=C(Cc1cccc(F)c1Cl)Nc1cnnc(Nc2ccccc2)c1] N-(6-anilinopyridazin-4-yl)-2-(2-chloro-3- fluorophenyl)acetamide | Method 1 Rₜ = 1.10 min m/z = 357 [M + H]⁺ |
| Int. 125 | 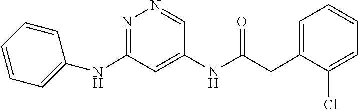 [SMILES: O=C(Cc1ccccc1Cl)Nc1cnnc(Nc2ccccc2)c1] N-(6-anilinopyridazin-4-yl)-2-(2- chlorophenyl)acetamide | Method 1 Rₜ = 1.05 min m/z = 337 [M − H]⁻ |
| Int. 126 | 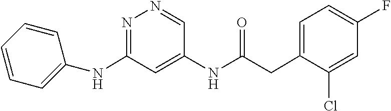 [SMILES: O=C(Cc1ccc(F)cc1Cl)Nc1cnnc(Nc2ccccc2)c1] N-(6-anilinopyridazin-4-yl)-2-(2-chloro-4- fluorophenyl)acetamide | Method 1 Rₜ = 1.08 min m/z = 357 [M + H]⁺ |
| Int. 127 | 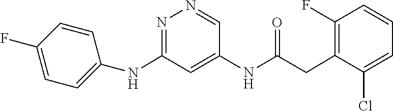 [SMILES: O=C(Cc1c(F)cccc1Cl)Nc1cnnc(Nc2ccc(F)cc2)c1] 2-(2-chloro-6-fluorophenyl)-N-[6-(4- fluoroanilino)pyridazin-4-yl]acetamide | Method 1 Rₜ = 1.13 min m/z = 375 [M + H]⁺ |
| Int. 128 | 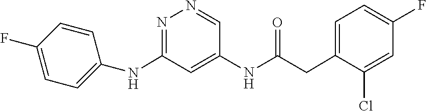 [SMILES: O=C(Cc1ccc(F)cc1Cl)Nc1cnnc(Nc2ccc(F)cc2)c1] 2-(2-chloro-4-fluorophenyl)-N-[6-(4- fluoroanilino)pyridazin-4-yl]acetamide | Method 1 Rₜ = 1.10 min m/z = 375 [M + H]⁺ |
| Int. 129 | 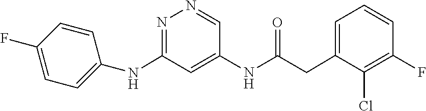 [SMILES: O=C(Cc1cccc(F)c1Cl)Nc1cnnc(Nc2ccc(F)cc2)c1] 2-(2-chloro-3-fluorophenyl)-N-[6-(4- fluoroanilino)pyridazin-4-yl]acetamide | Method 1 Rₜ = 1.13 min m/z = 375 [M + H]⁺ |
| Int. 130 | 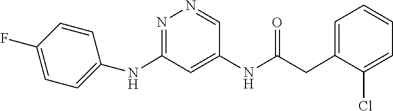 [SMILES: O=C(Cc1ccccc1Cl)Nc1cnnc(Nc2ccc(F)cc2)c1] 2-(2-chlorophenyl)-N-[6-(4- fluoroanilino)pyridazin-4-yl]acetamide | Method 1 Rₜ = 1.12 min m/z = 357 [M + H]⁺ |
| Int. 131 | 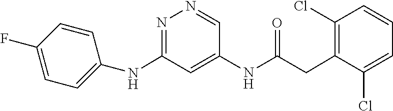 [SMILES: O=C(Cc1c(Cl)cccc1Cl)Nc1cnnc(Nc2ccc(F)cc2)c1] 2-(2,6-dichlorophenyl)-N-[6-(4- fluoroanilino)pyridazin-4-yl]acetamide | Method 1 Rₜ = 1.11 min m/z = 391 [M + H]⁺ |
| Int. 132 | 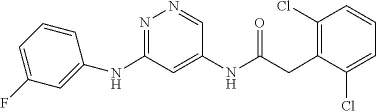 [SMILES: O=C(Cc1c(Cl)cccc1Cl)Nc1cnnc(Nc2cccc(F)c2)c1] 2-(2,6-dichlorophenyl)-N-[6-(3- fluoroanilino)pyridazin-4-yl]acetamide | Method 1 Rₜ = 1.14 min m/z = 391 [M + H]⁺ |
| Int. 133 | 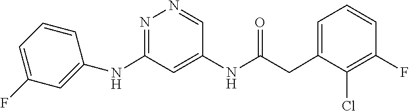 [SMILES: O=C(Cc1cccc(F)c1Cl)Nc1cnnc(Nc2cccc(F)c2)c1] 2-(2-chloro-3-fluorophenyl)-N-[6-(3- fluoroanilino)pyridazin-4-yl]acetamide | Method 1 Rₜ = 1.15 min m/z = 375 [M + H]⁺! |
| Int. 134 | 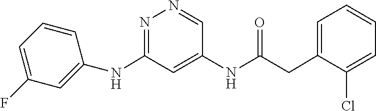 [SMILES: O=C(Cc1ccccc1Cl)Nc1cnnc(Nc2cccc(F)c2)c1] 2-(2-chlorophenyl)-N-[6-(3- fluoroanilino)pyridazin-4-yl]acetamide | Method 1 Rₜ = 1.08 min m/z = 357 [M + H]⁺ |
| Int. 135 | 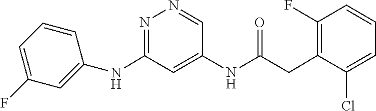 [SMILES: O=C(Cc1c(F)cccc1Cl)Nc1cnnc(Nc2cccc(F)c2)c1] 2-(2-chloro-6-fluorophenyl)-N-[6-(3- fluoroanilino)pyridazin-4-yl]acetamide | Method 1 Rₜ = 1.15 min m/z = 375 [M + H]⁺ |
| Int. 136 | 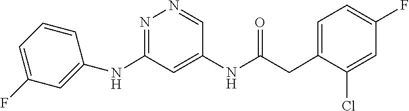 [SMILES: O=C(Cc1ccc(F)cc1Cl)Nc1cnnc(Nc2cccc(F)c2)c1] 2-(2-chloro-4-fluorophenyl)-N-[6-(3- fluoroanilino)pyridazin-4-yl]acetamide | Method 1 Rₜ = 1.12 min m/z = 375 [M + H]⁺ |
| Int. 137 | 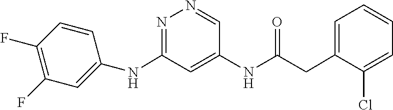 [SMILES: O=C(Cc1ccccc1Cl)Nc1cnnc(Nc2ccc(F)c(F)c2)c1] 2-(2-chlorophenyl)-N-[6-(3,4- difluoroanilino)pyridazin-4-yl]acetamide | Method 1 Rₜ = 1.16 min m/z = 375 [M + H]⁺ |
| Int. 138 | 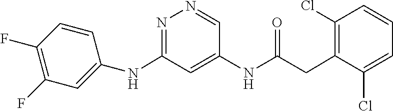 [SMILES: O=C(Cc1c(Cl)cccc1Cl)Nc1cnnc(Nc2ccc(F)c(F)c2)c1] 2-(2,6-dichlorophenyl)-N-[6-(3,4- difluoroanilino)pyridazin-4-yl]acetamide | Method 1 Rₜ = 1.18 min m/z = 407 [M − H]⁻ |
| Int. 139 | 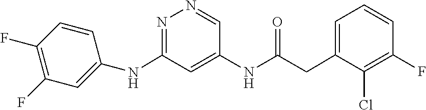 [SMILES: O=C(Cc1cccc(F)c1Cl)Nc1cnnc(Nc2ccc(F)c(F)c2)c1] 2-(2-chloro-3-fluorophenyl)-N-[6-(3,4- difluoroanilino)pyridazin-4-yl]acetamide | Method 1 Rₜ = 1.17 min m/z = 393 [M + H]⁺ |
| Int. 140 | 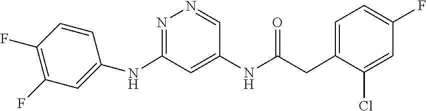 [SMILES: O=C(Cc1ccc(F)cc1Cl)Nc1cnnc(Nc2ccc(F)c(F)c2)c1] 2-(2-chloro-4-fluorophenyl)-N-[6-(3,4- difluoroanilino)pyridazin-4-yl]acetamide | Method 1 Rₜ = 1.14 min m/z = 393 [M + H]⁺ |
| Int. 141 | 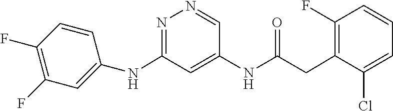 [SMILES: O=C(Cc1c(F)cccc1Cl)Nc1cnnc(Nc2ccc(F)c(F)c2)c1] 2-(2-chloro-6-fluorophenyl)-N-[6-(3,4- difluoroanilino)pyridazin-4-yl]acetamide | Method 1 Rₜ = 1.13 min m/z = 391 [M − H]⁻ |
| Int. 142 | 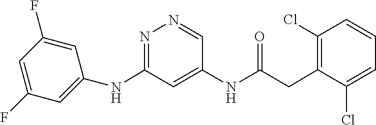 [SMILES: O=C(Cc1c(Cl)cccc1Cl)Nc1cnnc(Nc2cc(F)cc(F)c2)c1] 2-(2,6-dichlorophenyl)-N-[6-(3,5- difluoroanilino)pyridazin-4-yl]acetamide | Method 1 Rₜ = 1.14 min m/z = 393 [M + H]⁺ |
| Int. 143 | 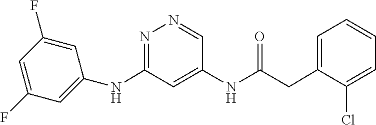 [SMILES: O=C(Cc1ccccc1Cl)Nc1cnnc(Nc2cc(F)cc(F)c2)c1] 2-(2-chlorophenyl)-N-[6-(3,5- difluoroanilino)pyridazin-4-yl]acetamide | Method 1 Rₜ = 1.19 min m/z = 375 [M + H]⁺ |
| Int. 144 | 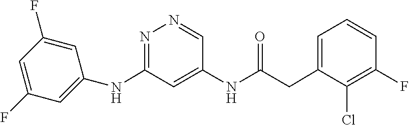 [SMILES: O=C(Cc1cccc(F)c1Cl)Nc1cnnc(Nc2cc(F)cc(F)c2)c1] 2-(2-chloro-3-fluorophenyl)-N-[6-(3,5- difluoroanilino)pyridazin-4-yl]acetamide | Rₜ = 1.20 min m/z = 393 [M + H]⁺ |
| Int. 145 | 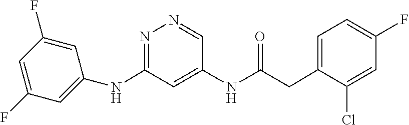 [SMILES: O=C(Cc1ccc(F)cc1Cl)Nc1cnnc(Nc2cc(F)cc(F)c2)c1] 2-(2-chloro-4-fluorophenyl)-N-[6-(3,5- difluoroanilino)pyridazin-4-yl]acetamide | Method 1 Rₜ = 1.17 min m/z = 393 [M + H]⁺ |
| Int. 146 | 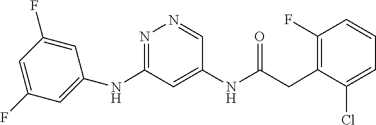 [SMILES: O=C(Cc1c(F)cccc1Cl)Nc1cnnc(Nc2cc(F)cc(F)c2)c1] 2-(2-chloro-6-fluorophenyl)-N-[6-(3,5- difluoroanilino)pyridazin-4-yl]acetamide | Method 1 Rₜ = 1.20 min m/z = 391 [M − H]⁻ |

### Intermediate 147

### N²-(4-fluorophenyl)pyridine-2,4-diamine

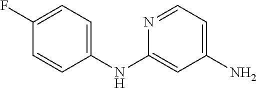 [SMILES: Nc1ccnc(Nc2ccc(F)cc2)c1]

110 mL (1.5 eq., 1.2 mol) of 4-fluoroaniline were dissolved in 500 mL of sulfolane. 24 mL (1.0 eq., 780 mmol) of aqueous conc. HCl were added and the suspension was heated up to 60° C. 100 g (1.0 eq., 778 mmol) of 2-chloropyridin-4-amine (1.0 eq., 778 mmol) were added in portions. The reaction solution was stirred at 130° C. for 18 h. The still warm reaction mixture was diluted with water and the pH value was adjusted to pH=10-11 using semi-concentrated aqueous NaOH solution. The mixture was poured into 4000 mL of water and stirred vigorously for 2 h. The precipitate was filtered off and it was washed intensively with water. The solid material was dried at 50° C. under vacuum. 159 g of the title compound (63% of theory) were obtained as a lilac solid.

¹H NMR (400 MHz, DMSO-d6) δ[ppm] 5.76 (s, 2H), 5.90 (d, J=1.52 Hz, 1H), 6.00 (dd, J=5.70, 1.90 Hz, 1H), 6.79-7.17 (m, 2H), 7.42-7.78 (m, 3H), 8.47 (s, 1H).

LCMS (Method 1): Rt=0.86 min, MS (ESIpos) m/z=204 [M+H]⁺

### Intermediate 148

### 2-(2-chloro-3-fluorophenyl)-N-[2-(4-fluoroanilino)pyridin-4-yl]acetamide

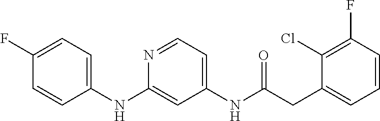 [SMILES: O=C(Cc1cccc(F)c1Cl)Nc1ccnc(Nc2ccc(F)cc2)c1]

115 g (1.15 eq., 610 mmol) of 2-(2-chloro-3-fluorophenyl)acetic acid were dissolved in 700 mL of N,N-dimethylacetamide and 103 g (1.2 eq., 636 mmol) of 1,1′-carbonyldiimidazole were added in portions at room temperature. The reaction mixture was heated up to 40° C. for 4 h. 108 g (1.0 eq., 530 mmol) of N²-(4-fluorophenyl)pyridine-2,4-diamine (Int. 147) were added in portions and the mixture was stirred at 40° C. for 18 h. The mixture was diluted with 5000 mL of water and extracted with ethyl acetate. The combined organic phases were washed with water. After drying over magnesium sulfate and evaporation of the organic phase the remaining residue was triturated with dichloromethane to colorless and finally triturated with n-hexane. 154 g of the title compound (68% of theory) were obtained as a white solid after drying at 50° C.

¹H NMR (400 MHz, DMSO-d6) δ[ppm] 3.93 (s, 2H), 6.71-6.86 (m, 1H), 6.99-7.13 (m, 2H), 7.21-7.45 (m, 4H), 7.57-7.73 (m, 2H), 7.99 (d, J=5.58 Hz, 1H), 8.99 (s, 1H), 10.38-10.53 (m, 1H).

LCMS (Method 1): Rt=1.25 min, MS (ESIpos) m/z=374 [M+H]⁺

### Experimental Section—Examples

### Example 1

### N-{4-[2-(2-chlorophenyl)acetamido]pyridin-2-yl}-N-(3,4-difluorophenyl)acetamide

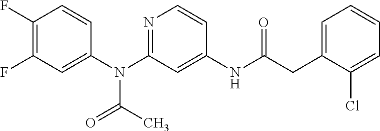 [SMILES: CC(=O)N(c1ccc(F)c(F)c1)c1cc(NC(=O)Cc2ccccc2Cl)ccn1]

According to GP H, N-(4-aminopyridin-2-yl)-N-(3,4-difluorophenyl)acetamide (Int. 79, 70 mg, 0.27 mmol) and 2-chlorophenylacetic acid (54 mg, 1.2 eq.) were dissolved in DMF (2 mL) and HATU (121 mg, 0.32 mmol, 1.2 eq.) and triethylamine (135 mg, 1.33 mmol, 5 eq.) were added. The mixture was stirred at rt for 2 h, then it was poured into water, extracted with ethyl acetate, the combined organic layers washed with brine, dried with sodium sulfate and the solvents evaporated. The crude product was purified by flash chromatography to yield 30 mg (24% of theory) of the title compound as pale yellow foam.

¹H NMR (400 MHz, DMSO-d6) δ[ppm] 2.00 (s, 3H), 3.88 (s, 2H), 7.10-7.14 (m, 1H), 7.29-7.34 (m, 2H), 7.39-7.55 (m, 5H), 7.71 (s, br, 1H), 8.28 (d, 1H), 10.8 (s, 1H).

LC-MS (method 1): Rt=1.13 min; MS (ESIpos): m/z=416 [M+H]⁺

### Example 2

### N-{4-[2-(2-chloro-3-fluorophenyl)acetamido]pyridin-2-yl}-N-(4-fluorophenyl)acetamide

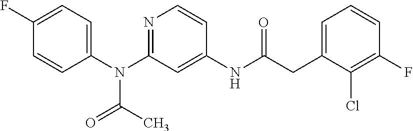 [SMILES: CC(=O)N(c1ccc(F)cc1)c1cc(NC(=O)Cc2cccc(F)c2Cl)ccn1]

According to GP H, N-(4-aminopyridin-2-yl)-N-(4-fluorophenyl)acetamide (Int. 80, 200 mg, 0.82 mmol) and 2-(2-chloro-3-fluorophenyl)acetic acid (154 mg, 1 eq.) were dissolved in DMF (10 mL) and T3P (778 mg, 2.45 mmol, 3 eq.) and triethylamine (495 mg, 4.89 mmol, 6 eq.) were added. The mixture was stirred at rt for 18 h, then it was poured into water, extracted with ethyl acetate, the combined organic layers washed with brine, dried with sodium sulfate and the solvents evaporated. The crude product was purified by preparative HPLC to yield 191 mg (56% of theory) of the title compound as a pale yellow solid.

In an alternative procedure, 250 g (1.0 eq., 669 mmol) of 2-(2-chloro-3-fluorophenyl)-N-[2-(4-fluoroanilino)pyridin-4-yl]acetamide (Int. 148) and 160 mL (1.4 eq., 940 mmol) of N,N-diisopropylethylamine were dissolved in 2000 mL of rac-2-methyltetrahydrofuran. At room temperature 71 mL (1.5 eq., 1.0 mol) of acetyl chloride were added dropwise and the reaction mixture was stirred at room temperature for 18 h. The reaction mixture was diluted with ethyl acetate and quenched by adding water. The organic phase was washed with saturated NaHCO₃-solution and water once each. After drying over magnesium sulfate the filtrate was concentrated under vacuum and the remaining residue was purified via column chromatography (Biotage autopurifier system (Isolera LS®), 375 g Biotage SNAP cartridge KP-NH®, hexane/dichloromethane (50%) to hexane/dichloromethane (75%) to dichloromethane (100%) to dichloromethane/ethyl acetate (80%)) followed by a second chromatography (Biotage autopurifier system (Isolera LS®), 1500 g Biotage SNAP cartridge KP-Sil®, hexane (100%) to hexane/ethyl acetate (30%) to ethyl acetate (100%)). The material was triturated with 2-methoxy-2-methylpropane and finally filtered. After drying at 50° C. 219 g of the title compound (79% theoretical yield) were obtained as a white solid.

¹H NMR (400 MHz, DMSO-d6) δ[ppm] 1.99 (s, 3H), 3.94 (s, 2H), 7.21-7.28 (m, 3H), 7.31-7.37 (m, 4H), 7.46 (dd, 1H), 7.68 (s, 1H), 8.27 (d, 1H), 10.8 (s, 1H).

LC-MS (method 1): Rt=1.09 min; MS (ESIpos): m/z=416 [M+H]⁺

TABLE 8
Examples 3-184: The following examples were generated by amide coupling
of amines with the corresponding carboxylic acids, using an appropriate general
procedure (GP H, GP I)

| | Structure |
|---|---|
| | IUPAC-Name |
| | LC-MS (method): Retention time; Mass found |
| Example | ¹H-NMR |
| 3 | 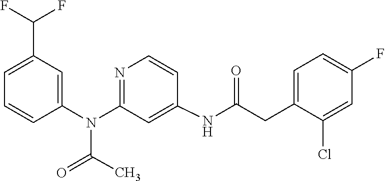 [SMILES: CC(=O)N(c1cccc(C(F)F)c1)c1cc(NC(=O)Cc2ccc(F)cc2Cl)ccn1] |
| | N-{4-[2-(2-chloro-4-fluorophenyl)acetamido]pyridin-2-yl}-N-[3- |
| | (difluoromethyl)phenyl]acetamide |
| | LC-MS (Method 2): Rₜ = 1.14 min; MS (ESIpos): m/z = 448 [M + H]⁺ |
| | ¹H-NMR (400 MHz, DMSO-d6) δ [ppm]: 2.005 (16.00), 2.518 (0.46), |
| | 3.337 (6.30), 6.899 (1.33), 7.038 (2.69), 7.178 (1.25), 7.183 (1.01), |
| | 7.190 (0.97), 7.204 (1.61), 7.211 (1.88), 7.226 (0.97), 7.233 (0.99), |
| | 7.421 (0.93), 7.428 (2.11), 7.434 (2.42), 7.442 (2.57), 7.450 (2.01), |
| | 7.457 (2.81), 7.464 (1.45), 7.480 (3.22), 7.488 (2.61), 7.493 (2.60), |
| | 7.502 (1.78), 7.507 (2.50), 7.512 (1.76), 7.537 (1.62), 7.556 (1.74), |
| | 7.575 (0.56), 7.657 (1.68), 8.297 (2.20), 8.312 (2.08), 10.771 (2.53). |
| 4 | 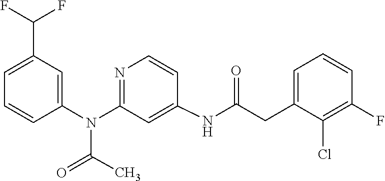 [SMILES: CC(=O)N(c1cccc(C(F)F)c1)c1cc(NC(=O)Cc2cccc(F)c2Cl)ccn1] |
| | N-{4-[2-(2-chloro-3-fluorophenyl)acetamido]pyridin-2-yl}-N-[3- |
| | (difluoromethyl)phenyl]acetamide |
| | LC-MS (Method 2): Rₜ = 1.13 min; MS (ESIneg): m/z = 446 [M − H]⁻ |
| | ¹H-NMR (400 MHZ, DMSO-d6) δ [ppm]: 2.005 (16.00), 2.518 (0.99), |
| | 2.523 (0.67), 3.938 (7.22), 6.899 (1.27), 7.038 (2.57), 7.178 (1.16), |
| | 7.258 (0.81), 7.265 (1.21), 7.271 (0.85), 7.275 (0.93), 7.281 (1.25), |
| | 7.321 (0.48), 7.336 (1.99), 7.340 (2.32), 7.357 (4.47), 7.364 (1.43), |
| | 7.371 (1.30), 7.423 (0.79), 7.442 (1.09), 7.480 (1.93), 7.488 (2.57), |
| | 7.492 (2.45), 7.502 (1.85), 7.507 (2.34), 7.513 (1.64), 7.538 (1.55), |
| | 7.557 (1.67), 7.576 (0.53), 7.657 (1.59), 8.300 (2.13), 8.314 (2.01), |
| | 10.809 (2.42). |
| 5 | 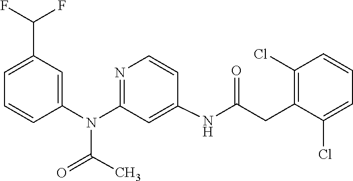 [SMILES: CC(=O)N(c1cccc(C(F)F)c1)c1cc(NC(=O)Cc2c(Cl)cccc2Cl)ccn1] |
| | N-{4-[2-(2,6-dichlorophenyl)acetamido]pyridin-2-yl}-N-[3- |
| | (difluoromethyl)phenyl]acetamide |
| | LC-MS (Method 2): Rₜ = 1.16 min; MS (ESIneg): m/z = 462 [M − H]⁻ |
| | ¹H-NMR (400 MHz, DMSO-d6) δ [ppm]: 2.003 (16.00), 2.518 (0.80), |
| | 2.523 (0.54), 4.077 (7.90), 6.899 (1.36), 7.038 (2.77), 7.178 (1.24), |
| | 7.333 (1.67), 7.351 (2.11), 7.354 (2.35), 7.373 (2.59), 7.426 (0.85), |
| | 7.445 (1.18), 7.465 (1.81), 7.470 (1.86), 7.480 (3.29), 7.484 (11.02), |
| | 7.504 (4.62), 7.513 (1.72), 7.537 (1.61), 7.557 (1.72), 7.576 (0.54), |
| | 7.657 (1.60), 8.300 (2.20), 8.315 (2.08), 10.886 (2.53). |
| 6 | 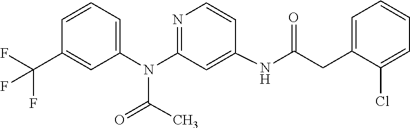 [SMILES: CC(=O)N(c1cccc(C(F)(F)F)c1)c1cc(NC(=O)Cc2ccccc2Cl)ccn1] |
| | N-{4-[2-(2-chlorophenyl)acetamido]pyridin-2-yl}-N-[3- |
| | (trifluoromethyl)phenyl]acetamide |
| | LC-MS (Method 2): Rₜ = 1.20 min; MS (ESIneg): m/z = 446 [M − H]⁻ |
| | ¹H-NMR (400 MHz, DMSO-d6) δ [ppm]: 2.013 (16.00), 2.518 (1.32), |
| | 2.523 (0.87), 3.877 (7.80), 7.300 (3.34), 7.309 (2.69), 7.314 (2.76), |
| | 7.323 (4.65), 7.333 (0.65), 7.394 (1.68), 7.402 (0.84), 7.404 (1.06), |
| | 7.409 (1.24), 7.418 (1.06), 7.423 (0.42), 7.433 (1.98), 7.442 (1.62), |
| | 7.447 (1.25), 7.457 (1.36), 7.514 (2.15), 7.519 (2.47), 7.528 (1.91), |
| | 7.533 (2.38), 7.538 (1.16), 7.615 (0.69), 7.635 (1.73), 7.654 (1.50), |
| | 7.663 (1.94), 7.683 (2.06), 7.703 (1.96), 8.304 (1.96), 8.318 (1.84), |
| | 10.782 (2.45). |
| 7 | 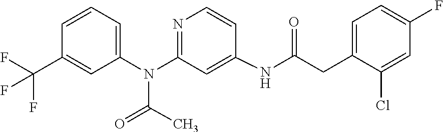 [SMILES: CC(=O)N(c1cccc(C(F)(F)F)c1)c1cc(NC(=O)Cc2ccc(F)cc2Cl)ccn1] |
| | N-{4-[2-(2-chloro-4-fluorophenyl)acetamido]pyridin-2-yl}-N-[3- |
| | (trifluoromethyl)phenyl]acetamide |
| | LC-MS (Method 2): Rₜ = 1.23 min; MS (ESIneg): m/z = 464 [M − H]⁻ |
| | ¹H-NMR (400 MHZ, DMSO-d6) δ [ppm]: 2.012 (16.00), 2.518 (2.28), |
| | 2.522 (1.41), 3.819 (0.41), 3.868 (7.17), 7.185 (0.83), 7.192 (0.94), |
| | 7.206 (1.69), 7.212 (1.93), 7.227 (0.95), 7.234 (1.02), 7.429 (1.78), |
| | 7.436 (1.98), 7.443 (1.76), 7.451 (1.95), 7.458 (3.20), 7.464 (1.58), |
| | 7.480 (1.36), 7.505 (1.89), 7.510 (2.00), 7.519 (2.71), 7.524 (2.57), |
| | 7.538 (1.28), 7.616 (0.77), 7.635 (1.93), 7.655 (1.68), 7.664 (2.30), |
| | 7.680 (2.15), 7.702 (2.29), 8.304 (2.15), 8.317 (2.05), 10.779 (2.79). |
| 8 | 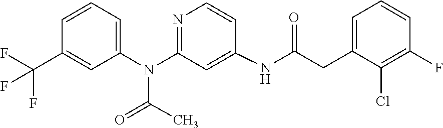 [SMILES: CC(=O)N(c1cccc(C(F)(F)F)c1)c1cc(NC(=O)Cc2cccc(F)c2Cl)ccn1] |
| | N-{4-[2-(2-chloro-3-fluorophenyl)acetamido]pyridin-2-yl}-N-[3- |
| | (trifluoromethyl)phenyl]acetamide |
| | LC-MS (Method 2): Rₜ = 1.22 min; MS (ESIneg): m/z = 464 [M − H]⁻ |
| | ¹H-NMR (400 MHZ, DMSO-d6) δ [ppm]: 2.012 (16.00), 2.518 (1.22), |
| | 2.522 (0.79), 3.941 (7.50), 7.258 (0.80), 7.266 (1.23), 7.273 (0.88), |
| | 7.276 (0.91), 7.283 (1.27), 7.322 (0.56), 7.336 (2.10), 7.341 (2.45), |
| | 7.357 (4.71), 7.365 (1.48), 7.372 (1.34), 7.506 (1.73), 7.511 (1.89), |
| | 7.521 (2.55), 7.525 (2.43), 7.540 (1.10), 7.617 (0.71), 7.636 (1.75), |
| | 7.655 (1.49), 7.665 (1.95), 7.684 (2.03), 7.705 (1.99), 8.307 (2.01), |
| | 8.321 (1.91), 10.820 (2.51). |
| 9 | 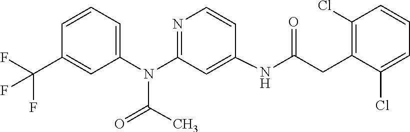 [SMILES: CC(=O)N(c1cccc(C(F)(F)F)c1)c1cc(NC(=O)Cc2c(Cl)cccc2Cl)ccn1] |
| | N-{4-[2-(2,6-dichlorophenyl)acetamido]pyridin-2-yl}-N-[3- |
| | (trifluoromethyl)phenyl]acetamide |
| | LC-MS (Method 2): Rₜ = 1.25 min; MS (ESIneg): m/z = 480 [M − H]⁻ |
| | ¹H-NMR (400 MHZ, DMSO-d6) δ [ppm]: 2.010 (16.00), 2.332 (0.46), |
| | 2.518 (2.20), 2.523 (1.39), 2.673 (0.46), 4.030 (0.44), 4.079 (7.73), |
| | 7.334 (1.65), 7.352 (2.17), 7.355 (2.36), 7.374 (2.64), 7.469 (0.51), |
| | 7.485 (8.52), 7.489 (2.37), 7.499 (1.77), 7.504 (5.31), 7.525 (0.81), |
| | 7.544 (1.08), 7.616 (0.70), 7.636 (1.77), 7.655 (1.50), 7.664 (1.98), |
| | 7.683 (1.75), 7.709 (1.97), 8.307 (1.93), 8.321 (1.84), 10.896 (2.49). |
| 10 | 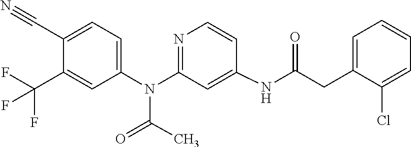 [SMILES: CC(=O)N(c1ccc(C#N)c(C(F)(F)F)c1)c1cc(NC(=O)Cc2ccccc2Cl)ccn1] |
| | N-{4-[2-(2-chlorophenyl)acetamido]pyridin-2-yl}-N-[4-cyano-3- |
| | (trifluoromethyl)phenyl]acetamide |
| | LC-MS (Method 2): Rₜ = 1.20 min; MS (ESIpos): m/z = 473 [M + H]⁺ |
| | ¹H-NMR (400 MHZ, DMSO-d6) δ [ppm]: 2.036 (16.00), 2.518 (1.94), |
| | 2.522 (1.26), 3.887 (6.84), 7.300 (3.07), 7.309 (2.40), 7.315 (2.50), |
| | 7.324 (4.15), 7.334 (0.56), 7.395 (1.47), 7.402 (0.74), 7.405 (0.95), |
| | 7.409 (1.13), 7.418 (0.95), 7.424 (0.41), 7.433 (1.72), 7.443 (1.35), |
| | 7.447 (1.14), 7.457 (1.22), 7.563 (1.52), 7.568 (1.79), 7.578 (2.06), |
| | 7.582 (1.80), 7.594 (0.73), 7.710 (1.51), 8.012 (1.91), 8.017 (1.87), |
| | 8.143 (1.64), 8.164 (1.52), 8.376 (1.60), 8.390 (1.51), 10.846 (2.14). |
| 11 | 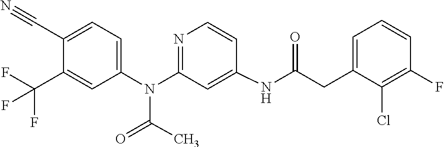 [SMILES: CC(=O)N(c1ccc(C#N)c(C(F)(F)F)c1)c1cc(NC(=O)Cc2cccc(F)c2Cl)ccn1] |
| | N-{4-[2-(2-chloro-3-fluorophenyl)acetamido]pyridin-2-yl}-N-[4- |
| | cyano-3-(trifluoromethyl)phenyl]acetamide |
| | LC-MS (Method 2): Rₜ = 1.21 min; MS (ESIpos): m/z = 491 [M + H]⁺ |
| | ¹H-NMR (400 MHz, DMSO-d6) δ [ppm]: 2.035 (16.00), 2.518 (3.85), |
| | 2.522 (2.47), 3.950 (6.51), 7.260 (0.68), 7.267 (1.08), 7.273 (0.76), |
| | 7.278 (0.82), 7.282 (1.12), 7.338 (1.74), 7.342 (2.04), 7.345 (1.44), |
| | 7.359 (4.32), 7.366 (1.24), 7.373 (1.20), 7.556 (1.49), 7.561 (1.53), |
| | 7.570 (1.68), 7.575 (2.20), 7.599 (0.74), 7.709 (1.54), 8.013 (1.95), |
| | 8.019 (1.92), 8.146 (1.70), 8.167 (1.57), 8.378 (1.60), 8.392 (1.52), |
| | 10.883 (2.23). |
| 12 | 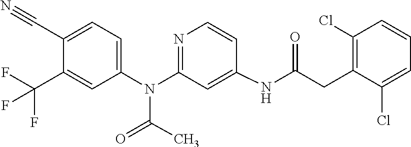 [SMILES: CC(=O)N(c1ccc(C#N)c(C(F)(F)F)c1)c1cc(NC(=O)Cc2c(Cl)cccc2Cl)ccn1] |
| | N-[4-cyano-3-(trifluoromethyl)phenyl]-N-{4-[2-(2,6- |
| | dichlorophenyl)acetamido]pyridin-2-yl}acetamide |
| | LC-MS (Method 2): Rₜ = 1.24 min; MS (ESIneg): m/z = 505 [M − H]⁻ |
| | ¹H-NMR (400 MHZ, DMSO-d6) δ [ppm]: 2.034 (16.00), 2.518 (2.88), |
| | 2.522 (1.94), 4.088 (7.11), 7.334 (1.47), 7.352 (1.93), 7.355 (2.11), |
| | 7.374 (2.31), 7.485 (6.51), 7.504 (4.00), 7.535 (1.44), 7.540 (1.52), |
| | 7.549 (1.50), 7.554 (1.52), 7.579 (0.74), 7.597 (0.78), 7.707 (1.57), |
| | 8.019 (2.10), 8.024 (2.06), 8.143 (1.77), 8.163 (1.66), 8.379 (1.68), |
| | 8.393 (1.59), 10.963 (2.35). |
| 13 | 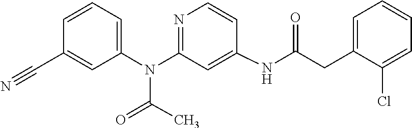 [SMILES: CC(=O)N(c1cccc(C#N)c1)c1cc(NC(=O)Cc2ccccc2Cl)ccn1] |
| | N-{4-[2-(2-chlorophenyl)acetamido]pyridin-2-yl}-N-(3- |
| | cyanophenyl)acetamide |
| | LC-MS (Method 1): Rₜ = 1.07 min; MS (ESIpos): m/z = 405 [M + H]⁺ |
| | 1H-NMR (400 MHZ, DMSO-d6) δ [ppm]: 0.967 (0.17), 1.108 (12.87), |
| | 1.987 (0.18), 2.014 (16.00), 2.331 (0.30), 2.518 (1.94), 2.523 (1.21), |
| | 2.673 (0.30), 3.880 (7.97), 4.190 (1.25), 7.291 (0.32), 7.302 (3.17), |
| | 7.311 (2.32), 7.315 (2.39), 7.325 (4.39), 7.336 (0.56), 7.388 (0.28), |
| | 7.396 (1.73), 7.404 (0.91), 7.407 (1.09), 7.411 (1.17), 7.420 (1.10), |
| | 7.425 (0.45), 7.435 (1.97), 7.444 (1.67), 7.448 (1.19), 7.458 (1.35), |
| | 7.467 (0.19), 7.496 (1.78), 7.501 (1.71), 7.510 (1.74), 7.515 (1.83), |
| | 7.578 (0.30), 7.583 (0.32), 7.594 (3.00), 7.596 (3.54), 7.603 (1.51), |
| | 7.609 (2.28), 7.629 (0.51), 7.702 (1.55), 7.765 (1.09), 7.769 (1.30), |
| | 7.776 (0.93), 7.781 (0.94), 7.784 (1.04), 7.787 (0.92), 7.791 (0.89), |
| | 7.844 (2.46), 8.285 (1.95), 8.299 (1.86), 10.783 (2.55). |
| 14 | 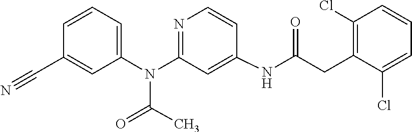 [SMILES: CC(=O)N(c1cccc(C#N)c1)c1cc(NC(=O)Cc2c(Cl)cccc2Cl)ccn1] |
| | N-(3-cyanophenyl)-N-{4-[2-(2,6-dichlorophenyl)acetamido]pyridin- |
| | 2-yl)acetamide |
| | LC-MS (Method 1): Rₜ = 1.12 min; MS (ESIpos): m/z = 439 [M + H]⁺ |
| | 1H-NMR (400 MHz, DMSO-d6) δ [ppm]: 0.967 (0.66), 1.054 (0.18), |
| | 1.071 (0.18), 1.107 (13.00), 1.144 (0.43), 1.208 (0.29), 1.232 (0.69), |
| | 1.983 (1.11), 2.011 (16.00), 2.332 (0.55), 2.336 (0.26), 2.518 (5.29), |
| | 2.522 (3.62), 2.673 (0.57), 4.081 (8.40), 4.189 (1.24), 6.461 (0.23), |
| | 7.335 (1.56), 7.354 (2.17), 7.356 (2.29), 7.375 (2.68), 7.464 (1.76), |
| | 7.469 (1.83), 7.479 (1.93), 7.486 (7.91), 7.506 (4.66), 7.583 (0.29), |
| | 7.596 (3.34), 7.599 (3.62), 7.603 (1.95), 7.610 (2.74), 7.612 (2.56), |
| | 7.630 (0.44), 7.701 (1.63), 7.758 (0.21), 7.765 (1.12), 7.769 (1.26), |
| | 7.773 (0.98), 7.780 (1.25), 7.782 (1.14), 7.787 (0.95), 7.791 (0.98), |
| | 7.848 (2.56), 8.288 (2.08), 8.302 (2.02), 10.899 (2.80). |
| 15 | 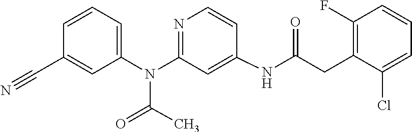 [SMILES: CC(=O)N(c1cccc(C#N)c1)c1cc(NC(=O)Cc2c(F)cccc2Cl)ccn1] |
| | N-{4-[2-(2-chloro-6-fluorophenyl)acetamido]pyridin-2-yl}-N-(3- |
| | cyanophenyl)acetamide |
| | LC-MS (Method 1): Rₜ = 1.08 min; MS (ESIpos): m/z = 423 [M + H]⁺ |
| | 1H-NMR (400 MHz, DMSO-d6) δ [ppm]: 0.899 (0.23), 0.916 (0.56), |
| | 0.936 (0.38), 0.939 (0.29), 0.967 (0.26), 1.107 (9.47), 1.220 (0.25), |
| | 1.239 (1.25), 1.254 (1.28), 1.269 (0.70), 1.482 (0.39), 1.985 (0.18), |
| | 2.011 (16.00), 2.332 (0.54), 2.336 (0.23), 2.518 (2.96), 2.522 (1.88), |
| | 2.673 (0.54), 2.678 (0.23), 3.880 (0.47), 3.925 (4.44), 3.929 (4.35), |
| | 7.226 (0.63), 7.231 (0.65), 7.244 (0.90), 7.249 (1.51), 7.252 (0.82), |
| | 7.269 (0.90), 7.273 (0.93), 7.302 (0.21), 7.325 (0.27), 7.337 (0.72), |
| | 7.341 (0.86), 7.357 (3.56), 7.363 (2.98), 7.377 (1.29), 7.382 (1.27), |
| | 7.397 (1.47), 7.402 (0.47), 7.417 (0.48), 7.469 (1.63), 7.473 (1.67), |
| | 7.483 (1.66), 7.488 (1.67), 7.495 (0.16), 7.580 (0.25), 7.585 (0.29), |
| | 7.594 (3.15), 7.597 (3.42), 7.605 (1.59), 7.610 (2.34), 7.630 (0.44), |
| | 7.704 (1.51), 7.765 (1.04), 7.769 (1.25), 7.772 (0.97), 7.777 (0.93), |
| | 7.780 (0.97), 7.783 (1.03), 7.787 (0.90), 7.791 (0.90), 7.846 (2.43), |
| | 8.289 (1.92), 8.303 (1.84), 10.889 (2.41). |
| 16 | 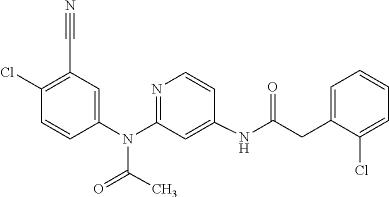 [SMILES: CC(=O)N(c1ccc(Cl)c(C#N)c1)c1cc(NC(=O)Cc2ccccc2Cl)ccn1] |
| | N-(4-chloro-3-cyanophenyl)-N-{4-[2-(2- |
| | chlorophenyl)acetamido]pyridin-2-yl}acetamide |
| | LC-MS (Method 2): Rₜ = 1.14 min; MS (ESIpos): m/z = 439 [M + H]⁺ |
| | ¹H-NMR (400 MHZ, DMSO-d6) δ [ppm]: 2.022 (16.00), 2.518 (1.54), |
| | 2.522 (1.01), 3.884 (7.46), 7.303 (3.52), 7.311 (1.87), 7.313 (2.11), |
| | 7.317 (2.05), 7.319 (2.08), 7.327 (4.33), 7.336 (0.43), 7.338 (0.50), |
| | 7.399 (1.60), 7.406 (0.78), 7.410 (0.99), 7.414 (1.03), 7.423 (1.02), |
| | 7.437 (1.89), 7.445 (1.57), 7.450 (1.07), 7.452 (0.74), 7.460 (1.31), |
| | 7.502 (1.70), 7.508 (1.63), 7.516 (1.64), 7.521 (1.77), 7.605 (1.01), |
| | 7.611 (1.04), 7.627 (1.26), 7.633 (1.30), 7.728 (1.29), 7.760 (3.69), |
| | 7.782 (2.75), 8.009 (2.25), 8.015 (2.19), 8.284 (1.69), 8.298 (1.59), |
| | 10.794 (2.36). |
| 17 | 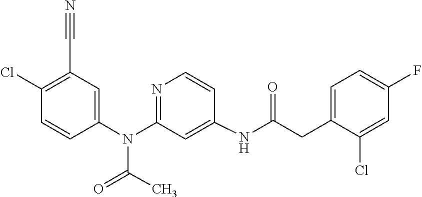 [SMILES: CC(=O)N(c1ccc(Cl)c(C#N)c1)c1cc(NC(=O)Cc2ccc(F)cc2Cl)ccn1] |
| | N-(4-chloro-3-cyanophenyl)-N-{4-[2-(2-chloro-4- |
| | fluorophenyl)acetamido]pyridin-2-yl)acetamide |
| | LC-MS (Method 2): Rₜ = 1.17 min; MS (ESIpos): m/z = 457 [M + H]⁺ |
| | ¹H-NMR (400 MHz, DMSO-d6) δ [ppm]: 2.021 (16.00), 2.518 (3.28), |
| | 2.523 (2.32), 3.875 (6.40), 7.189 (0.75), 7.196 (0.84), 7.210 (1.58), |
| | 7.217 (1.88), 7.232 (0.92), 7.239 (0.99), 7.433 (1.64), 7.439 (1.66), |
| | 7.448 (1.51), 7.455 (1.84), 7.462 (2.14), 7.464 (1.88), 7.470 (1.48), |
| | 7.485 (1.28), 7.494 (1.78), 7.498 (1.80), 7.508 (1.78), 7.513 (1.86), |
| | 7.605 (1.02), 7.612 (1.08), 7.627 (1.35), 7.634 (1.45), 7.726 (1.39), |
| | 7.762 (3.79), 7.783 (2.90), 8.009 (2.26), 8.015 (2.33), 8.283 (1.74), |
| | 8.297 (1.72), 10.793 (2.56). |
| 18 | 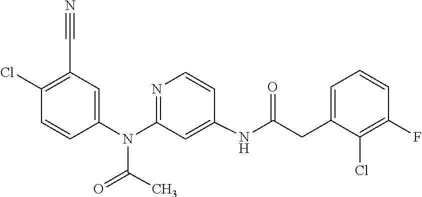 [SMILES: CC(=O)N(c1ccc(Cl)c(C#N)c1)c1cc(NC(=O)Cc2cccc(F)c2Cl)ccn1] |
| | N-(4-chloro-3-cyanophenyl)-N-{4-[2-(2-chloro-3- |
| | fluorophenyl)acetamido]pyridin-2-yl}acetamide |
| | LC-MS (Method 2): Rₜ = 1.16 min; MS (ESIpos): m/z = 457 [M + H]⁺ |
| | ¹H-NMR (400 MHz, DMSO-d6) δ [ppm]: 2.021 (16.00), 2.669 (0.41), |
| | 3.947 (8.83), 7.263 (1.04), 7.270 (1.34), 7.278 (1.27), 7.286 (1.79), |
| | 7.325 (0.43), 7.340 (2.31), 7.345 (2.90), 7.361 (4.48), 7.368 (1.65), |
| | 7.376 (1.38), 7.382 (0.50), 7.495 (2.03), 7.500 (1.78), 7.510 (2.16), |
| | 7.514 (1.87), 7.608 (1.26), 7.614 (1.25), 7.629 (1.68), 7.636 (1.63), |
| | 7.728 (2.10), 7.762 (3.76), 7.784 (2.91), 8.011 (2.83), 8.016 (2.65), |
| | 8.285 (2.19), 8.299 (2.14), 10.833 (3.45). |
| 19 | 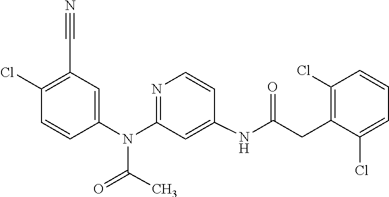 [SMILES: CC(=O)N(c1ccc(Cl)c(C#N)c1)c1cc(NC(=O)Cc2c(Cl)cccc2Cl)ccn1] |
| | N-(4-chloro-3-cyanophenyl)-N-{4-[2-(2,6- |
| | dichlorophenyl)acetamido]pyridin-2-yl}acetamide |
| | LC-MS (Method 2): Rₜ = 1.20 min; MS (ESIpos): m/z = 474 [M + H]⁺ |
| | ¹H-NMR (400 MHZ, DMSO-d6) δ [ppm]: 2.019 (16.00), 2.518 (4.03), |
| | 2.523 (2.87), 4.086 (7.51), 7.336 (1.56), 7.355 (2.01), 7.358 (2.31), |
| | 7.376 (2.48), 7.471 (1.57), 7.476 (1.72), 7.488 (7.72), 7.507 (4.33), |
| | 7.610 (0.99), 7.617 (1.07), 7.632 (1.35), 7.638 (1.45), 7.728 (1.33), |
| | 7.760 (3.81), 7.782 (2.86), 8.011 (2.26), 8.018 (2.32), 8.288 (1.72), |
| | 8.302 (1.69), 10.911 (2.55). |
| 20 | 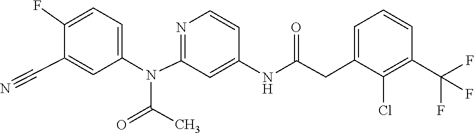 [SMILES: CC(=O)N(c1ccc(F)c(C#N)c1)c1cc(NC(=O)Cc2cccc(C(F)(F)F)c2Cl)ccn1] |
| | N-(4-{2-[2-chloro-3-(trifluoromethyl)phenyl]acetamido}pyridin-2- |
| | yl)-N-(3-cyano-4-fluorophenyl)acetamide |
| | LC-MS (Method 2): Rₜ = 1.20 min; MS (ESIpos): m/z = 491 [M + H]⁺ |
| | ¹H-NMR (400 MHz, DMSO-d6) δ [ppm]: 2.006 (16.00), 2.331 (0.41), |
| | 2.518 (1.86), 2.522 (1.19), 4.033 (7.17), 7.469 (1.75), 7.474 (1.84), |
| | 7.484 (1.79), 7.488 (1.86), 7.529 (0.85), 7.549 (3.17), 7.571 (3.50), |
| | 7.593 (1.72), 7.694 (0.67), 7.700 (0.77), 7.706 (0.79), 7.713 (0.82), |
| | 7.723 (0.66), 7.728 (0.62), 7.744 (1.69), 7.764 (2.56), 7.797 (1.65), |
| | 7.800 (1.50), 7.816 (1.42), 7.820 (1.24), 7.974 (1.13), 7.980 (1.16), |
| | 7.988 (1.18), 7.995 (1.08), 8.270 (1.90), 8.283 (1.82), 10.870 (2.75). |
| 21 | 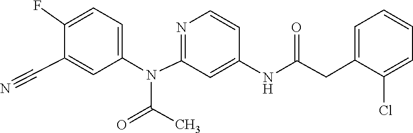 [SMILES: CC(=O)N(c1ccc(F)c(C#N)c1)c1cc(NC(=O)Cc2ccccc2Cl)ccn1] |
| | N-{4-[2-(2-chlorophenyl)acetamido]pyridin-2-yl}-N-(3-cyano-4- |
| | fluorophenyl)acetamide |
| | LC-MS (Method 2): Rₜ = 1.09 min; MS (ESIpos): m/z = 423 [M + H]⁺ |
| | ¹H-NMR (400 MHZ, DMSO-d6) δ [ppm]: 2.010 (16.00), 2.518 (2.76), |
| | 2.522 (1.74), 3.885 (8.30), 7.305 (3.60), 7.312 (2.01), 7.314 (2.21), |
| | 7.318 (2.22), 7.320 (2.31), 7.328 (4.65), 7.339 (0.55), 7.400 (1.79), |
| | 7.407 (0.85), 7.411 (1.10), 7.415 (1.17), 7.424 (1.15), 7.438 (2.04), |
| | 7.447 (1.63), 7.451 (1.14), 7.461 (1.46), 7.486 (1.80), 7.491 (1.87), |
| | 7.500 (1.84), 7.505 (1.94), 7.548 (1.27), 7.570 (3.03), 7.593 (1.71), |
| | 7.691 (0.68), 7.698 (0.77), 7.704 (0.80), 7.710 (0.81), 7.721 (0.66), |
| | 7.726 (0.61), 7.733 (0.64), 7.751 (1.42), 7.973 (1.12), 7.980 (1.15), |
| | 7.987 (1.19), 7.994 (1.08), 8.266 (1.92), 8.280 (1.81), 10.787 (2.65). |
| 22 | 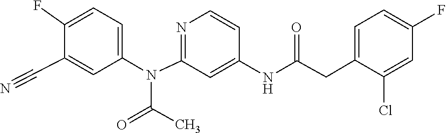 [SMILES: CC(=O)N(c1ccc(F)c(C#N)c1)c1cc(NC(=O)Cc2ccc(F)cc2Cl)ccn1] |
| | N-{4-[2-(2-chloro-4-fluorophenyl)acetamido]pyridin-2-yl}-N-(3- |
| | cyano-4-fluorophenyl)acetamide |
| | LC-MS (Method 2): Rₜ = 1.12 min; MS (ESIpos): m/z = 441 [M + H]⁺ |
| | ¹H-NMR (400 MHZ, DMSO-d6) δ [ppm]: 2.009 (16.00), 2.518 (0.89), |
| | 2.522 (0.56), 3.876 (7.01), 7.190 (0.82), 7.197 (0.92), 7.212 (1.68), |
| | 7.218 (1.86), 7.233 (0.92), 7.240 (1.01), 7.433 (1.80), 7.440 (1.81), |
| | 7.449 (1.61), 7.455 (1.94), 7.462 (2.17), 7.465 (1.84), 7.471 (1.51), |
| | 7.478 (1.93), 7.483 (2.03), 7.486 (1.57), 7.492 (1.84), 7.497 (1.93), |
| | 7.548 (1.29), 7.571 (3.06), 7.593 (1.69), 7.691 (0.70), 7.698 (0.80), |
| | 7.704 (0.82), 7.710 (0.84), 7.721 (0.68), 7.726 (0.63), 7.733 (0.69), |
| | 7.749 (1.43), 7.973 (1.17), 7.979 (1.19), 7.987 (1.20), 7.993 (1.09), |
| | 8.265 (1.96), 8.279 (1.85), 10.785 (2.75). |
| 23 | 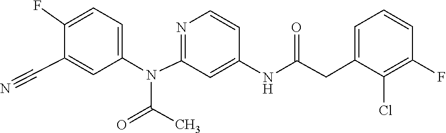 [SMILES: CC(=O)N(c1ccc(F)c(C#N)c1)c1cc(NC(=O)Cc2cccc(F)c2Cl)ccn1] |
| | N-{4-[2-(2-chloro-3-fluorophenyl)acetamido]pyridin-2-yl}-N-(3- |
| | cyano-4-fluorophenyl)acetamide |
| | LC-MS (Method 2): Rₜ = 1.11 min; MS (ESIpos): m/z = 441 [M + H]⁺ |
| | ¹H-NMR (400 MHz, DMSO-d6) δ [ppm]: 2.009 (16.00), 2.518 (2.32), |
| | 2.522 (1.51), 3.948 (7.79), 7.265 (0.85), 7.272 (1.25), 7.279 (0.93), |
| | 7.283 (0.99), 7.289 (1.36), 7.327 (0.45), 7.341 (2.02), 7.346 (2.71), |
| | 7.362 (4.42), 7.370 (1.46), 7.377 (1.30), 7.479 (1.79), 7.483 (1.86), |
| | 7.493 (1.84), 7.497 (1.87), 7.549 (1.29), 7.572 (3.03), 7.594 (1.70), |
| | 7.693 (0.68), 7.700 (0.81), 7.705 (0.79), 7.712 (0.85), 7.723 (0.67), |
| | 7.728 (0.65), 7.735 (0.67), 7.751 (1.41), 7.974 (1.14), 7.981 (1.17), |
| | 7.988 (1.19), 7.995 (1.08), 8.268 (1.93), 8.282 (1.82), 10.825 (2.68). |
| 24 | 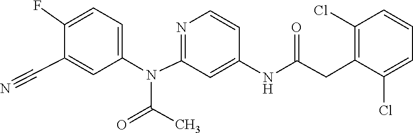 [SMILES: CC(=O)N(c1ccc(F)c(C#N)c1)c1cc(NC(=O)Cc2c(Cl)cccc2Cl)ccn1] |
| | N-(3-cyano-4-fluorophenyl)-N-{4-[2-(2,6- |
| | dichlorophenyl)acetamido]pyridin-2-yl}acetamide |
| | LC-MS (Method 2): Rₜ = 1.15 min; MS (ESIneg): m/z = 455 [M − H]⁻ |
| | ¹H-NMR (400 MHz, DMSO-d6) δ [ppm]: 2.007 (16.00), 2.074 (1.24), |
| | 2.518 (1.47), 2.523 (0.97), 4.087 (8.24), 7.337 (1.62), 7.356 (2.19), |
| | 7.358 (2.28), 7.377 (2.69), 7.453 (1.80), 7.457 (1.77), 7.467 (1.77), |
| | 7.472 (1.81), 7.489 (7.48), 7.509 (4.64), 7.547 (1.33), 7.569 (3.01), |
| | 7.592 (1.75), 7.619 (0.48), 7.697 (0.70), 7.703 (0.80), 7.709 (0.81), |
| | 7.716 (0.86), 7.726 (0.70), 7.731 (0.67), 7.738 (0.75), 7.752 (1.41), |
| | 7.975 (1.15), 7.981 (1.19), 7.989 (1.21), 7.996 (1.09), 8.270 (1.94), |
| | 8.284 (1.85), 10.902 (2.77). |
| 25 | 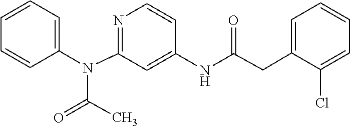 [SMILES: CC(=O)N(c1ccccc1)c1cc(NC(=O)Cc2ccccc2Cl)ccn1] |
| | N-{4-[2-(2-chlorophenyl)acetamido]pyridin-2-yl}-N- |
| | phenylacetamide |
| | ¹H NMR (250 MHz, DMSO-d6) δ [ppm]: 1.98 (s, 3H), 3.86 (s, 2H), |
| | 7.17-7.54 (m, 10H), 7.65 (d, J = 1.6, 1H), 8.27 (d, J = 5.6, 1H), 10.72 (s, |
| | 1H). |
| 26 | 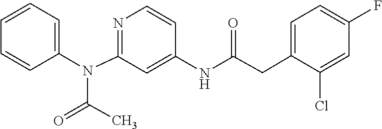 [SMILES: CC(=O)N(c1ccccc1)c1cc(NC(=O)Cc2ccc(F)cc2Cl)ccn1] |
| | N-{4-[2-(2-chloro-4-fluorophenyl)acetamido]pyridin-2-yl}-N- |
| | phenylacetamide |
| | LC-MS (Method 2): Rₜ = 1.09 min; MS (ESIpos): m/z = 308 [M + H]⁺ |
| | ¹H-NMR (400 MHz, DMSO-d6) δ [ppm]: 1.986 (16.00), 2.518 (3.27), |
| | 2.523 (2.08), 3.860 (6.30), 5.759 (2.71), 7.184 (0.69), 7.190 (0.79), |
| | 7.205 (1.51), 7.212 (1.74), 7.226 (0.84), 7.233 (0.92), 7.254 (2.07), |
| | 7.257 (2.45), 7.269 (0.85), 7.275 (3.42), 7.278 (2.92), 7.283 (1.03), |
| | 7.297 (0.69), 7.302 (1.82), 7.317 (0.94), 7.320 (1.34), 7.323 (0.68), |
| | 7.380 (0.48), 7.386 (2.75), 7.390 (1.13), 7.405 (3.43), 7.419 (0.69), |
| | 7.424 (1.50), 7.426 (1.20), 7.428 (1.83), 7.435 (1.73), 7.440 (1.60), |
| | 7.445 (2.02), 7.450 (2.95), 7.457 (3.23), 7.463 (2.56), 7.478 (1.19), |
| | 7.649 (1.82), 7.653 (1.83), 8.266 (2.29), 8.279 (2.22), 10.737 (2.33). |
| 27 | 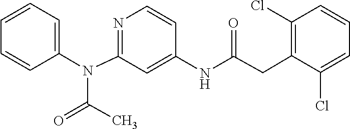 [SMILES: CC(=O)N(c1ccccc1)c1cc(NC(=O)Cc2c(Cl)cccc2Cl)ccn1] |
| | N-{4-[2-(2,6-dichlorophenyl)acetamido]pyridin-2-yl}-N- |
| | phenylacetamide |
| | LC-MS (Method 2): Rₜ = 1.12 min; MS (ESIpos): m/z = 415 [M + H]⁺ |
| | ¹H-NMR (400 MHZ, DMSO-d6) δ [ppm]: 1.959 (2.02), 1.985 (16.00), |
| | 2.518 (2.49), 2.522 (1.59), 2.532 (1.38), 2.686 (8.92), 4.072 (7.21), |
| | 5.758 (5.72), 7.261 (2.47), 7.274 (0.85), 7.279 (3.39), 7.283 (3.08), |
| | 7.297 (0.72), 7.302 (1.83), 7.308 (0.66), 7.317 (1.14), 7.321 (1.44), |
| | 7.324 (0.83), 7.333 (1.91), 7.351 (1.94), 7.354 (2.14), 7.373 (2.35), |
| | 7.381 (0.56), 7.386 (2.67), 7.390 (1.16), 7.405 (3.46), 7.420 (2.58), |
| | 7.425 (3.05), 7.434 (1.85), 7.439 (1.91), 7.484 (6.29), 7.503 (3.82), |
| | 7.520 (0.73), 7.541 (0.43), 7.653 (1.74), 7.656 (1.72), 8.270 (2.27), |
| | 8.284 (2.16), 10.854 (2.26). |
| 28 | 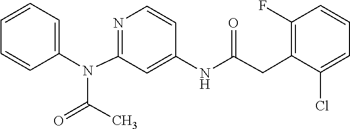 [SMILES: CC(=O)N(c1ccccc1)c1cc(NC(=O)Cc2c(F)cccc2Cl)ccn1] |
| | N-{4-[2-(2-chloro-6-fluorophenyl)acetamido]pyridin-2-yl}-N- |
| | phenylacetamide |
| | LC-MS (Method 1): Rₜ = 1.07 min; MS (ESIpos): m/z = 398 [M + H]⁺ |
| | ¹H-NMR (400 MHZ, DMSO-d6) δ [ppm]: 1.232 (0.55), 1.959 (1.27), |
| | 1.969 (0.64), 1.985 (16.00), 2.331 (0.78), 2.518 (3.95), 2.523 (2.60), |
| | 2.673 (0.78), 2.686 (0.67), 3.159 (4.24), 3.172 (3.97), 3.915 (4.24), |
| | 3.918 (4.08), 4.096 (0.91), 4.109 (0.95), 7.224 (0.66), 7.229 (0.69), |
| | 7.243 (0.96), 7.247 (1.57), 7.250 (1.09), 7.259 (2.58), 7.267 (1.20), |
| | 7.271 (1.62), 7.277 (3.44), 7.280 (3.06), 7.283 (1.67), 7.297 (0.66), |
| | 7.302 (1.82), 7.306 (0.75), 7.317 (1.06), 7.320 (1.37), 7.324 (0.84), |
| | 7.329 (0.53), 7.335 (0.84), 7.339 (0.89), 7.355 (3.44), 7.361 (2.68), |
| | 7.375 (1.42), 7.380 (1.73), 7.386 (2.77), 7.390 (1.22), 7.395 (1.80), |
| | 7.405 (3.48), 7.409 (1.07), 7.415 (0.91), 7.419 (0.96), 7.426 (2.64), |
| | 7.430 (1.89), 7.440 (1.84), 7.445 (1.73), 7.652 (1.75), 8.270 (2.26), |
| | 8.283 (2.11), 10.843 (2.18). |
| 29 | 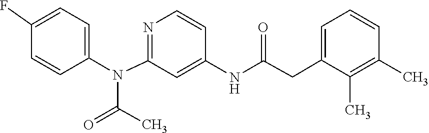 [SMILES: CC(=O)N(c1ccc(F)cc1)c1cc(NC(=O)Cc2cccc(C)c2C)ccn1] |
| | N-{4-[2-(2,3-dimethylphenyl)acetamido]pyridin-2-yl}-N-(4- |
| | fluorophenyl)acetamide |
| | L LC-MS (Method 1): Rₜ = 1.18 min; MS (ESIpos): m/z = 392 [M + H]⁺ |
| | ¹H-NMR (400 MHz, DMSO-d6) δ [ppm]: 1.011 (0.32), 1.029 (0.70), |
| | 1.047 (0.32), 1.090 (0.28), 1.108 (0.63), 1.126 (0.27), 1.981 (16.00), |
| | 2.044 (1.01), 2.077 (0.23), 2.139 (11.41), 2.212 (0.23), 2.234 (11.07), |
| | 2.308 (0.46), 2.332 (0.48), 2.336 (0.21), 2.518 (2.41), 2.523 (1.66), |
| | 2.530 (0.66), 2.673 (0.49), 2.678 (0.21), 3.271 (0.26), 3.288 (0.27), |
| | 3.349 (0.34), 3.639 (0.65), 3.730 (6.84), 6.980 (0.21), 6.993 (0.40), |
| | 6.998 (0.18), 7.012 (0.76), 7.017 (0.60), 7.028 (2.54), 7.043 (3.80), |
| | 7.057 (0.94), 7.062 (0.69), 7.067 (0.69), 7.209 (1.74), 7.214 (0.62), |
| | 7.226 (0.79), 7.231 (3.92), 7.236 (0.77), 7.240 (0.42), 7.247 (0.72), |
| | 7.253 (2.85), 7.261 (0.32), 7.299 (0.28), 7.307 (2.27), 7.312 (0.86), |
| | 7.319 (2.39), 7.324 (1.00), 7.329 (1.61), 7.336 (0.60), 7.342 (1.41), |
| | 7.458 (1.64), 7.463 (1.77), 7.472 (1.69), 7.476 (1.71), 7.678 (1.56), |
| | 7.681 (1.55), 8.250 (2.15), 8.264 (2.02), 10.293 (0.23), 10.639 (2.18). |
| 30 | 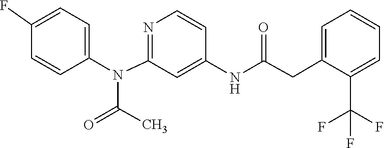 [SMILES: CC(=O)N(c1ccc(F)cc1)c1cc(NC(=O)Cc2ccccc2C(F)(F)F)ccn1] |
| | N-(4-fluorophenyl)-N-(4-{2-[2- |
| | (trifluoromethyl)phenyl]acetamido}pyridin-2-yl)acetamide |
| | LC-MS (Method 1): Rₜ = 1.16 min; MS (ESIpos): m/z = 432 [M + H]⁺ |
| | ¹H-NMR (400 MHz, DMSO-d6) δ [ppm]: 0.994 (0.27), 1.012 (0.62), |
| | 1.030 (0.27), 1.130 (0.23), 1.148 (0.55), 1.166 (0.24), 1.983 (16.00), |
| | 2.331 (0.17), 2.518 (0.95), 2.523 (0.60), 2.673 (0.17), 3.261 (0.22), |
| | 3.278 (0.22), 3.355 (0.23), 3.373 (0.21), 3.839 (0.27), 3.960 (3.41), |
| | 7.211 (1.83), 7.217 (0.66), 7.228 (0.81), 7.234 (3.91), 7.238 (0.80), |
| | 7.242 (0.44), 7.249 (0.76), 7.255 (2.87), 7.264 (0.31), 7.303 (0.28), |
| | 7.312 (2.24), 7.317 (0.88), 7.324 (2.40), 7.330 (0.99), 7.334 (1.68), |
| | 7.341 (0.62), 7.346 (1.43), 7.431 (1.74), 7.436 (1.82), 7.445 (1.74), |
| | 7.450 (1.91), 7.479 (0.55), 7.498 (2.48), 7.518 (2.44), 7.633 (0.86), |
| | 7.650 (1.27), 7.675 (1.73), 7.702 (1.44), 7.721 (1.25), 8.256 (2.20), |
| | 8.270 (2.10), 10.731 (2.26). |
| 31 | 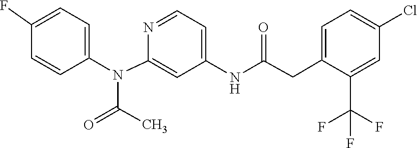 [SMILES: CC(=O)N(c1ccc(F)cc1)c1cc(NC(=O)Cc2ccc(Cl)cc2C(F)(F)F)ccn1] |
| | N-(4-{2-[4-chloro-2-(trifluoromethyl)phenyl]acetamido}pyridin-2- |
| | yl)-N-(4-fluorophenyl)acetamide |
| | LC-MS (Method 2): Rₜ = 1.23 min; MS (ESIpos): m/z = 466 [M + H]⁺ |
| | ¹H-NMR (400 MHz, DMSO-d6) δ [ppm]: 1.981 (16.00), 2.518 (2.06), |
| | 2.522 (1.43), 3.967 (3.64), 7.212 (1.87), 7.218 (0.71), 7.229 (0.92), |
| | 7.235 (4.24), 7.239 (0.93), 7.243 (0.54), 7.250 (0.83), 7.256 (3.04), |
| | 7.310 (2.44), 7.316 (1.03), 7.323 (2.64), 7.328 (1.17), 7.333 (1.87), |
| | 7.340 (0.75), 7.346 (1.61), 7.419 (1.61), 7.424 (1.63), 7.433 (1.63), |
| | 7.438 (1.64), 7.547 (1.56), 7.567 (1.88), 7.667 (1.79), 7.747 (1.09), |
| | 7.752 (1.44), 7.773 (1.62), 7.779 (3.06), 7.784 (1.83), 8.255 (2.34), |
| | 8.269 (2.24), 10.744 (2.47). |
| 32 | 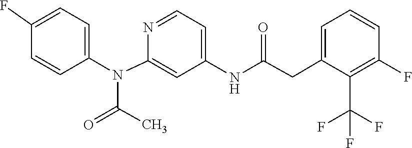 [SMILES: CC(=O)N(c1ccc(F)cc1)c1cc(NC(=O)Cc2cccc(F)c2C(F)(F)F)ccn1] |
| | N-(4-fluorophenyl)-N-(4-{2-[3-fluoro-2- |
| | (trifluoromethyl)phenyl]acetamido}pyridin-2-yl)acetamide |
| | LC-MS (Method 1): Rₜ = 1.15 min; MS (ESIpos): m/z = 450 [M + H]⁺ |
| | ¹H-NMR (400 MHz, DMSO-d6) δ [ppm]: 1.984 (16.00), 2.518 (0.89), |
| | 2.522 (0.58), 4.033 (2.59), 4.039 (2.53), 7.212 (1.84), 7.218 (0.69), |
| | 7.229 (0.88), 7.235 (4.17), 7.240 (0.85), 7.243 (0.47), 7.251 (0.78), |
| | 7.256 (2.93), 7.307 (1.48), 7.314 (2.60), 7.320 (1.22), 7.327 (3.86), |
| | 7.337 (1.80), 7.344 (0.68), 7.350 (1.51), 7.398 (0.68), 7.419 (0.94), |
| | 7.426 (2.08), 7.431 (1.80), 7.440 (1.51), 7.445 (1.91), 7.657 (2.16), |
| | 7.670 (0.76), 7.676 (0.94), 7.691 (0.88), 8.254 (2.26), 8.268 (2.12), |
| | 10.726 (2.22). |
| 33 | 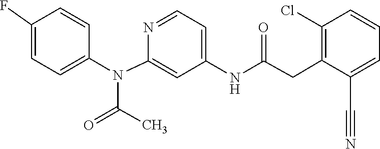 [SMILES: CC(=O)N(c1ccc(F)cc1)c1cc(NC(=O)Cc2c(Cl)cccc2C#N)ccn1] |
| | N-{4-[2-(2-chloro-6-cyanophenyl)acetamido]pyridin-2-yl}-N-(4- |
| | fluorophenyl)acetamide |
| | ¹H NMR (500 MHZ, DMSO-d6) δ [ppm]: 1.99 (s, 3H), 4.13 (s, 2H), |
| | 7.21-7.26 (m, 2H), 7.32-7.38 (m, 2H), 7.45 (dd, J = 5.7, 1.9, 1H), 7.50- |
| | 7.57 (m, 1H), 7.69 (s, 1H), 7.85 (dd, J = 8.2, 1.1, 1H), 7.88 (dd, J = 7.7, |
| | 1.1, 1H), 8.29 (d, J = 5.6, 1H), 10.99 (s, 1H). |
| 34 | 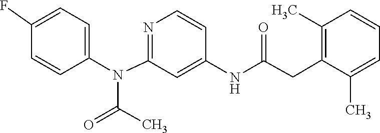 [SMILES: CC(=O)N(c1ccc(F)cc1)c1cc(NC(=O)Cc2c(C)cccc2C)ccn1] |
| | N-{4-[2-(2,6-dimethylphenyl)acetamido]pyridin-2-yl}-N-(4- |
| | fluorophenyl)acetamide |
| | LC-MS (Method 1): Rₜ = 1.18 min; MS (ESIpos): m/z = 392 [M + H]⁺ |
| | ¹H-NMR (400 MHz, DMSO-d6) δ [ppm]: 1.107 (10.95), 1.980 (10.74), |
| | 2.235 (16.00), 2.332 (0.31), 2.518 (1.57), 2.523 (1.02), 2.539 (1.50), |
| | 2.673 (0.32), 3.772 (4.37), 4.190 (0.89), 6.989 (0.57), 6.997 (0.70), |
| | 7.012 (3.14), 7.026 (2.23), 7.038 (0.78), 7.048 (0.52), 7.061 (0.31), |
| | 7.209 (1.19), 7.214 (0.44), 7.225 (0.54), 7.231 (2.59), 7.236 (0.51), |
| | 7.240 (0.28), 7.247 (0.50), 7.253 (1.83), 7.261 (0.20), 7.300 (0.19), |
| | 7.309 (1.47), 7.314 (0.57), 7.321 (1.58), 7.327 (0.63), 7.331 (1.05), |
| | 7.338 (0.39), 7.344 (0.93), 7.447 (1.13), 7.451 (1.09), 7.461 (1.12), |
| | 7.466 (1.13), 7.687 (1.04), 7.691 (1.00), 8.254 (1.44), 8.268 (1.36), |
| | 10.715 (1.44). |
| 35 | 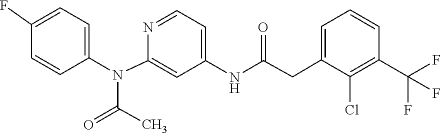 [SMILES: CC(=O)N(c1ccc(F)cc1)c1cc(NC(=O)Cc2cccc(C(F)(F)F)c2Cl)ccn1] |
| | N-(4-{2-[2-chloro-3-(trifluoromethyl)phenyl]acetamido}pyridin-2- |
| | yl)-N-(4-fluorophenyl)acetamide |
| | LC-MS (Method 2): Rₜ = 1.19 min; MS (ESIpos): m/z = 466 [M + H]⁺ |
| | ¹H-NMR (400 MHZ, DMSO-d6) δ [ppm]: 1.984 (16.00), 2.014 (2.14), |
| | 2.518 (0.82), 2.522 (0.54), 4.022 (6.96), 7.212 (1.84), 7.218 (0.69), |
| | 7.229 (0.89), 7.234 (4.38), 7.239 (0.86), 7.243 (0.47), 7.250 (0.81), |
| | 7.256 (2.92), 7.264 (0.46), 7.286 (0.43), 7.307 (0.52), 7.314 (2.65), |
| | 7.319 (1.08), 7.326 (2.59), 7.332 (1.21), 7.336 (1.99), 7.343 (0.70), |
| | 7.349 (1.59), 7.437 (1.79), 7.442 (1.88), 7.452 (1.77), 7.456 (1.88), |
| | 7.524 (0.83), 7.543 (1.84), 7.563 (1.07), 7.694 (1.83), 7.698 (1.88), |
| | 7.738 (1.51), 7.757 (1.18), 7.793 (1.55), 7.796 (1.44), 7.813 (1.35), |
| | 7.816 (1.20), 8.263 (2.44), 8.277 (2.31), 10.834 (2.61). |
| 36 | 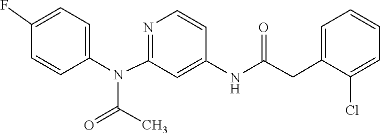 [SMILES: CC(=O)N(c1ccc(F)cc1)c1cc(NC(=O)Cc2ccccc2Cl)ccn1] |
| | N-{4-[2-(2-chlorophenyl)acetamido]pyridin-2-yl}-N-(4- |
| | fluorophenyl)acetamide |
| | LC-MS (Method 1): Rₜ = 0.81 min; MS (ESIpos): m/z = 398 [M + H]⁺ |
| | ¹H-NMR (400 MHZ, DMSO-d6) δ [ppm]: 1.986 (16.00), 2.518 (1.96), |
| | 2.522 (1.23), 3.873 (7.55), 5.759 (1.67), 7.212 (1.74), 7.218 (0.70), |
| | 7.229 (0.93), 7.234 (4.08), 7.240 (0.86), 7.243 (0.49), 7.250 (0.85), |
| | 7.256 (2.86), 7.264 (0.42), 7.291 (0.45), 7.301 (3.19), 7.311 (3.99), |
| | 7.316 (3.25), 7.324 (6.59), 7.334 (2.12), 7.341 (0.68), 7.347 (1.43), |
| | 7.386 (0.45), 7.395 (1.65), 7.405 (1.17), 7.409 (1.21), 7.417 (1.02), |
| | 7.424 (0.46), 7.434 (1.83), 7.443 (1.63), 7.448 (1.29), 7.452 (2.10), |
| | 7.457 (2.57), 7.466 (1.81), 7.471 (1.81), 7.681 (1.71), 8.260 (2.25), |
| | 8.273 (2.12), 10.749 (2.32). |
| 37 | 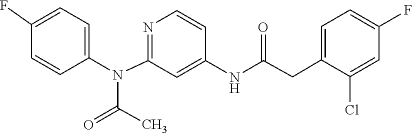 [SMILES: CC(=O)N(c1ccc(F)cc1)c1cc(NC(=O)Cc2ccc(F)cc2Cl)ccn1] |
| | N-{4-[2-(2-chloro-4-fluorophenyl)acetamido]pyridin-2-yl}-N-(4- |
| | fluorophenyl)acetamide |
| | LC-MS (Method 1): Rₜ = 1.12 min; MS (ESIpos): m/z = 416 [M + H]⁺ |
| | ¹H-NMR (400 MHz, DMSO-d6) δ [ppm]: 1.154 (0.49), 1.172 (0.96), |
| | 1.189 (0.46), 1.985 (16.00), 2.518 (1.16), 2.523 (0.69), 2.727 (8.49), |
| | 2.729 (8.55), 2.888 (10.69), 3.865 (6.13), 4.017 (0.40), 5.758 (0.59), |
| | 7.186 (0.75), 7.193 (0.82), 7.207 (1.61), 7.214 (2.83), 7.218 (0.83), |
| | 7.229 (1.55), 7.235 (4.83), 7.240 (0.90), 7.243 (0.50), 7.250 (0.79), |
| | 7.257 (2.86), 7.311 (2.29), 7.317 (0.94), 7.324 (2.47), 7.329 (1.03), |
| | 7.333 (1.64), 7.341 (0.64), 7.346 (1.42), 7.429 (1.57), 7.436 (1.73), |
| | 7.443 (3.13), 7.448 (2.10), 7.452 (1.92), 7.459 (4.02), 7.462 (2.48), |
| | 7.481 (1.13), 7.678 (1.65), 7.950 (1.30), 8.259 (2.22), 8.273 (2.11), |
| | 10.752 (2.30). |
| 38 | 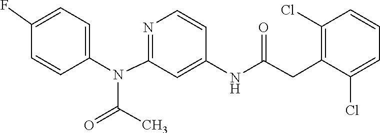 [SMILES: CC(=O)N(c1ccc(F)cc1)c1cc(NC(=O)Cc2c(Cl)cccc2Cl)ccn1] |
| | N-{4-[2-(2,6-dichlorophenyl)acetamido]pyridin-2-yl}-N-(4- |
| | fluorophenyl)acetamide |
| | LC-MS (Method 2): Rₜ = 1.15 min; MS (ESIpos): m/z = 432 [M + H]⁺ |
| | ¹H-NMR (400 MHZ, DMSO-d6) δ [ppm]: 1.172 (0.62), 1.984 (16.00), |
| | 2.518 (3.36), 2.523 (2.16), 4.076 (7.64), 5.759 (5.64), 7.212 (1.76), |
| | 7.218 (0.68), 7.229 (0.90), 7.235 (4.17), 7.250 (0.81), 7.256 (2.90), |
| | 7.316 (2.33), 7.321 (1.03), 7.329 (2.52), 7.335 (2.56), 7.338 (1.89), |
| | 7.353 (2.63), 7.356 (2.29), 7.374 (2.38), 7.418 (1.74), 7.423 (1.78), |
| | 7.432 (1.77), 7.437 (1.79), 7.486 (6.72), 7.506 (4.18), 7.682 (1.71), |
| | 8.264 (2.28), 8.278 (2.19), 10.862 (2.43). |
| 39 | 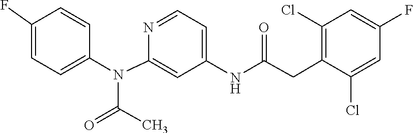 [SMILES: CC(=O)N(c1ccc(F)cc1)c1cc(NC(=O)Cc2c(Cl)cc(F)cc2Cl)ccn1] |
| | N-{4-[2-(2,6-dichloro-4-fluorophenyl)acetamido]pyridin-2-yl}-N-(4- |
| | fluorophenyl)acetamide |
| | ¹H NMR (500 MHz, DMSO-d6) δ [ppm]: 1.98 (s, 3H), 4.04 (s, 2H), |
| | 7.20-7.28 (m, 2H), 7.29-7.38 (m, 2H), 7.43 (dd, J = 5.6, 1.9, 1H), 7.56 (d, |
| | J = 8.5, 2H), 7.68 (s, 1H), 8.27 (d, J= 5.6, 1H), 10.84 (s, 1H). |
| 40 | 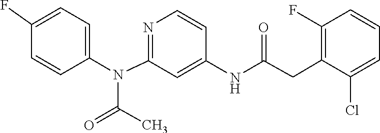 [SMILES: CC(=O)N(c1ccc(F)cc1)c1cc(NC(=O)Cc2c(F)cccc2Cl)ccn1] |
| | N-{4-[2-(2-chloro-6-fluorophenyl)acetamido]pyridin-2-yl}-N-(4- |
| | fluorophenyl)acetamide |
| | LC-MS (Method 1): Rₜ = 0.85 min; MS (ESIpos): m/z = 416 [M + H]⁺ |
| | ¹H-NMR (400 MHZ, DMSO-d6) δ [ppm]: 1.984 (16.00), 2.518 (2.98), |
| | 2.522 (1.92), 3.919 (4.37), 3.923 (4.16), 5.759 (5.50), 7.212 (1.82), |
| | 7.217 (0.71), 7.226 (0.94), 7.234 (4.15), 7.239 (0.96), 7.244 (1.19), |
| | 7.250 (2.12), 7.256 (3.02), 7.264 (0.45), 7.269 (0.95), 7.272 (0.94), |
| | 7.314 (2.24), 7.319 (0.95), 7.326 (2.41), 7.332 (1.10), 7.336 (2.35), |
| | 7.341 (1.18), 7.349 (1.62), 7.357 (3.49), 7.362 (3.01), 7.376 (1.33), |
| | 7.381 (1.21), 7.396 (1.33), 7.402 (0.45), 7.417 (0.51), 7.424 (1.79), |
| | 7.428 (1.71), 7.438 (1.74), 7.443 (1.78), 7.682 (1.66), 8.263 (2.24), |
| | 8.277 (2.14), 10.851 (2.27). |
| 41 | 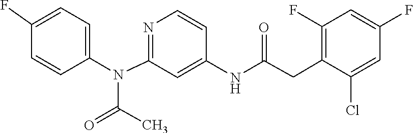 [SMILES: CC(=O)N(c1ccc(F)cc1)c1cc(NC(=O)Cc2c(F)cc(F)cc2Cl)ccn1] |
| | N-{4-[2-(2-chloro-4,6-difluorophenyl)acetamido]pyridin-2-yl}-N-(4- |
| | fluorophenyl)acetamide |
| | LC-MS (Method 1): Rₜ = 1.16 min; MS (ESIpos): m/z = 434 [M]⁺ |
| | ¹H-NMR (400 MHz, DMSO-d6) δ [ppm]: 1.983 (16.00), 2.518 (1.49), |
| | 2.523 (0.95), 3.887 (3.83), 3.890 (3.78), 7.212 (1.79), 7.218 (0.65), |
| | 7.230 (0.83), 7.235 (4.09), 7.240 (0.83), 7.244 (0.45), 7.251 (0.77), |
| | 7.257 (3.08), 7.314 (2.25), 7.320 (0.97), 7.327 (2.41), 7.331 (1.03), |
| | 7.336 (1.75), 7.344 (1.16), 7.350 (2.02), 7.367 (0.90), 7.374 (1.17), |
| | 7.391 (0.58), 7.397 (0.97), 7.404 (1.10), 7.408 (1.01), 7.414 (0.84), |
| | 7.419 (1.94), 7.425 (2.41), 7.433 (2.22), 7.438 (2.09), 7.681 (1.63), |
| | 8.263 (2.25), 8.277 (2.14), 10.863 (2.14). |
| 42 | 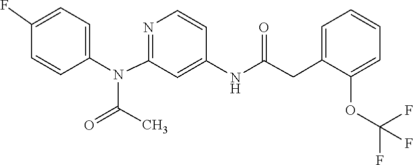 [SMILES: CC(=O)N(c1ccc(F)cc1)c1cc(NC(=O)Cc2ccccc2OC(F)(F)F)ccn1] |
| | N-(4-fluorophenyl)-N-(4-{2-[2- |
| | (trifluoromethoxy)phenyl]acetamido}pyridin-2-yl)acetamide |
| | LC-MS (Method 1): Rₜ = 1.18 min; MS (ESIpos): m/z = 448 [M + H]⁺ |
| | ¹H-NMR (400 MHz, DMSO-d6) δ [ppm]: 1.012 (0.26), 1.127 (0.25), |
| | 1.984 (16.00), 2.518 (1.36), 2.522 (0.83), 3.710 (0.28), 3.819 (6.90), |
| | 7.212 (1.85), 7.218 (0.68), 7.229 (0.83), 7.235 (4.12), 7.240 (0.83), |
| | 7.244 (0.45), 7.250 (0.79), 7.257 (2.88), 7.265 (0.32), 7.304 (0.30), |
| | 7.312 (2.38), 7.317 (0.99), 7.324 (2.57), 7.330 (1.20), 7.335 (2.29), |
| | 7.342 (1.56), 7.347 (2.26), 7.354 (0.99), 7.358 (1.46), 7.362 (2.80), |
| | 7.380 (1.83), 7.383 (1.10), 7.400 (1.30), 7.405 (1.72), 7.419 (0.99), |
| | 7.425 (1.50), 7.439 (0.56), 7.445 (2.15), 7.449 (1.88), 7.459 (2.80), |
| | 7.463 (2.64), 7.476 (1.15), 7.480 (0.83), 7.675 (1.64), 8.257 (2.28), |
| | 8.271 (2.15), 10.736 (2.30). |
| 43 | 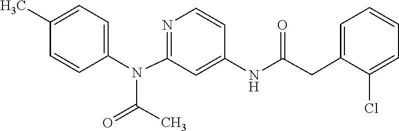 [SMILES: CC(=O)N(c1ccc(C)cc1)c1cc(NC(=O)Cc2ccccc2Cl)ccn1] |
| | N-{4-[2-(2-chlorophenyl)acetamido]pyridin-2-yl}-N-(4- |
| | methylphenyl)acetamide |
| | LC-MS (Method 2): Rt = 1.10 min; MS (ESIneg): m/z = 392 [M − H]⁻ |
| | ¹H-NMR (400 MHz, DMSO-d6) δ [ppm]: 1.975 (16.00), 2.301 (10.88), |
| | 2.518 (3.41), 2.523 (2.15), 3.866 (7.50), 7.131 (2.45), 7.147 (1.44), |
| | 7.151 (5.05), 7.191 (3.97), 7.211 (2.00), 7.299 (3.32), 7.308 (2.52), |
| | 7.314 (2.57), 7.323 (4.22), 7.333 (0.58), 7.391 (1.66), 7.401 (1.06), |
| | 7.405 (1.21), 7.414 (1.03), 7.422 (0.49), 7.433 (2.20), 7.435 (2.12), |
| | 7.440 (2.51), 7.449 (2.21), 7.454 (2.32), 7.641 (1.87), 7.644 (1.83), |
| | 8.244 (2.42), 8.257 (2.32), 10.724 (2.31). |
| 44 | 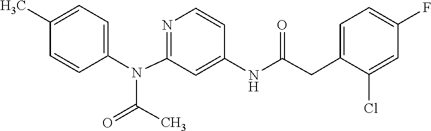 [SMILES: CC(=O)N(c1ccc(C)cc1)c1cc(NC(=O)Cc2ccc(F)cc2Cl)ccn1] |
| | N-{4-[2-(2-chloro-4-fluorophenyl)acetamido]pyridin-2-yl}-N-(4- |
| | methylphenyl)acetamide |
| | LC-MS (Method 2): Rₜ = 1.13 min; MS (ESIpos): m/z = 412 [M + H]⁺ |
| | ¹H-NMR (400 MHZ, DMSO-d6) δ [ppm]: 1.974 (16.00), 2.301 (10.75), |
| | 2.518 (1.66), 2.522 (1.07), 3.806 (0.58), 3.858 (6.18), 7.131 (2.47), |
| | 7.135 (1.00), 7.146 (1.51), 7.151 (5.04), 7.184 (1.37), 7.191 (4.79), |
| | 7.205 (2.16), 7.212 (3.59), 7.227 (0.95), 7.233 (0.95), 7.428 (2.80), |
| | 7.433 (2.27), 7.436 (2.04), 7.441 (2.55), 7.447 (1.99), 7.451 (1.92), |
| | 7.457 (2.25), 7.461 (1.37), 7.477 (1.13), 7.637 (1.89), 7.641 (1.84), |
| | 8.243 (2.39), 8.257 (2.26), 10.721 (2.36). |
| 45 | 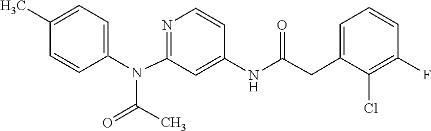 [SMILES: CC(=O)N(c1ccc(C)cc1)c1cc(NC(=O)Cc2cccc(F)c2Cl)ccn1] |
| | N-{4-[2-(2-chloro-3-fluorophenyl)acetamido]pyridin-2-yl}-N-(4- |
| | methylphenyl)acetamide |
| | LC-MS (Method 2): Rₜ = 1.12 min; MS (ESIpos): m/z = 412 [M + H]⁺ |
| | ¹H-NMR (400 MHZ, DMSO-d6) δ [ppm]: 1.975 (16.00), 2.301 (10.84), |
| | 2.518 (1.14), 2.523 (0.74), 3.930 (6.96), 7.132 (2.49), 7.137 (0.97), |
| | 7.148 (1.38), 7.153 (5.14), 7.192 (3.99), 7.212 (2.04), 7.256 (0.77), |
| | 7.263 (1.12), 7.269 (0.80), 7.274 (0.88), 7.279 (1.18), 7.322 (0.48), |
| | 7.336 (1.88), 7.340 (2.29), 7.357 (4.52), 7.364 (1.35), 7.372 (1.24), |
| | 7.429 (1.75), 7.434 (1.75), 7.443 (1.64), 7.448 (1.81), 7.640 (1.88), |
| | 7.644 (1.81), 8.246 (2.44), 8.260 (2.28), 10.762 (2.33). |
| 46 | 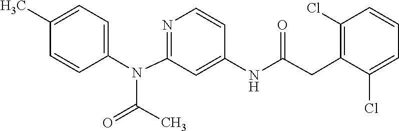 [SMILES: CC(=O)N(c1ccc(C)cc1)c1cc(NC(=O)Cc2c(Cl)cccc2Cl)ccn1] |
| | N-{4-[2-(2,6-dichlorophenyl)acetamido]pyridin-2-yl}-N-(4- |
| | methylphenyl)acetamide |
| | LC-MS (Method 2): Rₜ = 1.16 min; MS (ESIpos): m/z = 429 [M + H]⁺ |
| | ¹H-NMR (400 MHZ, DMSO-d6) δ [ppm]: 1.973 (16.00), 2.301 (10.94), |
| | 2.518 (2.42), 2.522 (1.55), 4.016 (0.53), 4.069 (7.37), 7.134 (2.50), |
| | 7.139 (0.94), 7.150 (1.33), 7.155 (5.20), 7.191 (4.08), 7.212 (2.05), |
| | 7.333 (1.48), 7.351 (1.97), 7.355 (2.10), 7.373 (2.43), 7.400 (1.69), |
| | 7.405 (1.74), 7.414 (1.72), 7.419 (1.70), 7.466 (0.58), 7.485 (6.85), |
| | 7.504 (4.04), 7.641 (1.82), 7.645 (1.77), 8.247 (2.41), 8.261 (2.28), |
| | 10.837 (2.35). |
| 47 | 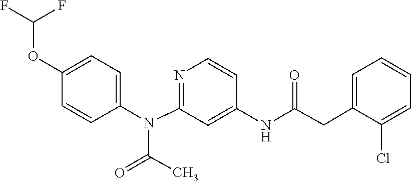 [SMILES: CC(=O)N(c1ccc(OC(F)F)cc1)c1cc(NC(=O)Cc2ccccc2Cl)ccn1] |
| | N-{4-[2-(2-chlorophenyl)acetamido]pyridin-2-yl}-N-[4- |
| | (difluoromethoxy)phenyl]acetamide |
| | LC-MS (Method 2): Rₜ = 1.12 min; MS (ESIpos): m/z = 446 [M + H]⁺ |
| | ¹H-NMR (500 MHz, DMSO-d6) δ [ppm]: 1.990 (16.00), 2.514 (2.04), |
| | 2.518 (2.06), 2.522 (1.64), 3.874 (8.51), 7.114 (1.80), 7.190 (3.62), |
| | 7.194 (1.11), 7.203 (1.31), 7.207 (4.61), 7.214 (0.43), 7.262 (3.86), |
| | 7.303 (3.53), 7.311 (2.32), 7.316 (2.84), 7.322 (9.41), 7.327 (1.44), |
| | 7.336 (1.20), 7.340 (3.35), 7.396 (1.71), 7.402 (0.85), 7.404 (1.10), |
| | 7.408 (1.40), 7.410 (1.90), 7.414 (1.20), 7.436 (1.98), 7.443 (1.68), |
| | 7.447 (1.06), 7.455 (1.56), 7.458 (1.98), 7.462 (2.04), 7.470 (1.88), |
| | 7.474 (1.96), 7.678 (1.53), 8.268 (2.20), 8.280 (2.13), 10.754 (2.65). |
| 48 | 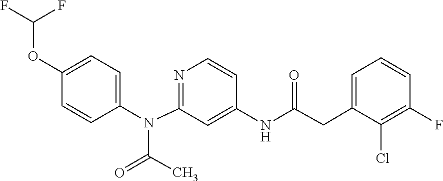 [SMILES: CC(=O)N(c1ccc(OC(F)F)cc1)c1cc(NC(=O)Cc2cccc(F)c2Cl)ccn1] |
| | N-{4-[2-(2-chloro-3-fluorophenyl)acetamido]pyridin-2-yl}-N-[4- |
| | (difluoromethoxy)phenyl]acetamide |
| | LC-MS (Method 2): Rₜ = 1.13 min; MS (ESIpos): m/z = 464 [M + H]⁺ |
| | ¹H-NMR (500 MHz, DMSO-d6) δ [ppm]: 1.990 (16.00), 2.514 (1.80), |
| | 2.518 (1.80), 2.522 (1.46), 3.937 (8.12), 7.115 (1.75), 7.191 (3.70), |
| | 7.195 (1.13), 7.204 (1.35), 7.209 (4.71), 7.215 (0.44), 7.263 (4.47), |
| | 7.267 (1.37), 7.273 (0.93), 7.276 (1.01), 7.280 (1.31), 7.317 (0.52), |
| | 7.323 (4.80), 7.328 (1.82), 7.341 (4.23), 7.345 (3.44), 7.358 (3.25), |
| | 7.362 (1.64), 7.371 (1.26), 7.375 (0.45), 7.411 (1.66), 7.452 (1.83), |
| | 7.456 (1.86), 7.463 (1.78), 7.467 (1.86), 7.678 (1.57), 8.271 (2.27), |
| | 8.282 (2.15), 10.791 (2.74). |
| 49 | 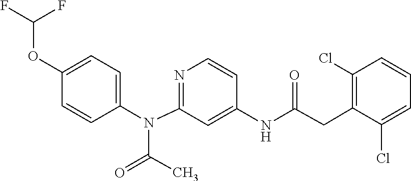 [SMILES: CC(=O)N(c1ccc(OC(F)F)cc1)c1cc(NC(=O)Cc2c(Cl)cccc2Cl)ccn1] |
| | N-{4-[2-(2,6-dichlorophenyl)acetamido]pyridin-2-yl}-N-[4- |
| | (difluoromethoxy)phenyl]acetamide |
| | LC-MS (Method 2): Rₜ = 1.17 min; MS (ESIpos): m/z = 481 [M + H]⁺ |
| | ¹H-NMR (400 MHZ, DMSO-d6) δ [ppm]: 1.988 (16.00), 2.518 (9.92), |
| | 2.523 (7.01), 4.076 (7.76), 7.079 (1.99), 7.188 (3.36), 7.193 (1.27), |
| | 7.204 (1.35), 7.210 (4.67), 7.218 (0.70), 7.264 (3.83), 7.317 (0.53), |
| | 7.325 (4.93), 7.331 (1.67), 7.334 (2.13), 7.342 (1.39), 7.348 (3.73), |
| | 7.353 (2.57), 7.356 (2.78), 7.375 (2.77), 7.424 (1.82), 7.428 (1.85), |
| | 7.438 (1.92), 7.443 (1.98), 7.448 (2.06), 7.486 (7.11), 7.506 (4.32), |
| | 7.681 (1.79), 8.271 (2.35), 8.285 (2.29), 10.870 (2.60). |
| 50 | 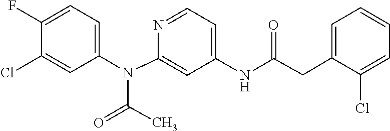 [SMILES: CC(=O)N(c1ccc(F)c(Cl)c1)c1cc(NC(=O)Cc2ccccc2Cl)ccn1] |
| | N-(3-chloro-4-fluorophenyl)-N-{4-[2-(2- |
| | chlorophenyl)acetamido]pyridin-2-yl}acetamide |
| | LC-MS (Method 2): Rₜ = 1.18 min; MS (ESIpos): m/z = 432 [M + H]⁺ |
| | ¹H-NMR (400 MHz, DMSO-d6) δ [ppm]: 2.005 (16.00), 2.518 (0.42), |
| | 3.337 (1.52), 7.275 (0.51), 7.281 (0.60), 7.285 (0.64), 7.291 (0.85), |
| | 7.297 (0.88), 7.302 (3.87), 7.309 (2.19), 7.311 (2.38), 7.315 (2.43), |
| | 7.317 (2.22), 7.325 (4.18), 7.334 (0.41), 7.336 (0.48), 7.398 (1.58), |
| | 7.405 (0.78), 7.409 (1.00), 7.413 (1.04), 7.421 (1.04), 7.430 (1.86), |
| | 7.435 (1.91), 7.444 (1.50), 7.448 (1.25), 7.452 (3.29), 7.458 (1.39), |
| | 7.475 (1.30), 7.485 (1.53), 7.489 (1.58), 7.499 (1.55), 7.503 (1.56), |
| | 7.622 (1.19), 7.629 (1.19), 7.639 (1.21), 7.646 (1.14), 7.706 (1.44), |
| | 8.272 (1.96), 8.286 (1.86), 10.771 (2.20). |
| 51 | 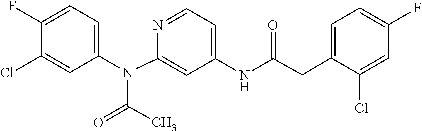 [SMILES: CC(=O)N(c1ccc(F)c(Cl)c1)c1cc(NC(=O)Cc2ccc(F)cc2Cl)ccn1] |
| | N-(3-chloro-4-fluorophenyl)-N-{4-[2-(2-chloro-4- |
| | fluorophenyl)acetamido]pyridin-2-yl}acetamide |
| | LC-MS (Method 2): Rₜ = 1.21 min; MS (ESIpos): m/z = 450 [M + H]⁺ |
| | ¹H-NMR (400 MHZ, DMSO-d6) δ [ppm]: 2.004 (16.00), 2.518 (0.42), |
| | 3.339 (2.08), 7.186 (0.72), 7.193 (0.81), 7.208 (1.49), 7.214 (1.64), |
| | 7.229 (0.81), 7.236 (0.92), 7.275 (0.47), 7.281 (0.57), 7.285 (0.57), |
| | 7.291 (0.58), 7.296 (0.72), 7.303 (0.76), 7.307 (0.69), 7.314 (0.65), |
| | 7.430 (3.24), 7.436 (1.63), 7.447 (1.54), 7.452 (4.16), 7.458 (1.78), |
| | 7.463 (1.53), 7.468 (1.36), 7.475 (2.87), 7.480 (1.91), 7.484 (1.38), |
| | 7.490 (1.67), 7.494 (1.74), 7.622 (1.17), 7.628 (1.18), 7.639 (1.21), |
| | 7.645 (1.15), 7.703 (1.43), 8.271 (2.01), 8.285 (1.90), 10.768 (2.32). |
| 52 | 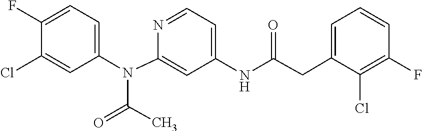 [SMILES: CC(=O)N(c1ccc(F)c(Cl)c1)c1cc(NC(=O)Cc2cccc(F)c2Cl)ccn1] |
| | N-(3-chloro-4-fluorophenyl)-N-{4-[2-(2-chloro-3- |
| | fluorophenyl)acetamido]pyridin-2-yl}acetamide |
| | LC-MS (Method 2): Rₜ = 1.20 min; MS (ESIpos): m/z = 450 [M + H]⁺ |
| | ¹H-NMR (400 MHZ, DMSO-d6) δ [ppm]: 2.005 (16.00), 3.943 (7.72), |
| | 7.263 (0.91), 7.270 (1.34), 7.276 (1.51), 7.287 (2.07), 7.293 (0.87), |
| | 7.298 (1.03), 7.305 (1.03), 7.309 (0.95), 7.316 (0.92), 7.322 (0.56), |
| | 7.337 (1.91), 7.343 (2.79), 7.359 (4.27), 7.366 (1.60), 7.374 (1.33), |
| | 7.380 (0.47), 7.431 (1.82), 7.453 (3.02), 7.476 (2.78), 7.482 (1.89), |
| | 7.491 (1.77), 7.495 (1.88), 7.624 (1.34), 7.630 (1.38), 7.641 (1.38), |
| | 7.647 (1.31), 7.706 (1.80), 8.274 (2.29), 8.288 (2.20), 10.808 (2.70). |
| 53 | 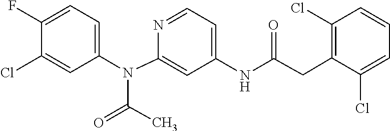 [SMILES: CC(=O)N(c1ccc(F)c(Cl)c1)c1cc(NC(=O)Cc2c(Cl)cccc2Cl)ccn1] |
| | N-(3-chloro-4-fluorophenyl)-N-{4-[2-(2,6- |
| | dichlorophenyl)acetamido]pyridin-2-yl}acetamide |
| | LC-MS (Method 2): Rₜ = 1.20 min; MS (ESIpos): m/z = 466 [M + H]⁺ |
| | ¹H-NMR (400 MHz, DMSO-d6) δ [ppm]: 2.002 (16.00), 4.083 (6.98), |
| | 7.280 (0.48), 7.287 (0.57), 7.291 (0.59), 7.297 (0.62), 7.302 (0.73), |
| | 7.309 (0.77), 7.312 (0.72), 7.319 (0.68), 7.333 (1.50), 7.352 (1.96), |
| | 7.355 (2.07), 7.374 (2.50), 7.429 (1.73), 7.451 (4.09), 7.456 (1.66), |
| | 7.466 (1.58), 7.471 (1.79), 7.473 (1.63), 7.485 (6.66), 7.505 (3.99), |
| | 7.627 (1.20), 7.633 (1.20), 7.644 (1.19), 7.650 (1.13), 7.708 (1.39), |
| | 8.276 (1.99), 8.290 (1.88), 10.885 (2.30). |
| 54 | 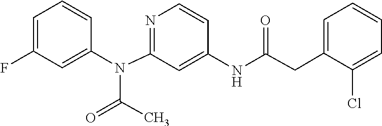 [SMILES: CC(=O)N(c1cccc(F)c1)c1cc(NC(=O)Cc2ccccc2Cl)ccn1] |
| | N-{4-[2-(2-chlorophenyl)acetamido]pyridin-2-yl}-N-(3- |
| | fluorophenyl)acetamide |
| | LC-MS (Method 1): Rₜ = 1.09 min; MS (ESIpos): m/z = 398 [M + H]⁺ |
| | ¹H-NMR (400 MHZ, DMSO-d6) δ [ppm]: 1.232 (0.42), 1.988 (1.61), |
| | 2.007 (16.00), 2.518 (4.82), 2.523 (3.23), 3.875 (6.91), 5.759 (3.23), |
| | 7.058 (0.90), 7.081 (1.01), 7.140 (0.52), 7.155 (0.78), 7.161 (1.00), |
| | 7.176 (0.46), 7.183 (0.59), 7.202 (0.73), 7.208 (1.00), 7.214 (0.63), |
| | 7.228 (0.73), 7.233 (1.01), 7.239 (0.61), 7.301 (3.22), 7.310 (2.30), |
| | 7.316 (2.45), 7.324 (4.08), 7.335 (0.54), 7.386 (0.46), 7.395 (1.57), |
| | 7.402 (1.68), 7.409 (1.24), 7.419 (1.74), 7.423 (1.59), 7.434 (1.84), |
| | 7.439 (1.49), 7.443 (2.09), 7.448 (1.19), 7.457 (1.49), 7.482 (1.59), |
| | 7.487 (1.63), 7.496 (1.55), 7.501 (1.68), 7.670 (1.63), 8.288 (2.05), |
| | 8.302 (1.93), 10.765 (2.11). |
| 55 | 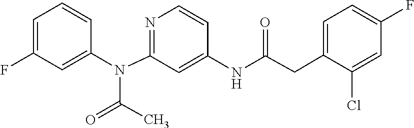 [SMILES: CC(=O)N(c1cccc(F)c1)c1cc(NC(=O)Cc2ccc(F)cc2Cl)ccn1] |
| | N-{4-[2-(2-chloro-4-fluorophenyl)acetamido]pyridin-2-yl}-N-(3- |
| | fluorophenyl)acetamide |
| | LC-MS (Method 1): Rₜ = 1.12 min; MS (ESIpos): m/z = 416 [M + H]⁺ |
| | ¹H-NMR (400 MHZ, DMSO-d6) δ [ppm]: 1.232 (0.99), 1.986 (0.88), |
| | 2.005 (16.00), 2.518 (13.82), 2.523 (8.62), 3.866 (6.18), 5.760 (1.51), |
| | 7.058 (0.99), 7.078 (1.09), 7.141 (0.57), 7.164 (1.09), 7.186 (1.25), |
| | 7.193 (0.88), 7.207 (2.44), 7.214 (2.13), 7.228 (1.51), 7.235 (1.56), |
| | 7.403 (0.83), 7.423 (1.45), 7.431 (1.61), 7.444 (2.18), 7.453 (1.71), |
| | 7.459 (3.48), 7.464 (1.51), 7.473 (1.66), 7.478 (1.97), 7.487 (1.71), |
| | 7.492 (1.77), 7.667 (1.77), 8.288 (2.13), 8.302 (2.13), 10.763 (2.29). |
| 56 | 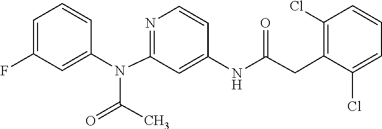 [SMILES: CC(=O)N(c1cccc(F)c1)c1cc(NC(=O)Cc2c(Cl)cccc2Cl)ccn1] |
| | N-{4-[2-(2,6-dichlorophenyl)acetamido]pyridin-2-yl}-N-(3- |
| | fluorophenyl)acetamide |
| | LC-MS (Method 1): Rₜ = 1.15 min; MS (ESIpos): m/z = 433 [M + H]⁺ |
| | ¹H-NMR (400 MHz, DMSO-d6) δ [ppm]: 1.232 (0.44), 1.985 (2.42), |
| | 2.004 (16.00), 2.518 (5.61), 2.523 (3.68), 4.078 (7.36), 5.759 (3.88), |
| | 7.067 (1.02), 7.086 (1.11), 7.136 (0.44), 7.141 (0.55), 7.158 (0.86), |
| | 7.162 (1.06), 7.177 (0.49), 7.184 (0.62), 7.207 (0.78), 7.212 (1.06), |
| | 7.218 (0.64), 7.233 (0.78), 7.238 (1.09), 7.244 (0.64), 7.279 (0.49), |
| | 7.335 (1.53), 7.354 (2.02), 7.356 (2.22), 7.375 (2.48), 7.387 (0.40), |
| | 7.403 (1.06), 7.420 (1.29), 7.424 (1.68), 7.440 (1.51), 7.450 (1.66), |
| | 7.454 (1.66), 7.464 (1.75), 7.469 (1.73), 7.486 (6.96), 7.506 (4.45), |
| | 7.668 (1.71), 8.292 (2.15), 8.306 (2.06), 10.880 (2.30). |
| 57 | 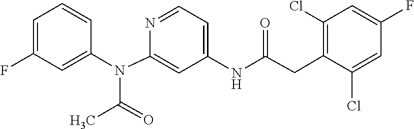 [SMILES: CC(=O)N(c1cccc(F)c1)c1cc(NC(=O)Cc2c(Cl)cc(F)cc2Cl)ccn1] |
| | N-{4-[2-(2,6-dichloro-4-fluorophenyl)acetamido]pyridin-2-yl}-N-(3- |
| | fluorophenyl)acetamide |
| | LC-MS (Method 1): Rₜ = 1.19 min; MS (ESIpos): m/z = 450 [M + H]⁺ |
| | ¹H-NMR (400 MHZ, DMSO-d6) δ [ppm]: 1.172 (0.67), 1.232 (0.83), |
| | 1.988 (1.19), 2.003 (16.00), 2.331 (1.03), 2.336 (0.45), 2.518 (5.22), |
| | 2.523 (3.41), 2.673 (1.03), 2.678 (0.45), 4.041 (6.32), 5.759 (3.65), |
| | 7.064 (0.95), 7.067 (0.95), 7.087 (1.05), 7.138 (0.41), 7.142 (0.55), |
| | 7.144 (0.50), 7.159 (0.86), 7.163 (1.07), 7.179 (0.48), 7.185 (0.64), |
| | 7.207 (0.79), 7.213 (1.03), 7.218 (0.64), 7.233 (0.76), 7.238 (1.00), |
| | 7.244 (0.60), 7.403 (0.79), 7.421 (0.95), 7.424 (1.34), 7.441 (1.48), |
| | 7.445 (2.38), 7.450 (1.69), 7.459 (1.79), 7.464 (1.79), 7.565 (6.37), |
| | 7.587 (6.03), 7.664 (1.62), 8.291 (2.12), 8.305 (2.00), 10.879 (2.31). |
| 58 | 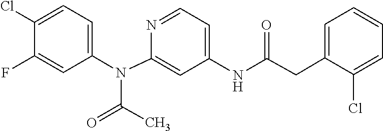 [SMILES: CC(=O)N(c1ccc(Cl)c(F)c1)c1cc(NC(=O)Cc2ccccc2Cl)ccn1] |
| | N-(4-chloro-3-fluorophenyl)-N-{4-[2-(2- |
| | chlorophenyl)acetamido]pyridin-2-yl}acetamide |
| | LC-MS (Method 2): Rₜ = 1.20 min; MS (ESIneg): m/z = 430 [M − H]⁻ |
| | ¹H-NMR (400 MHz, DMSO-d6) δ [ppm]: 2.017 (16.00), 2.518 (0.47), |
| | 3.340 (1.01), 7.091 (0.83), 7.094 (0.84), 7.113 (0.90), 7.116 (0.91), |
| | 7.301 (3.20), 7.310 (2.24), 7.316 (2.30), 7.324 (4.25), 7.334 (0.52), |
| | 7.396 (1.66), 7.403 (0.84), 7.407 (1.05), 7.411 (1.13), 7.420 (1.07), |
| | 7.424 (0.43), 7.434 (1.87), 7.443 (1.50), 7.448 (1.05), 7.457 (1.36), |
| | 7.469 (1.37), 7.474 (1.26), 7.492 (2.02), 7.497 (2.40), 7.500 (1.45), |
| | 7.506 (1.79), 7.511 (1.74), 7.586 (1.59), 7.607 (2.97), 7.629 (1.42), |
| | 7.693 (1.62), 8.289 (2.08), 8.302 (1.96), 10.781 (2.37). |
| 59 | 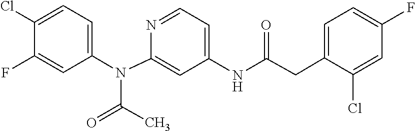 [SMILES: CC(=O)N(c1ccc(Cl)c(F)c1)c1cc(NC(=O)Cc2ccc(F)cc2Cl)ccn1] |
| | N-(4-chloro-3-fluorophenyl)-N-{4-[2-(2-chloro-4- |
| | fluorophenyl)acetamido]pyridin-2-yl}acetamide |
| | LC-MS (Method 2): Rₜ = 1.22 min; MS (ESIpos): m/z = 450 [M + H]⁺ |
| | ¹H-NMR (400 MHZ, DMSO-d6) δ [ppm]: 2.016 (16.00), 2.518 (0.42), |
| | 3.341 (1.00), 7.091 (0.84), 7.094 (0.85), 7.112 (0.91), 7.116 (0.93), |
| | 7.186 (0.74), 7.193 (0.80), 7.207 (1.48), 7.214 (1.71), 7.228 (0.84), |
| | 7.235 (0.91), 7.429 (1.58), 7.436 (1.64), 7.445 (1.47), 7.451 (1.71), |
| | 7.458 (2.01), 7.467 (2.16), 7.474 (1.38), 7.483 (2.68), 7.488 (1.90), |
| | 7.494 (1.58), 7.497 (2.24), 7.502 (2.23), 7.586 (1.64), 7.607 (2.90), |
| | 7.629 (1.48), 7.690 (1.64), 8.287 (2.08), 8.302 (1.98), 10.778 (2.44). |
| 60 | 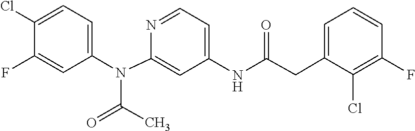 [SMILES: CC(=O)N(c1ccc(Cl)c(F)c1)c1cc(NC(=O)Cc2cccc(F)c2Cl)ccn1] |
| | N-(4-chloro-3-fluorophenyl)-N-{4-[2-(2-chloro-3- |
| | fluorophenyl)acetamido]pyridin-2-yl}acetamide |
| | LC-MS (Method 2): Rₜ = 1.21 min; MS (ESIpos): m/z = 450 [M + H]⁺ |
| | ¹H-NMR (400 MHZ, DMSO-d6) δ [ppm]: 2.017 (16.00), 3.340 (0.76), |
| | 3.943 (6.21), 7.093 (0.75), 7.097 (0.78), 7.099 (0.71), 7.115 (0.83), |
| | 7.118 (0.83), 7.121 (0.75), 7.261 (0.68), 7.268 (1.03), 7.275 (0.76), |
| | 7.278 (0.81), 7.285 (1.12), 7.336 (1.65), 7.341 (2.19), 7.357 (3.72), |
| | 7.362 (0.68), 7.364 (1.28), 7.373 (1.16), 7.469 (1.19), 7.475 (1.18), |
| | 7.485 (1.51), 7.490 (1.66), 7.495 (1.39), 7.499 (2.00), 7.502 (1.77), |
| | 7.504 (1.96), 7.587 (1.52), 7.608 (2.77), 7.629 (1.32), 7.693 (1.45), |
| | 8.291 (1.87), 8.305 (1.76), 10.818 (2.12). |
| 61 | 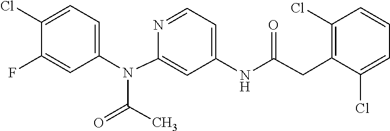 [SMILES: CC(=O)N(c1ccc(Cl)c(F)c1)c1cc(NC(=O)Cc2c(Cl)cccc2Cl)ccn1] |
| | N-(4-chloro-3-fluorophenyl)-N-{4-[2-(2,6- |
| | dichlorophenyl)acetamido]pyridin-2-yl)acetamide |
| | LC-MS (Method 2): Rₜ = 1.25 min; MS (ESIneg): m/z = 464 [M − H]⁻ |
| | ¹H-NMR (400 MHZ, DMSO-d6) δ [ppm]: 2.014 (16.00), 4.082 (7.19), |
| | 7.096 (0.83), 7.100 (0.83), 7.118 (0.88), 7.122 (0.89), 7.332 (1.45), |
| | 7.351 (1.88), 7.354 (2.02), 7.373 (2.39), 7.461 (1.63), 7.466 (1.67), |
| | 7.472 (1.60), 7.476 (2.22), 7.480 (2.47), 7.484 (6.80), 7.498 (1.46), |
| | 7.504 (5.12), 7.585 (1.53), 7.606 (2.92), 7.627 (1.43), 7.693 (1.55), |
| | 8.292 (2.03), 8.306 (1.92), 10.896 (2.36). |
| 62 | 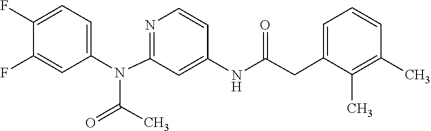 [SMILES: CC(=O)N(c1ccc(F)c(F)c1)c1cc(NC(=O)Cc2cccc(C)c2C)ccn1] |
| | N-(3,4-difluorophenyl)-N-{4-[2-(2,3- |
| | dimethylphenyl)acetamido]pyridin-2-yl}acetamide |
| | LC-MS (Method 1): Rₜ = 1.20 min; MS (ESIpos): m/z = 410 [M + H]⁺ |
| | ¹H-NMR (400 MHZ, DMSO-d6) δ [ppm]: 1.998 (10.16), 2.168 (0.17), |
| | 2.230 (1.32), 2.239 (16.00), 2.331 (0.31), 2.336 (0.16), 2.518 (1.62), |
| | 2.523 (1.04), 2.673 (0.30), 3.455 (0.23), 3.602 (0.28), 3.778 (4.34), |
| | 6.976 (0.16), 6.983 (0.19), 6.991 (0.57), 6.999 (0.71), 7.013 (3.15), |
| | 7.027 (2.31), 7.039 (0.77), 7.049 (0.55), 7.062 (0.31), 7.105 (0.38), |
| | 7.116 (0.33), 7.123 (0.35), 7.127 (0.43), 7.426 (0.38), 7.448 (0.79), |
| | 7.452 (0.50), 7.469 (1.36), 7.474 (1.67), 7.483 (1.17), 7.488 (1.28), |
| | 7.497 (0.67), 7.509 (0.36), 7.515 (0.39), 7.519 (0.39), 7.525 (0.35), |
| | 7.538 (0.33), 7.543 (0.32), 7.712 (0.98), 8.266 (1.31), 8.280 (1.24), |
| | 10.734 (1.43). |
| 63 | 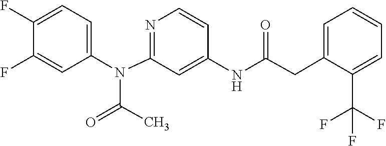 [SMILES: CC(=O)N(c1ccc(F)c(F)c1)c1cc(NC(=O)Cc2ccccc2C(F)(F)F)ccn1] |
| | N-(3,4-difluorophenyl)-N-(4-{2-[2- |
| | (trifluoromethyl)phenyl]acetamido}pyridin-2-yl)acetamide |
| | LC-MS (Method 1): Rₜ = 1.19 min; MS (ESIpos): m/z = 450 [M + H]⁺ |
| | ¹H-NMR (400 MHZ, DMSO-d6) δ [ppm]: 2.000 (16.00), 2.331 (0.28), |
| | 2.518 (1.60), 2.523 (1.00), 2.673 (0.28), 3.615 (0.26), 3.965 (3.48), |
| | 7.108 (0.59), 7.120 (0.52), 7.127 (0.55), 7.131 (0.66), 7.429 (0.60), |
| | 7.453 (2.39), 7.458 (1.90), 7.467 (1.77), 7.472 (2.23), 7.478 (1.65), |
| | 7.500 (3.27), 7.521 (2.87), 7.533 (0.64), 7.545 (0.55), 7.551 (0.52), |
| | 7.635 (0.87), 7.652 (1.28), 7.672 (0.56), 7.704 (2.95), 7.723 (1.32), |
| | 8.266 (2.06), 8.280 (1.95), 10.749 (2.29). |
| 64 | 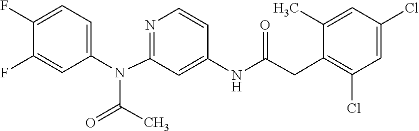 [SMILES: CC(=O)N(c1ccc(F)c(F)c1)c1cc(NC(=O)Cc2c(C)cc(Cl)cc2Cl)ccn1] |
| | N-{4-[2-(2,4-dichloro-6-methylphenyl)acetamido]pyridin-2-yl}-N- |
| | (3,4-difluorophenyl)acetamide |
| | LC-MS (Method 1): Rₜ = 1.30 min; MS (ESIpos): m/z = 464 [M + H]⁺ |
| | ¹H-NMR (400 MHz, DMSO-d6) δ [ppm]: 1.154 (3.97), 1.172 (7.59), |
| | 1.190 (3.96), 1.987 (16.00), 1.998 (11.38), 2.302 (8.22), 2.518 (1.58), |
| | 2.522 (0.99), 3.926 (4.51), 3.999 (1.14), 4.017 (3.45), 4.035 (3.43), |
| | 4.053 (1.11), 5.758 (4.90), 7.108 (0.45), 7.131 (0.50), 7.333 (1.48), |
| | 7.337 (1.55), 7.428 (0.45), 7.449 (3.18), 7.454 (2.85), 7.463 (1.34), |
| | 7.467 (1.32), 7.473 (0.62), 7.477 (0.95), 7.495 (0.43), 7.500 (0.73), |
| | 7.513 (0.41), 7.519 (0.46), 7.523 (0.45), 7.530 (0.40), 7.705 (1.13), |
| | 8.269 (1.52), 8.283 (1.45), 10.835 (1.62). |
| 65 | 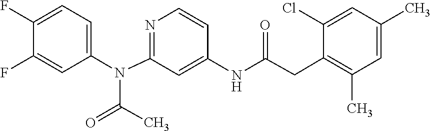 [SMILES: CC(=O)N(c1ccc(F)c(F)c1)c1cc(NC(=O)Cc2c(C)cc(C)cc2Cl)ccn1] |
| | N-{4-[2-(2-chloro-4,6-dimethylphenyl)acetamido]pyridin-2-yl}-N- |
| | (3,4-difluorophenyl)acetamide |
| | LC-MS (Method 1): Rₜ = 1.27 min; MS (ESIpos): m/z = 444 [M + H]⁺ |
| | ¹H-NMR (400 MHz, DMSO-d6) δ [ppm]: 2.000 (11.92), 2.250 (16.00), |
| | 2.518 (1.94), 2.523 (1.32), 2.540 (0.41), 3.882 (4.73), 7.003 (1.65), |
| | 7.112 (2.02), 7.128 (0.54), 7.426 (0.48), 7.449 (0.97), 7.453 (0.69), |
| | 7.459 (1.36), 7.463 (1.39), 7.473 (1.76), 7.477 (1.73), 7.493 (0.46), |
| | 7.498 (0.80), 7.511 (0.44), 7.518 (0.48), 7.521 (0.48), 7.528 (0.43), |
| | 7.540 (0.42), 7.546 (0.40), 7.698 (1.20), 8.266 (1.62), 8.279 (1.52), |
| | 10.774 (1.79). |
| 66 | 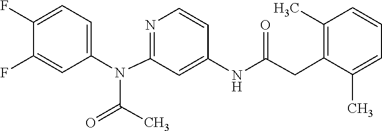 [SMILES: CC(=O)N(c1ccc(F)c(F)c1)c1cc(NC(=O)Cc2c(C)cccc2C)ccn1] |
| | N-(3,4-difluorophenyl)-N-{4-[2-(2,6- |
| | dimethylphenyl)acetamido]pyridin-2-yl}acetamide |
| | LC-MS (Method 1): Rₜ = 1.21 min; MS (ESIpos): m/z = 410 [M + H]⁺ |
| | ¹H-NMR (400 MHz, DMSO-d6) δ [ppm]: 1.998 (16.00), 2.091 (0.17), |
| | 2.142 (11.40), 2.235 (11.01), 2.518 (1.54), 2.522 (0.97), 3.599 (0.26), |
| | 3.736 (6.71), 6.994 (0.39), 7.007 (0.55), 7.017 (0.87), 7.030 (2.43), |
| | 7.046 (4.59), 7.060 (1.11), 7.068 (0.87), 7.102 (0.58), 7.113 (0.50), |
| | 7.120 (0.56), 7.124 (0.65), 7.426 (0.58), 7.448 (1.18), 7.452 (0.76), |
| | 7.471 (0.73), 7.474 (1.29), 7.480 (1.71), 7.485 (1.78), 7.494 (2.07), |
| | 7.498 (2.28), 7.509 (0.58), 7.516 (0.59), 7.520 (0.60), 7.526 (0.54), |
| | 7.538 (0.52), 7.545 (0.50), 7.704 (1.51), 8.262 (2.00), 8.275 (1.89), |
| | 10.658 (2.20). |
| 67 | 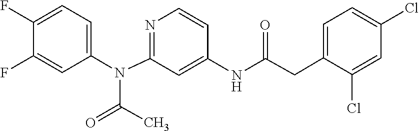 [SMILES: CC(=O)N(c1ccc(F)c(F)c1)c1cc(NC(=O)Cc2ccc(Cl)cc2Cl)ccn1] |
| | N-{4-[2-(2,4-dichlorophenyl)acetamido]pyridin-2-yl}-N-(3,4- |
| | difluorophenyl)acetamide |
| | LC-MS (Method 2): Rₜ = 1.24 min; MS (ESIpos): m/z = 451 [M + H]⁺ |
| | ¹H-NMR (400 MHZ, DMSO-d6) δ [ppm]: 1.154 (0.61), 1.172 (1.18), |
| | 1.190 (0.63), 1.987 (2.51), 2.002 (16.00), 2.518 (1.65), 2.523 (1.08), |
| | 3.885 (6.08), 4.017 (0.51), 4.035 (0.52), 7.109 (0.54), 7.120 (0.47), |
| | 7.127 (0.50), 7.131 (0.62), 7.403 (0.89), 7.408 (0.83), 7.424 (2.78), |
| | 7.429 (3.36), 7.440 (3.67), 7.453 (1.23), 7.457 (0.91), 7.461 (1.34), |
| | 7.463 (1.79), 7.468 (1.76), 7.478 (2.25), 7.482 (1.88), 7.497 (0.54), |
| | 7.502 (0.92), 7.515 (0.53), 7.521 (0.57), 7.526 (0.57), 7.532 (0.51), |
| | 7.544 (0.50), 7.550 (0.48), 7.617 (2.53), 7.622 (2.52), 7.700 (1.40), |
| | 8.270 (1.90), 8.284 (1.79), 10.779 (2.14). |
| 68 | 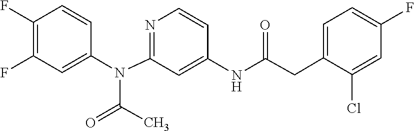 [SMILES: CC(=O)N(c1ccc(F)c(F)c1)c1cc(NC(=O)Cc2ccc(F)cc2Cl)ccn1] |
| | N-{4-[2-(2-chloro-4-fluorophenyl)acetamido]pyridin-2-yl}-N-(3,4- |
| | difluorophenyl)acetamide |
| | LC-MS (Method 1): Rₜ = 1.16 min; MS (ESIpos): m/z = 434 [M + H]⁺ |
| | ¹H-NMR (400 MHZ, DMSO-d6) δ [ppm]: 1.154 (1.17), 1.171 (2.56), |
| | 1.189 (1.31), 1.986 (4.84), 2.002 (16.00), 2.518 (1.23), 2.522 (0.82), |
| | 3.870 (5.94), 3.998 (0.34), 4.016 (0.97), 4.034 (0.95), 4.052 (0.31), |
| | 7.108 (0.57), 7.119 (0.49), 7.126 (0.54), 7.130 (0.65), 7.187 (0.71), |
| | 7.194 (0.79), 7.209 (1.42), 7.215 (1.66), 7.230 (0.82), 7.237 (0.89), |
| | 7.430 (1.96), 7.437 (1.65), 7.445 (1.48), 7.452 (2.63), 7.459 (2.37), |
| | 7.464 (2.34), 7.467 (1.96), 7.469 (2.16), 7.474 (1.00), 7.478 (2.86), |
| | 7.483 (2.83), 7.495 (0.57), 7.501 (1.05), 7.513 (0.55), 7.520 (0.60), |
| | 7.524 (0.60), 7.530 (0.54), 7.542 (0.52), 7.549 (0.49), 7.704 (1.51), |
| | 8.269 (2.03), 8.283 (1.92), 10.765 (2.26). |
| 69 | 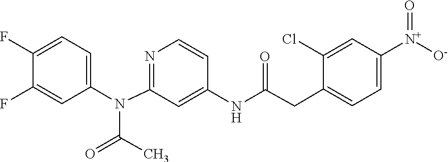 [SMILES: CC(=O)N(c1ccc(F)c(F)c1)c1cc(NC(=O)Cc2ccc([N+](=O)[O-])cc2Cl)ccn1] |
| | N-{4-[2-(2-chloro-4-nitrophenyl)acetamido]pyridin-2-yl}-N-(3,4- |
| | difluorophenyl)acetamide |
| | LC-MS (Method 1): Rₜ = 1.15 min; MS (ESIpos): m/z = 461 [M + H]⁺ |
| | ¹H-NMR (400 MHz, DMSO-d6) δ [ppm]: 1.987 (0.49), 2.004 (16.00), |
| | 2.030 (0.62), 2.518 (2.07), 2.522 (1.35), 4.057 (6.46), 5.758 (3.46), |
| | 7.114 (0.62), 7.125 (0.54), 7.136 (0.70), 7.433 (0.62), 7.455 (1.24), |
| | 7.459 (0.88), 7.465 (1.73), 7.470 (1.81), 7.480 (2.34), 7.484 (2.16), |
| | 7.504 (0.90), 7.520 (0.60), 7.526 (0.65), 7.530 (0.66), 7.536 (0.59), |
| | 7.548 (0.57), 7.554 (0.55), 7.707 (1.60), 7.725 (2.60), 7.746 (2.72), |
| | 8.188 (1.79), 8.194 (2.02), 8.209 (1.58), 8.215 (1.86), 8.276 (2.16), |
| | 8.290 (2.05), 8.301 (3.52), 8.307 (2.99), 10.873 (2.43). |
| 70 | 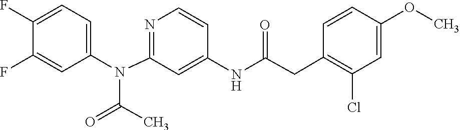 [SMILES: COc1ccc(CC(=O)Nc2ccnc(N(C(C)=O)c3ccc(F)c(F)c3)c2)c(Cl)c1] |
| | N-{4-[2-(2-chloro-4-methoxyphenyl)acetamido]pyridin-2-yl}-N-(3,4- |
| | difluorophenyl)acetamide |
| | LC-MS (Method 1): Rₜ = 1.14 min; MS (ESIpos): m/z = 446 [M + H]⁺ |
| | ¹H-NMR (400 MHZ, DMSO-d6) δ [ppm]: 1.154 (1.77), 1.172 (2.94), |
| | 1.190 (1.49), 1.231 (0.40), 1.987 (6.03), 1.996 (14.46), 2.518 (2.13), |
| | 2.522 (1.33), 3.625 (6.07), 3.721 (0.77), 3.827 (16.00), 3.999 (0.42), |
| | 4.017 (1.24), 4.035 (1.23), 7.084 (2.16), 7.105 (3.35), 7.123 (0.72), |
| | 7.216 (1.47), 7.222 (1.49), 7.238 (1.07), 7.243 (1.12), 7.367 (2.71), |
| | 7.372 (2.52), 7.425 (0.57), 7.448 (1.17), 7.471 (2.26), 7.475 (2.32), |
| | 7.485 (1.67), 7.490 (2.13), 7.497 (1.18), 7.509 (0.59), 7.516 (0.65), |
| | 7.519 (0.65), 7.526 (0.55), 7.538 (0.54), 7.544 (0.52), 7.701 (1.65), |
| | 8.260 (1.99), 8.274 (1.90), 10.657 (2.15). |
| 71 | 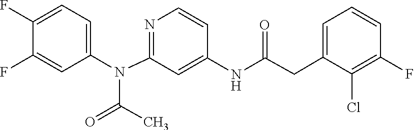 [SMILES: CC(=O)N(c1ccc(F)c(F)c1)c1cc(NC(=O)Cc2cccc(F)c2Cl)ccn1] |
| | N-{4-[2-(2-chloro-3-fluorophenyl)acetamido]pyridin-2-yl}-N-(3,4- |
| | difluorophenyl)acetamide |
| | LC-MS (Method 1): Rₜ = 1.16 min; MS (ESIpos): m/z = 434 [M + H]⁺ |
| | ¹H-NMR (400 MHZ, DMSO-d6) δ [ppm]: 2.003 (16.00), 2.518 (1.09), |
| | 2.522 (0.69), 3.635 (0.31), 3.943 (6.79), 7.110 (0.58), 7.122 (0.52), |
| | 7.129 (0.55), 7.132 (0.67), 7.262 (0.73), 7.269 (1.11), 7.276 (0.82), |
| | 7.280 (0.89), 7.284 (1.17), 7.324 (0.40), 7.338 (1.81), 7.343 (2.36), |
| | 7.360 (3.90), 7.367 (1.34), 7.375 (1.22), 7.381 (0.37), 7.396 (0.19), |
| | 7.430 (0.58), 7.453 (1.21), 7.457 (0.80), 7.467 (1.74), 7.472 (1.93), |
| | 7.475 (1.01), 7.481 (2.17), 7.486 (1.86), 7.498 (0.64), 7.502 (0.86), |
| | 7.517 (0.56), 7.523 (0.60), 7.527 (0.61), 7.533 (0.55), 7.545 (0.53), |
| | 7.552 (0.50), 7.709 (1.53), 8.273 (2.04), 8.287 (1.95), 10.805 (2.26). |
| 72 | 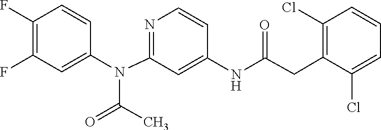 [SMILES: CC(=O)N(c1ccc(F)c(F)c1)c1cc(NC(=O)Cc2c(Cl)cccc2Cl)ccn1] |
| | N-{4-[2-(2,6-dichlorophenyl)acetamido]pyridin-2-yl}-N-(3,4- |
| | difluorophenyl)acetamide |
| | LC-MS (Method 1): Rₜ = 1.20 min; MS (ESIneg): m/z = 448 [M − H]⁻ |
| | ¹H-NMR (400 MHz, DMSO-d6) δ [ppm]: 2.001 (16.00), 2.518 (2.14), |
| | 2.523 (1.36), 3.159 (1.08), 3.172 (1.23), 4.082 (7.79), 4.095 (0.44), |
| | 7.114 (0.66), 7.125 (0.60), 7.136 (0.77), 7.336 (1.49), 7.355 (2.06), |
| | 7.357 (2.12), 7.376 (2.40), 7.429 (0.66), 7.441 (1.84), 7.445 (1.88), |
| | 7.455 (2.55), 7.459 (1.93), 7.474 (0.92), 7.478 (1.45), 7.487 (6.79), |
| | 7.500 (1.41), 7.507 (4.83), 7.519 (0.70), 7.525 (0.72), 7.529 (0.69), |
| | 7.535 (0.61), 7.548 (0.59), 7.554 (0.53), 7.709 (1.74), 8.274 (2.21), |
| | 8.288 (2.09), 10.885 (2.43). |
| 73 | 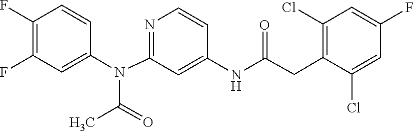 [SMILES: CC(=O)N(c1ccc(F)c(F)c1)c1cc(NC(=O)Cc2c(Cl)cc(F)cc2Cl)ccn1] |
| | N-{4-[2-(2,6-dichloro-4-fluorophenyl)acetamido]pyridin-2-yl}-N- |
| | (3,4-difluorophenyl)acetamide |
| | LC-MS (Method 1): Rₜ = 1.22 min; MS (ESIpos): m/z = 469 [M + H]⁺ |
| | ¹H-NMR (400 MHz, DMSO-d6) δ [ppm]: 1.232 (0.79), 1.987 (0.73), |
| | 2.000 (16.00), 2.332 (0.83), 2.518 (5.12), 2.522 (3.21), 2.673 (0.83), |
| | 4.045 (6.58), 5.758 (2.77), 7.115 (0.62), 7.126 (0.56), 7.136 (0.69), |
| | 7.431 (0.77), 7.435 (1.73), 7.440 (1.67), 7.449 (1.75), 7.454 (2.85), |
| | 7.475 (0.75), 7.480 (1.25), 7.502 (1.00), 7.507 (0.58), 7.519 (0.58), |
| | 7.526 (0.63), 7.530 (0.65), 7.536 (0.58), 7.548 (0.58), 7.554 (0.58), |
| | 7.567 (6.31), 7.589 (6.06), 7.707 (1.56), 8.273 (2.12), 8.288 (2.02), |
| | 10.880 (2.48). |
| 74 | 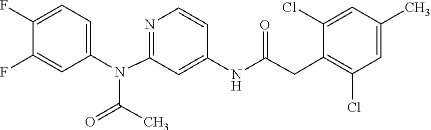 [SMILES: CC(=O)N(c1ccc(F)c(F)c1)c1cc(NC(=O)Cc2c(Cl)cc(C)cc2Cl)ccn1] |
| | N-{4-[2-(2,6-dichloro-4-methylphenyl)acetamido]pyridin-2-yl}-N- |
| | (3,4-difluorophenyl)acetamide |
| | LC-MS (Method 1): Rₜ = 1.27 min; MS (ESIpos): m/z = 464 [M + H]⁺ |
| | ¹H-NMR (400 MHz, DMSO-d6) δ [ppm]: 1.154 (1.72), 1.172 (3.12), |
| | 1.189 (1.52), 1.987 (5.73), 2.001 (16.00), 2.304 (10.37), 2.322 (0.42), |
| | 2.326 (0.48), 2.518 (1.85), 2.522 (1.14), 2.668 (0.42), 3.999 (0.49), |
| | 4.018 (7.26), 4.035 (1.30), 4.053 (0.41), 7.111 (0.64), 7.122 (0.59), |
| | 7.129 (0.62), 7.133 (0.73), 7.333 (7.34), 7.428 (0.61), 7.444 (1.81), |
| | 7.449 (2.54), 7.454 (1.15), 7.458 (1.85), 7.463 (1.83), 7.472 (0.80), |
| | 7.477 (1.26), 7.499 (1.01), 7.504 (0.59), 7.516 (0.60), 7.523 (0.66), |
| | 7.527 (0.65), 7.533 (0.58), 7.545 (0.57), 7.552 (0.53), 7.695 (1.66), |
| | 8.270 (2.14), 8.284 (2.05), 10.856 (2.09). |
| 75 | 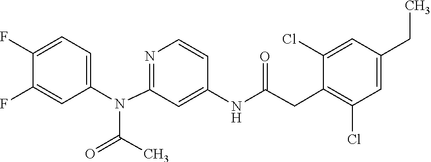 [SMILES: CCc1cc(Cl)c(CC(=O)Nc2ccnc(N(C(C)=O)c3ccc(F)c(F)c3)c2)c(Cl)c1] |
| | N-{4-[2-(2,6-dichloro-4-ethylphenyl)acetamido]pyridin-2-yl}-N-(3,4- |
| | difluorophenyl)acetamide |
| | LC-MS (Method 1): Rₜ = 1.36 min; MS (ESIpos): m/z = 478 [M + H]⁺ |
| | ¹H-NMR (400 MHZ, DMSO-d6) δ [ppm]: 1.155 (4.47), 1.174 (9.99), |
| | 1.188 (1.72), 1.193 (4.62), 1.206 (0.42), 2.001 (16.00), 2.331 (0.56), |
| | 2.518 (3.03), 2.522 (1.85), 2.580 (1.12), 2.599 (3.12), 2.618 (2.98), |
| | 2.637 (0.95), 2.673 (0.63), 4.023 (6.98), 7.112 (0.76), 7.133 (0.83), |
| | 7.357 (9.79), 7.429 (0.72), 7.440 (1.95), 7.445 (1.96), 7.454 (2.67), |
| | 7.459 (2.02), 7.474 (1.01), 7.478 (1.34), 7.497 (1.35), 7.516 (0.71), |
| | 7.523 (0.80), 7.526 (0.76), 7.533 (0.65), 7.545 (0.65), 7.551 (0.56), |
| | 7.698 (1.98), 8.271 (2.26), 8.285 (2.13), 10.848 (2.78). |
| 76 | 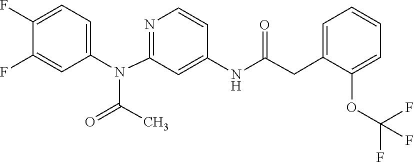 [SMILES: CC(=O)N(c1ccc(F)c(F)c1)c1cc(NC(=O)Cc2ccccc2OC(F)(F)F)ccn1] |
| | N-(3,4-difluorophenyl)-N-(4-{2-[2- |
| | (trifluoromethoxy)phenyl]acetamido}pyridin-2-yl)acetamide |
| | LC-MS (Method 1): Rₜ = 1.22 min; MS (ESIpos): m/z = 466 [M + H]⁺ |
| | ¹H-NMR (400 MHZ, DMSO-d6) δ [ppm]: 1.012 (0.21), 1.127 (0.21), |
| | 2.002 (16.00), 2.518 (2.05), 2.522 (1.28), 2.673 (0.35), 2.678 (0.16), |
| | 3.709 (0.22), 3.824 (6.51), 7.109 (0.57), 7.120 (0.50), 7.127 (0.53), |
| | 7.131 (0.65), 7.335 (0.62), 7.339 (0.84), 7.344 (0.96), 7.348 (0.85), |
| | 7.355 (0.85), 7.359 (1.36), 7.363 (2.64), 7.381 (1.71), 7.385 (1.02), |
| | 7.402 (1.23), 7.407 (1.61), 7.421 (0.99), 7.427 (1.46), 7.430 (0.75), |
| | 7.441 (0.52), 7.446 (0.52), 7.453 (1.26), 7.459 (1.89), 7.465 (2.81), |
| | 7.470 (1.95), 7.479 (3.72), 7.484 (2.48), 7.496 (0.58), 7.502 (1.02), |
| | 7.514 (0.56), 7.521 (0.59), 7.525 (0.59), 7.531 (0.53), 7.543 (0.53), |
| | 7.550 (0.51), 7.701 (1.47), 8.269 (1.99), 8.283 (1.91), 10.754 (2.20). |
| 77 | 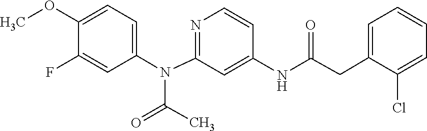 [SMILES: COc1ccc(N(C(C)=O)c2cc(NC(=O)Cc3ccccc3Cl)ccn2)cc1F] |
| | N-{4-[2-(2-chlorophenyl)acetamido]pyridin-2-yl}-N-(3-fluoro-4- |
| | methoxyphenyl)acetamide |
| | LC-MS (Method 2): Rₜ = 1.07 min; MS (ESIpos): m/z = 428 [M + H]⁺ |
| | ¹H-NMR (400 MHz, DMSO-d6) δ [ppm]: 1.987 (14.56), 2.518 (1.73), |
| | 2.523 (1.16), 3.841 (16.00), 3.874 (6.88), 7.031 (0.66), 7.035 (0.59), |
| | 7.053 (0.95), 7.057 (0.83), 7.145 (1.27), 7.167 (1.71), 7.190 (0.94), |
| | 7.244 (1.21), 7.250 (1.17), 7.275 (1.22), 7.280 (1.21), 7.302 (3.00), |
| | 7.311 (1.93), 7.316 (2.03), 7.317 (1.99), 7.325 (3.96), 7.336 (0.48), |
| | 7.396 (1.47), 7.403 (0.71), 7.407 (0.88), 7.411 (0.99), 7.419 (0.91), |
| | 7.435 (1.70), 7.444 (1.43), 7.449 (1.09), 7.452 (1.95), 7.457 (2.22), |
| | 7.466 (1.74), 7.471 (1.70), 7.677 (1.62), 7.681 (1.60), 8.247 (2.18), |
| | 8.260 (2.06), 10.737 (2.16). |
| 78 | 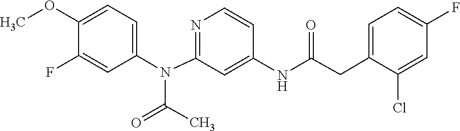 [SMILES: COc1ccc(N(C(C)=O)c2cc(NC(=O)Cc3ccc(F)cc3Cl)ccn2)cc1F] |
| | N-{4-[2-(2-chloro-4-fluorophenyl)acetamido]pyridin-2-yl}-N-(3- |
| | fluoro-4-methoxyphenyl)acetamide |
| | LC-MS (LC-ES+): Rₜ = 1.04 min; MS (ESIpos): m/z = 446 [M + H]⁺ |
| | ¹H-NMR (400 MHz, DMSO-d6) δ [ppm]: 1.986 (13.64), 2.518 (1.92), |
| | 2.523 (1.25), 3.842 (16.00), 3.865 (5.62), 7.031 (0.68), 7.053 (0.93), |
| | 7.145 (1.24), 7.168 (1.70), 7.187 (0.82), 7.190 (1.06), 7.193 (0.89), |
| | 7.208 (1.36), 7.215 (1.54), 7.230 (0.76), 7.237 (0.85), 7.244 (1.23), |
| | 7.250 (1.12), 7.274 (1.18), 7.280 (1.12), 7.431 (1.42), 7.438 (1.50), |
| | 7.444 (2.16), 7.447 (1.89), 7.453 (1.71), 7.460 (3.19), 7.466 (1.31), |
| | 7.482 (1.05), 7.674 (1.62), 7.677 (1.60), 8.246 (2.14), 8.260 (2.03), |
| | 10.733 (2.17). |
| 79 | 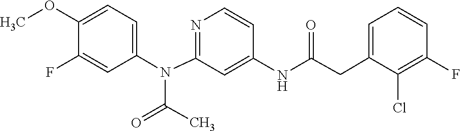 [SMILES: COc1ccc(N(C(C)=O)c2cc(NC(=O)Cc3cccc(F)c3Cl)ccn2)cc1F] |
| | N-{4-[2-(2-chloro-3-fluorophenyl)acetamido]pyridin-2-yl}-N-(3- |
| | fluoro-4-methoxyphenyl)acetamide |
| | LC-MS (Method 2): Rₜ = 1.09 min; MS (ESIneg): m/z = 444 [M − H]⁻ |
| | ¹H-NMR (400 MHZ, DMSO-d6) δ [ppm]: 1.987 (14.51), 2.518 (0.89), |
| | 2.523 (0.56), 3.842 (16.00), 3.938 (6.32), 7.031 (0.64), 7.033 (0.67), |
| | 7.037 (0.60), 7.053 (0.88), 7.055 (0.94), 7.058 (0.83), 7.145 (1.30), |
| | 7.168 (1.70), 7.191 (0.93), 7.246 (1.24), 7.252 (1.18), 7.261 (0.74), |
| | 7.268 (1.09), 7.276 (1.69), 7.283 (2.16), 7.338 (1.69), 7.343 (2.14), |
| | 7.359 (3.72), 7.366 (1.23), 7.374 (1.07), 7.445 (1.55), 7.450 (1.59), |
| | 7.459 (1.48), 7.464 (1.63), 7.677 (1.60), 7.681 (1.57), 8.250 (2.16), |
| | 8.264 (2.04), 10.775 (2.17). |
| 80 | 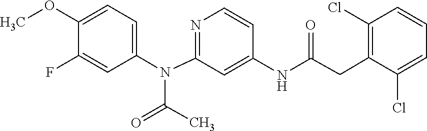 [SMILES: COc1ccc(N(C(C)=O)c2cc(NC(=O)Cc3c(Cl)cccc3Cl)ccn2)cc1F] |
| | N-{4-[2-(2,6-dichlorophenyl)acetamido]pyridin-2-yl}-N-(3-fluoro-4- |
| | methoxyphenyl)acetamide |
| | LC-MS (Method 2): Rₜ = 1.13 min; MS (ESIneg): m/z = 460 [M − H]⁻ |
| | ¹H-NMR (400 MHZ, DMSO-d6) δ [ppm]: 1.985 (13.60), 2.332 (0.55), |
| | 2.518 (2.48), 2.523 (1.63), 2.673 (0.58), 3.842 (16.00), 4.076 (6.43), |
| | 7.036 (0.65), 7.058 (0.92), 7.062 (0.79), 7.145 (1.26), 7.168 (1.65), |
| | 7.190 (0.90), 7.248 (1.15), 7.254 (1.13), 7.278 (1.14), 7.284 (1.09), |
| | 7.335 (1.34), 7.354 (1.73), 7.357 (1.89), 7.375 (2.11), 7.416 (1.48), |
| | 7.421 (1.46), 7.430 (1.46), 7.435 (1.52), 7.487 (5.93), 7.506 (3.60), |
| | 7.679 (1.51), 8.250 (2.11), 8.264 (1.97), 10.849 (2.08). |
| 81 | 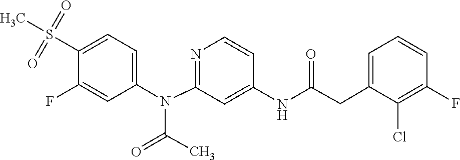 [SMILES: CC(=O)N(c1ccc(S(C)(=O)=O)c(F)c1)c1cc(NC(=O)Cc2cccc(F)c2Cl)ccn1] |
| | N-{4-[2-(2-chloro-3-fluorophenyl)acetamido]pyridin-2-yl}-N-[3- |
| | fluoro-4-(methanesulfonyl)phenyl]acetamide |
| | LC-MS (Method 2): Rₜ = 1.03 min; MS (ESIpos): m/z = 494 [M + H]⁺ |
| | ¹H-NMR (400 MHZ, DMSO-d6) δ [ppm]: 2.038 (13.07), 2.518 (3.33), |
| | 2.522 (2.37), 3.334 (16.00), 3.340 (13.48), 3.949 (5.75), 7.193 (0.87), |
| | 7.198 (0.91), 7.215 (0.96), 7.219 (1.00), 7.261 (0.65), 7.268 (0.99), |
| | 7.275 (0.73), 7.279 (0.78), 7.285 (1.12), 7.338 (1.53), 7.343 (1.84), |
| | 7.359 (3.54), 7.367 (1.28), 7.374 (1.14), 7.537 (1.32), 7.542 (1.65), |
| | 7.551 (1.99), 7.556 (1.61), 7.574 (1.06), 7.578 (1.07), 7.682 (1.61), |
| | 7.685 (1.63), 7.823 (1.17), 7.844 (2.05), 7.864 (1.10), 8.367 (1.68), |
| | 8.381 (1.66), 10.872 (2.13). |
| 82 | 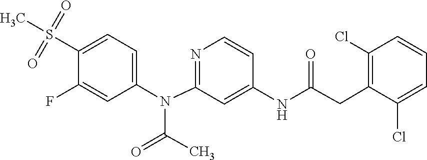 [SMILES: CC(=O)N(c1ccc(S(C)(=O)=O)c(F)c1)c1cc(NC(=O)Cc2c(Cl)cccc2Cl)ccn1] |
| | N-{4-[2-(2,6-dichlorophenyl)acetamido]pyridin-2-yl}-N-[3-fluoro-4- |
| | (methanesulfonyl)phenyl]acetamide |
| | LC-MS (Method 2): Rₜ = 1.07 min; MS (ESIpos): m/z = 510 [M + H]⁺ |
| | ¹H-NMR (400 MHZ, DMSO-d6) δ [ppm]: 2.036 (5.17), 2.518 (2.11), |
| | 2.522 (1.49), 3.332 (16.00), 3.339 (5.42), 4.088 (2.45), 7.334 (0.50), |
| | 7.353 (0.63), 7.356 (0.74), 7.375 (0.80), 7.486 (2.26), 7.505 (1.42), |
| | 7.517 (0.59), 7.522 (0.58), 7.531 (0.54), 7.536 (0.61), 7.548 (0.45), |
| | 7.552 (0.45), 7.577 (0.41), 7.581 (0.43), 7.679 (0.64), 7.682 (0.64), |
| | 7.821 (0.48), 7.842 (0.84), 7.863 (0.45), 8.368 (0.68), 8.382 (0.67), |
| | 10.951 (0.87). |
| 83 | 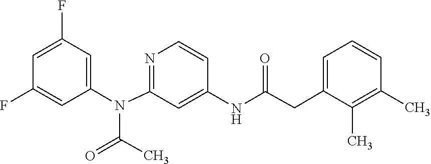 [SMILES: CC(=O)N(c1cc(F)cc(F)c1)c1cc(NC(=O)Cc2cccc(C)c2C)ccn1] |
| | N-(3,5-difluorophenyl)-N-{4-[2-(2,3- |
| | dimethylphenyl)acetamido]pyridin-2-yl}acetamide |
| | LC-MS (Method 1): Rₜ = 1.23 min; MS (ESIpos): m/z = 410 [M + H]⁺ |
| | ¹H-NMR (400 MHz, DMSO-d6) δ [ppm]: 1.108 (5.83), 2.014 (16.00), |
| | 2.142 (10.68), 2.235 (10.48), 2.327 (0.61), 2.331 (0.45), 2.336 (0.20), |
| | 2.518 (2.77), 2.523 (1.64), 2.539 (2.07), 2.669 (0.62), 2.673 (0.46), |
| | 2.678 (0.20), 3.740 (6.40), 4.191 (0.52), 6.994 (0.40), 7.008 (0.59), |
| | 7.017 (0.85), 7.030 (2.28), 7.047 (4.73), 7.061 (2.64), 7.068 (1.34), |
| | 7.077 (1.49), 7.082 (1.45), 7.093 (0.29), 7.188 (0.32), 7.194 (0.60), |
| | 7.200 (0.36), 7.212 (0.66), 7.217 (1.19), 7.223 (0.68), 7.235 (0.33), |
| | 7.241 (0.59), 7.247 (0.32), 7.511 (1.48), 7.515 (1.52), 7.524 (1.49), |
| | 7.530 (1.53), 7.695 (1.57), 7.698 (1.54), 8.296 (1.85), 8.310 (1.76), |
| | 10.681 (2.10). |
| 84 | 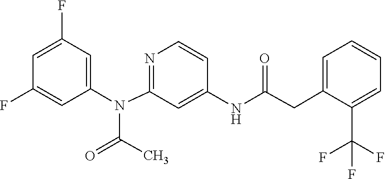 [SMILES: CC(=O)N(c1cc(F)cc(F)c1)c1cc(NC(=O)Cc2ccccc2C(F)(F)F)ccn1] |
| | N-(3,5-difluorophenyl)-N-(4-{2-[2- |
| | (trifluoromethyl)phenyl]acetamido}pyridin-2-yl)acetamide |
| | LC-MS (Method 1): Rₜ = 1.21 min; MS (ESIpos): m/z = 450 [M + H]⁺ |
| | ¹H-NMR (400 MHz, DMSO-d6) δ [ppm]: 0.967 (0.22), 1.108 (8.14), |
| | 2.016 (16.00), 2.337 (0.19), 2.518 (2.29), 2.523 (1.61), 2.540 (2.42), |
| | 2.659 (0.20), 2.678 (0.20), 3.969 (3.05), 4.191 (0.75), 7.053 (0.25), |
| | 7.063 (1.20), 7.069 (1.45), 7.084 (1.35), 7.089 (1.33), 7.100 (0.26), |
| | 7.193 (0.30), 7.198 (0.57), 7.205 (0.35), 7.216 (0.67), 7.222 (1.15), |
| | 7.228 (0.62), 7.240 (0.32), 7.245 (0.57), 7.251 (0.29), 7.483 (1.86), |
| | 7.488 (1.64), 7.497 (2.29), 7.502 (3.50), 7.522 (2.03), 7.635 (0.75), |
| | 7.653 (1.09), 7.673 (0.49), 7.691 (1.48), 7.695 (1.48), 7.704 (1.39), |
| | 7.724 (1.12), 8.301 (1.80), 8.315 (1.70), 10.774 (2.00). |
| 85 | 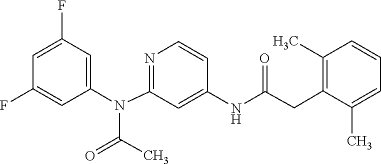 [SMILES: CC(=O)N(c1cc(F)cc(F)c1)c1cc(NC(=O)Cc2c(C)cccc2C)ccn1] |
| | N-(3,5-difluorophenyl)-N-{4-[2-(2,6- |
| | dimethylphenyl)acetamido]pyridin-2-yl}acetamide |
| | LC-MS (Method 1): Rₜ = 1.22 min; MS (ESIpos): m/z = 410 [M + H]⁺ |
| | ¹H-NMR (400 MHZ, DMSO-d6) δ [ppm]: 0.967 (0.20), 1.108 (9.09), |
| | 2.014 (12.16), 2.240 (16.00), 2.327 (0.49), 2.331 (0.35), 2.518 (1.73), |
| | 2.523 (1.22), 2.540 (0.30), 2.669 (0.49), 2.673 (0.34), 3.782 (4.33), |
| | 4.191 (0.75), 6.991 (0.56), 6.999 (0.69), 7.013 (3.11), 7.027 (2.29), |
| | 7.040 (0.86), 7.050 (0.76), 7.056 (0.89), 7.062 (1.25), 7.076 (0.95), |
| | 7.082 (0.95), 7.093 (0.18), 7.186 (0.23), 7.192 (0.42), 7.198 (0.25), |
| | 7.210 (0.49), 7.215 (0.85), 7.221 (0.46), 7.233 (0.24), 7.239 (0.42), |
| | 7.245 (0.23), 7.502 (1.06), 7.507 (1.07), 7.516 (1.01), 7.521 (1.12), |
| | 7.702 (1.04), 7.705 (1.01), 8.301 (1.27), 8.315 (1.20), 10.758 (1.42). |
| 86 | 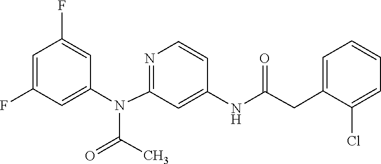 [SMILES: CC(=O)N(c1cc(F)cc(F)c1)c1cc(NC(=O)Cc2ccccc2Cl)ccn1] |
| | N-{4-[2-(2-chlorophenyl)acetamido]pyridin-2-yl}-N-(3,5- |
| | difluorophenyl)acetamide |
| | LC-MS (Method 1): Rₜ = 1.16 min; MS (ESIpos): m/z = 416 [M + H]⁺ |
| | ¹H-NMR (400 MHz, DMSO-d6) δ [ppm]: 1.987 (0.59), 2.020 (16.00), |
| | 2.518 (2.19), 2.522 (1.39), 3.884 (7.33), 7.062 (1.38), 7.068 (1.65), |
| | 7.083 (1.55), 7.088 (1.53), 7.198 (0.64), 7.215 (0.70), 7.221 (1.22), |
| | 7.227 (0.69), 7.245 (0.61), 7.303 (2.90), 7.312 (2.11), 7.318 (2.20), |
| | 7.326 (3.86), 7.336 (0.49), 7.399 (1.53), 7.406 (0.76), 7.409 (0.96), |
| | 7.414 (1.06), 7.422 (0.98), 7.437 (1.71), 7.445 (1.37), 7.450 (1.00), |
| | 7.459 (1.20), 7.505 (1.51), 7.510 (1.60), 7.520 (1.56), 7.524 (1.62), |
| | 7.698 (1.72), 8.305 (2.01), 8.319 (1.90), 10.791 (2.26). |
| 87 | 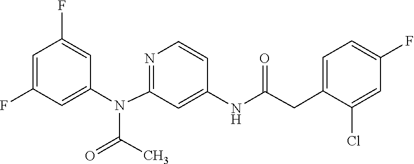 [SMILES: CC(=O)N(c1cc(F)cc(F)c1)c1cc(NC(=O)Cc2ccc(F)cc2Cl)ccn1] |
| | N-{4-[2-(2-chloro-4-fluorophenyl)acetamido]pyridin-2-yl}-N-(3,5- |
| | difluorophenyl)acetamide |
| | LC-MS (Method 1): Rₜ = 1.19 min; MS (ESIpos): m/z = 434 [M + H]⁺ |
| | ¹H-NMR (400 MHZ, DMSO-d6) δ [ppm]: 2.019 (16.00), 2.518 (1.21), |
| | 2.522 (0.79), 3.874 (6.02), 7.062 (1.33), 7.067 (1.60), 7.083 (1.49), |
| | 7.088 (1.48), 7.188 (0.72), 7.194 (0.97), 7.199 (0.72), 7.205 (0.49), |
| | 7.209 (1.49), 7.216 (2.24), 7.222 (1.34), 7.228 (0.89), 7.230 (1.01), |
| | 7.237 (1.01), 7.245 (0.64), 7.431 (1.46), 7.438 (1.51), 7.447 (1.36), |
| | 7.454 (1.58), 7.461 (1.82), 7.469 (1.24), 7.485 (1.13), 7.497 (1.58), |
| | 7.502 (1.56), 7.511 (1.54), 7.516 (1.66), 7.694 (1.64), 7.698 (1.61), |
| | 8.304 (1.99), 8.319 (1.87), 10.788 (2.25). |
| 88 | 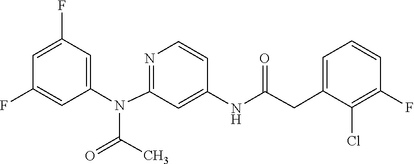 [SMILES: CC(=O)N(c1cc(F)cc(F)c1)c1cc(NC(=O)Cc2cccc(F)c2Cl)ccn1] |
| | N-{4-[2-(2-chloro-3-fluorophenyl)acetamido]pyridin-2-yl}-N-(3,5- |
| | difluorophenyl)acetamide |
| | LC-MS (Method 1): Rₜ = 1.18 min; MS (ESIpos): m/z = 434 [M + H]⁺ |
| | ¹H-NMR (400 MHZ, DMSO-d6) δ [ppm]: 0.967 (0.20), 1.107 (11.67), |
| | 1.144 (0.18), 2.019 (16.00), 2.327 (0.57), 2.331 (0.40), 2.336 (0.18), |
| | 2.518 (2.29), 2.523 (1.45), 2.540 (0.16), 2.669 (0.58), 2.673 (0.42), |
| | 2.678 (0.19), 3.946 (6.42), 4.191 (1.19), 7.053 (0.27), 7.065 (1.29), |
| | 7.070 (1.55), 7.085 (1.45), 7.090 (1.44), 7.101 (0.28), 7.194 (0.32), |
| | 7.200 (0.61), 7.206 (0.35), 7.217 (0.68), 7.223 (1.20), 7.229 (0.66), |
| | 7.241 (0.34), 7.247 (0.62), 7.252 (0.34), 7.263 (0.68), 7.270 (1.04), |
| | 7.277 (0.75), 7.281 (0.81), 7.286 (1.12), 7.325 (0.38), 7.339 (1.67), |
| | 7.344 (2.05), 7.360 (3.83), 7.368 (1.23), 7.376 (1.12), 7.381 (0.33), |
| | 7.396 (0.18), 7.498 (1.52), 7.503 (1.48), 7.512 (1.49), 7.517 (1.58), |
| | 7.697 (1.57), 7.700 (1.54), 8.308 (1.91), 8.321 (1.81), 10.829 (2.14). |
| 89 | 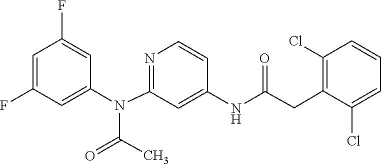 [SMILES: CC(=O)N(c1cc(F)cc(F)c1)c1cc(NC(=O)Cc2c(Cl)cccc2Cl)ccn1] |
| | N-{4-[2-(2,6-dichlorophenyl)acetamido]pyridin-2-yl}-N-(3,5- |
| | difluorophenyl)acetamide |
| | LC-MS (Method 1): Rₜ = 1.22 min; MS (ESIpos): m/z = 450 [M + H]⁺ |
| | ¹H-NMR (400 MHZ, DMSO-d6) δ [ppm]: 2.017 (16.00), 2.518 (2.37), |
| | 2.522 (1.50), 4.086 (7.45), 7.068 (1.49), 7.074 (1.76), 7.088 (1.66), |
| | 7.093 (1.63), 7.198 (0.64), 7.216 (0.75), 7.221 (1.29), 7.227 (0.71), |
| | 7.245 (0.64), 7.336 (1.36), 7.355 (1.86), 7.357 (2.02), 7.376 (2.23), |
| | 7.475 (1.62), 7.480 (1.76), 7.488 (7.59), 7.494 (1.94), 7.508 (4.06), |
| | 7.695 (1.79), 8.308 (2.07), 8.323 (1.99), 10.906 (2.33). |
| 90 | 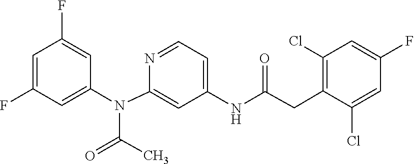 [SMILES: CC(=O)N(c1cc(F)cc(F)c1)c1cc(NC(=O)Cc2c(Cl)cc(F)cc2Cl)ccn1] |
| | N-{4-[2-(2,6-dichloro-4-fluorophenyl)acetamido]pyridin-2-yl}-N- |
| | (3,5-difluorophenyl)acetamide |
| | ¹H NMR (500 MHz, DMSO-d6) δ [ppm]: 2.02 (s, 3H), 4.05 (s, 2H), |
| | 7.04-7.12 (m, 2H), 7.17 - 7.25 (m, 1H), 7.48 (dd, J = 5.6, 1.9, 1H), 7.57 (d, |
| | J = 8.5, 2H), 7.69 (s, 1H), 8.32 (d, J = 5.6, 1H), 10.89 (s, 1H). |
| 91 | 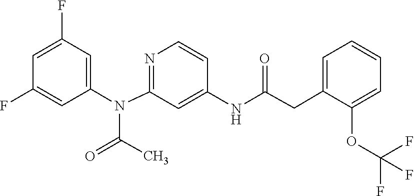 [SMILES: CC(=O)N(c1cc(F)cc(F)c1)c1cc(NC(=O)Cc2ccccc2OC(F)(F)F)ccn1] |
| | N-(3,5-difluorophenyl)-N-(4-{2-[2- |
| | (trifluoromethoxy)phenyl]acetamido}pyridin-2-yl)acetamide |
| | LC-MS (Method 1): Rₜ = 1.24 min; MS (ESIpos): m/z = 466 [M + H]⁺ |
| | ¹H-NMR (400 MHZ, DMSO-d6) δ [ppm]: 1.108 (1.32), 2.019 (16.00), |
| | 2.331 (0.47), 2.336 (0.20), 2.518 (2.65), 2.523 (1.81), 2.540 (1.58), |
| | 2.673 (0.45), 2.678 (0.20), 3.828 (5.95), 7.049 (0.25), 7.060 (1.20), |
| | 7.066 (1.45), 7.081 (1.35), 7.086 (1.35), 7.098 (0.26), 7.193 (0.31), |
| | 7.199 (0.57), 7.205 (0.35), 7.216 (0.66), 7.222 (1.11), 7.228 (0.63), |
| | 7.240 (0.32), 7.245 (0.56), 7.252 (0.31), 7.336 (0.53), 7.339 (0.73), |
| | 7.344 (0.85), 7.348 (0.72), 7.355 (0.79), 7.360 (1.21), 7.363 (2.38), |
| | 7.381 (1.56), 7.385 (0.92), 7.403 (1.11), 7.408 (1.42), 7.422 (0.91), |
| | 7.427 (1.27), 7.441 (0.41), 7.446 (0.41), 7.461 (1.45), 7.466 (1.04), |
| | 7.480 (1.02), 7.484 (0.73), 7.496 (1.51), 7.500 (1.51), 7.510 (1.52), |
| | 7.515 (1.51), 7.691 (1.48), 7.694 (1.42), 8.304 (1.80), 8.318 (1.71), |
| | 10.780 (1.99). |
| 92 | 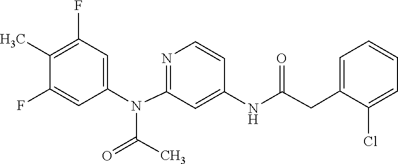 [SMILES: CC(=O)N(c1cc(F)c(C)c(F)c1)c1cc(NC(=O)Cc2ccccc2Cl)ccn1] |
| | N-{4-[2-(2-chlorophenyl)acetamido]pyridin-2-yl}-N-(3,5-difluoro-4- |
| | methylphenyl)acetamide |
| | LC-MS (Method 2): Rₜ = 1.21 min; MS (ESIneg): m/z = 428 [M − H]⁻ |
| | ¹H-NMR (400 MHZ, DMSO-d6) δ [ppm]: 2.014 (16.00), 2.139 (7.39), |
| | 3.881 (7.97), 7.041 (2.73), 7.060 (2.83), 7.303 (2.92), 7.312 (2.45), |
| | 7.317 (2.59), 7.326 (4.22), 7.336 (0.70), 7.398 (1.66), 7.408 (1.13), |
| | 7.412 (1.24), 7.421 (1.12), 7.426 (0.50), 7.436 (1.85), 7.445 (1.53), |
| | 7.450 (1.19), 7.459 (1.38), 7.485 (1.69), 7.489 (1.77), 7.498 (1.77), |
| | 7.504 (1.82), 7.702 (2.01), 8.279 (2.25), 8.294 (2.20), 10.773 (2.63). |
| 93 | 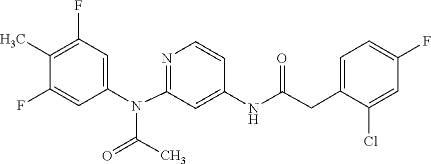 [SMILES: CC(=O)N(c1cc(F)c(C)c(F)c1)c1cc(NC(=O)Cc2ccc(F)cc2Cl)ccn1] |
| | N-{4-[2-(2-chloro-4-fluorophenyl)acetamido]pyridin-2-yl}-N-(3,5- |
| | difluoro-4-methylphenyl)acetamide |
| | LC-MS (Method 2): Rₜ = 1.24 min; MS (ESIneg): m/z = 446 [M − H]⁻ |
| | ¹H-NMR (400 MHz, DMSO-d6) δ [ppm]: 2.012 (16.00), 2.139 (6.03), |
| | 2.518 (0.82), 2.523 (0.55), 3.871 (5.72), 7.039 (2.29), 7.059 (2.30), |
| | 7.187 (0.68), 7.194 (0.74), 7.208 (1.37), 7.215 (1.60), 7.230 (0.80), |
| | 7.237 (0.85), 7.431 (1.48), 7.437 (1.49), 7.446 (1.32), 7.453 (1.57), |
| | 7.460 (2.04), 7.468 (1.23), 7.476 (1.65), 7.482 (2.09), 7.490 (1.54), |
| | 7.495 (1.65), 7.696 (1.58), 7.699 (1.56), 8.278 (1.98), 8.293 (1.87), |
| | 10.769 (2.20). |
| 94 | 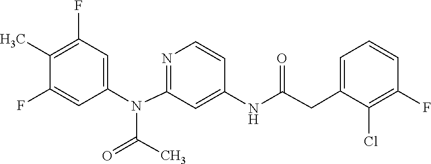 [SMILES: CC(=O)N(c1cc(F)c(C)c(F)c1)c1cc(NC(=O)Cc2cccc(F)c2Cl)ccn1] |
| | N-{4-[2-(2-chloro-3-fluorophenyl)acetamido]pyridin-2-yl}-N-(3,5- |
| | difluoro-4-methylphenyl)acetamide |
| | LC-MS (Method 2): Rₜ = 1.22 min; MS (ESIneg): m/z = 446 [M − H]⁻ |
| | ¹H-NMR (400 MHz, DMSO-d6) δ [ppm]: 2.013 (16.00), 2.139 (6.10), |
| | 2.518 (1.79), 2.523 (1.17), 3.943 (6.68), 7.042 (2.32), 7.062 (2.33), |
| | 7.263 (0.70), 7.270 (1.06), 7.276 (0.79), 7.280 (0.83), 7.286 (1.14), |
| | 7.324 (0.42), 7.339 (1.74), 7.343 (2.23), 7.360 (4.07), 7.367 (1.24), |
| | 7.375 (1.19), 7.477 (1.65), 7.482 (1.65), 7.491 (1.67), 7.496 (1.65), |
| | 7.698 (1.59), 7.701 (1.56), 8.281 (2.03), 8.295 (1.91), 10.809 (2.20). |
| 95 | 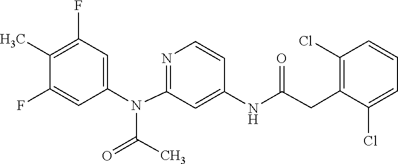 [SMILES: CC(=O)N(c1cc(F)c(C)c(F)c1)c1cc(NC(=O)Cc2c(Cl)cccc2Cl)ccn1] |
| | N-{4-[2-(2,6-dichlorophenyl)acetamido]pyridin-2-yl}-N-(3,5- |
| | difluoro-4-methylphenyl)acetamide |
| | LC-MS (Method 2): Rₜ = 1.26 min; MS (ESIneg): m/z = 462 [M − H]⁻ |
| | ¹H-NMR (400 MHz, DMSO-d6) δ [ppm]: 2.010 (16.00), 2.138 (6.70), |
| | 2.518 (5.23), 2.522 (3.78), 4.082 (7.30), 7.045 (2.48), 7.065 (2.60), |
| | 7.078 (0.44), 7.335 (1.37), 7.354 (1.96), 7.357 (2.07), 7.376 (2.46), |
| | 7.451 (1.57), 7.455 (1.72), 7.465 (1.70), 7.469 (1.79), 7.487 (6.67), |
| | 7.507 (4.12), 7.702 (1.73), 8.283 (2.11), 8.297 (2.05), 10.887 (2.41). |
| 96 | 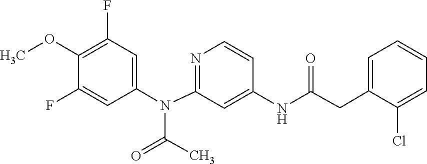 [SMILES: COc1c(F)cc(N(C(C)=O)c2cc(NC(=O)Cc3ccccc3Cl)ccn2)cc1F] |
| | N-{4-[2-(2-chlorophenyl)acetamido]pyridin-2-yl}-N-(3,5-difluoro-4- |
| | methoxyphenyl)acetamide |
| | LC-MS (Method 2): Rₜ = 1.15 min; MS (ESIpos): m/z = 446 [M + H]⁺ |
| | ¹H-NMR (400 MHz, DMSO-d6) δ [ppm]: 2.005 (16.00), 2.518 (1.69), |
| | 2.522 (1.09), 3.883 (7.23), 3.929 (11.28), 7.159 (2.56), 7.183 (2.61), |
| | 7.303 (3.01), 7.312 (1.82), 7.314 (1.91), 7.317 (2.04), 7.319 (2.05), |
| | 7.327 (4.04), 7.336 (0.41), 7.338 (0.48), 7.400 (1.53), 7.407 (0.75), |
| | 7.411 (0.93), 7.415 (1.00), 7.423 (0.98), 7.437 (1.79), 7.446 (1.44), |
| | 7.450 (0.97), 7.453 (0.69), 7.461 (1.22), 7.492 (1.63), 7.497 (1.56), |
| | 7.506 (1.59), 7.511 (1.67), 7.702 (1.59), 8.280 (2.09), 8.294 (1.97), |
| | 10.773 (2.26). |
| 97 | 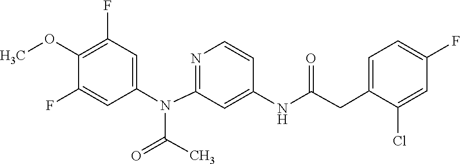 [SMILES: COc1c(F)cc(N(C(C)=O)c2cc(NC(=O)Cc3ccc(F)cc3Cl)ccn2)cc1F] |
| | N-{4-[2-(2-chloro-4-fluorophenyl)acetamido]pyridin-2-yl}-N-(3,5- |
| | difluoro-4-methoxyphenyl)acetamide |
| | LC-MS (Method 2): Rₜ = 1.18 min; MS (ESIpos): m/z = 464 [M + H]⁺ |
| | ¹H-NMR (400 MHz, DMSO-d6) δ [ppm]: 2.000 (16.00), 3.870 (8.19), |
| | 3.925 (14.27), 7.143 (0.54), 7.157 (3.38), 7.179 (3.66), 7.192 (1.59), |
| | 7.207 (1.78), 7.213 (1.96), 7.228 (0.96), 7.234 (1.06), 7.429 (1.62), |
| | 7.436 (1.80), 7.445 (1.83), 7.451 (2.10), 7.460 (2.60), 7.466 (1.94), |
| | 7.484 (3.13), 7.494 (1.99), 7.498 (2.19), 7.698 (2.62), 8.275 (2.59), |
| | 8.289 (2.54), 10.768 (3.36). |
| 98 | 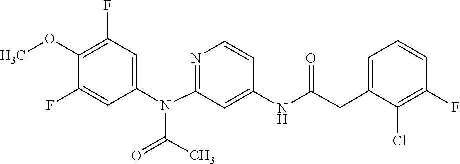 [SMILES: COc1c(F)cc(N(C(C)=O)c2cc(NC(=O)Cc3cccc(F)c3Cl)ccn2)cc1F] |
| | N-{4-[2-(2-chloro-3-fluorophenyl)acetamido]pyridin-2-yl}-N-(3,5- |
| | difluoro-4-methoxyphenyl)acetamide |
| | LC-MS (Method 2): Rₜ = 1.16 min; MS (ESIpos): m/z = 464 [M + H]⁺ |
| | ¹H-NMR (400 MHZ, DMSO-d6) δ [ppm]: 2.005 (16.00), 2.518 (1.53), |
| | 2.523 (0.97), 3.930 (11.22), 3.946 (6.54), 7.162 (2.51), 7.185 (2.52), |
| | 7.265 (0.69), 7.272 (1.05), 7.279 (0.77), 7.283 (0.83), 7.287 (1.14), |
| | 7.340 (1.74), 7.345 (2.29), 7.361 (3.74), 7.369 (1.27), 7.377 (1.11), |
| | 7.484 (1.50), 7.489 (1.60), 7.499 (1.55), 7.503 (1.61), 7.702 (1.52), |
| | 8.283 (2.01), 8.297 (1.91), 10.810 (2.22). |
| 99 | 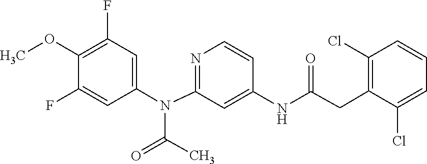 [SMILES: COc1c(F)cc(N(C(C)=O)c2cc(NC(=O)Cc3c(Cl)cccc3Cl)ccn2)cc1F] |
| | N-{4-[2-(2,6-dichlorophenyl)acetamido]pyridin-2-yl}-N-(3,5- |
| | difluoro-4-methoxyphenyl)acetamide |
| | LC-MS (Method 2): Rₜ = 1.20 min; MS (ESIneg): m/z = 478 [M − H]⁻ |
| | ¹H-NMR (400 MHZ, DMSO-d6) δ [ppm]: 2.002 (16.00), 2.518 (1.89), |
| | 2.523 (1.30), 3.929 (11.02), 4.085 (6.96), 7.165 (2.46), 7.188 (2.55), |
| | 7.337 (1.39), 7.355 (1.89), 7.359 (2.00), 7.377 (2.32), 7.459 (1.55), |
| | 7.463 (1.49), 7.473 (1.54), 7.478 (1.63), 7.489 (6.28), 7.509 (3.82), |
| | 7.703 (1.48), 8.284 (2.01), 8.298 (1.92), 10.887 (2.23). |
| 100 | 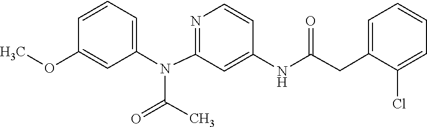 [SMILES: COc1cccc(N(C(C)=O)c2cc(NC(=O)Cc3ccccc3Cl)ccn2)c1] |
| | N-{4-[2-(2-chlorophenyl)acetamido]pyridin-2-yl}-N-(3- |
| | methoxyphenyl)acetamide |
| | LC-MS (Method 2): Rₜ = 1.06 min; MS (ESIneg): m/z = 408 [M − H]⁻ |
| | ¹H-NMR (400 MHZ, DMSO-d6) δ [ppm]: 1.996 (16.00), 2.518 (0.51), |
| | 3.336 (5.17), 3.872 (7.46), 6.800 (1.05), 6.803 (1.11), 6.804 (1.06), |
| | 6.820 (1.07), 6.822 (1.24), 6.824 (1.13), 6.860 (1.24), 6.866 (2.48), |
| | 6.871 (1.75), 6.877 (1.41), 6.879 (1.46), 6.884 (0.70), 6.886 (0.65), |
| | 6.898 (1.30), 6.900 (1.25), 6.904 (0.99), 6.906 (0.87), 7.285 (1.67), |
| | 7.299 (3.19), 7.305 (3.33), 7.314 (2.40), 7.323 (4.40), 7.333 (0.55), |
| | 7.394 (1.61), 7.401 (0.80), 7.404 (0.98), 7.408 (1.14), 7.417 (1.01), |
| | 7.423 (0.40), 7.433 (1.86), 7.442 (1.53), 7.447 (1.07), 7.457 (1.33), |
| | 7.464 (1.71), 7.469 (1.72), 7.478 (1.58), 7.483 (1.72), 7.650 (1.94), |
| | 7.654 (1.87), 8.265 (2.44), 8.279 (2.31), 10.738 (2.37). |
| 101 | 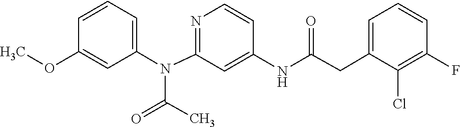 [SMILES: COc1cccc(N(C(C)=O)c2cc(NC(=O)Cc3cccc(F)c3Cl)ccn2)c1] |
| | N-{4-[2-(2-chloro-3-fluorophenyl)acetamido]pyridin-2-yl}-N-(3- |
| | methoxyphenyl)acetamide |
| | LC-MS (Method 2): Rₜ = 1.07 min; MS (ESIneg): m/z = 426 [M − H]⁻ |
| | ¹H-NMR (400 MHZ, DMSO-d6) δ [ppm]: 1.995 (16.00), 2.518 (0.78), |
| | 2.522 (0.48), 3.333 (10.83), 3.934 (6.61), 6.801 (1.00), 6.803 (1.03), |
| | 6.805 (1.02), 6.818 (0.90), 6.820 (0.99), 6.823 (1.18), 6.825 (1.08), |
| | 6.860 (1.17), 6.866 (2.36), 6.871 (1.67), 6.879 (1.32), 6.881 (1.36), |
| | 6.885 (0.70), 6.887 (0.64), 6.899 (1.24), 6.901 (1.17), 6.905 (0.96), |
| | 6.908 (0.83), 7.258 (0.70), 7.265 (1.09), 7.272 (0.78), 7.276 (0.83), |
| | 7.282 (1.22), 7.286 (1.72), 7.306 (2.64), 7.322 (0.48), 7.326 (1.31), |
| | 7.336 (1.74), 7.341 (2.07), 7.357 (4.12), 7.365 (1.26), 7.372 (1.20), |
| | 7.455 (1.60), 7.460 (1.65), 7.470 (1.53), 7.474 (1.72), 7.648 (1.77), |
| | 7.652 (1.73), 8.267 (2.30), 8.281 (2.17), 10.775 (2.23). |
| 102 | 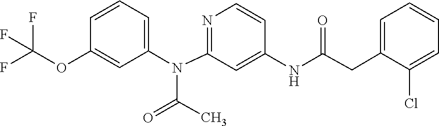 [SMILES: CC(=O)N(c1cccc(OC(F)(F)F)c1)c1cc(NC(=O)Cc2ccccc2Cl)ccn1] |
| | N-{4-[2-(2-chlorophenyl)acetamido]pyridin-2-yl}-N-[3- |
| | (trifluoromethoxy)phenyl]acetamide |
| | LC-MS (Method 2): Rₜ = 1.23 min; MS (ESIneg): m/z = 462 [M − H]⁻ |
| | ¹H-NMR (400 MHz, DMSO-d6) δ [ppm]: 2.005 (16.00), 2.518 (0.44), |
| | 3.336 (6.99), 7.244 (0.91), 7.247 (0.93), 7.264 (1.04), 7.266 (1.09), |
| | 7.300 (3.89), 7.309 (2.57), 7.315 (3.05), 7.323 (5.24), 7.333 (0.61), |
| | 7.366 (1.60), 7.395 (1.59), 7.403 (0.80), 7.406 (0.99), 7.410 (1.15), |
| | 7.419 (1.02), 7.424 (0.41), 7.434 (1.83), 7.443 (1.52), 7.447 (1.13), |
| | 7.457 (1.29), 7.501 (1.86), 7.509 (1.67), 7.514 (1.76), 7.521 (3.30), |
| | 7.523 (2.10), 7.528 (1.80), 7.541 (1.32), 7.675 (1.61), 8.305 (2.04), |
| | 8.319 (1.92), 10.782 (2.36). |
| 103 | 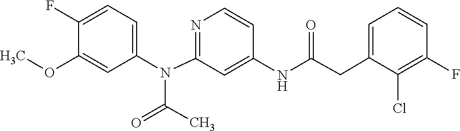 [SMILES: COc1cc(N(C(C)=O)c2cc(NC(=O)Cc3cccc(F)c3Cl)ccn2)ccc1F] |
| | N-{4-[2-(2-chloro-3-fluorophenyl)acetamido]pyridin-2-yl}-N-(4- |
| | fluoro-3-methoxyphenyl)acetamide |
| | LC-MS (Method 2): Rₜ = 1.10 min; MS (ESIpos): m/z = 446 [M + H]⁺ |
| | ¹H-NMR (400 MHZ, DMSO-d6) δ [ppm]: 2.003 (13.52), 2.518 (0.96), |
| | 2.522 (0.58), 3.331 (16.00), 3.941 (5.80), 6.802 (0.48), 6.808 (0.57), |
| | 6.812 (0.56), 6.818 (0.53), 6.823 (0.60), 6.830 (0.65), 6.834 (0.61), |
| | 6.839 (0.56), 7.157 (1.02), 7.163 (1.04), 7.177 (1.05), 7.183 (0.99), |
| | 7.207 (1.35), 7.228 (1.28), 7.235 (1.36), 7.256 (1.23), 7.263 (0.66), |
| | 7.270 (0.95), 7.277 (0.68), 7.281 (0.72), 7.286 (1.02), 7.339 (1.55), |
| | 7.344 (2.01), 7.360 (3.36), 7.367 (1.16), 7.376 (1.05), 7.456 (1.42), |
| | 7.461 (1.44), 7.470 (1.37), 7.475 (1.51), 7.700 (1.40), 7.703 (1.38), |
| | 8.252 (2.00), 8.267 (1.88), 10.777 (1.96). |
| 104 | 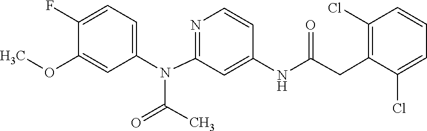 [SMILES: COc1cc(N(C(C)=O)c2cc(NC(=O)Cc3c(Cl)cccc3Cl)ccn2)ccc1F] |
| | N-{4-[2-(2,6-dichlorophenyl)acetamido]pyridin-2-yl}-N-(4-fluoro-3- |
| | methoxyphenyl)acetamide |
| | LC-MS (Method 2): Rₜ = 1.13 min; MS (ESIneg): m/z = 460 [M − H]⁻ |
| | ¹H-NMR (400 MHZ, DMSO-d6) δ [ppm]: 2.000 (13.82), 2.522 (1.06), |
| | 3.804 (16.00), 4.080 (7.13), 6.806 (0.56), 6.813 (0.71), 6.816 (0.72), |
| | 6.823 (0.66), 6.828 (0.73), 6.834 (0.83), 6.837 (0.76), 6.844 (0.64), |
| | 7.158 (1.20), 7.164 (1.22), 7.177 (1.23), 7.184 (1.13), 7.206 (1.43), |
| | 7.228 (1.39), 7.234 (1.45), 7.256 (1.27), 7.336 (1.29), 7.355 (1.82), |
| | 7.357 (1.88), 7.376 (2.04), 7.429 (1.59), 7.434 (1.53), 7.443 (1.57), |
| | 7.448 (1.57), 7.488 (6.26), 7.508 (4.04), 7.701 (1.80), 8.253 (2.31), |
| | 8.267 (2.16), 10.852 (2.44). |
| 105 | 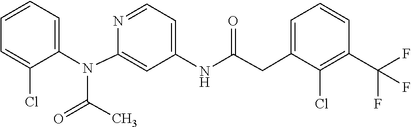 [SMILES: CC(=O)N(c1cc(NC(=O)Cc2cccc(C(F)(F)F)c2Cl)ccn1)c1ccccc1Cl] |
| | N-(2-chlorophenyl)-N-(4-{2-[2-chloro-3- |
| | (trifluoromethyl)phenyl]acetamido}pyridin-2-yl)acetamide |
| | LC-MS (Method 2): Rₜ = 1.23 min; MS (ESIneg): m/z = 480 [M − H]⁻ |
| | ¹H-NMR (400 MHz, DMSO-d6) δ [ppm]: 1.991 (16.00), 2.518 (1.43), |
| | 2.522 (0.91), 4.027 (14.23), 7.404 (0.78), 7.416 (5.07), 7.422 (3.84), |
| | 7.428 (4.48), 7.433 (4.39), 7.438 (9.76), 7.443 (5.03), 7.452 (4.91), |
| | 7.457 (4.40), 7.473 (2.98), 7.479 (1.62), 7.485 (1.89), 7.497 (1.28), |
| | 7.527 (1.66), 7.547 (3.75), 7.566 (2.20), 7.589 (0.66), 7.597 (4.08), |
| | 7.605 (2.23), 7.609 (3.51), 7.615 (2.22), 7.622 (3.12), 7.629 (0.44), |
| | 7.745 (3.08), 7.761 (2.39), 7.764 (2.42), 7.795 (3.30), 7.799 (2.97), |
| | 7.815 (2.95), 7.818 (2.59), 7.855 (1.27), 8.175 (3.48), 8.189 (3.32), |
| | 10.819 (5.42). |
| 106 | 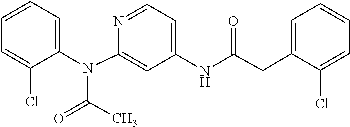 [SMILES: CC(=O)N(c1cc(NC(=O)Cc2ccccc2Cl)ccn1)c1ccccc1Cl] |
| | N-(2-chlorophenyl)-N-{4-[2-(2-chlorophenyl)acetamido]pyridin-2- |
| | yl}acetamide |
| | LC-MS (Method 2): Rₜ = 1.12 min; MS (ESIneg): m/z = 412 [M − H]⁻ |
| | ¹H-NMR (400 MHZ, DMSO-d6) δ [ppm]: 1.995 (16.00), 2.518 (0.97), |
| | 2.522 (0.61), 3.337 (15.72), 7.292 (0.61), 7.303 (6.72), 7.311 (3.77), |
| | 7.313 (4.39), 7.317 (4.38), 7.319 (4.47), 7.326 (9.06), 7.335 (0.87), |
| | 7.337 (1.04), 7.392 (0.55), 7.401 (3.96), 7.407 (2.02), 7.414 (6.87), |
| | 7.420 (4.17), 7.425 (6.02), 7.431 (4.47), 7.437 (10.45), 7.446 (3.77), |
| | 7.451 (6.06), 7.456 (4.92), 7.461 (3.88), 7.466 (4.82), 7.470 (7.21), |
| | 7.476 (1.80), 7.482 (1.90), 7.494 (1.23), 7.589 (0.60), 7.597 (4.04), |
| | 7.604 (2.19), 7.608 (3.40), 7.614 (2.07), 7.620 (3.06), 7.628 (0.41), |
| | 7.846 (1.32), 8.174 (3.48), 8.188 (3.32), 10.739 (5.26). |
| 107 | 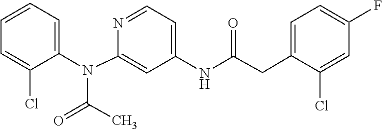 [SMILES: CC(=O)N(c1cc(NC(=O)Cc2ccc(F)cc2Cl)ccn1)c1ccccc1Cl] |
| | N-{4-[2-(2-chloro-4-fluorophenyl)acetamido]pyridin-2-yl}-N-(2- |
| | chlorophenyl)acetamide |
| | LC-MS (Method 2): Rₜ = 1.15 min; MS (ESIneg): m/z = 430 [M − H]⁻ |
| | ¹H-NMR (400 MHZ, DMSO-d6) δ [ppm]: 1.993 (16.00), 2.518 (1.25), |
| | 2.523 (0.86), 3.345 (7.57), 7.188 (1.60), 7.195 (1.81), 7.210 (3.37), |
| | 7.216 (3.68), 7.231 (1.84), 7.238 (2.04), 7.403 (0.75), 7.414 (5.25), |
| | 7.421 (3.81), 7.427 (4.28), 7.432 (7.58), 7.439 (10.87), 7.446 (5.32), |
| | 7.450 (4.75), 7.456 (7.07), 7.461 (7.74), 7.465 (4.38), 7.470 (5.15), |
| | 7.475 (1.86), 7.482 (2.10), 7.487 (3.55), 7.493 (1.27), 7.589 (0.63), |
| | 7.597 (3.99), 7.606 (2.18), 7.608 (2.96), 7.615 (2.03), 7.621 (3.04), |
| | 7.629 (0.40), 7.841 (1.30), 8.172 (3.47), 8.186 (3.29), 10.736 (5.27). |
| 108 | 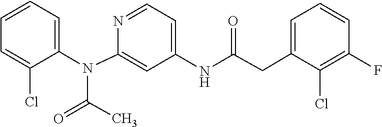 [SMILES: CC(=O)N(c1cc(NC(=O)Cc2cccc(F)c2Cl)ccn1)c1ccccc1Cl] |
| | N-{4-[2-(2-chloro-3-fluorophenyl)acetamido]pyridin-2-yl}-N-(2- |
| | chlorophenyl)acetamide |
| | LC-MS (Method 2): Rₜ = 1.14 min; MS (ESIneg): m/z = 430 [M − H]⁻ |
| | ¹H-NMR (400 MHz, DMSO-d6) δ [ppm]: 1.993 (16.00), 2.518 (1.04), |
| | 2.522 (0.65), 3.942 (15.18), 7.266 (1.64), 7.273 (2.42), 7.280 (1.80), |
| | 7.283 (1.97), 7.289 (2.68), 7.324 (0.96), 7.339 (4.05), 7.345 (5.95), |
| | 7.361 (8.37), 7.367 (2.93), 7.376 (2.69), 7.381 (0.83), 7.397 (0.71), |
| | 7.404 (0.76), 7.415 (5.18), 7.422 (3.69), 7.428 (4.31), 7.433 (4.41), |
| | 7.440 (6.92), 7.444 (4.71), 7.448 (4.37), 7.457 (4.41), 7.463 (4.38), |
| | 7.471 (3.00), 7.478 (1.59), 7.484 (1.85), 7.495 (1.20), 7.590 (0.60), |
| | 7.598 (4.07), 7.606 (2.30), 7.609 (3.28), 7.615 (2.09), 7.622 (3.18), |
| | 7.629 (0.42), 7.846 (1.27), 8.175 (3.46), 8.189 (3.28), 10.776 (5.17). |
| 109 | 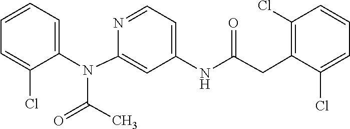 [SMILES: CC(=O)N(c1cc(NC(=O)Cc2c(Cl)cccc2Cl)ccn1)c1ccccc1Cl] |
| | N-(2-chlorophenyl)-N-{4-[2-(2,6-dichlorophenyl)acetamido]pyridin- |
| | 2-yl}acetamide |
| | LC-MS (Method 2): Rₜ = 1.18 min; MS (ESIneg): m/z = 446 [M − H]⁻ |
| | ¹H-NMR (400 MHZ, DMSO-d6) δ [ppm]: 1.991 (15.63), 2.296 (0.47), |
| | 2.518 (0.95), 2.522 (0.62), 3.957 (0.44), 4.081 (15.81), 7.336 (3.32), |
| | 7.355 (4.21), 7.357 (4.66), 7.376 (5.34), 7.404 (0.80), 7.416 (5.20), |
| | 7.421 (6.28), 7.426 (6.60), 7.433 (5.86), 7.440 (10.22), 7.446 (1.02), |
| | 7.451 (1.40), 7.459 (0.51), 7.473 (2.95), 7.480 (1.85), 7.489 (16.00), |
| | 7.497 (1.53), 7.508 (9.06), 7.590 (0.60), 7.599 (3.93), 7.606 (2.24), |
| | 7.610 (3.32), 7.615 (2.18), 7.622 (3.10), 7.629 (0.42), 7.844 (1.21), |
| | 8.176 (3.37), 8.189 (3.19), 10.848 (5.18). |
| 110 | 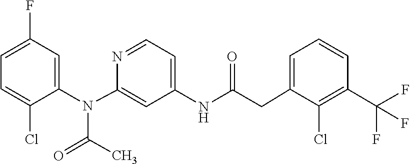 [SMILES: CC(=O)N(c1cc(NC(=O)Cc2cccc(C(F)(F)F)c2Cl)ccn1)c1cc(F)ccc1Cl] |
| | N-(2-chloro-5-fluorophenyl)-N-(4-{2-[2-chloro-3- |
| | (trifluoromethyl)phenyl]acetamido}pyridin-2-yl)acetamide |
| | LC-MS (Method 2): Rₜ = 1.27 min; MS (ESIneg): m/z = 498 [M − H]⁻ |
| | ¹H-NMR (400 MHZ, DMSO-d6) δ [ppm]: 2.017 (16.00), 2.518 (1.52), |
| | 2.522 (0.99), 4.033 (14.28), 7.321 (1.52), 7.328 (1.84), 7.341 (2.00), |
| | 7.343 (2.17), 7.348 (2.31), 7.351 (2.58), 7.364 (1.73), 7.371 (2.02), |
| | 7.450 (3.78), 7.454 (3.76), 7.464 (3.80), 7.468 (3.77), 7.507 (1.64), |
| | 7.514 (1.69), 7.530 (3.41), 7.537 (1.76), 7.549 (4.07), 7.569 (2.31), |
| | 7.648 (3.69), 7.661 (3.83), 7.670 (3.68), 7.684 (3.41), 7.749 (3.24), |
| | 7.768 (2.53), 7.797 (3.44), 7.801 (3.12), 7.816 (3.00), 7.820 (2.64), |
| | 7.893 (1.07), 8.176 (3.29), 8.189 (3.09), 10.834 (5.60). |
| 111 | 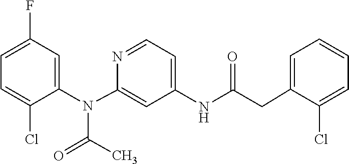 [SMILES: CC(=O)N(c1cc(NC(=O)Cc2ccccc2Cl)ccn1)c1cc(F)ccc1Cl] |
| | N-(2-chloro-5-fluorophenyl)-N-{4-[2-(2- |
| | chlorophenyl)acetamido]pyridin-2-yl}acetamide |
| | LC-MS (Method 1): Rₜ = 1.15 min; MS (ESIpos): m/z = 432 [M + H]⁺ |
| | ¹H-NMR (400 MHz, DMSO-d6) δ [ppm]: 2.023 (16.00), 2.518 (0.80), |
| | 2.522 (0.50), 3.337 (5.74), 7.294 (0.69), 7.297 (0.48), 7.305 (7.49), |
| | 7.312 (4.17), 7.315 (4.93), 7.318 (6.19), 7.321 (5.02), 7.328 (10.09), |
| | 7.340 (3.10), 7.347 (2.51), 7.348 (2.73), 7.361 (1.88), 7.369 (2.17), |
| | 7.396 (0.51), 7.405 (3.70), 7.411 (1.83), 7.416 (2.45), 7.420 (2.25), |
| | 7.428 (2.79), 7.439 (4.68), 7.447 (3.07), 7.452 (2.49), 7.455 (1.89), |
| | 7.464 (4.88), 7.469 (3.94), 7.478 (3.72), 7.483 (3.91), 7.502 (1.66), |
| | 7.510 (1.66), 7.525 (1.69), 7.532 (1.56), 7.646 (3.82), 7.660 (3.94), |
| | 7.668 (3.79), 7.682 (3.51), 7.883 (1.10), 8.174 (3.26), 8.188 (3.12), |
| | 10.754 (5.38). |
| 112 | 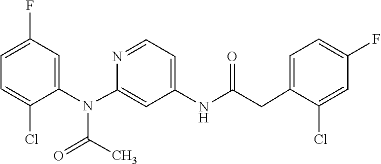 [SMILES: CC(=O)N(c1cc(NC(=O)Cc2ccc(F)cc2Cl)ccn1)c1cc(F)ccc1Cl] |
| | N-(2-chloro-5-fluorophenyl)-N-{4-[2-(2-chloro-4- |
| | fluorophenyl)acetamido]pyridin-2-yl}acetamide |
| | LC-MS (Method 1): Rₜ = 1.18 min; MS (ESIpos): m/z = 450 [M + H]⁺ |
| | ¹H-NMR (400 MHz, DMSO-d6) δ [ppm]: 2.021 (16.00), 2.518 (0.70), |
| | 2.522 (0.46), 3.342 (3.91), 7.190 (1.70), 7.197 (1.87), 7.211 (3.46), |
| | 7.218 (3.95), 7.232 (1.93), 7.239 (2.12), 7.319 (1.58), 7.326 (1.94), |
| | 7.339 (2.05), 7.341 (2.20), 7.347 (2.40), 7.349 (2.57), 7.361 (1.80), |
| | 7.369 (2.03), 7.433 (3.65), 7.439 (3.85), 7.455 (8.68), 7.461 (6.90), |
| | 7.469 (7.07), 7.475 (6.68), 7.491 (2.82), 7.502 (1.67), 7.509 (1.67), |
| | 7.525 (1.68), 7.532 (1.55), 7.646 (3.73), 7.660 (3.76), 7.668 (3.62), |
| | 7.682 (3.46), 7.880 (1.12), 8.173 (3.32), 8.187 (3.14), 10.751 (5.63). |
| 113 | 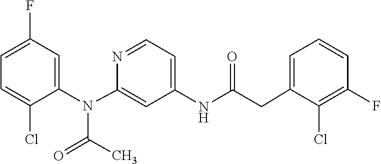 [SMILES: CC(=O)N(c1cc(NC(=O)Cc2cccc(F)c2Cl)ccn1)c1cc(F)ccc1Cl] |
| | N-(2-chloro-5-fluorophenyl)-N-{4-[2-(2-chloro-3- |
| | fluorophenyl)acetamido]pyridin-2-yl}acetamide |
| | LC-MS (Method 1): Rₜ = 1.17 min; MS (ESIneg): m/z = 448 [M − H]⁻ |
| | ¹H-NMR (400 MHZ, DMSO-d6) δ [ppm]: 2.021 (16.00), 2.518 (1.23), |
| | 2.522 (0.78), 3.948 (15.79), 7.270 (1.74), 7.276 (2.55), 7.283 (1.90), |
| | 7.287 (2.09), 7.293 (2.88), 7.321 (1.90), 7.328 (2.23), 7.341 (6.24), |
| | 7.347 (7.54), 7.363 (9.87), 7.369 (3.98), 7.378 (2.86), 7.384 (0.92), |
| | 7.399 (0.51), 7.456 (3.88), 7.460 (4.11), 7.469 (4.06), 7.474 (4.23), |
| | 7.506 (1.63), 7.513 (1.63), 7.528 (1.67), 7.535 (1.54), 7.648 (3.79), |
| | 7.661 (3.82), 7.670 (3.61), 7.684 (3.48), 7.884 (1.08), 8.176 (3.32), |
| | 8.189 (3.14), 10.790 (5.55). |
| 114 | 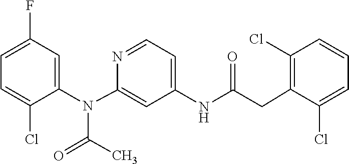 [SMILES: CC(=O)N(c1cc(NC(=O)Cc2c(Cl)cccc2Cl)ccn1)c1cc(F)ccc1Cl] |
| | N-(2-chloro-5-fluorophenyl)-N-{4-[2-(2,6- |
| | dichlorophenyl)acetamido]pyridin-2-yl}acetamide |
| | LC-MS (Method 1): Rₜ = 1.21 min; MS (ESIpos): m/z = 466 [M + H]⁺ |
| | ¹H-NMR (400 MHZ, DMSO-d6) δ [ppm]: 2.018 (16.00), 2.073 (2.49), |
| | 2.518 (0.72), 2.522 (0.43), 4.088 (15.85), 7.319 (1.55), 7.328 (1.90), |
| | 7.336 (3.87), 7.340 (2.41), 7.342 (2.37), 7.347 (2.48), 7.350 (2.81), |
| | 7.355 (4.91), 7.358 (5.14), 7.362 (2.20), 7.370 (2.20), 7.377 (5.79), |
| | 7.435 (3.38), 7.439 (3.49), 7.449 (3.50), 7.453 (3.56), 7.489 (15.61), |
| | 7.509 (11.06), 7.530 (1.69), 7.537 (1.56), 7.647 (3.64), 7.661 (3.66), |
| | 7.670 (3.60), 7.684 (3.41), 7.884 (1.03), 8.135 (0.56), 8.176 (3.20), |
| | 8.190 (3.03), 10.863 (5.37). |
| 115 | 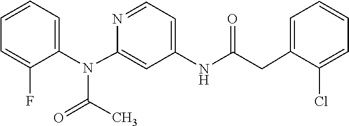 [SMILES: CC(=O)N(c1cc(NC(=O)Cc2ccccc2Cl)ccn1)c1ccccc1F] |
| | N-{4-[2-(2-chlorophenyl)acetamido]pyridin-2-yl}-N-(2- |
| | fluorophenyl)acetamide |
| | LC-MS (Method 2): Rₜ = 1.09 min; MS (ESIneg): m/z = 396 [M − H]⁻ |
| | ¹H-NMR (400 MHZ, DMSO-d6) δ [ppm]: 2.031 (16.00), 2.518 (0.64), |
| | 2.522 (0.42), 3.335 (9.06), 7.229 (0.85), 7.232 (0.94), 7.248 (1.79), |
| | 7.251 (2.00), 7.266 (1.35), 7.270 (1.46), 7.292 (0.41), 7.302 (4.11), |
| | 7.312 (2.92), 7.316 (2.97), 7.318 (3.17), 7.321 (1.49), 7.326 (6.45), |
| | 7.336 (0.85), 7.342 (1.58), 7.346 (1.84), 7.350 (1.05), 7.357 (0.93), |
| | 7.361 (1.12), 7.367 (1.64), 7.371 (1.60), 7.376 (1.55), 7.380 (1.68), |
| | 7.390 (0.60), 7.399 (3.57), 7.409 (2.08), 7.413 (2.67), 7.422 (1.79), |
| | 7.426 (1.40), 7.433 (1.26), 7.437 (2.93), 7.445 (2.39), 7.452 (3.19), |
| | 7.457 (2.53), 7.459 (2.55), 7.467 (2.18), 7.471 (2.24), 7.757 (1.53), |
| | 8.215 (2.61), 8.229 (2.49), 10.753 (3.22). |
| 116 | 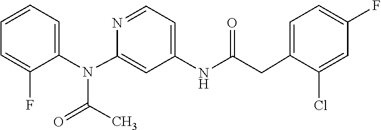 [SMILES: CC(=O)N(c1cc(NC(=O)Cc2ccc(F)cc2Cl)ccn1)c1ccccc1F] |
| | N-{4-[2-(2-chloro-4-fluorophenyl)acetamido]pyridin-2-yl}-N-(2- |
| | fluorophenyl)acetamide |
| | LC-MS (Method 2): Rₜ = 1.11 min; MS (ESIneg): m/z = 414 [M − H]⁻ |
| | ¹H-NMR (400 MHZ, DMSO-d6) δ [ppm]: 2.030 (16.00), 2.518 (0.49), |
| | 3.338 (7.76), 7.187 (1.02), 7.194 (1.10), 7.209 (2.05), 7.215 (2.38), |
| | 7.230 (1.89), 7.232 (1.19), 7.237 (1.38), 7.249 (1.76), 7.252 (1.91), |
| | 7.267 (1.31), 7.270 (1.39), 7.322 (0.78), 7.325 (0.85), 7.343 (1.49), |
| | 7.347 (1.76), 7.351 (1.03), 7.357 (0.93), 7.361 (1.10), 7.368 (1.63), |
| | 7.372 (1.60), 7.376 (1.52), 7.380 (1.64), 7.395 (1.45), 7.400 (1.38), |
| | 7.409 (0.98), 7.413 (1.33), 7.416 (0.96), 7.420 (0.74), 7.426 (0.99), |
| | 7.431 (2.97), 7.438 (2.71), 7.445 (2.96), 7.449 (4.00), 7.453 (2.86), |
| | 7.459 (4.09), 7.464 (4.40), 7.469 (1.86), 7.485 (1.61), 7.753 (1.51), |
| | 8.214 (2.63), 8.228 (2.49), 10.751 (3.27). |
| 117 | 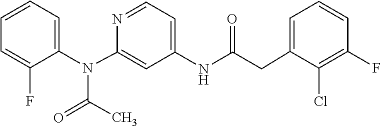 [SMILES: CC(=O)N(c1cc(NC(=O)Cc2cccc(F)c2Cl)ccn1)c1ccccc1F] |
| | N-{4-[2-(2-chloro-3-fluorophenyl)acetamido]pyridin-2-yl}-N-(2- |
| | fluorophenyl)acetamide |
| | LC-MS (Method 2): Rₜ = 1.10 min; MS (ESIneg): m/z = 414 [M − H]⁻ |
| | ¹H-NMR (400 MHz, DMSO-d6) δ [ppm]: 2.031 (16.00), 2.518 (0.65), |
| | 2.523 (0.41), 3.941 (9.77), 7.230 (0.88), 7.233 (0.96), 7.250 (1.93), |
| | 7.252 (2.08), 7.271 (3.03), 7.278 (1.25), 7.282 (1.35), 7.287 (1.85), |
| | 7.323 (1.28), 7.326 (1.10), 7.338 (2.56), 7.343 (5.08), 7.352 (1.44), |
| | 7.359 (6.46), 7.367 (2.81), 7.374 (2.52), 7.381 (1.98), 7.397 (1.47), |
| | 7.401 (1.36), 7.409 (1.06), 7.414 (1.35), 7.428 (0.90), 7.430 (0.92), |
| | 7.435 (0.93), 7.440 (0.57), 7.446 (2.21), 7.450 (2.28), 7.460 (2.09), |
| | 7.464 (2.12), 7.758 (1.60), 8.217 (2.68), 8.231 (2.55), 10.791 (3.36). |
| 118 | 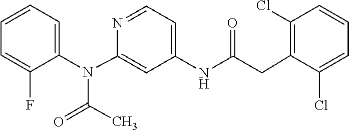 [SMILES: CC(=O)N(c1cc(NC(=O)Cc2c(Cl)cccc2Cl)ccn1)c1ccccc1F] |
| | N-{4-[2-(2,6-dichlorophenyl)acetamido]pyridin-2-yl}-N-(2- |
| | fluorophenyl)acetamide |
| | LC-MS (Method 2): Rₜ = 1.14 min; MS (ESIneg): m/z = 430 [M − H]⁻ |
| | ¹H-NMR (400 MHZ, DMSO-d6) δ [ppm]: 2.028 (16.00), 2.518 (0.64), |
| | 2.522 (0.41), 4.080 (9.85), 7.230 (0.87), 7.233 (0.93), 7.250 (1.77), |
| | 7.252 (1.96), 7.268 (1.32), 7.271 (1.43), 7.322 (0.80), 7.326 (0.89), |
| | 7.335 (2.08), 7.343 (1.58), 7.347 (1.86), 7.354 (3.00), 7.357 (3.10), |
| | 7.361 (1.10), 7.369 (1.87), 7.376 (3.81), 7.381 (1.57), 7.385 (1.67), |
| | 7.396 (1.03), 7.400 (1.36), 7.405 (0.93), 7.409 (1.10), 7.414 (1.36), |
| | 7.417 (1.12), 7.422 (2.59), 7.427 (2.85), 7.436 (2.54), 7.441 (2.34), |
| | 7.448 (0.52), 7.487 (9.21), 7.507 (5.63), 7.752 (1.44), 8.217 (2.58), |
| | 8.231 (2.44), 10.865 (3.23). |
| 119 | 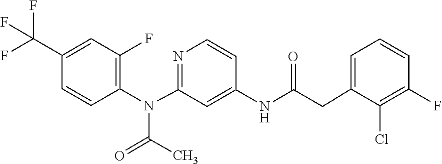 [SMILES: CC(=O)N(c1cc(NC(=O)Cc2cccc(F)c2Cl)ccn1)c1ccc(C(F)(F)F)cc1F] |
| | N-{4-[2-(2-chloro-3-fluorophenyl)acetamido]pyridin-2-yl}-N-[2- |
| | fluoro-4-(trifluoromethyl)phenyl]acetamide |
| | LC-MS (Method 2): Rₜ = 1.26 min; MS (ESIneg): m/z = 482 [M − H]⁻ |
| | ¹H-NMR (400 MHZ, DMSO-d6) δ [ppm]: 2.080 (16.00), 2.523 (1.96), |
| | 3.948 (10.66), 7.267 (1.15), 7.274 (1.71), 7.280 (1.32), 7.284 (1.43), |
| | 7.290 (2.00), 7.327 (0.62), 7.341 (2.78), 7.346 (3.64), 7.362 (6.16), |
| | 7.369 (2.32), 7.377 (1.97), 7.383 (0.72), 7.473 (2.42), 7.478 (2.62), |
| | 7.488 (2.60), 7.492 (2.77), 7.611 (0.48), 7.632 (1.35), 7.650 (4.15), |
| | 7.654 (3.48), 7.671 (0.78), 7.676 (0.83), 7.776 (1.32), 7.870 (1.60), |
| | 7.873 (1.63), 7.896 (1.83), 8.239 (2.28), 8.253 (2.24), 10.827 (3.81). |
| 120 | 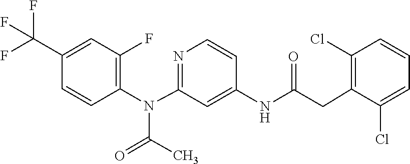 [SMILES: CC(=O)N(c1cc(NC(=O)Cc2c(Cl)cccc2Cl)ccn1)c1ccc(C(F)(F)F)cc1F] |
| | N-{4-[2-(2,6-dichlorophenyl)acetamido]pyridin-2-yl}-N-[2-fluoro-4- |
| | (trifluoromethyl)phenyl]acetamide |
| | LC-MS (Method 2): Rₜ = 1.30 min; MS (ESIneg): m/z = 498 [M − H]⁻ |
| | ¹H-NMR (400 MHZ, DMSO-d6) δ [ppm]: 2.077 (16.00), 2.522 (3.26), |
| | 4.087 (11.22), 7.338 (2.23), 7.357 (2.98), 7.360 (3.36), 7.378 (3.65), |
| | 7.454 (2.35), 7.459 (2.55), 7.469 (2.54), 7.473 (2.72), 7.490 (10.30), |
| | 7.509 (6.32), 7.613 (0.46), 7.634 (1.39), 7.650 (4.73), 7.653 (3.96), |
| | 7.671 (0.78), 7.675 (0.83), 7.769 (1.25), 7.869 (1.63), 7.872 (1.67), |
| | 7.895 (1.91), 8.239 (2.24), 8.253 (2.23), 10.902 (3.89). |
| 121 | 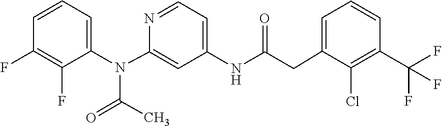 [SMILES: CC(=O)N(c1cc(NC(=O)Cc2cccc(C(F)(F)F)c2Cl)ccn1)c1cccc(F)c1F] |
| | N-(4-{2-[2-chloro-3-(trifluoromethyl)phenyl]acetamido}pyridin-2- |
| | yl)-N-(2,3-difluorophenyl)acetamide |
| | LC-MS (Method 2): Rₜ = 1.23 min; MS (ESIpos): m/z = 484 [M + H]⁺ |
| | ¹H-NMR (400 MHZ, DMSO-d6) δ [ppm]: 2.065 (16.00), 2.518 (0.86), |
| | 2.522 (0.53), 4.032 (8.64), 7.195 (0.44), 7.215 (1.03), 7.230 (0.97), |
| | 7.237 (1.11), 7.249 (0.70), 7.253 (0.70), 7.257 (0.95), 7.260 (0.95), |
| | 7.270 (0.95), 7.274 (0.91), 7.445 (0.43), 7.450 (0.48), 7.459 (2.35), |
| | 7.464 (2.85), 7.474 (2.73), 7.478 (2.62), 7.490 (0.93), 7.494 (0.79), |
| | 7.511 (0.41), 7.515 (0.41), 7.528 (0.99), 7.547 (2.24), 7.566 (1.31), |
| | 7.746 (1.89), 7.765 (1.61), 7.784 (1.35), 7.796 (2.25), 7.799 (2.00), |
| | 7.816 (1.73), 7.820 (1.52), 8.240 (2.30), 8.254 (2.18), 10.860 (3.23). |
| 122 | 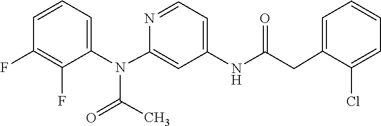 [SMILES: CC(=O)N(c1cc(NC(=O)Cc2ccccc2Cl)ccn1)c1cccc(F)c1F] |
| | N-{4-[2-(2-chlorophenyl)acetamido]pyridin-2-yl}-N-(2,3- |
| | difluorophenyl)acetamide |
| | LC-MS (Method 1): Rₜ = 1.12 min; MS (ESIpos): m/z = 416 [M + H]⁺ |
| | ¹H-NMR (400 MHz, DMSO-d6) δ [ppm]: 2.069 (16.00), 3.339 (3.05), |
| | 7.192 (0.44), 7.212 (1.03), 7.227 (0.95), 7.236 (0.97), 7.248 (0.70), |
| | 7.252 (0.71), 7.256 (0.95), 7.259 (0.92), 7.268 (0.94), 7.273 (0.90), |
| | 7.279 (0.41), 7.289 (0.42), 7.293 (0.69), 7.304 (4.24), 7.312 (2.48), |
| | 7.314 (2.76), 7.317 (2.81), 7.319 (2.80), 7.328 (5.36), 7.336 (0.55), |
| | 7.338 (0.62), 7.402 (2.09), 7.409 (1.01), 7.412 (1.31), 7.417 (1.39), |
| | 7.425 (1.40), 7.438 (2.55), 7.447 (2.25), 7.450 (1.55), 7.453 (1.13), |
| | 7.461 (2.15), 7.469 (1.35), 7.474 (2.86), 7.478 (2.56), 7.488 (3.19), |
| | 7.492 (3.07), 7.509 (0.42), 7.775 (1.35), 8.239 (2.28), 8.253 (2.18), |
| | 10.778 (3.08). |
| 123 | 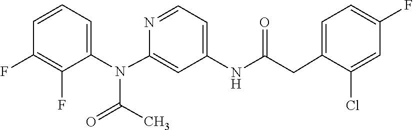 [SMILES: CC(=O)N(c1cc(NC(=O)Cc2ccc(F)cc2Cl)ccn1)c1cccc(F)c1F] |
| | N-{4-[2-(2-chloro-4-fluorophenyl)acetamido]pyridin-2-yl}-N-(2,3- |
| | difluorophenyl)acetamide |
| | LC-MS (Method 1): Rₜ = 1.14 min; MS (ESIpos): m/z = 434 [M + H]⁺ |
| | ¹H-NMR (400 MHZ, DMSO-d6) δ [ppm]: 2.068 (16.00), 2.073 (3.48), |
| | 3.340 (1.70), 7.188 (1.25), 7.195 (1.47), 7.209 (2.88), 7.216 (3.04), |
| | 7.230 (1.90), 7.237 (2.13), 7.248 (0.72), 7.252 (0.70), 7.256 (0.96), |
| | 7.259 (0.94), 7.268 (0.95), 7.272 (0.90), 7.279 (0.40), 7.431 (2.18), |
| | 7.438 (2.20), 7.444 (0.63), 7.453 (2.98), 7.460 (2.56), 7.466 (4.61), |
| | 7.471 (3.94), 7.480 (2.63), 7.485 (2.97), 7.488 (2.76), 7.509 (0.41), |
| | 7.772 (1.34), 8.134 (0.40), 8.238 (2.27), 8.252 (2.17), 10.776 (3.11). |
| 124 | 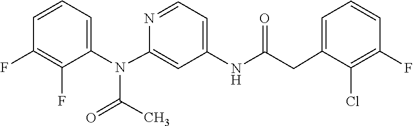 [SMILES: CC(=O)N(c1cc(NC(=O)Cc2cccc(F)c2Cl)ccn1)c1cccc(F)c1F] |
| | N-{4-[2-(2-chloro-3-fluorophenyl)acetamido]pyridin-2-yl}-N-(2,3- |
| | difluorophenyl)acetamide |
| | LC-MS (Method 1): Rₜ = 1.13 min; MS (ESIpos): m/z = 434 [M + H]⁺ |
| | ¹H-NMR (400 MHZ, DMSO-d6) δ [ppm]: 2.069 (16.00), 2.073 (4.84), |
| | 3.949 (9.25), 7.195 (0.45), 7.215 (1.04), 7.229 (0.99), 7.237 (1.15), |
| | 7.249 (0.76), 7.252 (0.76), 7.257 (1.01), 7.259 (1.02), 7.269 (1.57), |
| | 7.273 (2.34), 7.281 (1.44), 7.285 (1.32), 7.290 (2.01), 7.323 (0.56), |
| | 7.338 (2.33), 7.344 (3.64), 7.360 (4.70), 7.366 (1.84), 7.375 (1.58), |
| | 7.381 (0.50), 7.444 (0.43), 7.449 (0.45), 7.468 (3.10), 7.473 (2.88), |
| | 7.482 (2.74), 7.487 (2.97), 7.509 (0.42), 7.777 (1.35), 8.241 (2.31), |
| | 8.255 (2.20), 10.817 (3.16). |
| 125 | 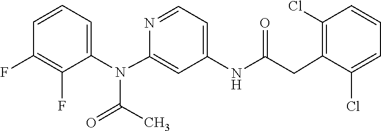 [SMILES: CC(=O)N(c1cc(NC(=O)Cc2c(Cl)cccc2Cl)ccn1)c1cccc(F)c1F] |
| | N-{4-[2-(2,6-dichlorophenyl)acetamido]pyridin-2-yl}-N-(2,3- |
| | difluorophenyl)acetamide |
| | LC-MS (Method 1): Rₜ = 1.17 min; MS (ESIpos): m/z = 450 [M + H]⁺ |
| | ¹H-NMR (400 MHZ, DMSO-d6) δ [ppm]: 2.066 (16.00), 2.073 (1.96), |
| | 2.518 (0.41), 3.341 (0.54), 4.087 (9.39), 7.197 (0.44), 7.217 (1.04), |
| | 7.231 (1.03), 7.237 (1.44), 7.249 (0.75), 7.252 (0.75), 7.256 (0.97), |
| | 7.259 (0.96), 7.270 (0.94), 7.273 (0.96), 7.293 (0.47), 7.336 (2.03), |
| | 7.354 (2.64), 7.357 (2.91), 7.376 (3.23), 7.446 (2.23), 7.451 (2.16), |
| | 7.461 (2.27), 7.465 (2.59), 7.473 (0.74), 7.488 (9.93), 7.507 (5.75), |
| | 7.770 (1.25), 8.241 (2.21), 8.255 (2.09), 10.895 (3.02). |
| 126 | 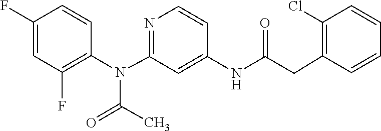 [SMILES: CC(=O)N(c1cc(NC(=O)Cc2ccccc2Cl)ccn1)c1ccc(F)cc1F] |
| | N-{4-[2-(2-chlorophenyl)acetamido]pyridin-2-yl}-N-(2,4- |
| | difluorophenyl)acetamide |
| | LC-MS (Method 1): Rₜ = 1.14 min; MS (ESIpos): m/z = 416 [M + H]⁺ |
| | ¹H-NMR (400 MHz, DMSO-d6) δ [ppm]: 2.032 (16.00), 2.074 (0.57), |
| | 2.332 (0.87), 2.518 (3.87), 2.522 (2.48), 2.673 (0.88), 3.880 (10.71), |
| | 7.128 (0.54), 7.132 (0.64), 7.135 (0.61), 7.148 (1.08), 7.154 (1.14), |
| | 7.171 (0.63), 7.174 (0.67), 7.178 (0.64), 7.295 (0.40), 7.305 (4.71), |
| | 7.315 (2.98), 7.319 (2.93), 7.321 (3.02), 7.329 (5.97), 7.339 (0.68), |
| | 7.401 (2.78), 7.410 (1.83), 7.416 (1.62), 7.425 (2.42), 7.428 (1.74), |
| | 7.432 (1.46), 7.435 (1.54), 7.438 (3.08), 7.447 (2.83), 7.450 (4.14), |
| | 7.455 (4.09), 7.464 (4.07), 7.469 (3.10), 7.480 (1.23), 7.487 (0.68), |
| | 7.502 (0.56), 7.782 (1.42), 8.209 (2.61), 8.223 (2.44), 10.759 (3.35). |
| 127 | 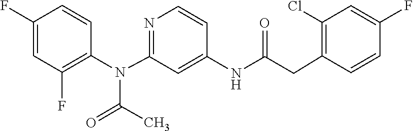 [SMILES: CC(=O)N(c1cc(NC(=O)Cc2ccc(F)cc2Cl)ccn1)c1ccc(F)cc1F] |
| | N-{4-[2-(2-chloro-4-fluorophenyl)acetamido]pyridin-2-yl}-N-(2,4- |
| | difluorophenyl)acetamide |
| | LC-MS (Method 1): Rₜ = 1.17 min; MS (ESIpos): m/z = 434 [M + H]⁺ |
| | ¹H-NMR (400 MHZ, DMSO-d6) δ [ppm]: 2.031 (16.00), 2.074 (0.90), |
| | 2.332 (0.62), 2.518 (2.96), 2.522 (1.87), 2.673 (0.63), 3.872 (8.66), |
| | 7.129 (0.54), 7.133 (0.63), 7.136 (0.60), 7.149 (1.08), 7.155 (1.15), |
| | 7.169 (0.62), 7.171 (0.64), 7.176 (0.68), 7.179 (0.66), 7.190 (1.04), |
| | 7.197 (1.14), 7.211 (2.15), 7.218 (2.47), 7.233 (1.20), 7.240 (1.25), |
| | 7.404 (0.92), 7.411 (0.99), 7.426 (1.23), 7.429 (1.34), 7.434 (3.32), |
| | 7.442 (5.11), 7.447 (3.32), 7.450 (3.01), 7.456 (5.71), 7.461 (4.26), |
| | 7.463 (4.34), 7.471 (2.10), 7.480 (1.33), 7.487 (2.23), 7.502 (0.58), |
| | 7.779 (1.41), 8.208 (2.58), 8.222 (2.47), 10.758 (3.39). |
| 128 | 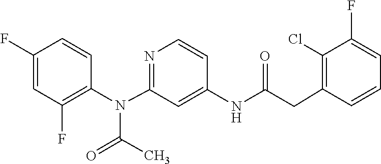 [SMILES: CC(=O)N(c1cc(NC(=O)Cc2cccc(F)c2Cl)ccn1)c1ccc(F)cc1F] |
| | N-{4-[2-(2-chloro-3-fluorophenyl)acetamido]pyridin-2-yl}-N-(2,4- |
| | difluorophenyl)acetamide |
| | LC-MS (Method 1): Rₜ = 1.15 min; MS (ESIpos): m/z = 434 [M + H]⁺ |
| | ¹H-NMR (400 MHz, DMSO-d6) δ [ppm]: 2.031 (16.00), 2.074 (0.56), |
| | 2.518 (2.20), 2.522 (1.42), 2.673 (0.45), 3.944 (10.16), 7.129 (0.56), |
| | 7.133 (0.63), 7.137 (0.61), 7.150 (1.09), 7.155 (1.16), 7.169 (0.62), |
| | 7.173 (0.63), 7.176 (0.70), 7.180 (0.66), 7.266 (1.07), 7.273 (1.61), |
| | 7.280 (1.15), 7.284 (1.27), 7.290 (1.74), 7.327 (0.59), 7.341 (2.59), |
| | 7.347 (3.50), 7.362 (5.86), 7.369 (1.96), 7.378 (1.80), 7.383 (0.54), |
| | 7.404 (0.92), 7.411 (0.96), 7.426 (1.17), 7.430 (1.21), 7.433 (1.27), |
| | 7.437 (1.25), 7.444 (3.27), 7.448 (2.90), 7.452 (1.38), 7.457 (3.41), |
| | 7.463 (3.31), 7.466 (1.42), 7.482 (1.23), 7.489 (0.68), 7.504 (0.55), |
| | 7.784 (1.40), 8.212 (2.64), 8.226 (2.51), 10.798 (3.39). |
| 129 | 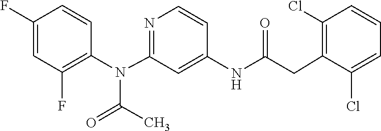 [SMILES: CC(=O)N(c1cc(NC(=O)Cc2c(Cl)cccc2Cl)ccn1)c1ccc(F)cc1F] |
| | N-{4-[2-(2,6-dichlorophenyl)acetamido]pyridin-2-yl}-N-(2,4- |
| | difluorophenyl)acetamide |
| | LC-MS (Method 1): Rₜ = 1.20 min; MS (ESIpos): m/z = 450 [M + H]⁺ |
| | ¹H-NMR (400 MHZ, DMSO-d6) δ [ppm]: 1.867 (0.44), 2.029 (16.00), |
| | 2.074 (0.80), 2.332 (0.56), 2.518 (2.76), 2.522 (1.74), 2.673 (0.58), |
| | 4.083 (10.68), 7.126 (0.53), 7.129 (0.56), 7.133 (0.63), 7.136 (0.62), |
| | 7.150 (1.11), 7.155 (1.16), 7.169 (0.63), 7.172 (0.64), 7.176 (0.70), |
| | 7.179 (0.64), 7.338 (2.23), 7.357 (2.82), 7.360 (3.14), 7.379 (3.43), |
| | 7.403 (0.92), 7.411 (1.00), 7.421 (2.74), 7.426 (3.61), 7.435 (3.40), |
| | 7.440 (2.88), 7.447 (0.83), 7.452 (1.16), 7.459 (1.21), 7.463 (0.87), |
| | 7.469 (1.26), 7.490 (10.42), 7.510 (6.18), 7.779 (1.33), 8.212 (2.60), |
| | 8.226 (2.44), 10.872 (3.40). |
| 130 | 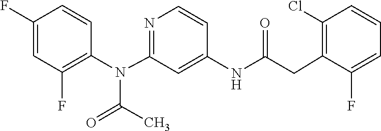 [SMILES: CC(=O)N(c1cc(NC(=O)Cc2c(F)cccc2Cl)ccn1)c1ccc(F)cc1F] |
| | N-{4-[2-(2-chloro-6-fluorophenyl)acetamido]pyridin-2-yl}-N-(2,4- |
| | difluorophenyl)acetamide |
| | LC-MS (Method 1): Rₜ = 1.15 min; MS (ESIpos): m/z = 434 [M + H]⁺ |
| | ¹H-NMR (400 MHz, DMSO-d6) δ [ppm]: 2.030 (16.00), 2.518 (2.62), |
| | 2.522 (1.68), 3.927 (6.11), 3.930 (6.01), 7.125 (0.52), 7.128 (0.55), |
| | 7.132 (0.66), 7.135 (0.61), 7.148 (1.09), 7.154 (1.18), 7.168 (0.63), |
| | 7.171 (0.64), 7.175 (0.69), 7.178 (0.65), 7.230 (0.90), 7.235 (0.90), |
| | 7.249 (1.29), 7.254 (2.09), 7.257 (1.16), 7.273 (1.32), 7.277 (1.25), |
| | 7.341 (0.94), 7.345 (1.21), 7.361 (4.99), 7.366 (4.60), 7.380 (1.87), |
| | 7.385 (1.73), 7.400 (2.24), 7.405 (0.88), 7.409 (1.05), 7.420 (0.95), |
| | 7.425 (3.78), 7.430 (3.32), 7.435 (1.46), 7.439 (2.83), 7.444 (3.24), |
| | 7.450 (1.19), 7.458 (1.28), 7.468 (1.23), 7.483 (1.20), 7.489 (0.67), |
| | 7.505 (0.54), 7.780 (1.38), 8.213 (2.61), 8.227 (2.47), 10.860 (3.34). |
| 131 | 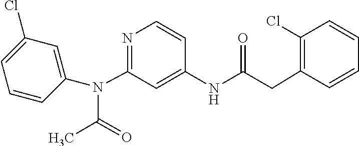 [SMILES: CC(=O)N(c1cccc(Cl)c1)c1cc(NC(=O)Cc2ccccc2Cl)ccn1] |
| | N-(3-chlorophenyl)-N-{4-[2-(2-chlorophenyl)acetamido]pyridin-2- |
| | yl}acetamide |
| | LC-MS (Method 2): Rₜ = 1.12 min; MS (ESIpos): m/z = 414 [M + H]⁺ |
| | ¹H-NMR (400 MHz, DMSO-d6) δ [ppm]: 2.005 (16.00), 2.518 (4.97), |
| | 2.522 (3.57), 3.877 (7.48), 7.197 (0.69), 7.201 (0.99), 7.205 (0.85), |
| | 7.217 (0.88), 7.221 (1.26), 7.224 (1.00), 7.301 (3.10), 7.310 (2.38), |
| | 7.316 (2.58), 7.324 (4.42), 7.334 (0.73), 7.363 (0.53), 7.366 (0.81), |
| | 7.371 (0.82), 7.383 (1.44), 7.388 (2.20), 7.391 (1.73), 7.395 (1.90), |
| | 7.403 (1.05), 7.408 (3.22), 7.417 (2.32), 7.422 (3.14), 7.427 (4.17), |
| | 7.434 (2.24), 7.443 (1.86), 7.448 (1.95), 7.458 (1.46), 7.490 (1.66), |
| | 7.495 (1.74), 7.504 (1.67), 7.509 (1.88), 7.667 (1.66), 8.290 (2.11), |
| | 8.304 (2.07), 10.771 (2.33). |
| 132 | 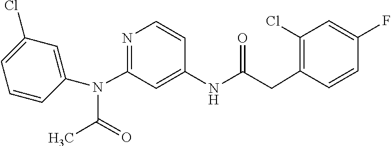 [SMILES: CC(=O)N(c1cccc(Cl)c1)c1cc(NC(=O)Cc2ccc(F)cc2Cl)ccn1] |
| | N-{4-[2-(2-chloro-4-fluorophenyl)acetamido]pyridin-2-yl}-N-(3- |
| | chlorophenyl)acetamide |
| | LC-MS (Method 2): Rₜ = 1.15 min; MS (ESIpos): m/z = 432 [M + H]+ |
| | ¹H-NMR (400 MHZ, DMSO-d6) δ [ppm]: 2.003 (16.00), 2.518 (3.83), |
| | 2.522 (2.43), 3.867 (6.87), 7.186 (0.75), 7.193 (0.96), 7.197 (0.88), |
| | 7.201 (1.22), 7.207 (2.04), 7.214 (2.14), 7.220 (1.54), 7.224 (1.17), |
| | 7.228 (1.10), 7.235 (1.04), 7.363 (0.51), 7.368 (0.83), 7.372 (0.78), |
| | 7.384 (1.40), 7.388 (2.15), 7.392 (1.62), 7.408 (2.58), 7.416 (1.85), |
| | 7.421 (2.98), 7.427 (3.76), 7.437 (1.90), 7.445 (1.74), 7.447 (1.60), |
| | 7.453 (1.86), 7.459 (3.01), 7.466 (1.55), 7.481 (2.91), 7.486 (1.87), |
| | 7.495 (1.71), 7.500 (1.87), 7.663 (1.85), 8.289 (2.32), 8.303 (2.22), |
| | 10.770 (2.65). |
| 133 | 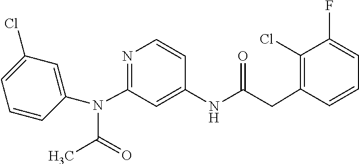 [SMILES: CC(=O)N(c1cccc(Cl)c1)c1cc(NC(=O)Cc2cccc(F)c2Cl)ccn1] |
| | N-{4-[2-(2-chloro-3-fluorophenyl)acetamido]pyridin-2-yl}-N-(3- |
| | chlorophenyl)acetamide |
| | LC-MS (Method 2): Rₜ = 1.13 min; MS (ESIpos): m/z = 432 [M + H]⁺ |
| | ¹H-NMR (400 MHz, DMSO-d6) δ [ppm]: 2.004 (16.00), 2.522 (6.38), |
| | 3.939 (7.00), 7.199 (0.68), 7.202 (1.00), 7.207 (0.82), 7.222 (1.26), |
| | 7.226 (0.96), 7.260 (0.75), 7.267 (1.16), 7.274 (0.85), 7.277 (0.91), |
| | 7.284 (1.31), 7.324 (0.41), 7.338 (1.71), 7.342 (2.12), 7.358 (4.39), |
| | 7.367 (1.86), 7.373 (1.92), 7.379 (0.63), 7.385 (1.44), 7.389 (2.13), |
| | 7.393 (1.69), 7.409 (2.45), 7.418 (1.67), 7.423 (2.81), 7.428 (3.86), |
| | 7.448 (1.00), 7.483 (1.61), 7.488 (1.66), 7.497 (1.64), 7.502 (1.82), |
| | 7.666 (1.64), 8.292 (2.09), 8.306 (2.05), 10.810 (2.33). |
| 134 | 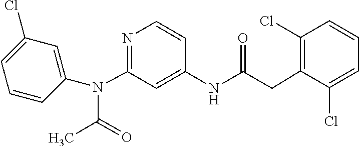 [SMILES: CC(=O)N(c1cccc(Cl)c1)c1cc(NC(=O)Cc2c(Cl)cccc2Cl)ccn1] |
| | N-(3-chlorophenyl)-N-{4-[2-(2,6-dichlorophenyl)acetamido]pyridin- |
| | 2-yl}acetamide |
| | LC-MS (Method 2): Rₜ = 1.17 min; MS (ESIneg): m/z = 446 [M − H]⁻ |
| | ¹H-NMR (400 MHz, DMSO-d6) δ [ppm]: 1.868 (0.85), 1.952 (0.68), |
| | 2.002 (16.00), 2.518 (1.24), 2.523 (0.90), 4.079 (7.43), 7.202 (0.72), |
| | 7.206 (0.99), 7.211 (0.81), 7.226 (1.19), 7.230 (0.87), 7.335 (1.57), |
| | 7.353 (2.04), 7.356 (2.27), 7.364 (0.58), 7.367 (0.85), 7.374 (2.63), |
| | 7.384 (1.48), 7.386 (1.29), 7.388 (2.23), 7.392 (1.47), 7.409 (2.43), |
| | 7.421 (1.67), 7.427 (5.32), 7.431 (1.50), 7.447 (0.97), 7.459 (1.62), |
| | 7.464 (1.71), 7.473 (1.66), 7.478 (1.84), 7.485 (6.72), 7.505 (4.06), |
| | 7.663 (1.52), 8.293 (2.11), 8.307 (2.01), 10.886 (2.30). |
| 135 | 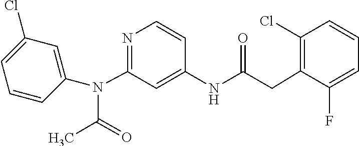 [SMILES: CC(=O)N(c1cccc(Cl)c1)c1cc(NC(=O)Cc2c(F)cccc2Cl)ccn1] |
| | N-{4-[2-(2-chloro-6-fluorophenyl)acetamido]pyridin-2-yl}-N-(3- |
| | chlorophenyl)acetamide |
| | LC-MS (Method 2): Rₜ = 1.13 min; MS (ESIpos): m/z = 432 [M + H]⁺ |
| | ¹H-NMR (400 MHZ, DMSO-d6) δ [ppm]: 2.003 (16.00), 2.323 (0.68), |
| | 2.327 (0.80), 2.665 (0.72), 2.669 (0.81), 3.922 (7.00), 7.203 (1.55), |
| | 7.223 (2.30), 7.245 (1.62), 7.249 (1.91), 7.268 (1.34), 7.337 (1.00), |
| | 7.341 (0.97), 7.357 (4.50), 7.362 (3.98), 7.372 (1.61), 7.377 (2.21), |
| | 7.383 (3.20), 7.387 (2.92), 7.391 (2.35), 7.396 (1.85), 7.403 (1.74), |
| | 7.407 (2.72), 7.425 (5.20), 7.447 (1.12), 7.463 (2.25), 7.468 (1.80), |
| | 7.478 (2.36), 7.482 (1.82), 7.664 (2.71), 8.293 (2.50), 8.307 (2.43), |
| | 10.874 (3.31). |
| 136 | 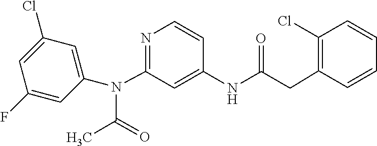 [SMILES: CC(=O)N(c1cc(F)cc(Cl)c1)c1cc(NC(=O)Cc2ccccc2Cl)ccn1] |
| | N-(3-chloro-5-fluorophenyl)-N-{4-[2-(2- |
| | chlorophenyl)acetamido]pyridin-2-yl}acetamide |
| | LC-MS (Method 1): Rₜ = 1.23 min; MS (ESIpos): m/z = 432 [M + H]⁺ |
| | ¹H-NMR (400 MHZ, DMSO-d6) δ [ppm]: 2.018 (16.00), 2.332 (0.67), |
| | 2.518 (3.33), 2.522 (2.15), 2.673 (0.68), 3.884 (6.78), 7.211 (0.48), |
| | 7.216 (0.84), 7.221 (0.64), 7.240 (0.88), 7.245 (0.81), 7.256 (1.75), |
| | 7.303 (2.78), 7.312 (1.96), 7.318 (2.02), 7.326 (3.91), 7.337 (0.49), |
| | 7.399 (1.80), 7.407 (1.38), 7.414 (1.11), 7.419 (0.97), 7.423 (1.88), |
| | 7.430 (0.89), 7.437 (1.74), 7.445 (1.45), 7.450 (0.99), 7.460 (1.20), |
| | 7.510 (1.41), 7.515 (1.55), 7.525 (1.48), 7.529 (1.58), 7.697 (1.45), |
| | 8.304 (1.83), 8.317 (1.75), 10.796 (2.08). |
| 137 | 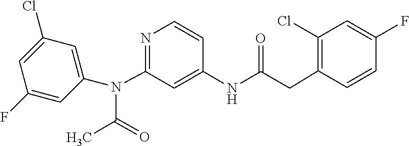 [SMILES: CC(=O)N(c1cc(F)cc(Cl)c1)c1cc(NC(=O)Cc2ccc(F)cc2Cl)ccn1] |
| | N-(3-chloro-5-fluorophenyl)-N-{4-[2-(2-chloro-4- |
| | fluorophenyl)acetamido]pyridin-2-yl)acetamide |
| | LC-MS (Method 2): Rₜ = 1.20 min; MS (ESIpos): m/z = 450 [M + H]⁺ |
| | ¹H-NMR (400 MHz, DMSO-d6) δ [ppm]: 1.161 (0.60), 1.179 (1.29), |
| | 1.197 (0.62), 2.017 (16.00), 2.518 (6.09), 2.523 (4.28), 3.792 (0.84), |
| | 3.875 (6.20), 4.084 (0.51), 4.102 (0.51), 7.189 (0.72), 7.195 (0.82), |
| | 7.210 (1.90), 7.217 (2.66), 7.227 (0.48), 7.231 (1.11), 7.238 (1.62), |
| | 7.246 (1.03), 7.256 (2.03), 7.399 (0.80), 7.405 (1.28), 7.410 (0.88), |
| | 7.421 (0.88), 7.426 (1.36), 7.433 (1.90), 7.440 (1.73), 7.448 (1.66), |
| | 7.455 (1.71), 7.462 (2.08), 7.470 (1.62), 7.486 (1.28), 7.502 (1.60), |
| | 7.507 (1.74), 7.516 (1.67), 7.521 (1.76), 7.697 (1.61), 8.303 (1.97), |
| | 8.317 (1.93), 10.795 (2.35). |
| 138 | 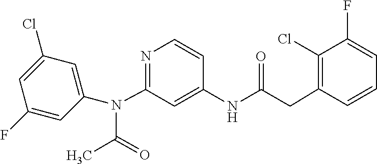 [SMILES: CC(=O)N(c1cc(F)cc(Cl)c1)c1cc(NC(=O)Cc2cccc(F)c2Cl)ccn1] |
| | N-(3-chloro-5-fluorophenyl)-N-{4-[2-(2-chloro-3- |
| | fluorophenyl)acetamido]pyridin-2-yl}acetamide |
| | LC-MS (Method 1): Rₜ = 1.23 min; MS (ESIpos): m/z = 450 [M + H]⁺ |
| | ¹H-NMR (400 MHz, DMSO-d6) δ [ppm]: 2.017 (16.00), 2.332 (0.44), |
| | 2.518 (2.54), 2.522 (1.61), 2.673 (0.44), 3.947 (6.22), 7.212 (0.48), |
| | 7.218 (0.83), 7.223 (0.64), 7.242 (0.87), 7.247 (0.83), 7.257 (1.76), |
| | 7.263 (1.44), 7.271 (1.07), 7.278 (0.75), 7.282 (0.78), 7.288 (1.08), |
| | 7.339 (1.58), 7.344 (2.07), 7.361 (3.72), 7.367 (1.20), 7.376 (1.10), |
| | 7.399 (0.75), 7.404 (1.15), 7.409 (0.80), 7.421 (0.76), 7.426 (1.15), |
| | 7.431 (0.73), 7.503 (1.38), 7.508 (1.51), 7.517 (1.46), 7.521 (1.52), |
| | 7.697 (1.39), 8.306 (1.79), 8.320 (1.71), 10.834 (2.05). |
| 139 | 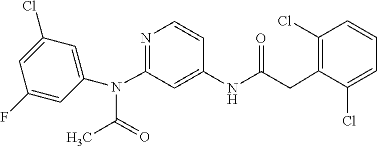 [SMILES: CC(=O)N(c1cc(F)cc(Cl)c1)c1cc(NC(=O)Cc2c(Cl)cccc2Cl)ccn1] |
| | N-(3-chloro-5-fluorophenyl)-N-{4-[2-(2,6- |
| | dichlorophenyl)acetamido]pyridin-2-yl}acetamide |
| | LC-MS (Method 1): Rₜ = 1.28 min; MS (ESIneg): m/z = 466 [M − H]⁻ |
| | ¹H-NMR (400 MHz, DMSO-d6) δ [ppm]: 1.147 (0.52), 1.162 (0.50), |
| | 2.016 (16.00), 2.332 (0.70), 2.518 (3.80), 2.522 (2.48), 2.673 (0.72), |
| | 4.086 (6.88), 7.221 (0.87), 7.226 (0.66), 7.245 (0.89), 7.250 (0.77), |
| | 7.263 (1.78), 7.336 (1.46), 7.355 (1.81), 7.358 (2.07), 7.377 (2.24), |
| | 7.397 (0.79), 7.403 (1.23), 7.408 (0.84), 7.419 (0.80), 7.425 (1.23), |
| | 7.430 (0.98), 7.480 (1.62), 7.485 (2.06), 7.488 (6.68), 7.494 (1.67), |
| | 7.499 (1.70), 7.508 (3.98), 7.695 (1.42), 8.306 (1.85), 8.321 (1.76), |
| | 10.913 (2.10). |
| 140 | 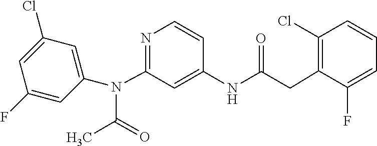 [SMILES: CC(=O)N(c1cc(F)cc(Cl)c1)c1cc(NC(=O)Cc2c(F)cccc2Cl)ccn1] |
| | N-(3-chloro-5-fluorophenyl)-N-{4-[2-(2-chloro-6- |
| | fluorophenyl)acetamido]pyridin-2-yl)acetamide |
| | LC-MS (Method 2): Rₜ = 1.18 min; MS (ESIpos): m/z = 450 [M + H]⁺ |
| | ¹H-NMR (400 MHZ, DMSO-d6) δ [ppm]: 1.182 (0.74), 2.016 (16.00), |
| | 2.518 (10.40), 2.523 (7.20), 3.930 (4.08), 3.934 (4.13), 7.219 (0.91), |
| | 7.224 (0.76), 7.228 (0.81), 7.233 (0.82), 7.243 (1.09), 7.247 (1.56), |
| | 7.252 (2.00), 7.255 (2.05), 7.260 (2.15), 7.271 (1.13), 7.275 (1.06), |
| | 7.339 (0.63), 7.343 (0.80), 7.360 (3.23), 7.365 (2.97), 7.379 (1.32), |
| | 7.384 (1.29), 7.398 (1.85), 7.403 (1.69), 7.408 (0.97), 7.419 (1.25), |
| | 7.425 (1.31), 7.430 (0.87), 7.484 (1.48), 7.488 (1.60), 7.498 (1.57), |
| | 7.502 (1.64), 7.700 (1.52), 8.308 (1.89), 8.321 (1.85), 10.900 (2.20). |
| 141 | 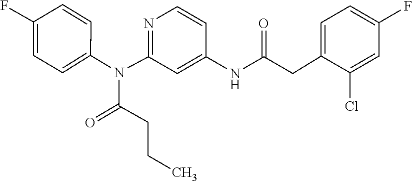 [SMILES: CCCC(=O)N(c1ccc(F)cc1)c1cc(NC(=O)Cc2ccc(F)cc2Cl)ccn1] |
| | N-{4-[2-(2-chloro-4-fluorophenyl)acetamido]pyridin-2-yl}-N-(4- |
| | fluorophenyl)butanamide |
| | LC-MS (Method 1): Rₜ = 1.25 min; MS (ESIpos): m/z = 444 [M + H]⁺ |
| | ¹H-NMR (400 MHz, DMSO-d6) δ [ppm]: 0.805 (6.68), 0.823 (16.00), |
| | 0.842 (7.54), 1.136 (1.19), 1.153 (1.22), 1.485 (0.50), 1.503 (2.27), |
| | 1.522 (4.31), 1.540 (4.42), 1.558 (2.27), 1.577 (0.46), 2.189 (3.54), |
| | 2.208 (6.36), 2.226 (3.22), 2.518 (2.12), 2.522 (1.30), 3.865 (10.61), |
| | 7.186 (1.20), 7.193 (1.37), 7.208 (2.88), 7.211 (3.35), 7.214 (3.41), |
| | 7.229 (2.66), 7.234 (6.97), 7.249 (1.32), 7.255 (4.68), 7.263 (0.65), |
| | 7.286 (0.53), 7.294 (3.96), 7.300 (1.68), 7.307 (4.30), 7.317 (2.71), |
| | 7.324 (1.09), 7.330 (2.31), 7.430 (2.68), 7.437 (3.04), 7.441 (3.48), |
| | 7.445 (5.07), 7.453 (3.58), 7.455 (3.82), 7.460 (6.96), 7.466 (2.33), |
| | 7.482 (1.95), 7.685 (3.23), 7.688 (3.14), 8.255 (3.88), 8.270 (3.70), |
| | 10.745 (4.18). |
| 142 | 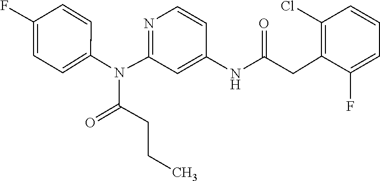 [SMILES: CCCC(=O)N(c1ccc(F)cc1)c1cc(NC(=O)Cc2c(F)cccc2Cl)ccn1] |
| | N-{4-[2-(2-chloro-6-fluorophenyl)acetamido]pyridin-2-yl}-N-(4- |
| | fluorophenyl)butanamide |
| | LC-MS (Method 1): Rₜ = 1.24 min; MS (ESIpos): m/z = 444 [M + H]⁺ |
| | ¹H-NMR (400 MHZ, DMSO-d6) δ [ppm]: 0.804 (7.10), 0.823 (16.00), |
| | 0.842 (7.69), 1.147 (0.70), 1.163 (0.77), 1.484 (0.52), 1.503 (2.41), |
| | 1.521 (4.63), 1.539 (4.71), 1.558 (2.42), 1.576 (0.50), 2.188 (3.80), |
| | 2.206 (6.82), 2.225 (3.43), 2.518 (3.07), 2.522 (1.96), 3.920 (7.94), |
| | 3.924 (7.57), 7.210 (2.89), 7.216 (1.13), 7.227 (2.46), 7.233 (7.57), |
| | 7.238 (1.61), 7.245 (2.05), 7.250 (3.73), 7.255 (5.64), 7.262 (0.77), |
| | 7.269 (1.63), 7.273 (1.61), 7.298 (4.28), 7.303 (1.98), 7.310 (4.49), |
| | 7.320 (2.96), 7.327 (1.20), 7.333 (2.57), 7.338 (1.46), 7.341 (1.72), |
| | 7.358 (5.94), 7.363 (5.24), 7.377 (2.29), 7.382 (2.07), 7.397 (2.29), |
| | 7.402 (0.75), 7.420 (3.11), 7.424 (2.88), 7.434 (2.97), 7.439 (3.01), |
| | 7.690 (3.37), 7.694 (3.25), 8.260 (4.13), 8.274 (3.94), 10.849 (4.35). |
| 143 | 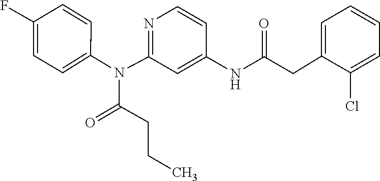 [SMILES: CCCC(=O)N(c1ccc(F)cc1)c1cc(NC(=O)Cc2ccccc2Cl)ccn1] |
| | N-{4-[2-(2-chlorophenyl)acetamido]pyridin-2-yl}-N-(4- |
| | fluorophenyl)butanamide |
| | LC-MS (Method 1): Rₜ = 1.23 min; MS (ESIpos): m/z = 426 [M + H]⁺ |
| | ¹H-NMR (400 MHz, DMSO-d6) δ [ppm]: 0.806 (6.70), 0.824 (16.00), |
| | 0.842 (7.55), 1.130 (1.28), 1.146 (1.29), 1.486 (0.50), 1.505 (2.23), |
| | 1.522 (4.28), 1.541 (4.36), 1.560 (2.24), 1.578 (0.46), 2.191 (3.61), |
| | 2.209 (6.45), 2.228 (3.19), 2.518 (2.03), 2.522 (1.25), 3.711 (0.42), |
| | 3.875 (12.49), 7.210 (2.65), 7.217 (1.01), 7.228 (1.50), 7.233 (6.60), |
| | 7.238 (1.58), 7.241 (0.88), 7.249 (1.56), 7.255 (4.84), 7.263 (0.85), |
| | 7.267 (0.49), 7.287 (0.65), 7.295 (4.17), 7.301 (6.87), 7.309 (6.33), |
| | 7.317 (6.20), 7.325 (8.11), 7.331 (2.71), 7.388 (0.55), 7.396 (2.73), |
| | 7.403 (1.43), 7.407 (1.83), 7.411 (2.00), 7.420 (1.73), 7.425 (0.70), |
| | 7.435 (3.09), 7.444 (2.72), 7.450 (4.11), 7.455 (3.27), 7.458 (2.65), |
| | 7.464 (2.98), 7.469 (3.07), 7.689 (3.21), 7.693 (3.11), 8.256 (3.86), |
| | 8.271 (3.66), 10.749 (4.02). |
| 144 | 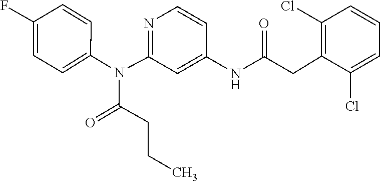 [SMILES: CCCC(=O)N(c1ccc(F)cc1)c1cc(NC(=O)Cc2c(Cl)cccc2Cl)ccn1] |
| | N-{4-[2-(2,6-dichlorophenyl)acetamido]pyridin-2-yl}-N-(4- |
| | fluorophenyl)butanamide |
| | LC-MS (Method 1): Rₜ = 1.28 min; MS (ESIneg): m/z = 458 [M − H]⁻ |
| | ¹H-NMR (400 MHz, DMSO-d6) δ [ppm]: 0.804 (7.05), 0.823 (16.00), |
| | 0.842 (7.57), 1.485 (0.51), 1.503 (2.28), 1.521 (4.35), 1.539 (4.42), |
| | 1.558 (2.30), 1.577 (0.48), 2.187 (3.66), 2.206 (6.46), 2.223 (3.27), |
| | 2.331 (0.61), 2.518 (3.57), 2.522 (2.22), 2.673 (0.65), 4.077 (12.51), |
| | 7.211 (2.76), 7.217 (1.07), 7.228 (1.44), 7.234 (6.77), 7.238 (1.41), |
| | 7.242 (0.82), 7.250 (1.30), 7.255 (4.83), 7.264 (0.62), 7.293 (0.56), |
| | 7.301 (3.94), 7.306 (1.66), 7.313 (4.25), 7.317 (1.88), 7.323 (2.87), |
| | 7.331 (1.16), 7.335 (4.52), 7.354 (3.40), 7.357 (3.49), 7.376 (4.21), |
| | 7.414 (2.84), 7.418 (2.90), 7.427 (3.07), 7.432 (2.96), 7.487 (11.31), |
| | 7.507 (6.94), 7.691 (3.06), 7.695 (3.01), 8.259 (3.94), 8.273 (3.76), |
| | 10.861 (4.10). |
| 145 | 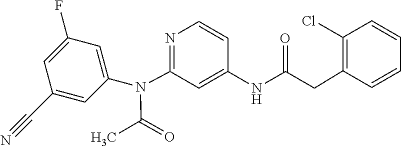 [SMILES: CC(=O)N(c1cc(F)cc(C#N)c1)c1cc(NC(=O)Cc2ccccc2Cl)ccn1] |
| | N-{4-[2-(2-Chlorphenyl)acetamido]pyridin-2-yl}-N-(3-cyan-5- |
| | fluorphenyl)acetamide |
| | LC-MS (Method 1): Rₜ = 1.14 min; MS (ESIpos): m/z = 423 [M + H]⁺ |
| | ¹H-NMR (400 MHZ, DMSO-d6) δ [ppm]: 2.026 (16.00), 2.074 (0.73), |
| | 2.518 (5.84), 2.523 (4.08), 3.888 (7.83), 7.305 (3.20), 7.314 (2.26), |
| | 7.319 (2.33), 7.328 (4.28), 7.338 (0.53), 7.402 (1.68), 7.408 (0.88), |
| | 7.412 (1.07), 7.416 (1.17), 7.425 (1.12), 7.438 (1.93), 7.447 (1.55), |
| | 7.451 (1.08), 7.461 (1.38), 7.514 (1.66), 7.518 (1.70), 7.528 (1.69), |
| | 7.532 (1.68), 7.636 (0.98), 7.660 (1.03), 7.690 (2.09), 7.736 (1.57), |
| | 7.814 (0.87), 7.817 (0.97), 7.819 (1.06), 7.823 (0.88), 7.835 (0.91), |
| | 7.838 (1.00), 7.840 (1.00), 7.844 (0.85), 8.296 (1.81), 8.309 (1.74), |
| | 10.808 (2.54). |
| 146 | 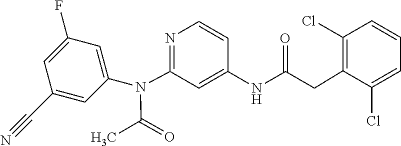 [SMILES: CC(=O)N(c1cc(F)cc(C#N)c1)c1cc(NC(=O)Cc2c(Cl)cccc2Cl)ccn1] |
| | N-(3-Cyan-5-fluorphenyl)-N-{4-[2-(2,6- |
| | dichlorphenyl)acetamido]pyridin-2-yl}acetamide |
| | LC-MS (Method 1): Rₜ = 1.18 min; MS (ESIpos): m/z = 457 [M + H]⁺ |
| | ¹H-NMR (400 MHz, DMSO-d6) δ [ppm]: 2.024 (16.00), 2.074 (6.32), |
| | 2.084 (13.82), 2.518 (4.15), 2.523 (2.91), 4.089 (7.58), 7.338 (1.46), |
| | 7.356 (1.95), 7.359 (2.14), 7.377 (2.32), 7.484 (1.86), 7.490 (7.78), |
| | 7.498 (1.85), 7.502 (1.90), 7.509 (4.37), 7.640 (0.95), 7.645 (0.71), |
| | 7.664 (0.96), 7.669 (0.73), 7.694 (1.98), 7.735 (1.42), 7.813 (0.85), |
| | 7.816 (0.93), 7.819 (1.01), 7.823 (0.86), 7.834 (0.88), 7.837 (0.98), |
| | 7.840 (0.97), 7.844 (0.81), 8.299 (1.77), 8.313 (1.69), 10.929 (2.30). |
| 147 | 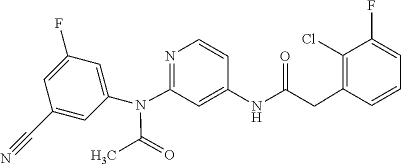 [SMILES: CC(=O)N(c1cc(F)cc(C#N)c1)c1cc(NC(=O)Cc2cccc(F)c2Cl)ccn1] |
| | N-{4-[2-(2-Chlor-3-fluorphenyl)acetamido]pyridin-2-yl}-N-(3-cyan- |
| | 5-fluorphenyl)acetamide |
| | LC-MS (Method 1): Rₜ = 1.16 min; MS (ESIpos): m/z = 441 [M + H]⁺ |
| | ¹H-NMR (400 MHz, DMSO-d6) δ [ppm]: 2.026 (16.00), 2.518 (5.41), |
| | 2.523 (3.77), 3.951 (7.44), 7.266 (0.81), 7.273 (1.20), 7.280 (0.89), |
| | 7.284 (0.98), 7.289 (1.34), 7.327 (0.42), 7.341 (1.87), 7.346 (2.41), |
| | 7.362 (4.27), 7.369 (1.42), 7.377 (1.29), 7.383 (0.41), 7.506 (1.66), |
| | 7.511 (1.67), 7.520 (1.68), 7.525 (1.72), 7.638 (0.99), 7.643 (0.74), |
| | 7.662 (0.98), 7.691 (2.06), 7.736 (1.54), 7.816 (0.89), 7.819 (0.96), |
| | 7.821 (1.04), 7.825 (0.88), 7.837 (0.92), 7.840 (1.00), 7.842 (1.02), |
| | 7.846 (0.83), 8.297 (1.80), 8.311 (1.72), 10.846 (2.57). |
| 148 | 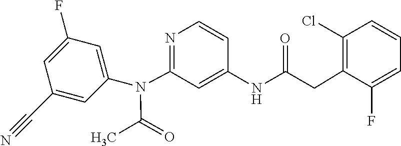 [SMILES: CC(=O)N(c1cc(F)cc(C#N)c1)c1cc(NC(=O)Cc2c(F)cccc2Cl)ccn1] |
| | N-{4-[2-(2-Chlor-6-fluorphenyl)acetamido]pyridin-2-yl}-N-(3-cyan- |
| | 5-fluorphenyl)acetamide |
| | LC-MS (Method 1): Rₜ = 1.15 min; MS (ESIpos): m/z = 441 [M + H]⁺ |
| | ¹H-NMR (400 MHZ, DMSO-d6) δ [ppm]: 2.023 (16.00), 2.074 (2.62), |
| | 2.518 (7.91), 2.523 (5.50), 3.933 (4.65), 3.936 (4.52), 7.229 (0.65), |
| | 7.234 (0.66), 7.247 (0.95), 7.252 (1.56), 7.272 (0.96), 7.275 (0.96), |
| | 7.340 (0.69), 7.344 (0.87), 7.360 (3.64), 7.365 (3.30), 7.379 (1.37), |
| | 7.384 (1.26), 7.399 (1.32), 7.405 (0.45), 7.420 (0.42), 7.486 (1.56), |
| | 7.491 (1.57), 7.500 (1.57), 7.505 (1.69), 7.636 (0.95), 7.661 (0.97), |
| | 7.691 (2.05), 7.736 (1.50), 7.818 (1.06), 7.822 (0.87), 7.833 (0.90), |
| | 7.837 (0.99), 7.839 (1.00), 8.299 (1.77), 8.314 (1.69), 10.911 (2.44). |
| 149 | 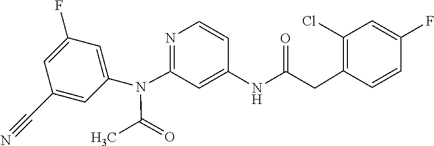 [SMILES: CC(=O)N(c1cc(F)cc(C#N)c1)c1cc(NC(=O)Cc2ccc(F)cc2Cl)ccn1] |
| | N-{4-[2-(2-Chlor-4-fluorphenyl)acetamido]pyridin-2-yl}-N-(3-cyan- |
| | 5-fluorphenyl)acetamide |
| | LC-MS (Method 1): Rₜ = 1.17 min; MS (ESIpos): m/z = 441 [M + H]⁺ |
| | ¹H-NMR (400 MHz, DMSO-d6) δ [ppm]: 2.025 (16.00), 2.074 (0.63), |
| | 2.518 (4.38), 2.523 (3.04), 3.878 (6.52), 7.190 (0.74), 7.196 (0.78), |
| | 7.211 (1.51), 7.218 (1.73), 7.233 (0.87), 7.240 (0.92), 7.434 (1.64), |
| | 7.440 (1.64), 7.450 (1.53), 7.456 (1.77), 7.463 (1.98), 7.471 (1.39), |
| | 7.487 (1.24), 7.504 (1.62), 7.509 (1.60), 7.519 (1.61), 7.523 (1.67), |
| | 7.634 (0.98), 7.659 (0.98), 7.664 (0.76), 7.688 (2.04), 7.733 (1.53), |
| | 7.814 (0.86), 7.818 (0.96), 7.820 (1.05), 7.823 (0.86), 7.835 (0.90), |
| | 7.838 (0.98), 7.841 (1.01), 7.844 (0.83), 8.294 (1.76), 8.309 (1.69), |
| | 10.806 (2.57). |
| 150 | 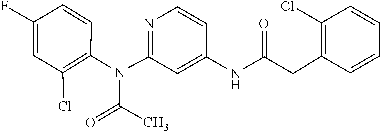 [SMILES: CC(=O)N(c1cc(NC(=O)Cc2ccccc2Cl)ccn1)c1ccc(F)cc1Cl] |
| | N-(2-Chlor-4-fluorphenyl)-N-{4-[2-(2- |
| | chlorphenyl)acetamido]pyridin-2-yl}acetamide |
| | LC-MS (Method 1): Rₜ = 1.19 min; MS (ESIpos): m/z = 432 [M + H]⁺ |
| | ¹H-NMR (400 MHZ, DMSO-d6) δ [ppm]: 2.000 (14.96), 2.518 (3.08), |
| | 2.522 (1.90), 3.881 (16.00), 7.295 (0.80), 7.298 (1.77), 7.305 (8.89), |
| | 7.313 (4.06), 7.316 (4.95), 7.319 (6.55), 7.321 (5.90), 7.329 (10.25), |
| | 7.338 (1.24), 7.340 (2.74), 7.348 (2.13), 7.394 (0.48), 7.402 (3.49), |
| | 7.409 (1.75), 7.414 (2.18), 7.418 (2.19), 7.426 (2.26), 7.439 (4.22), |
| | 7.449 (5.99), 7.454 (5.25), 7.463 (6.29), 7.468 (4.35), 7.546 (1.80), |
| | 7.560 (1.94), 7.568 (1.73), 7.582 (1.55), 7.622 (3.50), 7.629 (3.79), |
| | 7.643 (3.49), 7.650 (3.64), 7.867 (1.17), 8.170 (3.30), 8.184 (3.13), |
| | 10.745 (5.05). |
| 151 | 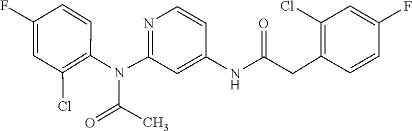 [SMILES: CC(=O)N(c1cc(NC(=O)Cc2ccc(F)cc2Cl)ccn1)c1ccc(F)cc1Cl] |
| | N-(2-Chlor-4-fluorphenyl)-N-{4-[2-(2-chlor-4- |
| | fluorphenyl)acetamido]pyridin-2-yl}acetamide |
| | LC-MS (Method 1): Rₜ = 1.21 min; MS (ESIpos): m/z = 450 [M + H]⁺ |
| | ¹H-NMR (400 MHZ, DMSO-d6) δ [ppm]: 1.998 (16.00), 2.332 (0.89), |
| | 2.518 (4.36), 2.523 (2.70), 2.673 (0.89), 3.944 (15.91), 7.268 (1.66), |
| | 7.275 (2.55), 7.282 (1.84), 7.286 (2.01), 7.290 (2.78), 7.300 (1.66), |
| | 7.307 (2.01), 7.322 (2.72), 7.328 (3.38), 7.342 (5.51), 7.347 (6.80), |
| | 7.363 (8.66), 7.370 (3.24), 7.379 (2.78), 7.385 (0.92), 7.399 (0.49), |
| | 7.442 (4.01), 7.447 (4.24), 7.456 (4.16), 7.461 (4.39), 7.548 (1.95), |
| | 7.563 (2.09), 7.570 (1.86), 7.585 (1.66), 7.623 (3.73), 7.630 (3.90), |
| | 7.644 (3.81), 7.651 (3.67), 7.868 (1.23), 8.172 (3.50), 8.186 (3.33), |
| | 10.783 (5.36). |
| 152 | 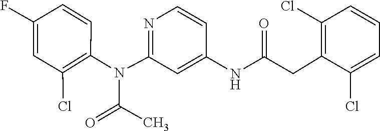 [SMILES: CC(=O)N(c1cc(NC(=O)Cc2c(Cl)cccc2Cl)ccn1)c1ccc(F)cc1Cl] |
| | N-(2-Chlor-4-fluorphenyl)-N-{4-[2-(2,6- |
| | dichlorphenyl)acetamido]pyridin-2-yl}acetamide |
| | LC-MS (Method 1): Rₜ = 1.24 min; MS (ESIpos): m/z = 466 [M + H]⁺ |
| | ¹H-NMR (400 MHZ, DMSO-d6) δ [ppm]: 1.996 (15.23), 2.332 (1.23), |
| | 2.336 (0.54), 2.518 (5.82), 2.523 (3.62), 2.673 (1.27), 2.678 (0.58), |
| | 4.083 (16.00), 7.300 (1.62), 7.307 (1.85), 7.322 (2.43), 7.329 (2.66), |
| | 7.339 (3.78), 7.342 (2.12), 7.350 (2.16), 7.357 (4.51), 7.361 (4.93), |
| | 7.379 (5.44), 7.419 (3.82), 7.424 (4.01), 7.433 (3.97), 7.438 (4.05), |
| | 7.491 (15.11), 7.511 (9.37), 7.551 (1.85), 7.565 (2.00), 7.572 (1.77), |
| | 7.587 (1.58), 7.623 (3.62), 7.630 (3.70), 7.644 (3.66), 7.651 (3.59), |
| | 7.865 (1.12), 8.172 (3.28), 8.186 (3.12), 10.854 (5.17). |
| 153 | 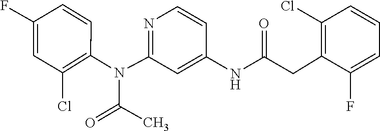 [SMILES: CC(=O)N(c1cc(NC(=O)Cc2c(F)cccc2Cl)ccn1)c1ccc(F)cc1Cl] |
| | N-(2-Chlor-4-fluorphenyl)-N-{4-[2-(2-chlor-6- |
| | fluorphenyl)acetamido]pyridin-2-yl}acetamide |
| | LC-MS (Method 1): Rₜ = 1.20 min; MS (ESIpos): m/z = 450 [M + H]⁺ |
| | ¹H-NMR (400 MHZ, DMSO-d6) δ [ppm]: 1.997 (16.00), 2.332 (0.54), |
| | 2.518 (2.42), 2.523 (1.51), 2.673 (0.54), 3.927 (9.72), 3.931 (9.42), |
| | 7.231 (1.40), 7.236 (1.47), 7.249 (2.07), 7.254 (3.40), 7.257 (1.80), |
| | 7.274 (2.14), 7.278 (2.03), 7.299 (1.61), 7.307 (1.86), 7.321 (2.47), |
| | 7.328 (2.80), 7.341 (3.40), 7.345 (2.35), 7.349 (2.29), 7.362 (8.19), |
| | 7.367 (7.47), 7.380 (3.03), 7.385 (2.82), 7.400 (3.03), 7.405 (0.95), |
| | 7.423 (4.06), 7.428 (4.20), 7.437 (4.11), 7.442 (4.24), 7.550 (1.94), |
| | 7.565 (2.07), 7.572 (1.84), 7.586 (1.65), 7.622 (3.68), 7.629 (3.96), |
| | 7.643 (3.66), 7.650 (3.78), 7.868 (1.21), 8.173 (3.45), 8.187 (3.26), |
| | 10.844 (5.23). |
| 154 | 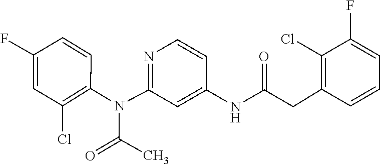 [SMILES: CC(=O)N(c1cc(NC(=O)Cc2cccc(F)c2Cl)ccn1)c1ccc(F)cc1Cl] |
| | N-(2-Chlor-4-fluorphenyl)-N-{4-[2-(2-chlor-3- |
| | fluorphenyl)acetamido]pyridin-2-yl}acetamide |
| | LC-MS (Method 1): Rₜ = 1.20 min; MS (ESIpos): m/z = 450 [M + H]⁺ |
| | ¹H-NMR (400 MHZ, DMSO-d6) δ [ppm]: 1.998 (16.00), 2.518 (2.66), |
| | 2.523 (1.67), 3.872 (14.07), 7.191 (1.70), 7.197 (1.93), 7.212 (3.63), |
| | 7.219 (4.12), 7.233 (1.99), 7.240 (2.05), 7.299 (1.60), 7.306 (1.93), |
| | 7.321 (2.51), 7.328 (2.82), 7.341 (1.94), 7.348 (2.15), 7.435 (3.94), |
| | 7.441 (7.75), 7.445 (4.66), 7.452 (3.96), 7.455 (6.01), 7.459 (5.30), |
| | 7.463 (4.39), 7.467 (3.54), 7.473 (2.91), 7.489 (2.63), 7.546 (1.96), |
| | 7.560 (2.11), 7.568 (1.87), 7.582 (1.67), 7.622 (3.78), 7.629 (3.96), |
| | 7.644 (3.81), 7.651 (3.84), 7.863 (1.27), 8.169 (3.54), 8.183 (3.35), |
| | 10.742 (5.54). |
| 155 | 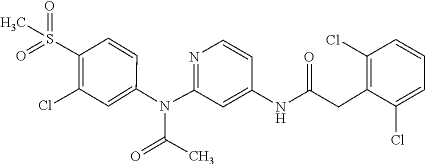 [SMILES: CC(=O)N(c1ccc(S(C)(=O)=O)c(Cl)c1)c1cc(NC(=O)Cc2c(Cl)cccc2Cl)ccn1] |
| | N-[3-Chlor-4-(methylsulfonyl)phenyl]-N-{4-[2-(2,6- |
| | dichlorphenyl)acetamido]pyridin-2-yl)acetamide |
| | LC-MS (Method 2): Rₜ = 1.14 min; MS (ESIpos): m/z = 528 [M + H]⁺ |
| | ¹H-NMR (400 MHZ, DMSO-d6) δ [ppm]: 2.518 (4.33), 2.523 (2.98), |
| | 3.858 (16.00), 6.804 (2.33), 6.824 (2.66), 7.150 (2.76), 7.170 (5.43), |
| | 7.189 (3.12), 7.210 (1.64), 7.228 (4.85), 7.231 (4.29), 7.245 (11.58), |
| | 7.256 (14.44), 7.268 (5.43), 7.272 (5.75), 7.281 (2.11), 7.288 (3.36), |
| | 7.299 (0.98), 7.305 (1.51), 7.310 (1.71), 7.325 (7.15), 7.332 (7.38), |
| | 7.348 (9.45), 7.353 (4.83), 7.364 (3.13), 7.370 (1.22), 7.384 (0.69), |
| | 7.483 (3.44), 7.487 (5.70), 7.492 (3.28), 10.282 (5.35). |
| 156 | 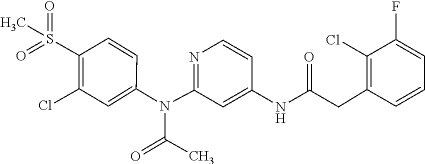 [SMILES: CC(=O)N(c1ccc(S(C)(=O)=O)c(Cl)c1)c1cc(NC(=O)Cc2cccc(F)c2Cl)ccn1] |
| | N-{4-[2-(2-Chlor-3-fluorphenyl)acetamido]pyridin-2-yl}-N-[3- |
| | chlor-4-(methylsulfonyl)phenyl]acetamide |
| | LC-MS (Method 2): Rₜ = 1.11 min; MS (ESIpos): m/z = 510 [M + H]⁺ |
| | ¹H-NMR (400 MHZ, DMSO-d6) δ [ppm]: 2.037 (14.26), 2.518 (2.64), |
| | 2.523 (1.85), 3.366 (0.50), 3.382 (16.00), 3.948 (6.37), 7.261 (0.71), |
| | 7.268 (1.04), 7.275 (0.76), 7.279 (0.83), 7.284 (1.17), 7.338 (1.69), |
| | 7.343 (1.98), 7.358 (4.13), 7.367 (2.23), 7.374 (1.29), 7.383 (1.14), |
| | 7.388 (1.14), 7.542 (1.33), 7.547 (1.42), 7.556 (1.38), 7.561 (1.47), |
| | 7.676 (1.71), 7.733 (2.68), 7.738 (2.57), 8.014 (3.26), 8.035 (3.04), |
| | 8.364 (1.80), 8.378 (1.69), 10.870 (2.35). |
| 157 | 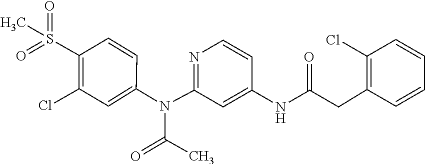 [SMILES: CC(=O)N(c1ccc(S(C)(=O)=O)c(Cl)c1)c1cc(NC(=O)Cc2ccccc2Cl)ccn1] |
| | N-[3-Chlor-4-(methylsulfonyl)phenyl]-N-{4-[2-(2- |
| | chlorphenyl)acetamido]pyridin-2-yl}acetamide |
| | LC-MS (Method 2): Rₜ = 1.09 min; MS (ESIpos): m/z = 492 [M + H]⁺ |
| | ¹H-NMR (400 MHZ, DMSO-d6) δ [ppm]: 2.038 (13.97), 2.518 (1.46), |
| | 2.522 (0.97), 3.381 (16.00), 3.885 (6.52), 7.301 (2.77), 7.310 (2.28), |
| | 7.315 (2.39), 7.324 (3.85), 7.334 (0.63), 7.360 (1.07), 7.365 (1.06), |
| | 7.381 (1.15), 7.386 (1.30), 7.396 (1.57), 7.407 (0.98), 7.411 (1.10), |
| | 7.420 (1.00), 7.434 (1.72), 7.443 (1.40), 7.448 (1.07), 7.457 (1.27), |
| | 7.549 (1.31), 7.554 (1.33), 7.563 (1.36), 7.568 (1.35), 7.678 (1.81), |
| | 7.733 (2.74), 7.738 (2.58), 8.013 (3.22), 8.034 (3.05), 8.362 (1.80), |
| | 8.376 (1.72), 10.833 (2.31). |
| 158 | 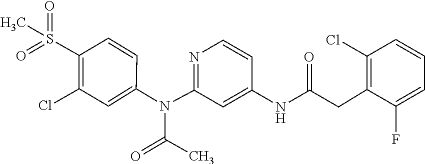 [SMILES: CC(=O)N(c1ccc(S(C)(=O)=O)c(Cl)c1)c1cc(NC(=O)Cc2c(F)cccc2Cl)ccn1] |
| | N-{4-[2-(2-Chlor-6-fluorphenyl)acetamido]pyridin-2-yl}-N-[3- |
| | chlor-4-(methylsulfonyl)phenyl]acetamide |
| | LC-MS (Method 2): Rₜ = 1.10 min; MS (ESIpos): m/z = 510 [M + H]⁺ |
| | ¹H-NMR (400 MHZ, DMSO-d6) δ [ppm]: 2.035 (14.96), 2.074 (0.67), |
| | 2.518 (1.91), 2.523 (1.37), 3.381 (16.00), 3.932 (4.06), 3.935 (3.95), |
| | 7.225 (0.59), 7.230 (0.60), 7.244 (0.88), 7.249 (1.38), 7.268 (0.87), |
| | 7.272 (0.83), 7.337 (0.65), 7.341 (0.80), 7.357 (3.47), 7.362 (3.78), |
| | 7.376 (1.44), 7.382 (1.91), 7.388 (1.23), 7.396 (1.38), 7.402 (0.46), |
| | 7.417 (0.44), 7.524 (1.35), 7.529 (1.36), 7.538 (1.35), 7.543 (1.44), |
| | 7.678 (1.69), 7.736 (2.74), 7.741 (2.67), 8.012 (3.54), 8.033 (3.24), |
| | 8.366 (1.80), 8.380 (1.73), 10.936 (2.33). |
| 159 | 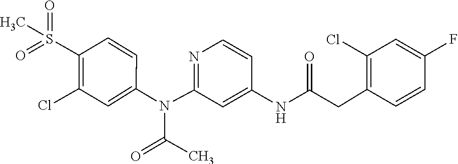 [SMILES: CC(=O)N(c1ccc(S(C)(=O)=O)c(Cl)c1)c1cc(NC(=O)Cc2ccc(F)cc2Cl)ccn1] |
| | N-{4-[2-(2-Chlor-4-fluorphenyl)acetamido]pyridin-2-yl}-N-[3- |
| | chlor-4-(methylsulfonyl)phenyl]acetamide |
| | LC-MS (Method 2): Rₜ = 1.12 min; MS (ESIpos): m/z = 510 [M + H]⁺ |
| | ¹H-NMR (400 MHz, DMSO-d6) δ [ppm]: 2.036 (14.66), 2.074 (0.76), |
| | 2.518 (1.80), 2.523 (1.27), 3.382 (16.00), 3.876 (5.64), 7.186 (0.65), |
| | 7.193 (0.71), 7.207 (1.38), 7.214 (1.55), 7.229 (0.79), 7.236 (0.82), |
| | 7.360 (1.04), 7.365 (1.05), 7.381 (1.09), 7.386 (1.10), 7.431 (1.51), |
| | 7.437 (1.54), 7.445 (1.34), 7.453 (1.66), 7.460 (2.50), 7.466 (1.25), |
| | 7.482 (1.08), 7.540 (1.32), 7.545 (1.35), 7.554 (1.33), 7.559 (1.40), |
| | 7.674 (1.78), 7.731 (2.74), 7.736 (2.65), 8.013 (3.36), 8.035 (3.15), |
| | 8.361 (1.81), 8.375 (1.71), 10.830 (2.34). |
| 160 | 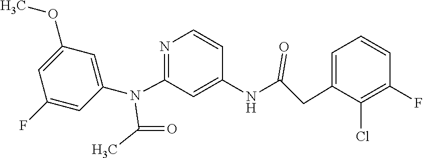 [SMILES: COc1cc(F)cc(N(C(C)=O)c2cc(NC(=O)Cc3cccc(F)c3Cl)ccn2)c1] |
| | N-{4-[2-(2-Chlor-3-fluorphenyl)acetamido]pyridin-2-yl}-N-(3-fluor- |
| | 5-methoxyphenyl)acetamide |
| | LC-MS (Method 2): Rₜ = 1.17 min; MS (ESIpos): m/z = 446 [M + H]⁺ |
| | ¹H-NMR (400 MHZ, DMSO-d6) δ [ppm]: 1.877 (0.56), 1.951 (0.47), |
| | 2.011 (14.54), 2.518 (1.38), 2.522 (0.90), 2.669 (0.42), 3.746 (16.00), |
| | 3.941 (6.17), 6.694 (1.68), 6.732 (0.62), 6.738 (1.04), 6.743 (0.66), |
| | 6.756 (0.63), 6.762 (0.98), 6.767 (0.64), 6.796 (0.76), 6.802 (1.32), |
| | 6.808 (0.66), 6.824 (0.77), 6.829 (1.30), 6.835 (0.63), 7.262 (0.66), |
| | 7.269 (1.01), 7.275 (0.72), 7.280 (0.78), 7.285 (1.06), 7.338 (1.66), |
| | 7.343 (1.98), 7.359 (3.78), 7.366 (1.21), 7.374 (1.09), 7.478 (1.52), |
| | 7.483 (1.49), 7.492 (1.44), 7.497 (1.56), 7.670 (1.62), 7.674 (1.55), |
| | 8.285 (2.02), 8.299 (1.92), 10.798 (2.07). |
| 161 | 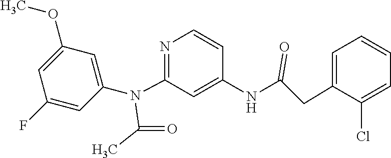 [SMILES: COc1cc(F)cc(N(C(C)=O)c2cc(NC(=O)Cc3ccccc3Cl)ccn2)c1] |
| | N-{4-[2-(2-Chlorphenyl)acetamido]pyridin-2-yl}-N-(3-fluor-5- |
| | methoxyphenyl)acetamide |
| | LC-MS (Method 2): Rₜ = 1.16 min; MS (ESIpos): m/z = 428 [M + H]⁺ |
| | ¹H-NMR (400 MHz, DMSO-d6) δ [ppm]: 1.868 (0.41), 2.012 (13.33), |
| | 2.518 (1.56), 2.522 (1.03), 3.746 (16.00), 3.878 (6.16), 6.692 (1.55), |
| | 6.731 (0.56), 6.736 (0.97), 6.741 (0.62), 6.755 (0.57), 6.760 (0.92), |
| | 6.765 (0.60), 6.795 (0.72), 6.800 (1.26), 6.806 (0.63), 6.822 (0.71), |
| | 6.827 (1.20), 6.833 (0.58), 7.302 (2.82), 7.311 (1.81), 7.317 (1.93), |
| | 7.325 (3.60), 7.335 (0.45), 7.397 (1.32), 7.404 (0.64), 7.407 (0.81), |
| | 7.411 (0.90), 7.420 (0.84), 7.435 (1.53), 7.444 (1.31), 7.449 (0.92), |
| | 7.450 (0.65), 7.458 (1.11), 7.486 (1.41), 7.490 (1.39), 7.500 (1.41), |
| | 7.505 (1.45), 7.671 (1.50), 7.674 (1.45), 8.282 (1.90), 8.297 (1.79), |
| | 10.760 (1.89). |
| 162 | 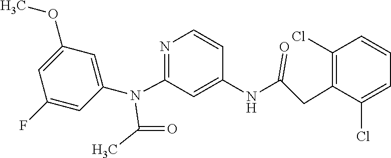 [SMILES: COc1cc(F)cc(N(C(C)=O)c2cc(NC(=O)Cc3c(Cl)cccc3Cl)ccn2)c1] |
| | N-{4-[2-(2,6-Dichlorphenyl)acetamido]pyridin-2-yl}-N-(3-fluor-5- |
| | methoxyphenyl)acetamide |
| | LC-MS (Method 2): Rₜ = 1.22 min; MS (ESIpos): m/z = 462 [M + H]⁺ |
| | ¹H-NMR (400 MHz, DMSO-d6) δ [ppm]: 2.009 (14.08), 2.518 (2.01), |
| | 2.522 (1.28), 3.746 (16.00), 4.080 (6.08), 6.696 (1.58), 6.736 (0.58), |
| | 6.741 (0.98), 6.746 (0.64), 6.760 (0.59), 6.765 (0.92), 6.770 (0.62), |
| | 6.795 (0.74), 6.801 (1.27), 6.807 (0.63), 6.822 (0.73), 6.828 (1.24), |
| | 6.834 (0.59), 7.335 (1.24), 7.354 (1.71), 7.357 (1.79), 7.375 (2.08), |
| | 7.453 (1.39), 7.458 (1.39), 7.467 (1.33), 7.472 (1.47), 7.487 (5.63), |
| | 7.506 (3.44), 7.669 (1.46), 7.672 (1.42), 8.286 (1.90), 8.300 (1.81), |
| | 10.873 (1.98). |
| 163 | 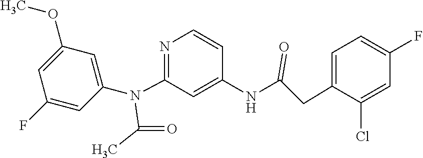 [SMILES: COc1cc(F)cc(N(C(C)=O)c2cc(NC(=O)Cc3ccc(F)cc3Cl)ccn2)c1] |
| | N-{4-[2-(2-Chlor-4-fluorphenyl)acetamido]pyridin-2-yl}-N-(3-fluor- |
| | 5-methoxyphenyl)acetamide |
| | LC-MS (Method 2): Rₜ = 1.19 min; MS (ESIpos): m/z = 446 [M + H]⁺ |
| | ¹H-NMR (400 MHZ, DMSO-d6) δ [ppm]: 2.011 (16.00), 2.518 (0.74), |
| | 2.522 (0.49), 3.337 (2.35), 3.868 (5.81), 6.692 (1.82), 6.731 (0.67), |
| | 6.736 (1.13), 6.741 (0.72), 6.755 (0.67), 6.760 (1.07), 6.765 (0.70), |
| | 6.795 (0.80), 6.801 (1.41), 6.807 (0.71), 6.823 (0.84), 6.829 (1.44), |
| | 6.834 (0.70), 7.186 (0.69), 7.193 (0.81), 7.207 (1.52), 7.214 (1.65), |
| | 7.229 (0.79), 7.235 (0.88), 7.430 (1.55), 7.437 (1.53), 7.445 (1.35), |
| | 7.452 (1.65), 7.459 (2.00), 7.467 (1.24), 7.478 (1.69), 7.483 (2.69), |
| | 7.492 (1.52), 7.497 (1.67), 7.668 (1.76), 7.672 (1.70), 8.282 (2.21), |
| | 8.296 (2.09), 10.758 (2.27). |
| 164 | 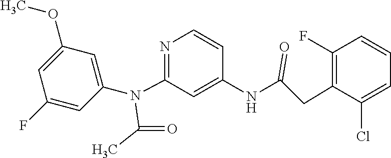 [SMILES: COc1cc(F)cc(N(C(C)=O)c2cc(NC(=O)Cc3c(F)cccc3Cl)ccn2)c1] |
| | N-{4-[2-(2-Chlor-6-fluorphenyl)acetamido]pyridin-2-yl}-N-(3-fluor- |
| | 5-methoxyphenyl)acetamide |
| | LC-MS (Method 2): Rₜ = 1.17 min; MS (ESIpos): m/z = 446 [M + H]⁺ |
| | ¹H-NMR (400 MHZ, DMSO-d6) δ [ppm]: 1.874 (0.59), 1.951 (0.51), |
| | 2.010 (16.00), 2.518 (0.72), 2.522 (0.47), 3.333 (4.52), 3.924 (4.02), |
| | 3.928 (3.79), 6.694 (1.75), 6.734 (0.63), 6.739 (1.08), 6.744 (0.68), |
| | 6.758 (0.64), 6.763 (1.03), 6.769 (0.66), 6.794 (0.78), 6.800 (1.35), |
| | 6.806 (0.68), 6.822 (0.81), 6.827 (1.39), 6.833 (0.67), 7.225 (0.57), |
| | 7.230 (0.58), 7.244 (0.85), 7.249 (1.34), 7.252 (0.73), 7.269 (0.84), |
| | 7.273 (0.81), 7.337 (0.60), 7.341 (0.77), 7.357 (3.19), 7.362 (2.81), |
| | 7.376 (1.22), 7.381 (1.14), 7.396 (1.22), 7.416 (0.41), 7.459 (1.60), |
| | 7.464 (1.52), 7.473 (1.55), 7.478 (1.63), 7.671 (1.65), 7.675 (1.60), |
| | 8.286 (2.14), 8.300 (2.02), 10.861 (2.16). |
| 165 | 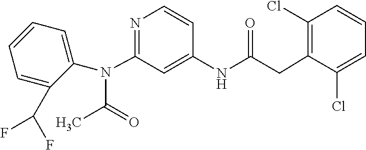 [SMILES: CC(=O)N(c1cc(NC(=O)Cc2c(Cl)cccc2Cl)ccn1)c1ccccc1C(F)F] |
| | N-{4-[2-(2,6-Dichlorphenyl)acetamido]pyridin-2-yl}-N-[2- |
| | (difluormethyl)phenyl]acetamide |
| | LC-MS (Method 2): Rₜ = 1.24 min; MS (ESIneg): m/z = 461 [M − H]⁻ |
| | ¹H-NMR (400 MHz, DMSO-d6) δ [ppm]: 1.970 (16.00), 2.332 (0.69), |
| | 2.518 (3.81), 2.522 (2.40), 2.673 (0.69), 4.082 (14.14), 6.910 (1.98), |
| | 7.047 (3.70), 7.184 (1.73), 7.337 (2.83), 7.356 (3.87), 7.359 (3.96), |
| | 7.378 (4.90), 7.430 (4.37), 7.435 (4.95), 7.444 (3.53), 7.449 (4.49), |
| | 7.489 (13.31), 7.509 (8.00), 7.537 (0.98), 7.540 (0.99), 7.556 (2.61), |
| | 7.558 (2.51), 7.575 (2.04), 7.612 (1.66), 7.631 (2.14), 7.649 (0.80), |
| | 7.703 (2.58), 7.720 (2.02), 7.796 (1.89), 8.221 (3.33), 8.235 (3.18), |
| | 10.875 (4.56). |
| 166 | 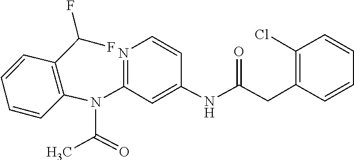 [SMILES: CC(=O)N(c1cc(NC(=O)Cc2ccccc2Cl)ccn1)c1ccccc1C(F)F] |
| | N-{4-[2-(2-Chlorphenyl)acetamido]pyridin-2-yl}-N-[2- |
| | (difluormethyl)phenyl]acetamide |
| | LC-MS (Method 2): Rₜ = 1.13 min; MS (ESIpos): m/z = 430 [M + H]⁺ |
| | ¹H-NMR (400 MHZ, DMSO-d6) δ [ppm]: 1.088 (0.42), 1.105 (0.44), |
| | 1.141 (0.43), 1.157 (0.41), 1.974 (16.00), 2.332 (0.56), 2.518 (2.76), |
| | 2.522 (1.74), 2.669 (0.77), 2.673 (0.55), 3.880 (13.98), 6.906 (2.00), |
| | 7.043 (3.74), 7.180 (1.71), 7.293 (0.60), 7.303 (6.02), 7.313 (3.97), |
| | 7.317 (4.28), 7.319 (4.26), 7.327 (8.31), 7.336 (0.90), 7.338 (1.04), |
| | 7.390 (0.48), 7.399 (3.12), 7.405 (1.63), 7.409 (1.94), 7.413 (2.18), |
| | 7.422 (2.76), 7.427 (2.63), 7.437 (4.07), 7.446 (5.32), 7.450 (3.42), |
| | 7.462 (4.02), 7.467 (3.43), 7.476 (3.25), 7.481 (3.25), 7.539 (0.97), |
| | 7.554 (2.59), 7.557 (2.47), 7.573 (2.04), 7.611 (1.64), 7.630 (2.11), |
| | 7.648 (0.79), 7.701 (2.53), 7.720 (1.98), 7.796 (1.98), 8.219 (3.36), |
| | 8.233 (3.18), 10.765 (4.42). |
| 167 | 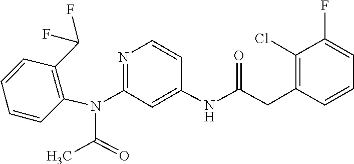 [SMILES: CC(=O)N(c1cc(NC(=O)Cc2cccc(F)c2Cl)ccn1)c1ccccc1C(F)F] |
| | N-{4-[2-(2-Chlor-3-fluorphenyl)acetamido]pyridin-2-yl}-N-[2- |
| | (difluormethyl)phenyl]acetamide |
| | LC-MS (Method 2): Rₜ = 1.14 min; MS (ESIpos): m/z = 448 [M + H]⁺ |
| | ¹H-NMR (400 MHZ, DMSO-d6) δ [ppm]: 1.092 (0.43), 1.109 (0.44), |
| | 1.164 (0.43), 1.180 (0.42), 1.972 (16.00), 2.518 (4.71), 2.522 (3.00), |
| | 3.943 (13.63), 6.904 (2.01), 7.041 (3.76), 7.178 (1.75), 7.263 (1.46), |
| | 7.270 (2.19), 7.277 (1.59), 7.281 (1.71), 7.285 (2.40), 7.326 (0.85), |
| | 7.340 (3.50), 7.345 (4.52), 7.361 (8.19), 7.368 (2.64), 7.376 (2.52), |
| | 7.382 (0.76), 7.397 (0.44), 7.428 (1.92), 7.448 (2.45), 7.454 (4.13), |
| | 7.459 (3.61), 7.468 (3.39), 7.473 (3.56), 7.539 (0.97), 7.556 (2.61), |
| | 7.558 (2.50), 7.575 (2.03), 7.612 (1.64), 7.631 (2.12), 7.649 (0.78), |
| | 7.702 (2.54), 7.720 (1.98), 7.795 (1.96), 8.221 (3.39), 8.234 (3.25), |
| | 10.803 (4.61). |
| 168 | 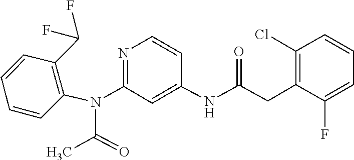 [SMILES: CC(=O)N(c1cc(NC(=O)Cc2c(F)cccc2Cl)ccn1)c1ccccc1C(F)F] |
| | N-{4-[2-(2-Chlor-6-fluorphenyl)acetamido]pyridin-2-yl}-N-[2- |
| | (difluormethyl)phenyl]acetamide |
| | LC-MS (Method 2): Rₜ = 1.13 min; MS (ESIpos): m/z = 448 [M + H]⁺ |
| | ¹H-NMR (400 MHz, DMSO-d6) δ [ppm]: 1.088 (0.42), 1.906 (0.56), |
| | 1.971 (16.00), 2.518 (13.92), 2.522 (9.48), 3.307 (0.84), 3.926 (8.60), |
| | 3.929 (8.39), 6.906 (1.96), 7.044 (3.69), 7.181 (1.73), 7.229 (1.17), |
| | 7.234 (1.18), 7.248 (1.74), 7.253 (2.84), 7.272 (1.77), 7.275 (1.71), |
| | 7.340 (1.37), 7.344 (1.62), 7.360 (6.48), 7.365 (5.76), 7.379 (2.57), |
| | 7.384 (2.28), 7.400 (2.59), 7.405 (0.81), 7.420 (0.95), 7.433 (4.95), |
| | 7.438 (3.96), 7.447 (4.73), 7.452 (5.52), 7.537 (0.98), 7.556 (2.67), |
| | 7.575 (2.13), 7.612 (1.73), 7.630 (2.21), 7.648 (0.86), 7.702 (2.64), |
| | 7.719 (2.13), 7.798 (2.03), 8.221 (3.34), 8.236 (3.23), 10.865 (4.67). |
| 169 | 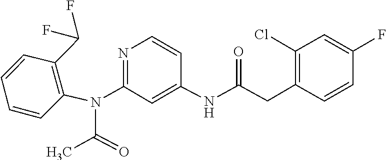 [SMILES: CC(=O)N(c1cc(NC(=O)Cc2ccc(F)cc2Cl)ccn1)c1ccccc1C(F)F] |
| | N-{4-[2-(2-Chlor-4-fluorphenyl)acetamido]pyridin-2-yl}-N-[2- |
| | (difluormethyl)phenyl]acetamide |
| | LC-MS (Method 2): Rₜ = 1.15 min; MS (ESIpos): m/z = 448 [M + H]⁺ |
| | ¹H-NMR (400 MHZ, DMSO-d6) δ [ppm]: 1.085 (0.56), 1.102 (0.55), |
| | 1.156 (0.52), 1.173 (0.53), 1.907 (0.45), 1.973 (16.00), 2.208 (0.58), |
| | 2.518 (6.69), 2.522 (4.57), 3.309 (0.43), 3.871 (12.17), 6.904 (1.93), |
| | 7.041 (3.65), 7.178 (1.72), 7.189 (1.42), 7.196 (1.57), 7.210 (2.91), |
| | 7.217 (3.21), 7.232 (1.58), 7.238 (1.72), 7.427 (2.12), 7.433 (3.64), |
| | 7.440 (3.82), 7.447 (5.28), 7.452 (4.81), 7.456 (5.57), 7.462 (5.90), |
| | 7.466 (4.83), 7.471 (4.64), 7.485 (2.35), 7.539 (0.98), 7.555 (2.68), |
| | 7.575 (2.09), 7.611 (1.75), 7.630 (2.26), 7.648 (0.88), 7.701 (2.65), |
| | 7.719 (2.12), 7.792 (2.10), 8.217 (3.37), 8.231 (3.20), 10.764 (4.73). |
| 170 | 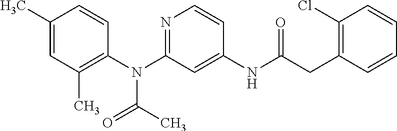 [SMILES: CC(=O)N(c1cc(NC(=O)Cc2ccccc2Cl)ccn1)c1ccc(C)cc1C] |
| | N-{4-[2-(2-Chlorphenyl)acetamido]pyridin-2-yl}-N-(2,4- |
| | dimethylphenyl)acetamide |
| | LC-MS (Method 1): Rₜ = 1.22 min; MS (ESIpos): m/z = 408 [M + H]⁺ |
| | ¹H-NMR (500 MHz, DMSO-d6) δ [ppm]: 1.957 (10.76), 2.099 (16.00), |
| | 2.285 (14.83), 2.514 (1.85), 2.518 (1.82), 2.522 (1.48), 3.869 (10.35), |
| | 7.039 (0.83), 7.052 (1.80), 7.054 (1.98), 7.075 (3.62), 7.091 (1.52), |
| | 7.119 (2.65), 7.305 (4.31), 7.311 (2.43), 7.313 (2.65), 7.316 (2.58), |
| | 7.317 (2.73), 7.323 (5.76), 7.332 (0.61), 7.397 (2.11), 7.402 (1.00), |
| | 7.405 (1.31), 7.409 (1.43), 7.416 (1.59), 7.418 (2.61), 7.422 (2.53), |
| | 7.429 (2.73), 7.433 (2.59), 7.438 (2.47), 7.445 (1.85), 7.448 (1.32), |
| | 7.450 (0.95), 7.456 (1.79), 7.721 (1.02), 8.179 (2.50), 8.190 (2.43), |
| | 10.697 (3.21). |
| 171 | 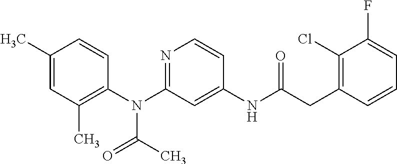 [SMILES: CC(=O)N(c1cc(NC(=O)Cc2cccc(F)c2Cl)ccn1)c1ccc(C)cc1C] |
| | N-{4-[2-(2-Chlor-3-fluorphenyl)acetamido]pyridin-2-yl}-N-(2,4- |
| | dimethylphenyl)acetamide |
| | LC-MS (Method 1): Rₜ = 1.23 min; MS (ESIpos): m/z = 426 [M + H]⁺ |
| | ¹H-NMR (500 MHz, DMSO-d6) δ [ppm]: 1.955 (10.87), 2.097 (16.00), |
| | 2.286 (14.66), 2.514 (1.10), 2.518 (1.09), 2.522 (0.88), 3.932 (9.85), |
| | 7.040 (0.84), 7.052 (1.81), 7.056 (1.97), 7.077 (3.63), 7.092 (1.52), |
| | 7.119 (2.65), 7.263 (1.06), 7.267 (1.45), 7.274 (1.09), 7.277 (1.21), |
| | 7.281 (1.54), 7.329 (0.57), 7.340 (2.28), 7.346 (3.34), 7.359 (3.61), |
| | 7.363 (2.04), 7.371 (1.51), 7.376 (0.54), 7.412 (2.41), 7.416 (2.36), |
| | 7.423 (2.42), 7.427 (2.49), 7.723 (1.01), 8.181 (2.58), 8.193 (2.46), |
| | 10.736 (3.25). |
| 172 | 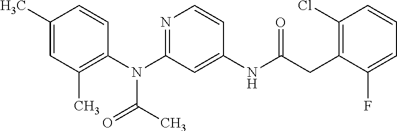 [SMILES: CC(=O)N(c1cc(NC(=O)Cc2c(F)cccc2Cl)ccn1)c1ccc(C)cc1C] |
| | N-{4-[2-(2-Chlor-6-fluorphenyl)acetamido]pyridin-2-yl}-N-(2,4- |
| | dimethylphenyl)acetamide |
| | LC-MS (Method 1): Rₜ = 1.22 min; MS (ESIpos): m/z = 426 [M + H]⁺ |
| | ¹H-NMR (500 MHz, DMSO-d6) δ [ppm]: 1.954 (11.00), 2.098 (16.00), |
| | 2.286 (15.07), 2.514 (2.15), 2.518 (2.02), 2.522 (1.65), 3.915 (5.94), |
| | 3.917 (5.82), 7.039 (0.87), 7.056 (1.99), 7.078 (3.57), 7.094 (1.58), |
| | 7.119 (2.70), 7.231 (0.83), 7.235 (0.87), 7.247 (1.20), 7.250 (1.94), |
| | 7.267 (1.08), 7.269 (1.11), 7.342 (1.04), 7.344 (1.12), 7.358 (3.64), |
| | 7.361 (2.33), 7.367 (1.69), 7.378 (1.54), 7.383 (1.68), 7.391 (2.49), |
| | 7.395 (4.10), 7.399 (0.80), 7.402 (2.32), 7.406 (2.35), 7.411 (0.65), |
| | 7.720 (1.02), 8.182 (2.60), 8.193 (2.48), 10.797 (3.27). |
| 173 | 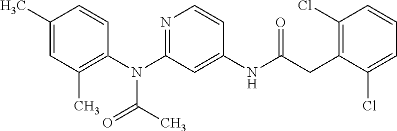 [SMILES: CC(=O)N(c1cc(NC(=O)Cc2c(Cl)cccc2Cl)ccn1)c1ccc(C)cc1C] |
| | N-{4-[2-(2,6-Dichlorphenyl)acetamido]pyridin-2-yl}-N-(2,4- |
| | dimethylphenyl)acetamide |
| | LC-MS (Method 1): Rₜ = 1.27 min; MS (ESIpos): m/z = 442 [M + H]⁺ |
| | ¹H-NMR (500 MHZ, DMSO-d6) δ [ppm]: 1.953 (10.84), 2.100 (16.00), |
| | 2.286 (14.91), 2.514 (1.61), 2.518 (1.55), 2.522 (1.25), 4.071 (10.40), |
| | 7.040 (0.89), 7.053 (1.82), 7.056 (1.97), 7.079 (3.62), 7.095 (1.61), |
| | 7.120 (2.68), 7.340 (2.01), 7.356 (2.63), 7.357 (2.94), 7.373 (2.94), |
| | 7.388 (2.25), 7.392 (2.25), 7.399 (2.19), 7.403 (2.24), 7.489 (8.81), |
| | 7.505 (6.54), 7.719 (1.00), 8.182 (2.55), 8.193 (2.46), 10.808 (3.30). |
| 174 | 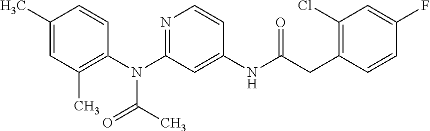 [SMILES: CC(=O)N(c1cc(NC(=O)Cc2ccc(F)cc2Cl)ccn1)c1ccc(C)cc1C] |
| | N-{4-[2-(2-Chlor-4-fluorphenyl)acetamido]pyridin-2-yl}-N-(2,4- |
| | dimethylphenyl)acetamide |
| | LC-MS (Method 1): Rₜ = 1.24 min; MS (ESIpos): m/z = 426 [M + H]⁺ |
| | ¹H-NMR (500 MHz, DMSO-d6) δ [ppm]: 1.955 (10.72), 2.097 (16.00), |
| | 2.285 (14.84), 2.514 (1.21), 2.518 (1.21), 2.522 (0.97), 3.859 (8.72), |
| | 7.039 (0.82), 7.051 (1.83), 7.055 (1.99), 7.075 (3.61), 7.091 (1.52), |
| | 7.119 (2.64), 7.192 (1.01), 7.197 (1.07), 7.209 (2.10), 7.214 (2.21), |
| | 7.226 (1.11), 7.231 (1.16), 7.410 (2.30), 7.414 (2.30), 7.421 (2.31), |
| | 7.425 (2.38), 7.434 (2.05), 7.439 (2.11), 7.447 (1.83), 7.451 (2.21), |
| | 7.457 (2.20), 7.460 (1.92), 7.465 (1.68), 7.477 (1.56), 7.719 (1.00), |
| | 8.178 (2.51), 8.189 (2.43), 10.695 (3.31). |
| 175 | 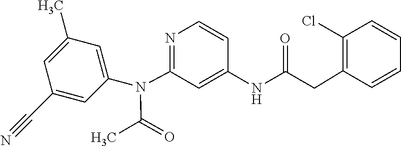 [SMILES: CC(=O)N(c1cc(C)cc(C#N)c1)c1cc(NC(=O)Cc2ccccc2Cl)ccn1] |
| | N-{4-[2-(2-Chlorphenyl)acetamido]pyridin-2-yl}-N-(3-cyan-5- |
| | methylphenyl)acetamide |
| | LC-MS (Method 1): Rₜ = 1.14 min; MS (ESIpos): m/z = 419 [M + H]⁺ |
| | ¹H-NMR (400 MHz, DMSO-d6) δ [ppm]: 2.005 (16.00), 2.335 (11.69), |
| | 2.518 (2.26), 2.523 (1.61), 2.669 (0.43), 3.880 (8.22), 7.303 (3.43), |
| | 7.313 (2.43), 7.319 (2.52), 7.327 (4.54), 7.337 (0.59), 7.398 (1.78), |
| | 7.406 (0.92), 7.409 (1.16), 7.414 (1.26), 7.422 (1.20), 7.426 (0.52), |
| | 7.437 (3.31), 7.445 (3.25), 7.450 (1.66), 7.460 (1.49), 7.495 (1.81), |
| | 7.500 (1.78), 7.509 (1.80), 7.514 (1.87), 7.616 (2.59), 7.618 (2.67), |
| | 7.633 (2.24), 7.685 (1.65), 8.277 (2.11), 8.292 (2.03), 10.777 (2.74). |
| 176 | 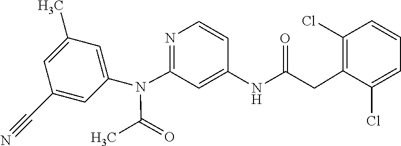 [SMILES: CC(=O)N(c1cc(C)cc(C#N)c1)c1cc(NC(=O)Cc2c(Cl)cccc2Cl)ccn1] |
| | N-(3-Cyan-5-methylphenyl)-N-{4-[2-(2,6- |
| | dichlorphenyl)acetamido]pyridin-2-yl}acetamide |
| | LC-MS (Method 2): Rₜ = 1.18 min; MS (ESIpos): m/z = 453 [M + H]⁺ |
| | ¹H-NMR (400 MHz, DMSO-d6) δ [ppm]: 2.002 (16.00), 2.074 (1.20), |
| | 2.084 (2.71), 2.335 (11.67), 2.518 (2.71), 2.523 (1.94), 2.669 (0.47), |
| | 4.082 (8.02), 7.336 (1.56), 7.355 (2.19), 7.358 (2.30), 7.376 (2.64), |
| | 7.447 (2.23), 7.464 (1.73), 7.469 (1.73), 7.478 (1.78), 7.483 (2.16), |
| | 7.488 (7.48), 7.508 (4.49), 7.616 (2.56), 7.618 (2.62), 7.637 (2.20), |
| | 7.685 (1.54), 8.281 (2.07), 8.295 (1.99), 10.892 (2.71). |
| 177 | 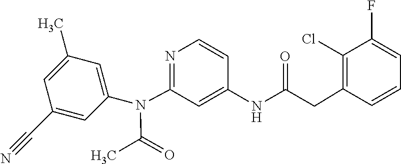 [SMILES: CC(=O)N(c1cc(C)cc(C#N)c1)c1cc(NC(=O)Cc2cccc(F)c2Cl)ccn1] |
| | N-{4-[2-(2-Chlor-3-fluorphenyl)acetamido]pyridin-2-yl}-N-(3-cyan-5- |
| | methylphenyl)acetamide |
| | LC-MS (Method 1): Rₜ = 1.16 min; MS (ESIpos): m/z = 437 [M + H]⁺ |
| | ¹H-NMR (400 MHz, DMSO-d6) δ [ppm]: 0.897 (0.43), 0.914 (1.09), |
| | 0.932 (0.81), 1.164 (0.52), 1.465 (0.76), 2.004 (16.00), 2.335 (12.34), |
| | 2.518 (4.49), 2.523 (3.09), 2.539 (7.30), 2.665 (0.56), 2.669 (0.78), |
| | 2.673 (0.56), 3.944 (5.44), 7.264 (0.85), 7.270 (1.16), 7.286 (1.36), |
| | 7.325 (0.48), 7.340 (1.98), 7.345 (2.54), 7.361 (4.39), 7.368 (1.58), |
| | 7.376 (1.29), 7.382 (0.43), 7.443 (2.37), 7.493 (1.15), 7.504 (1.18), |
| | 7.619 (2.92), 7.633 (2.39), 7.686 (1.71), 8.280 (1.86), 8.294 (1.78), |
| | 10.821 (2.00). |
| 178 | 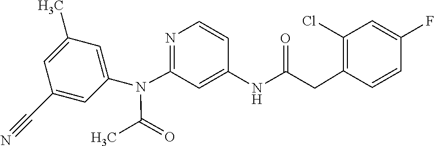 [SMILES: CC(=O)N(c1cc(C)cc(C#N)c1)c1cc(NC(=O)Cc2ccc(F)cc2Cl)ccn1] |
| | N-{4-[2-(2-Chlor-4-fluorphenyl)acetamido]pyridin-2-yl}-N-(3-cyan-5- |
| | methylphenyl)acetamide |
| | LC-MS (Method 1): Rₜ = 1.17 min; MS (ESIneg): m/z = 437 [M + H]⁺ |
| | ¹H-NMR (400 MHZ, DMSO-d6) δ [ppm]: 2.003 (16.00), 2.074 (0.63), |
| | 2.334 (11.55), 2.518 (2.36), 2.523 (1.63), 2.540 (1.50), 2.669 (0.40), |
| | 3.871 (6.80), 7.188 (0.76), 7.195 (0.87), 7.210 (1.65), 7.217 (1.78), |
| | 7.231 (0.89), 7.238 (0.98), 7.433 (2.05), 7.439 (3.55), 7.447 (2.78), |
| | 7.455 (1.95), 7.462 (2.72), 7.469 (1.51), 7.486 (2.21), 7.491 (1.74), |
| | 7.501 (1.66), 7.505 (1.75), 7.617 (2.62), 7.619 (2.76), 7.631 (2.27), |
| | 7.682 (1.64), 8.276 (2.09), 8.290 (1.99), 10.776 (2.69). |
| 179 | 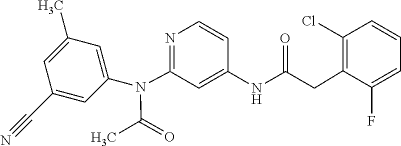 [SMILES: CC(=O)N(c1cc(C)cc(C#N)c1)c1cc(NC(=O)Cc2c(F)cccc2Cl)ccn1] |
| | N-{4-[2-(2-Chlor-6-fluorphenyl)acetamido]pyridin-2-yl}-N-(3-cyan-5- |
| | methylphenyl)acetamide |
| | LC-MS (Method 1): Rₜ = 1.15 min; MS (ESIpos): m/z = 437 [M + H]⁺ |
| | ¹H-NMR (400 MHz, DMSO-d6) δ [ppm]: 2.002 (16.00), 2.334 (12.21), |
| | 2.518 (6.68), 2.523 (4.72), 2.665 (0.83), 2.669 (1.14), 2.673 (0.81), |
| | 3.926 (5.10), 3.929 (4.99), 7.228 (0.69), 7.233 (0.73), 7.247 (1.00), |
| | 7.251 (1.65), 7.271 (1.04), 7.274 (1.00), 7.339 (0.73), 7.343 (0.94), |
| | 7.359 (3.76), 7.364 (3.48), 7.378 (1.48), 7.383 (1.35), 7.398 (1.48), |
| | 7.403 (0.47), 7.418 (0.48), 7.442 (2.31), 7.467 (1.70), 7.472 (1.74), |
| | 7.481 (1.70), 7.486 (1.68), 7.617 (2.80), 7.635 (2.31), 7.686 (1.67), |
| | 8.281 (2.11), 8.296 (2.04), 10.881 (2.66). |
| 180 | 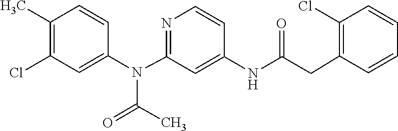 [SMILES: CC(=O)N(c1ccc(C)c(Cl)c1)c1cc(NC(=O)Cc2ccccc2Cl)ccn1] |
| | N-(3-Chlor-4-methylphenyl)-N-{4-[2-(2- |
| | chlorphenyl)acetamido]pyridin-2-yl}acetamide |
| | LC-MS (Method 2): Rₜ = 1.25 min; MS (ESIpos): m/z = 428 [M + H]⁺ |
| | ¹H-NMR (400 MHZ, DMSO-d6) δ [ppm]: 1.130 (0.92), 1.146 (0.94), |
| | 1.997 (16.00), 2.319 (11.24), 2.518 (1.45), 2.522 (0.96), 3.873 (7.48), |
| | 7.112 (1.11), 7.118 (1.11), 7.132 (1.28), 7.138 (1.28), 7.300 (3.36), |
| | 7.309 (2.46), 7.315 (2.49), 7.324 (4.38), 7.334 (0.61), 7.359 (2.02), |
| | 7.381 (1.98), 7.388 (2.84), 7.393 (3.93), 7.401 (1.02), 7.404 (1.13), |
| | 7.408 (1.28), 7.417 (1.03), 7.424 (0.44), 7.433 (1.83), 7.442 (1.52), |
| | 7.447 (1.15), 7.457 (1.32), 7.474 (1.70), 7.478 (1.70), 7.488 (1.72), |
| | 7.493 (1.73), 7.657 (1.73), 8.268 (2.22), 8.282 (2.10), 10.754 (2.38). |
| 181 | 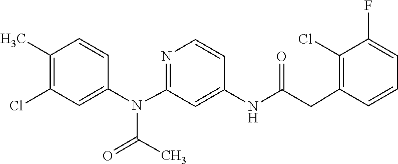 [SMILES: CC(=O)N(c1ccc(C)c(Cl)c1)c1cc(NC(=O)Cc2cccc(F)c2Cl)ccn1] |
| | N-{4-[2-(2-Chlor-3-fluorphenyl)acetamido]pyridin-2-yl}-N-(3-chlor- |
| | 4-methylphenyl)acetamide |
| | LC-MS (Method 2): Rₜ = 1.26 min; MS (ESIpos): m/z = 446 [M + H]⁺ |
| | ¹H-NMR (400 MHZ, DMSO-d6) δ [ppm]: 1.139 (0.87), 1.154 (0.89), |
| | 1.997 (16.00), 2.321 (11.17), 2.518 (1.22), 2.523 (0.82), 3.937 (6.95), |
| | 7.115 (1.09), 7.120 (1.14), 7.135 (1.26), 7.140 (1.29), 7.259 (0.83), |
| | 7.266 (1.15), 7.272 (0.84), 7.277 (0.96), 7.282 (1.28), 7.322 (0.42), |
| | 7.337 (1.80), 7.341 (2.23), 7.358 (4.81), 7.365 (1.80), 7.372 (1.35), |
| | 7.382 (1.92), 7.389 (2.61), 7.395 (2.56), 7.467 (1.57), 7.472 (1.69), |
| | 7.481 (1.65), 7.486 (1.70), 7.657 (1.68), 8.271 (2.20), 8.285 (2.10), |
| | 10.792 (2.36). |
| 182 | 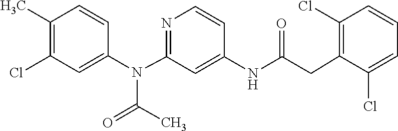 [SMILES: CC(=O)N(c1ccc(C)c(Cl)c1)c1cc(NC(=O)Cc2c(Cl)cccc2Cl)ccn1] |
| | N-(3-Chlor-4-methylphenyl)-N-{4-[2-(2,6- |
| | dichlorphenyl)acetamido]pyridin-2-yl}acetamide |
| | LC-MS (Method 2): Rₜ = 1.30 min; MS (ESIneg): m/z = 460 [M − H]⁻ |
| | ¹H-NMR (400 MHZ, DMSO-d6) δ [ppm]: 1.995 (16.00), 2.320 (11.25), |
| | 2.518 (1.66), 2.523 (1.19), 4.076 (7.36), 7.117 (1.10), 7.122 (1.12), |
| | 7.138 (1.27), 7.143 (1.32), 7.334 (1.53), 7.353 (1.98), 7.355 (2.43), |
| | 7.360 (2.17), 7.374 (2.63), 7.381 (1.83), 7.392 (2.59), 7.398 (2.48), |
| | 7.440 (1.69), 7.445 (1.65), 7.454 (1.68), 7.459 (1.77), 7.485 (6.72), |
| | 7.505 (4.06), 7.658 (1.64), 8.271 (2.20), 8.286 (2.10), 10.869 (2.40). |
| 183 | 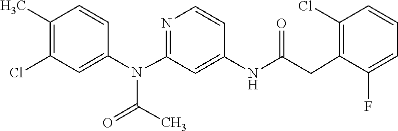 [SMILES: CC(=O)N(c1ccc(C)c(Cl)c1)c1cc(NC(=O)Cc2c(F)cccc2Cl)ccn1] |
| | N-{4-[2-(2-Chlor-6-fluorphenyl)acetamido]pyridin-2-yl}-N-(3-chlor- |
| | 4-methylphenyl)acetamide |
| | LC-MS (Method 2): Rₜ = 1.26 min; MS (ESIpos): m/z = 446 [M + H]⁺ |
| | 1H-NMR (400 MHZ, DMSO-d6) δ [ppm]: 1.995 (16.00), 2.320 (10.77), |
| | 2.518 (1.42), 2.523 (1.05), 3.920 (4.14), 3.923 (4.02), 7.115 (1.07), |
| | 7.120 (1.06), 7.135 (1.22), 7.140 (1.25), 7.224 (0.59), 7.229 (0.61), |
| | 7.243 (0.88), 7.248 (1.40), 7.268 (0.85), 7.272 (0.84), 7.336 (0.69), |
| | 7.340 (0.80), 7.356 (3.76), 7.361 (4.23), 7.376 (1.61), 7.381 (2.88), |
| | 7.390 (2.62), 7.396 (3.55), 7.416 (0.46), 7.445 (1.55), 7.450 (1.68), |
| | 7.460 (1.63), 7.464 (1.69), 7.659 (1.60), 8.272 (2.09), 8.286 (2.02), |
| | 10.856 (2.25) |
| 184 | 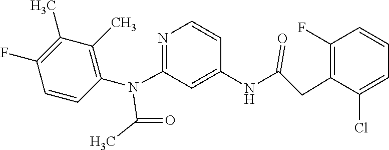 [SMILES: CC(=O)N(c1cc(NC(=O)Cc2c(F)cccc2Cl)ccn1)c1ccc(F)c(C)c1C] |
| | N-{4-[2-(2-Chlor-6-fluorphenyl)acetamido]pyridin-2-yl}-N-(4-fluor- |
| | 2,3-dimethylphenyl)acetamide |
| | LC-MS (Method 2): Rₜ = 1.23 min; MS (ESIpos): m/z = 444 [M + H]⁺ |
| | ¹H-NMR (400 MHZ, DMSO-d6) δ [ppm]: 1.958 (11.35), 2.094 (16.00), |
| | 2.169 (8.22), 2.174 (8.15), 2.332 (0.63), 2.518 (2.83), 2.522 (1.85), |
| | 2.673 (0.60), 3.919 (5.76), 3.923 (5.54), 7.034 (0.95), 7.057 (2.42), |
| | 7.079 (1.69), 7.110 (1.45), 7.124 (1.57), 7.131 (0.93), 7.145 (0.81), |
| | 7.228 (0.86), 7.233 (0.85), 7.247 (1.28), 7.252 (2.03), 7.256 (1.09), |
| | 7.272 (1.29), 7.276 (1.22), 7.340 (0.95), 7.343 (1.18), 7.360 (5.06), |
| | 7.365 (4.51), 7.378 (1.84), 7.384 (1.68), 7.399 (2.38), 7.401 (2.80), |
| | 7.405 (2.69), 7.415 (2.57), 7.419 (2.92), 7.746 (1.19), 8.193 (2.72), |
| | 8.207 (2.58), 10.813 (3.15). |

### Example 185 (Examples with Pyridazine Core)

### N-{5-[2-(2,6-dichlorophenyl)acetamido]pyridazin-3-yl}-N-phenylacetamide

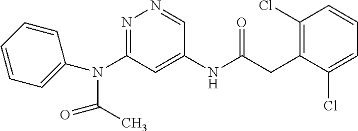 [SMILES: CC(=O)N(c1ccccc1)c1cc(NC(=O)Cc2c(Cl)cccc2Cl)cnn1]

According to GP J, N-(6-anilinopyridazin-4-yl)-2-(2,6-dichlorophenyl)acetamide (Int. 123, 56 mg, 0.15 mmol) was dissolved in dichloromethane (2 mL) and acetyl chloride (18 mg, 0.22 mmol, 1.5 eq) and triethylamine (27 mg, 0.27, 1.8 eq) were added. The mixture was stirred at rt for 18 h, then concentrated in vacuo and purified via preparative HPLC to yield 45 mg (73% of theory) of the title compound.

¹H-NMR (400 MHz, DMSO-d6) δ[ppm]: −0.009 (0.55), 0.008 (0.44), 1.109 (11.45), 2.016 (10.62), 2.324 (0.40), 2.329 (0.55), 2.334 (0.40), 2.520 (1.83), 2.525 (1.23), 2.542 (16.00), 2.666 (0.40), 2.671 (0.57), 2.676 (0.40), 4.130 (4.81), 4.196 (0.95), 7.346 (0.51), 7.349 (1.32), 7.360 (1.56), 7.363 (2.20), 7.368 (1.82), 7.371 (1.94), 7.379 (2.97), 7.382 (2.46), 7.390 (1.61), 7.431 (1.96), 7.446 (1.58), 7.449 (1.56), 7.455 (0.42), 7.468 (0.84), 7.500 (4.26), 7.519 (2.61), 8.102 (1.30), 8.107 (1.28), 9.124 (2.46), 9.129 (2.40), 11.166 (1.38).

LC-MS (Method 1): Rt=1.10 min; MS (ESIpos): m/z=415 [M+H]⁺

TABLE 9
Examples 186-208: According to GP J, the following examples were prepared

| | |
|---|---|
| 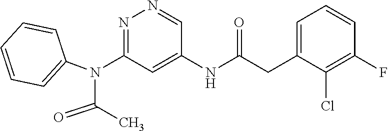 | 186 |
| N-{5-[2-(2-chloro-3-fluorophenyl)acetamido]pyridazin-3-yl}-N-phenylacetamide | |
| LC-MS (Method 1): Rₜ = 1.05 min; MS (ESIneg): m/z = 397 [M − H]⁻ | |
| ¹H-NMR (400 MHZ, DMSO-d6) δ [ppm]: −0.008 (1.19), 0.008 (1.10), | |
| 1.343 (0.45), 2.019 (16.00), 2.070 (0.67), 2.171 (0.41), 2.206 (1.22), | |
| 2.521 (2.84), 2.525 (1.93), 2.585 (0.42), 3.995 (6.91), 4.004 (0.81), | |
| 4.029 (0.49), 7.277 (0.73), 7.285 (1.15), 7.290 (0.81), 7.294 (0.88), | |
| 7.301 (1.23), 7.339 (0.50), 7.344 (0.45), 7.347 (0.94), 7.355 (3.75), | |
| 7.360 (2.71), 7.364 (2.39), 7.372 (5.23), 7.375 (4.21), 7.377 (3.93), | |
| 7.382 (4.08), 7.387 (1.87), 7.392 (0.46), 7.426 (0.59), 7.432 (2.69), | |
| 7.445 (1.03), 7.448 (1.93), 7.450 (2.57), 7.453 (1.64), 7.456 (0.67), | |
| 7.465 (0.59), 7.469 (1.27), 7.472 (0.70), 8.104 (2.03), 8.110 (2.01), | |
| 9.147 (3.89), 9.153 (3.83), 11.075 (1.19). | |
| 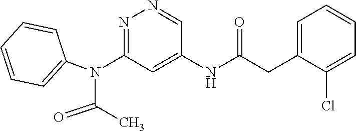 | 187 |
| N-{5-[2-(2-chlorophenyl)acetamido]pyridazin-3-yl}-N-phenylacetamide | |
| LC-MS (Method 2): Rₜ = 1.00 min; MS (ESIpos): m/z = 381 [M + H]⁺ | |
| ¹H-NMR (400 MHZ, DMSO-d6) δ [ppm]: −0.008 (0.69), 0.008 (0.72), | |
| 2.020 (16.00), 2.334 (0.50), 2.520 (2.25), 2.525 (1.39), 2.676 (0.44), | |
| 3.932 (7.34), 7.304 (0.43), 7.315 (2.71), 7.323 (2.73), 7.328 (3.00), | |
| 7.337 (3.85), 7.347 (1.19), 7.353 (1.92), 7.356 (2.26), 7.363 (2.17), | |
| 7.368 (1.67), 7.376 (3.67), 7.380 (2.69), 7.411 (1.56), 7.421 (1.30), | |
| 7.426 (1.69), 7.431 (3.03), 7.448 (3.65), 7.455 (1.61), 7.462 (1.67), | |
| 7.469 (1.52), 7.470 (1.89), 8.104 (2.05), 8.109 (2.05), 9.153 (3.41), | |
| 9.159 (3.33), 11.038 (2.24). | |
| 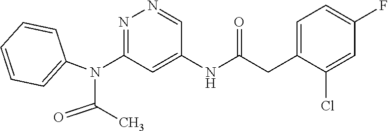 | 188 |
| N-{5-[2-(2-chloro-4-fluorophenyl)acetamido]pyridazin-3-yl}-N-phenylacetamide | |
| LC-MS (Method 2): Rₜ = 1.06 min; MS (ESIpos): m/z = 399 [M + H]⁺ | |
| ¹H-NMR (400 MHZ, DMSO-d6) δ [ppm]: −0.008 (1.19), 0.008 (1.12), | |
| 2.019 (16.00), 2.334 (0.71), 2.520 (3.36), 2.525 (2.18), 2.676 (0.71), | |
| 3.311 (0.44), 3.923 (6.92), 7.199 (0.71), 7.206 (0.81), 7.220 (1.53), | |
| 7.227 (1.68), 7.242 (0.84), 7.248 (0.89), 7.346 (0.92), 7.356 (2.37), | |
| 7.364 (2.20), 7.368 (1.83), 7.373 (3.68), 7.376 (3.73), 7.381 (2.37), | |
| 7.431 (2.97), 7.447 (3.64), 7.450 (3.27), 7.453 (3.02), 7.462 (1.78), | |
| 7.469 (2.92), 7.476 (2.57), 7.483 (1.36), 7.499 (1.21), 8.100 (2.23), | |
| 8.105 (2.22), 9.145 (3.88), 9.151 (3.83), 11.035 (2.12). | |
| 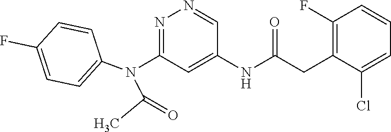 | 189 |
| N-{5-[2-(2-chloro-6-fluorophenyl)acetamido]pyridazin-3-yl}-N-(4- | |
| fluorophenyl)acetamide | |
| LC-MS (Method 1): Rₜ = 1.03 min; MS (ESIpos): m/z = 417 [M + H]⁺ | |
| ¹H-NMR (400 MHz, DMSO-d6) δ [ppm]: 2.011 (16.00), 2.465 (0.42), | |
| 2.470 (0.56), 2.520 (3.11), 2.525 (2.13), 3.298 (0.50), 3.304 (0.44), | |
| 3.312 (0.81), 3.320 (1.24), 3.350 (1.16), 3.355 (0.47), 3.978 (4.00), | |
| 3.982 (3.84), 7.241 (0.63), 7.246 (0.66), 7.258 (2.28), 7.265 (1.92), | |
| 7.268 (0.91), 7.275 (0.85), 7.281 (4.02), 7.285 (1.50), 7.288 (1.12), | |
| 7.296 (0.72), 7.302 (2.47), 7.352 (0.69), 7.356 (0.78), 7.372 (3.07), | |
| 7.377 (1.89), 7.379 (1.79), 7.393 (1.23), 7.398 (1.15), 7.413 (1.33), | |
| 7.418 (0.63), 7.427 (2.13), 7.433 (1.18), 7.440 (2.27), 7.444 (1.00), | |
| 7.450 (1.87), 7.457 (0.65), 7.463 (1.65), 8.137 (1.77), 8.142 (1.74), | |
| 9.130 (3.66), 9.135 (3.64), 11.146 (0.63). | |
| 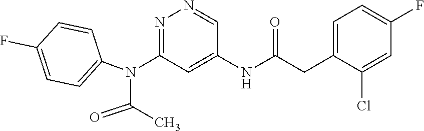 | 190 |
| N-{5-[2-(2-chloro-4-fluorophenyl)acetamido]pyridazin-3-yl}-N-(4- | |
| fluorophenyl)acetamide | |
| LC-MS (Method 1): Rₜ = 1.10 min; MS (ESIpos): m/z = 417 [M + H]⁺ | |
| ¹H-NMR (400 MHZ, DMSO-d6) δ [ppm]: 0.970 (0.41), 1.112 (0.56), | |
| 2.014 (16.00), 2.334 (0.63), 2.521 (2.91), 2.525 (1.85), 2.676 (0.63), | |
| 3.927 (6.54), 7.202 (0.73), 7.208 (0.85), 7.223 (1.61), 7.230 (1.79), | |
| 7.244 (0.94), 7.251 (1.04), 7.261 (2.12), 7.266 (0.78), 7.278 (0.88), | |
| 7.283 (4.44), 7.288 (0.86), 7.292 (0.42), 7.299 (0.77), 7.305 (2.68), | |
| 7.427 (2.33), 7.432 (0.91), 7.439 (2.49), 7.444 (1.26), 7.449 (3.67), | |
| 7.455 (2.27), 7.462 (2.14), 7.465 (1.73), 7.471 (1.89), 7.477 (1.88), | |
| 7.481 (1.63), 7.487 (1.36), 7.503 (1.21), 8.138 (1.97), 8.144 (1.99), | |
| 9.144 (4.00), 9.150 (3.94), 11.048 (1.27). | |
| 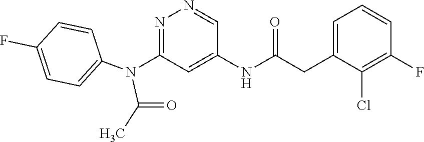 | 191 |
| N-{5-[2-(2-chloro-3-fluorophenyl)acetamido]pyridazin-3-yl}-N-(4- | |
| fluorophenyl)acetamide | |
| LC-MS (Method 1): Rₜ = 1.03 min; MS (ESIpos): m/z = 417 [M + H]⁺ | |
| ¹H-NMR (400 MHZ, DMSO-d6) δ [ppm]: 0.967 (0.88), 1.107 (11.11), | |
| 1.109 (2.26), 1.144 (0.57), 2.011 (16.00), 2.332 (0.89), 2.447 (0.48), | |
| 2.518 (3.94), 2.523 (2.56), 2.673 (0.89), 3.995 (6.64), 4.188 (0.91), | |
| 7.258 (2.05), 7.264 (0.79), 7.280 (4.25), 7.285 (2.00), 7.290 (1.12), | |
| 7.296 (1.27), 7.302 (3.41), 7.311 (0.45), 7.338 (0.42), 7.352 (2.07), | |
| 7.355 (2.12), 7.359 (1.42), 7.371 (4.24), 7.380 (1.33), 7.386 (1.32), | |
| 7.425 (2.12), 7.431 (0.82), 7.437 (2.25), 7.443 (1.03), 7.447 (1.94), | |
| 7.454 (0.69), 7.460 (1.73), 8.139 (1.79), 8.145 (1.74), 9.141 (3.72), | |
| 9.147 (3.68), 11.080 (1.21). | |
| 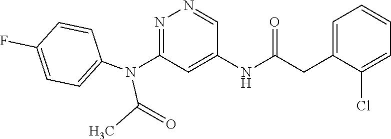 | 192 |
| N-{5-[2-(2-chlorophenyl)acetamido]pyridazin-3-yl}-N-(4- | |
| fluorophenyl)acetamide | |
| LC-MS (Method 1): Rₜ = 1.02 min; MS (ESIpos): m/z = 399 [M + H]⁺ | |
| ¹H-NMR (400 MHZ, DMSO-d6) δ [ppm]: 0.967 (0.93), 1.107 (8.33), | |
| 1.109 (2.29), 1.144 (0.56), 2.012 (16.00), 2.332 (0.68), 2.518 (2.98), | |
| 2.523 (2.00), 2.673 (0.69), 3.932 (7.28), 4.189 (0.66), 7.257 (2.11), | |
| 7.263 (0.77), 7.274 (0.96), 7.280 (4.07), 7.284 (0.87), 7.289 (0.43), | |
| 7.295 (0.85), 7.301 (2.81), 7.314 (3.38), 7.322 (3.28), 7.328 (3.57), | |
| 7.337 (4.31), 7.347 (0.79), 7.412 (1.68), 7.424 (2.70), 7.426 (2.02), | |
| 7.436 (3.40), 7.442 (1.24), 7.447 (3.62), 7.456 (1.79), 7.459 (2.12), | |
| 7.461 (1.83), 7.470 (1.29), 8.138 (1.87), 8.143 (1.79), 9.148 (4.11), | |
| 9.154 (3.98), 11.044 (1.34). | |
| 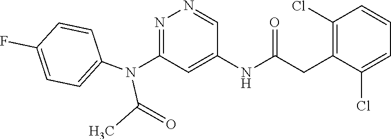 | 193 |
| N-{5-[2-(2,6-dichlorophenyl)acetamido]pyridazin-3-yl}-N-(4- | |
| fluorophenyl)acetamide | |
| LC-MS (Method 1): Rₜ = 1.11 min; MS (ESIpos): m/z = 433 [M + H]⁺ | |
| ¹H-NMR (400 MHZ, DMSO-d6) δ [ppm]: 1.107 (16.00), 2.007 (6.63), | |
| 2.518 (0.78), 2.522 (0.48), 4.130 (3.05), 4.191 (1.37), 7.256 (0.82), | |
| 7.278 (1.72), 7.299 (1.03), 7.348 (0.59), 7.367 (0.79), 7.369 (0.83), | |
| 7.388 (0.99), 7.427 (0.89), 7.440 (0.95), 7.445 (0.45), 7.450 (0.80), | |
| 7.462 (0.73), 7.498 (2.77), 7.518 (1.69), 8.138 (0.73), 8.143 (0.76), | |
| 9.117 (1.48), 9.123 (1.48). | |
| 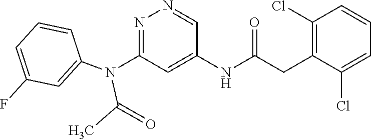 | 194 |
| N-{5-[2-(2,6-dichlorophenyl)acetamido]pyridazin-3-yl}-N-(3- | |
| fluorophenyl)acetamide | |
| LC-MS (Method 1): Rₜ = 1.07 min; MS (ESIpos): m/z = 433 [M + H]⁺ | |
| 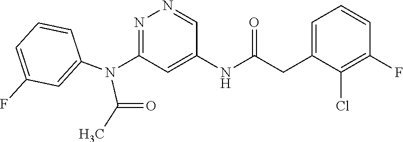 | 195 |
| N-{5-[2-(2-chloro-3-fluorophenyl)acetamido]pyridazin-3-yl}-N-(3- | |
| fluorophenyl)acetamide | |
| LC-MS (Method 1): Rₜ = 1.04 min; MS (ESIpos): m/z = 417 [M + H]⁺ | |
| 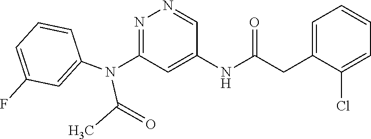 | 196 |
| N-{5-[2-(2-chlorophenyl)acetamido]pyridazin-3-yl}-N-(3- | |
| fluorophenyl)acetamide | |
| LC-MS (Method 1): Rₜ = 1.04 min; MS (ESIneg): m/z = 397 [M − H]⁻ | |
| ¹H-NMR (400 MHZ, DMSO-d6) δ [ppm]: 1.107 (16.00), 2.038 (8.05), | |
| 2.518 (0.52), 3.936 (3.60), 4.191 (1.35), 7.182 (0.49), 7.184 (0.47), | |
| 7.186 (0.45), 7.202 (0.71), 7.205 (0.74), 7.224 (0.45), 7.226 (0.43), | |
| 7.231 (0.54), 7.233 (0.47), 7.313 (1.45), 7.322 (1.39), 7.328 (1.58), | |
| 7.337 (2.31), 7.344 (0.44), 7.346 (0.45), 7.363 (0.52), 7.413 (0.76), | |
| 7.422 (0.55), 7.427 (0.59), 7.436 (0.63), 7.447 (0.98), 7.456 (0.57), | |
| 7.461 (0.73), 7.466 (0.55), 7.470 (1.24), 7.486 (0.63), 8.132 (0.88), | |
| 8.137 (0.89), 9.170 (1.82), 9.176 (1.78), 11.062 (0.82). | |
| 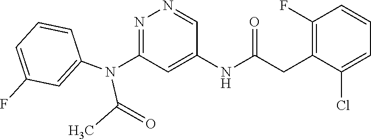 | 197 |
| N-{5-[2-(2-chloro-6-fluorophenyl)acetamido]pyridazin-3-yl}-N- | |
| (3-fluorophenyl)acetamide | |
| LC-MS (Method 1): Rₜ = 1.03 min; MS (ESIpos): m/z = 417 [M + H]⁺ | |
| 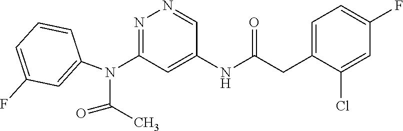 | 198 |
| N-{5-[2-(2-chloro-4-fluorophenyl)acetamido]pyridazin-3-yl}-N-(3- | |
| fluorophenyl)acetamide | |
| LC-MS (Method 1): Rₜ = 1.07 min; MS (ESIneg): m/z = 415 [M − H]⁻ | |
| ¹H-NMR (400 MHZ, DMSO-d6) δ [ppm]: 1.109 (1.18), 2.039 (3.88), | |
| 2.520 (1.43), 2.525 (0.95), 2.542 (16.00), 2.671 (0.44), 3.929 (1.46), | |
| 7.201 (0.40), 7.208 (0.53), 7.229 (0.62), 7.455 (0.44), 7.470 (0.59), | |
| 7.477 (0.44), 8.130 (0.43), 8.135 (0.43), 9.164 (0.91), 9.170 (0.90). | |
| 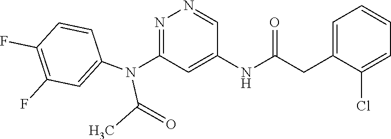 | 199 |
| N-{5-[2-(2-chlorophenyl)acetamido]pyridazin-3-yl}-N-(3,4- | |
| difluorophenyl)acetamide | |
| LC-MS (Method 1): Rₜ = 1.05 min; MS (ESIpos): m/z = 417 [M + H]⁺ | |
| 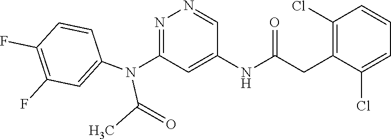 | 200 |
| N-{5-[2-(2,6-dichlorophenyl)acetamido]pyridazin-3-yl}-N-(3,4- | |
| difluorophenyl)acetamide | |
| LC-MS (Method 1): Rₜ = 1.13 min; MS (ESIneg): m/z = 449 [M − H]⁻ | |
| ¹H-NMR (400 MHz, DMSO-d6) δ [ppm]: 0.967 (0.56), 1.107 (16.00), | |
| 2.027 (10.25), 2.332 (0.40), 2.518 (2.07), 2.522 (1.27), 2.669 (0.56), | |
| 2.673 (0.41), 4.136 (4.80), 4.189 (1.55), 7.255 (0.42), 7.277 (0.49), | |
| 7.350 (1.09), 7.369 (1.27), 7.372 (1.49), 7.391 (1.62), 7.482 (0.41), | |
| 7.501 (4.55), 7.521 (2.69), 7.530 (0.84), 7.658 (0.43), 7.661 (0.43), | |
| 8.176 (1.08), 8.181 (1.10), 9.132 (2.02), 9.138 (2.03), 11.179 (1.42). | |
| 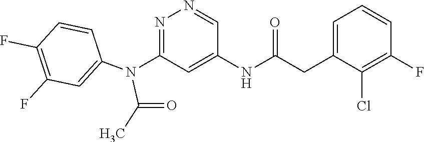 | 201 |
| N-{5-[2-(2-chloro-3-fluorophenyl)acetamido]pyridazin-3-yl}-N-(3,4- | |
| difluorophenyl)acetamide | |
| LC-MS (Method 1): Rₜ = 1.06 min; MS (ESIpos): m/z = 435 [M + H]⁺ | |
| ¹H-NMR (400 MHZ, DMSO-d6) δ [ppm]: 0.967 (1.23), 1.107 (6.07), | |
| 1.109 (2.38), 1.144 (0.75), 1.232 (0.42), 2.030 (16.00), 2.332 (1.17), | |
| 2.336 (0.49), 2.446 (0.51), 2.452 (0.53), 2.518 (5.28), 2.523 (3.48), | |
| 2.673 (1.17), 2.678 (0.51), 3.998 (6.02), 4.189 (0.47), 7.249 (0.60), | |
| 7.271 (0.76), 7.278 (1.04), 7.287 (1.12), 7.293 (0.82), 7.297 (0.85), | |
| 7.303 (1.14), 7.352 (1.88), 7.355 (1.98), 7.359 (1.32), 7.373 (4.11), | |
| 7.381 (1.27), 7.387 (1.22), 7.482 (0.56), 7.505 (1.11), 7.509 (0.74), | |
| 7.527 (0.69), 7.531 (1.09), 7.554 (0.51), 7.630 (0.48), 7.636 (0.48), | |
| 7.648 (0.53), 7.655 (0.59), 7.665 (0.51), 7.677 (0.51), 7.682 (0.47), | |
| 8.172 (1.48), 8.177 (1.45), 9.151 (2.86), 9.157 (2.77), 11.103 (0.56). | |
| 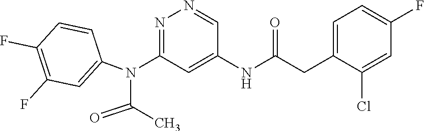 | 202 |
| N-{5-[2-(2-chloro-4-fluorophenyl)acetamido]pyridazin-3-yl}-N-(3,4- | |
| difluorophenyl)acetamide | |
| LC-MS (Method 1): Rₜ = 1.13 min; MS (ESIpos): m/z = 435 [M + H]⁺ | |
| ¹H-NMR (400 MHZ, DMSO-d6) δ [ppm]: 0.970 (0.44), 1.110 (1.23), | |
| 1.112 (0.74), 2.032 (16.00), 2.334 (0.65), 2.521 (2.99), 2.525 (1.94), | |
| 2.672 (0.91), 2.676 (0.66), 3.931 (5.84), 7.204 (0.70), 7.211 (0.75), | |
| 7.225 (1.42), 7.232 (1.66), 7.246 (1.20), 7.253 (1.36), 7.263 (0.54), | |
| 7.270 (0.55), 7.274 (0.71), 7.450 (1.52), 7.456 (1.50), 7.469 (1.42), | |
| 7.472 (1.70), 7.479 (1.60), 7.484 (1.77), 7.489 (1.32), 7.506 (1.55), | |
| 7.512 (0.78), 7.531 (0.66), 7.534 (1.17), 7.557 (0.54), 7.632 (0.50), | |
| 7.639 (0.51), 7.651 (0.56), 7.657 (0.61), 7.661 (0.63), 7.667 (0.55), | |
| 7.679 (0.53), 7.686 (0.51), 8.173 (1.58), 8.179 (1.60), 9.155 (3.45), | |
| 9.161 (3.40), 11.063 (0.61). | |
| 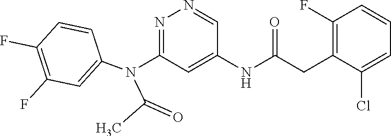 | 203 |
| N-{5-[2-(2-chloro-6-fluorophenyl)acetamido]pyridazin-3-yl}-N-(3,4- | |
| difluorophenyl)acetamide | |
| LC-MS (Method 1): Rₜ = 1.06 min; MS (ESIpos): m/z = 435 [M + H]⁺ | |
| ¹H-NMR (400 MHz, DMSO-d6) δ [ppm]: - 0.002 (1.32), 2.026 (16.00), | |
| 2.331 (0.91), 2.336 (0.41), 2.518 (3.89), 2.523 (2.55), 2.673 (0.91), | |
| 3.977 (3.88), 3.980 (3.72), 7.240 (0.87), 7.245 (1.01), 7.248 (0.65), | |
| 7.258 (1.24), 7.263 (1.82), 7.266 (1.26), 7.270 (0.76), 7.282 (1.01), | |
| 7.286 (0.89), 7.350 (0.66), 7.354 (0.79), 7.370 (3.28), 7.376 (2.34), | |
| 7.390 (1.18), 7.396 (1.10), 7.410 (1.27), 7.431 (0.41), 7.480 (0.58), | |
| 7.502 (1.16), 7.506 (0.73), 7.524 (0.66), 7.529 (1.15), 7.551 (0.52), | |
| 7.627 (0.47), 7.634 (0.48), 7.646 (0.52), 7.652 (0.58), 7.656 (0.58), | |
| 7.662 (0.53), 7.674 (0.50), 7.681 (0.47), 8.165 (1.44), 8.170 (1.44), | |
| 9.133 (2.59), 9.139 (2.55), 11.174 (0.57). | |
| 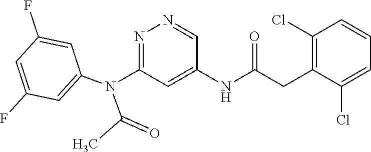 | 204 |
| N-{5-[2-(2,6-dichlorophenyl)acetamido]pyridazin-3-yl}-N-(3,5- | |
| difluorophenyl)acetamide | |
| LC-MS (Method 1): Rₜ = 1.11 min; MS (ESIneg): m/z = 449 [M − H]⁻ | |
| 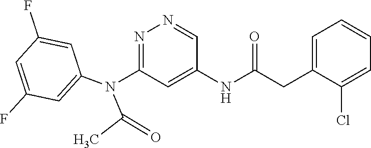 | 205 |
| N-{5-[2-(2-chlorophenyl)acetamido]pyridazin-3-yl}-N-(3,5- | |
| difluorophenyl)acetamide | |
| LC-MS (Method 1): Rₜ = 1.06 min; MS (ESIpos): m/z = 417 [M + H]⁺ | |
| 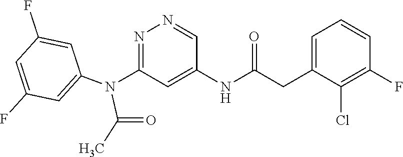 | 206 |
| N-{5-[2-(2-chloro-3-fluorophenyl)acetamido]pyridazin-3-yl}-N-(3,5- | |
| difluorophenyl)acetamide | |
| LC-MS (Method 1): Rₜ = 1.08 min; MS (ESIpos): m/z = 435 [M + H]⁺ | |
| 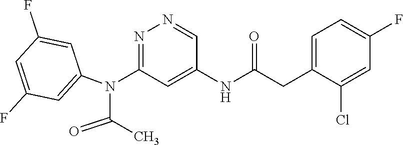 | 207 |
| N-{5-[2-(2-chloro-4-fluorophenyl)acetamido]pyridazin-3-yl}-N-(3,5- | |
| difluorophenyl)acetamide | |
| LC-MS (Method 1): Rₜ = 1.14 min; MS (ESIpos): m/z = 435 [M + H]⁺ | |
| ¹H-NMR (400 MHZ, DMSO-d6) δ [ppm]: - 0.008 (1.12), 0.008 (1.17), | |
| 0.970 (0.53), 1.109 (4.03), 2.058 (16.00), 2.334 (0.86), 2.520 (3.85), | |
| 2.525 (2.53), 2.676 (0.88), 3.933 (5.83), 7.203 (0.77), 7.210 (0.97), | |
| 7.224 (2.62), 7.231 (2.75), 7.246 (2.00), 7.253 (1.28), 7.283 (0.62), | |
| 7.301 (0.79), 7.306 (1.25), 7.313 (0.64), 7.330 (0.62), 7.450 (1.52), | |
| 7.456 (1.54), 7.469 (1.47), 7.472 (1.80), 7.479 (1.56), 7.484 (1.43), | |
| 7.490 (1.23), 7.506 (1.14), 8.160 (1.56), 8.165 (1.58), 9.174 (3.15), | |
| 9.180 (3.08), 11.079 (0.57). | |
| 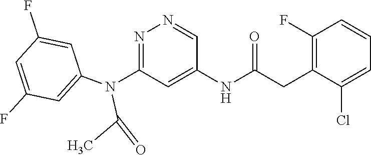 | 208 |
| N-{5-[2-(2-chloro-6-fluorophenyl)acetamido]pyridazin-3-yl}-N- | |
| (3,5-difluorophenyl)acetamide | |
| LC-MS (Method 1): Rₜ = 1.07 min; MS (ESIpos): m/z = 435 [M + H]⁺ | |
| ¹H-NMR (400 MHZ, DMSO-d6) δ [ppm]: - 0.002 (0.68), 2.052 (16.00), | |
| 2.331 (1.04), 2.336 (0.48), 2.518 (4.74), 2.523 (3.10), 2.673 (1.08), | |
| 2.678 (0.47), 3.982 (3.65), 3.985 (3.54), 7.221 (1.22), 7.226 (1.57), | |
| 7.240 (1.85), 7.245 (1.89), 7.258 (1.02), 7.263 (1.47), 7.272 (0.45), | |
| 7.278 (0.67), 7.284 (1.07), 7.287 (0.90), 7.295 (0.72), 7.301 (1.17), | |
| 7.307 (0.62), 7.325 (0.58), 7.351 (0.62), 7.355 (0.73), 7.372 (2.91), | |
| 7.377 (2.24), 7.391 (1.16), 7.396 (1.07), 7.412 (1.17), 8.154 (1.37), | |
| 8.160 (1.37), 9.157 (2.44), 9.163 (2.39), 11.190 (0.58). | |

### Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

### In Vitro Studies

The in vitro activity of the compounds of the present invention can be demonstrated in the following assays:

### Human P2X4 HEK Cell FLIPR Assay

Compounds were tested on a HEK293 cell line stably expressing human P2X4. Cells were cultured on poly-D-lysine-coated 384-well plates at a density of 15,000 cells/well and incubated overnight at 37° C., 5% CO₂. P2X4 function was assessed by measuring intracellular calcium fluxes caused by Benzoylbenzoyl-ATP (Bz-ATP) using the calcium-chelating dye Fluo8-AM (Molecular Devices) with the Fluorescent Imaging Plate Reader Tetra (FLIPR^{Tetra}; Molecular Devices CA). On the day of the assay, the medium was removed and the cells were incubated for 30 min at 37° C. and 5% CO₂ in 30 μl of dye buffer (Hank's balanced salt solution, 10 mM HEPES, 1.8 mM CaCl₂, 1 mM MgCl₂, 2 mM probenecid, 5 mM D-glucose monohydrate, 5 μM Fluo8-AM, pH=7.4). Compounds diluted in probenecid buffer (Hank's balanced salt solution, 10 mM HEPES, 1.8 mM CaCl₂, 1 mM MgCl₂, 2 mM probenecid, 5 mM D-glucose monohydrate, pH=7.4) at ten concentrations ranging from 25 μM to 1 nM (final concentration) were dispensed and incubated for 30 min at room temperature. The agonist, Bz-ATP (Tocris Bio-Techne GmbH, DE), was added at a final concentration of 3 μM, representing the EC80 routinely determined. The final assay volume was 50 μl and final DMSO concentration was 0.5%.

The fluorescence intensity reflecting intracellular calcium changes was recorded before and after Bz-ATP addition, at an excitation and emission wavelengths of 470-495 nm and 515-575 nm respectively.

The compounds were tested in triplicates and fluorescence intensity raw data normalized to the agonist control and fitted to the four-parameter logistic equation:

Y=Bottom+(Top−Bottom)/(1+10\hat{ }((Log IC50−X)*HillSlope))

The efficacy of saturating concentrations of the agonist BzATP (3 μM) was set as maximal response (100% Emax) and the bottom defined by the signal achieved with 0.5% DMSO.

Assay plate acceptance was based on the signal window (S/B)≥21.8, Z′≥20.5 and the reference compound pIC50 within ±3σ the mean of historic pIC50 of the compound. Failure to meet two of the three criteria determined exclusion of the plate's results.

### FLIPR Method for Rat P2X4 1321N1 Astrocytoma Cells

Compounds were tested on a 1321N1 cell line stably expressing rat P2X4. Cells were cultured on collagen-I-coated 384-well plates at a density of 10,000 cells/well and incubated overnight at 37° C., 5% CO₂. P2X4 function was assessed by measuring intracellular calcium fluxes caused by Magnesium-ATP (MgATP) using the calcium-chelating dye Fluo8-AM (Molecular Devices) with the Fluorescent Imaging Plate Reader Tetra (FLIPR^{Tetra}; Molecular Devices CA). On the day of the assay, the medium was removed and the cells were incubated for 30 minutes at 37° C. and 5% CO₂ in 30 μl of dye buffer (Hank's balanced salt solution, 10 mM HEPES, 1.8 mM CaCl₂, 1 mM MgCl₂, 2 mM probenecid, 5 mM D-glucose monohydrate, 5 μM Fluo8-AM, pH=7.4).

Compounds diluted in probenecid buffer (Hank's balanced salt solution, 10 mM HEPES, 1.8 mM CaCl₂, 1 mM MgCl₂, 2 mM probenecid, 5 mM D-glucose monohydrate, pH=7.4) at ten concentrations ranging from 25 μM to 1 nM (final concentration) were dispensed and incubated for 30 minutes at room temperature. The agonist, MgATP (Sigma-Aldrich Chemie GmbH, DE), was added at a final concentration of 5 μM, representing the EC₈₀ routinely determined. The final assay volume was 50 μl and final DMSO concentration was 0.5%.

The fluorescence intensity reflecting intracellular calcium changes was recorded before and after MgATP addition, at an excitation and emission wavelengths of 470-495 nm and 515-575 nm respectively.

The compounds were tested in triplicates and fluorescence intensity raw data normalized to the agonist control and fitted to the four-parameter logistic equation:

Y=Bottom+(Top−Bottom)/(1+10\hat{ }((Log IC50−X)*HillSlope))

The efficacy of saturating concentrations of the agonist MgATP (5 μM) was set as maximal response (100% Emax) and the bottom defined by the signal achieved with 0.5% DMSO.

Assay plate acceptance was based on the signal window (S/B)≥21.5, Z′≥20.5 and the reference compound pIC50 within ±3a the mean of historic pIC50 of the compound. Failure to meet two of the three criteria determined exclusion of the plate's results. In Table 10 below the results for the assays are reported

TABLE 10

| | | | Rat P2X4 1321N1 | Rat P2X4 1321N1 |
|---|---|---|---|---|
| | Human P2X4 HEK | Human P2X4 HEK | Astrocytoma Cells | Astrocytoma Cells |
| | Cells (FLIPR Assay) | Cells (FLIPR Assay) | (FLIPR Assay) | (FLIPR Assay) |
| Ex. N. | avg IC₅₀ [nM] | avg Efficacy [%] | avg IC₅₀ [nM] | avg Efficacy [%] |
| 1 | 224 | 89 | 96.7 | 82 |
| 2 | 212 | 97 | 358 | 95 |
| 3 | 657 | 98 | | |
| 4 | 397 | 100 | 855 | 100 |
| 5 | 386 | 102 | 440 | 100 |
| 6 | 689 | 97 | | |
| 7 | 846 | 97 | | |
| 8 | 681 | 97 | | |
| 9 | 749 | 99 | | |
| 10 | 875 | 94 | | |
| 11 | 977 | 100 | | |
| 12 | 973 | 101 | | |
| 13 | 289 | 96 | 790 | 93 |
| 14 | 243 | 100 | 201 | 98 |
| 15 | 408 | 94 | 441 | 88 |
| 16 | 388 | 95 | | |
| 17 | 638 | 99 | | |
| 18 | 437 | 99 | | |
| 19 | 542 | 103 | | |
| 20 | 748 | 99 | | |
| 21 | 343 | 91 | 374 | 90 |
| 22 | 482 | 98 | 217 | 95 |
| 23 | 356 | 99 | 483 | 96 |
| 24 | 346 | 100 | 174 | 99 |
| 25 | 524 | 100 | 851 | 100 |
| 26 | 660 | 102 | 649 | 101 |
| 27 | 242 | 97 | 652 | 99 |
| 28 | 777 | 91 | | |
| 29 | 391 | 91 | 92.5 | 77 |
| 30 | 384 | 98 | 727 | 96 |
| 31 | 909 | 100 | | |
| 32 | 355 | 101 | 801 | 100 |
| 33 | 723 | 93 | 1540 | 98 |
| 34 | 199 | 97 | 170 | 94 |
| 35 | 938 | 99 | | |
| 36 | 206 | 91 | 290 | 90 |
| 37 | 894 | 95 | 242 | 96 |
| 38 | 232 | 96 | 136 | 98 |
| 39 | 335 | 100 | 152 | 98 |
| 40 | 308 | 90 | 194 | 75 |
| 41 | 589 | 94 | 115 | 91 |
| 42 | 350 | 98 | 1360 | 96 |
| 43 | 406 | 100 | | |
| 44 | 607 | 99 | | |
| 45 | 485 | 102 | 1180 | 100 |
| 46 | 551 | 103 | | |
| 47 | 519 | 98 | | |
| 48 | 579 | 99 | | |
| 49 | 520 | 100 | | |
| 50 | 351 | 95 | 213 | 96 |
| 51 | 493 | 98 | 177 | 96 |
| 52 | 355 | 98 | 358 | 99 |
| 53 | 455 | 100 | 143 | 100 |
| 54 | 269 | 97 | 447 | 97 |
| 55 | 611 | 102 | 289 | 99 |
| 56 | 260 | 97 | 190 | 98 |
| 57 | 377 | 99 | 122 | 97 |
| 58 | 351 | 95 | 350 | 86 |
| 59 | 532 | 97 | 240 | 81 |
| 60 | 454 | 96 | 434 | 94 |
| 61 | 604 | 98 | | |
| 62 | 251 | 91 | 368 | 91 |
| 63 | 424 | 97 | 372 | 97 |
| 64 | 829 | 96 | 497 | 100 |
| 65 | 505 | 94 | 122 | 93 |
| 66 | 303 | 90 | 64.2 | 73 |
| 67 | 604 | 94 | 159 | 97 |
| 68 | 411 | 95 | 233 | 89 |
| 69 | 816 | 94 | 228 | 100 |
| 70 | 714 | 88 | 714 | 100 |
| 71 | 222 | 96 | 165 | 90 |
| 72 | 355 | 97 | 58.5 | 89 |
| 73 | 428 | 97 | 67.1 | 93 |
| 74 | 585 | 89 | 99.1 | 97 |
| 75 | 919 | 96 | | |
| 76 | 397 | 97 | 667 | 99 |
| 77 | 354 | 94 | 993 | 84 |
| 78 | 573 | 94 | | |
| 79 | 399 | 97 | 1110 | 96 |
| 80 | 451 | 99 | 338 | 97 |
| 81 | 895 | 97 | | |
| 82 | 504 | 97 | | |
| 83 | 350 | 97 | 143 | 90 |
| 84 | 314 | 100 | 1030 | 100 |
| 85 | 251 | 98 | 229 | 97 |
| 86 | 196 | 98 | 393 | 98 |
| 87 | 313 | 98 | 983 | 94 |
| 88 | 198 | 97 | 451 | 100 |
| 89 | 169 | 99 | 103 | 98 |
| 90 | 272 | 98 | 110 | 97 |
| 91 | 345 | 98 | 1630 | 100 |
| 92 | 568 | 97 | | |
| 93 | 705 | 98 | | |
| 94 | 710 | 97 | | |
| 95 | 694 | 100 | | |
| 96 | 362 | 89 | | |
| 97 | 461 | 89 | | |
| 98 | 296 | 92 | | |
| 99 | 449 | 95 | | |
| 100 | 408 | 98 | 965 | 98 |
| 101 | 406 | 97 | 971 | 100 |
| 102 | 937 | 99 | | |
| 103 | 767 | 97 | | |
| 104 | 846 | 96 | | |
| 105 | 770 | 100 | | |
| 106 | 765 | 95 | | |
| 107 | 550 | 98 | | |
| 108 | 769 | 97 | | |
| 109 | 643 | 100 | | |
| 110 | 745 | 95 | | |
| 111 | 204 | 96 | 320 | 100 |
| 112 | 403 | 96 | 408 | 97 |
| 113 | 286 | 97 | 567 | 100 |
| 114 | 256 | 97 | 181 | 99 |
| 115 | 391 | 96 | | |
| 116 | 520 | 97 | | |
| 117 | 789 | 95 | | |
| 118 | 545 | 94 | | |
| 119 | 941 | 101 | | |
| 120 | 871 | 102 | | |
| 121 | 843 | 97 | | |
| 122 | 357 | 95 | | |
| 123 | 435 | 97 | 633 | 89 |
| 124 | 289 | 94 | | |
| 125 | 577 | 95 | | |
| 126 | 176 | 96 | 377 | 83 |
| 127 | 283 | 97 | 261 | 72 |
| 128 | 184 | 101 | 454 | 87 |
| 129 | 197 | 104 | 212 | 96 |
| 130 | 259 | 97 | 1520 | 80 |
| 131 | 206 | 98 | 262 | 98 |
| 132 | 299 | 98 | 228 | 97 |
| 133 | 193 | 100 | 325 | 100 |
| 134 | 504 | 99 | | |
| 135 | 281 | 99 | 264 | 96 |
| 136 | 310 | 94 | | |
| 137 | 290 | 104 | | |
| 138 | 316 | 96 | | |
| 139 | 356 | 100 | | |
| 140 | 271 | 105 | | |
| 141 | 891 | 99 | 726 | 100 |
| 142 | 796 | 86 | 767 | 98 |
| 143 | 723 | 94 | 934 | 101 |
| 144 | 648 | 90 | 557 | 100 |
| 145 | 234 | 98 | | |
| 146 | 337 | 103 | | |
| 147 | 384 | 97 | | |
| 148 | 436 | 96 | | |
| 149 | 450 | 100 | | |
| 150 | 297 | 102 | | |
| 151 | 355 | 104 | | |
| 152 | 441 | 104 | | |
| 153 | 476 | 102 | | |
| 154 | 511 | 101 | | |
| 155 | 339 | 100 | | |
| 156 | 512 | 96 | | |
| 157 | 644 | 97 | | |
| 158 | 649 | 97 | | |
| 159 | 949 | 99 | | |
| 160 | 383 | 101 | | |
| 161 | 479 | 101 | | |
| 162 | 577 | 104 | | |
| 163 | 665 | 100 | | |
| 164 | 683 | 100 | | |
| 165 | 402 | 99 | | |
| 166 | 663 | 97 | | |
| 167 | 677 | 100 | | |
| 168 | 761 | 99 | | |
| 169 | 813 | 101 | | |
| 170 | 468 | 104 | | |
| 171 | 511 | 103 | | |
| 172 | 520 | 104 | | |
| 173 | 526 | 104 | | |
| 174 | 528 | 103 | | |
| 175 | 516 | 97 | | |
| 176 | 567 | 101 | | |
| 177 | 578 | 96 | | |
| 178 | 621 | 99 | | |
| 179 | 673 | 98 | | |
| 180 | 569 | 101 | | |
| 181 | 574 | 101 | | |
| 182 | 593 | 102 | | |
| 183 | 599 | 101 | | |
| 184 | 893 | 104 | | |
| 185 | 438 | 101 | | |
| 186 | 655 | 99 | | |
| 187 | 700 | 100 | | |
| 188 | 829 | 98 | | |
| 189 | 654 | 93 | | |
| 190 | 716 | 95 | | |
| 191 | 739 | 92 | | |
| 192 | 766 | 90 | | |
| 193 | 868 | 93 | | |
| 194 | 407 | 96 | | |
| 195 | 486 | 95 | | |
| 196 | 546 | 94 | | |
| 197 | 674 | 93 | | |
| 198 | 943 | 96 | | |
| 199 | 447 | 86 | | |
| 200 | 465 | 91 | | |
| 201 | 510 | 89 | | |
| 202 | 557 | 94 | | |
| 203 | 737 | 86 | | |
| 204 | 383 | 95 | | |
| 205 | 433 | 93 | | |
| 206 | 449 | 93 | | |
| 207 | 477 | 96 | | |
| 208 | 669 | 91 | | |

### In Vivo Studies

CFA Inflammation Model in Rats with Pain Behaviour Read Out

### Methods

Compound was tested in the model of intraplantar Complete Freund's Adjuvant (CFA)-induced acute (48-hours setting) inflammatory pain in male Sprague Dawley rats. Briefly, 25 μl of CFA at 1 mg/ml was injected into the plantar surface of one hind paw. Mechanical hyperalgesia was measured using the Pressure Application Measurement apparatus (Ugo Basile, Gemonio, Italy). A linearly increasing pressure was applied to an area of approximately 50 mm² of the plantar side of the hind paw until a behavioural response (paw withdrawal) was observed or until the pressure reached 1000 grams of force (gf). The pressure at which the behavioural response occurred was recorded as the “Paw Withdrawal Threshold” (PWT). Both CFA-injected and contralateral PWTs were determined for each rat, in each treatment group and at each time point of the studies. The measurements were performed blinded. Mechanical hyperalgesia testing was performed before injecting CFA, 46 hours after CFA injection (pre-drug baseline) and 2 hours after last treatment. Compound or vehicle (10% DMSO, 40% Solutol, 50% water for injection, vol/vol) were dosed via oral route (p.o.) once daily (QD) or twice daily (BID) during 3 days, starting before CFA injection. Data were expressed as the mean PWT for each treatment group and at each time point. PWT data were analysed by performing a two way ANOVA with repeated measures (time×treatment). Planned comparison of means (each versus vehicle) was performed by using a Dunnett's post hoc test, provided that a main effect was detected. For p values less than 0.05, the results were deemed to be statistically significant.

### Data with the Compound of Example 2

Intraplantar CFA in the rat induced acute inflammatory pain characterized by a robust reduction of PWT 48 hours after injection. Oral administration of the compound according to example 2, QD during 3 days, prevented the development of inflammatory pain after the injection of CFA. After treatment, the 50 and 100 mg/kg doses significantly reduced pain 2 and 4 hours after last administration (see Table 11).

TABLE 11
Rat CFA in vivo model data for Example 2

| | Dose p.o. | | PWT 2 hour-post | | PWT 4 hour-post | |
|---|---|---|---|---|---|---|
| Example | (n = 7-8) | | drug | | drug | |
| Vehicle | 5 | ml/kg QD | 527 ± | 101 gf | 545 | ± 63 gf |
| Ex. 2 | 50 | mg/kg QD | 727 ± | 87 gf (****) | 688 | ± 83 gf (*) |
| Ex. 2 | 100 | mg/kg QD | 763 ± | 97 gf (***) | 839 | ± 159 gf (****) |

Data were expressed as the mean PWT±standard deviation (SD) for each treatment group and at each time point. *p<0.05, ***p<0.001, ****p<0.0001, different from vehicle group at same time point (Dunnett's post-hoc test).

### Data with Example 68

Intraplantar CFA in the rat induced acute inflammatory pain characterized by a robust reduction of PWT 48 hours after injection. Oral administration of the compound according to example 68, BID during 3 days, prevented the development of inflammatory pain after the injection of CFA. After treatment, the 10, 30 and 100 mg/kg doses significantly reduced pain 2 hours after last administration (see Table 12).

TABLE 12
Rat CFA in vivo model data for Example 68

| Example | Dose p.o. (n = 8) | | PWT 2 hour-post drug | |
|---|---|---|---|---|
| Vehicle | 5 | mL/kg BID | 529 ± | 52 gf |
| Ex. 68 | 10 | mg/kg BID | 630 ± | 45 gf (**) |
| Ex.68 | 30 | mg/kg BID | 648 ± | 37 gf (***) |
| Ex. 68 | 100 | mg/kg BID | 759 ± | 77 gf (****) |

Data were expressed as the mean PWT±standard deviation (SD) for each treatment group and at each time point. *p<0.05, **p<0.01, ***p<0.001, ****p<0.0001, different from vehicle group at same time point (Dunnett's post-hoc test).

### Chronic Constriction Injury (CCI) Neuropathic Pain Model in Rats with Pain Behavior Read Out (Example 68)

Aim of the study was to test the efficacy of example 68 at ameliorating neuropathic mechanical allodynia. Male Sprague-Dawley rats were subject to chronic constriction injury of the sciatic nerve to induce neuropathic pain. Of those rats, 10 per group were assigned to treatment conditions involving BID drug administration for 10 days and behavioral assessment of mechanical sensitivity for allodynia with von Frey testing (vF). Behavior was assessed pre- and postdosing on Days 0, 2, 5, and 9. Rats were treated with Vehicle or test article BID on days 0 through 9, and rats in the gabapentin treatment group received gabapentin SID on days 0, 2, 5, and 9 in conjunction with behavioral assessment.

As vehicle Solutol/DMSO/WFI (40/10/50) was employed. Application volume for the per oral administration was 10 ml/kg.

### Experimental Timeline:

Day −12 to −14: model Creation (CCI Surgery)

### Day 0:

Mechanical allodynia (vF)

### Dosing

Mechanical allodynia testing (vF, time 2 hour)

### Day 1: Dosing

### Day 2:

Mechanical allodynia testing (vF)

### Dosing

Mechanical allodynia testing (vF, time 2 hour)

### Day 3: Dosing

### Day 4: Dosing

### Day 5:

Mechanical allodynia testing (vF)
Dosing (time 0 min)
Mechanical allodynia testing (vF, time 2 hour)

### Day 6: Dosing

### Day 7: Dosing

### Day 8: Dosing

### Day 9:

Mechanical allodynia testing (vF)

### Dosing

Mechanical allodynia testing (vF, time 2 hour).

### Method

Neuropathy was induced in male Sprague-Dawley rats by tying four loose ligatures of chromic gut around the common sciatic nerve in deeply anesthetized rats like described in Bennett, G. J., and Y. K. Xie. “A Peripheral Mononeuropathy in Rat That Produces Disorders of Pain Sensation like Those Seen in Man.” Pain 33 (1988): 87-107.

Mechanical allodynia was measured using 8 Semmes-Weinstein filaments (Stoelting©; Wood Dale, Ill., USA) with varying stiffness (0.4, 0.6, 1.0, 2.0, 4.0, 6.0, 8.0, and 15 g) according to the up-down method1. Animals were placed in individual acrylic chambers on a metal mesh surface and allowed to acclimate to their surroundings for a minimum of 15 minutes before testing. Each filament was presented perpendicular to the plantar surface with sufficient force to cause slight buckling against the paw and held for approximately 6 seconds or until a positive response is noted (paw sharply withdrawn).

Testing was initiated with the 2.0 g filament. In the absence of a paw withdrawal response, the next stronger stimulus was presented. In the event of paw withdrawal, the next weaker stimulus was used. This process was repeated until 4 responses after the initial change in response (no response to positive response or positive response to no response) were obtained. If the animal did not respond after reaching the strongest filament or if the animal responded after reaching the weakest filament, the testing was stopped for that time point.

The 50% response threshold is calculated using the formula:

50% response threshold(g)=(10(Xf+kδ))/10,000

Xf=value (in log units) of the final von Frey filament used
k=tabular value for the pattern of positive/negative responses (Chaplan et al. 1994, appendix 1, page 62).
δ=mean difference (in log units) between stimuli.

### Result

Success criteria for this study were to 1) create a model of mechanical sensitivity and to 2) demonstrate reversal of mechanical sensitivity by gabapentin. Both of these criteria were met. Vehicle treated animals demonstrated significant mechanical sensitivity evidenced by significantly lower 50% response thresholds at the ipsilateral paw compared

to contralateral 50% paw withdrawal thresholds. Gabapentin administration significantly increased paw withdrawal thresholds at all post-dose time points.

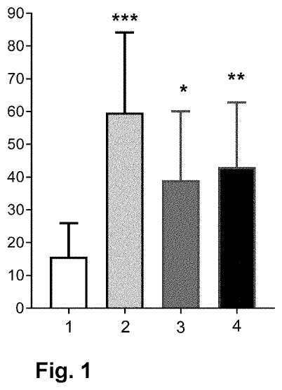

The study demonstrated that administration of the compound according to example 68 increased 50% paw withdrawal threshold significantly, the compound decreased mechanical allodynia induced by CCI surgery serving as neuropathic pain model (FIG. 1).

With reference to FIG. 1 showing the analgesic effect of compound 68 according to the invention (50% paw withdrawal threshold):

***p<0.005, **p<0.01, *p<0.05 ANOVA followed by Dunnefs posthoc test vs vehicle Y-axis represents: Mean 50% Paw withdrawal threshold AUC (day 14-23)+SD X-axis legend: 1=vehicle, 2=gabapentin 100 mg/kg body-weight, 3=example 68 30 mg/kg body-weight, 4=example 68 100 mg/kg body-weight.

### In Vivo Brain Plasma Ratio in Mouse

Penetration of test compounds into the brain was assessed in female CD mice after intravenous administration. Test compounds were administered at standard doses of 0.3 to 1 mg/kg formulated as solutions using DMSO/plasma formulations or solubilizers such as PEG400 in well-tolerated amounts. Separate groups of animals (3 animals per group) were sacrificed at least at 3 different time points (e.g. 0.5; 1 and 4 h) after dosing and blood and brain were sampled. Blood was collected into Lithium-Heparintubes (Monovetten®, Sarstedt) and centrifuged for 15 min at 3000 rpm. An aliquot of 100 μL from the supernatant (Plasma) was taken and precipitated by addition of 400 μL cold methanol and frozen at −20° C. over night. Brain samples were homogenized with 50 mM Tris-HCl buffer, pH7.5 (1:5 w/v), precipitated with methanol (1:5, v/v) and frozen at −20° C. over night. Plasma and brain samples were subsequently thawed and centrifuged at 3000 rpm, 4° C. for 20 minutes. Aliquots of the supernatants were taken for analytical testing using an Agilent 1200 HPLC-system with LCMS/MS detection.

From the concentration-time profiles the AUC (area under the concentration-time curve) in plasma and brain were calculated and the ratio AUC_{brain}/AUCₚₗₐₛₘₐ (total) was calculated. Second, the ratio of the unbound AUC_{brain}/AUCₚₗₐₛₘₐ (AUC multiplied with fraction unbound (fu)) was reported as the brain-plasma ratio or Kpuu (Partition coefficient Unbound to Unbound Concentration). For the Kpuu calculation the Protein Binding is measured in accordance to the method below. Due to residual blood in the non-perfused brain tissue the lower limit for the brain-plasma ratio by this method approximates 1-2%.

### Protein Binding:

Estimation of Plasma Protein Binding by Equilibrium Dialysis. Binding of test compounds to plasma proteins is measured by equilibrium dialysis in a 96-well format using ht-dialysis equipment made of Teflon and a semipermeable membrane (regenerated cellulose, MWCO 12-14K). The membrane separates the plasma and buffer side (50 mM phosphate buffer) filled with 150 μl each. The test compound is added in a concentration of 3 μM to the plasma side and binds to plasma proteins. The unbound fraction of the test compound passes the membrane and distributes on both sides until equilibrium is reached, which is usually the case after 6-8h at 37° C. Compound concentration of plasma and buffer side is measured by LC-MSMS analytics. For this both sides are diluted with buffer and plasma to achieve the same matrix (10% plasma) and subsequently are precipitated with methanol. From the quotient of buffer and plasma concentration the free (unbound) fraction (fu) is calculated. Stability and recovery controls are included. Additionally, the test compound is dialyzed in buffer against buffer in order to estimate non-specific binding to equipment and/or membrane and to investigate in the establishment of the equilibrium. Due to the osmotic pressure of the plasma proteins a dilution of the plasma takes place during the incubation (volume shift). The potential imprecision is addressed by inclusion of an empirical factor in the calculation of the fu. Establishment of equilibrium and stability in plasma should be at least 80% and the recovery in plasma should at least be 30%. A free fraction of <1% is designated as high, between 1 and 10% as moderate and of >10% as low plasma protein binding.

1. A compound of formula (I): 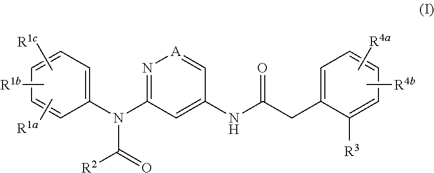
wherein
A is CH or N;
R^{1a}, R^{1b}, and R^{1c} are each independently a hydrogen atom, a halogen atom, cyano, (C₁-C₃)-alkyl, (C₁-C₃)-haloalkyl, or (C₁-C₃)-alkoxy;
R² is (C₁-C₃)-alkyl;
R³ is a halogen atom, cyano, (C₁-C₃)-alkyl, (C₁-C₃)-haloalkyl, or (C₁-C₃)-alkoxy;
R^{4a} and R^{4b} are each independently a hydrogen atom, a halogen atom, cyano, (C₁-C₃)-alkyl, (C₁-C₃)-haloalkyl, or (C₁-C₃)-alkoxy;
or a stereoisomer, a tautomer, an N-oxide, a hydrate, a solvate, or a salt thereof, or a mixture of same.

2. The compound according to claim 1, wherein:
R^{1a} and R^{1b} are each independently a halogen atom, cyano, (C₁-C₃)-alkyl, (C₁-C₃)-haloalkyl, or (C₁-C₃)-alkoxy; and R^{1c} is a hydrogen atom.

3. The compound according to claim 1, wherein:
R^{1a} is in position 4 of the phenyl ring and is a halogen atom, cyano, (C₁-C₃)-alkyl, (C₁-C₃)-haloalkyl, or (C₁-C₃)-alkoxy; R^{1b} is a hydrogen atom, a halogen atom, cyano, (C₁-C₃)-alkyl, (C₁-C₃)-haloalkyl, or (C₁-C₃)-alkoxy; and R^{1c} is a hydrogen atom.

4. The compound according to claim 1, wherein:
R^{1a} is in position 4 of the phenyl ring and is a halogen atom, cyano, (C₁-C₃)-alkyl, (C₁-C₃)-haloalkyl, or (C₁-C₃)-alkoxy; R^{1b} is in position 3 of the phenyl ring and is a hydrogen atom, a halogen atom, cyano, (C₁-C₃)-alkyl, (C₁-C₃)-haloalkyl, or (C₁-C₃)-alkoxy; and R^{1c} is a hydrogen atom.

5. The compound according to claim 1, wherein:
R^{1a} is a halogen atom, cyano, (C₁-C₃)-alkyl, (C₁-C₃)-haloalkyl, or (C₁-C₃)-alkoxy; and
R^{1b} and R^{1c} are a hydrogen atom.

6. The compound according to claim 1, wherein:
R² is a methyl.

7. The compound according to claim 1, wherein:
R³ is a chlorine, fluorine, cyano, or a hydrogen atom.

8. The compound according to claim 1, wherein:
R^{4a} is a halogen atom, cyano, (C₁-C₃)-alkyl, (C₁-C₃)-haloalkyl, or (C₁-C₃)-alkoxy; and R^{4b} is a hydrogen atom; or
R^{4a} is a halogen atom, cyano, (C₁-C₃)-alkyl, (C₁-C₃)-haloalkyl, or (C₁-C₃)-alkoxy; and R^{4b} is a halogen atom, cyano, (C₁-C₃)-alkyl, (C₁-C₃)-haloalkyl, or (C₁-C₃)-alkoxy.

9. The compound according to claim 1, wherein:
R³ is a chlorine, fluorine, or cyano, R^{4a} is a halogen atom, cyano, (C₁-C₃)-alkyl, (C₁-C₃)-haloalkyl, or (C₁-C₃)-alkoxy in position 3 or 6 of the phenyl group; and R^{4b} is a hydrogen atom; or
R³ is a chlorine, fluorine, or cyano, R^{4a} is a halogen atom, cyano, (C₁-C₃)-alkyl, (C₁-C₃)-haloalkyl, or (C₁-C₃)-alkoxy in position 6 of the phenyl group; and
R^{4b} is a halogen atom, cyano, (C₁-C₃)-alkyl, (C₁-C₃)-haloalkyl, or (C₁-C₃)-alkoxy in position 4 of the phenyl group.

10. The compound according to claim 1, wherein the compound is:
N-{4-[2-(2-chlorophenyl)acetamido]pyridin-2-yl}-N-(3,4-difluorophenyl)acetamide;
N-{4-[2-(2-chloro-3-fluorophenyl)acetamido]pyridin-2-yl}-N-(4-fluorophenyl)acetamide;
N-{4-[2-(2-chloro-4-fluorophenyl)acetamido]pyridin-2-yl}-N-[3-(difluoromethyl)phenyl]acetamide;
N-{4-[2-(2-chloro-3-fluorophenyl)acetamido]pyridin-2-yl}-N-[3-(difluoromethyl)phenyl]acetamide;
N-{4-[2-(2,6-dichlorophenyl)acetamido]pyridin-2-yl}-N-[3-(difluoromethyl)phenyl]acetamide;
N-{4-[2-(2-chlorophenyl)acetamido]pyridin-2-yl}-N-[3-(trifluoromethyl)phenyl]acetamide;
N-{4-[2-(2-chloro-4-fluorophenyl)acetamido]pyridin-2-yl}-N-[3-(trifluoromethyl)phenyl]acetamide;
N-{4-[2-(2-chloro-3-fluorophenyl)acetamido]pyridin-2-yl}-N-[3-(trifluoromethyl)phenyl]acetamide;
N-{4-[2-(2,6-dichlorophenyl)acetamido]pyridin-2-yl}-N-[3-(trifluoromethyl)phenyl]acetamide;
N-{4-[2-(2-chlorophenyl)acetamido]pyridin-2-yl}-N-[4-cyano-3-(trifluoromethyl)phenyl]acetamide;
N-{4-[2-(2-chloro-3-fluorophenyl)acetamido]pyridin-2-yl}-N-[4-cyano-3-(trifluoromethyl)phenyl]acetamide;
N-[4-cyano-3-(trifluoromethyl)phenyl]-N-{4-[2-(2,6-dichlorophenyl)acetamido]pyridin-2-yl}acetamide;
N-{4-[2-(2-chlorophenyl)acetamido]pyridin-2-yl}-N-(3-cyanophenyl)acetamide;
N-(3-cyanophenyl)-N-{4-[2-(2,6-dichlorophenyl)acetamido]pyridin-2-yl}acetamide;
N-{4-[2-(2-chloro-6-fluorophenyl)acetamido]pyridin-2-yl}-N-(3-cyanophenyl)acetamide;
N-(4-chloro-3-cyanophenyl)-N-{4-[2-(2-chlorophenyl)acetamido]pyridin-2-yl}acetamide;
N-(4-chloro-3-cyanophenyl)-N-{4-[2-(2-chloro-4-fluorophenyl)acetamido]pyridin-2-yl}acetamide;
N-(4-chloro-3-cyanophenyl)-N-{4-[2-(2-chloro-3-fluorophenyl)acetamido]pyridin-2-yl}acetamide;
N-(4-chloro-3-cyanophenyl)-N-{4-[2-(2,6-dichlorophenyl)acetamido]pyridin-2-yl}acetamide;
N-(4-{2-[2-chloro-3-(trifluoromethyl)phenyl]acetamido}pyridin-2-yl)-N-(3-cyano-4-fluorophenyl)acetamide;
N-{4-[2-(2-chlorophenyl)acetamido]pyridin-2-yl}-N-(3-cyano-4-fluorophenyl)acetamide;
N-{4-[2-(2-chloro-4-fluorophenyl)acetamido]pyridin-2-yl}-N-(3-cyano-4-fluorophenyl)acetamide;
N-{4-[2-(2-chloro-3-fluorophenyl)acetamido]pyridin-2-yl}-N-(3-cyano-4-fluorophenyl)acetamide;
N-(3-cyano-4-fluorophenyl)-N-{4-[2-(2,6-dichlorophenyl)acetamido]pyridin-2-yl}acetamide;
N-{4-[2-(2-chlorophenyl)acetamido]pyridin-2-yl}-N-phenylacetamide;
N-{4-[2-(2-chloro-4-fluorophenyl)acetamido]pyridin-2-yl}-N-phenylacetamide;
N-{4-[2-(2,6-dichlorophenyl)acetamido]pyridin-2-yl}-N-phenylacetamide;
N-{4-[2-(2-chloro-6-fluorophenyl)acetamido]pyridin-2-yl}-N-phenylacetamide;
N-{4-[2-(2,3-dimethylphenyl)acetamido]pyridin-2-yl}-N-(4-fluorophenyl)acetamide;
N-(4-fluorophenyl)-N-(4-{2-[2-(trifluoromethyl)phenyl]acetamido}pyridin-2-yl)acetamide;
N-(4-{2-[4-chloro-2-(trifluoromethyl)phenyl]acetamido}pyridin-2-yl)-N-(4-fluorophenyl)acetamide;
N-(4-fluorophenyl)-N-(4-{2-[3-fluoro-2-(trifluoromethyl)phenyl]acetamido}pyridin-2-yl)acetamide;
N-{4-[2-(2-chloro-6-cyanophenyl)acetamido]pyridin-2-yl}-N-(4-fluorophenyl)acetamide;
N-{4-[2-(2,6-dimethylphenyl)acetamido]pyridin-2-yl}-N-(4-fluorophenyl)acetamide;
N-(4-{2-[2-chloro-3-(trifluoromethyl)phenyl]acetamido}pyridin-2-yl)-N-(4-fluorophenyl)acetamide;
N-{4-[2-(2-chlorophenyl)acetamido]pyridin-2-yl}-N-(4-fluorophenyl)acetamide;
N-{4-[2-(2-chloro-4-fluorophenyl)acetamido]pyridin-2-yl}-N-(4-fluorophenyl)acetamide;
N-{4-[2-(2,6-dichlorophenyl)acetamido]pyridin-2-yl}-N-(4-fluorophenyl)acetamide;
N-{4-[2-(2,6-dichloro-4-fluorophenyl)acetamido]pyridin-2-yl}-N-(4-fluorophenyl)acetamide;
N-{4-[2-(2-chloro-6-fluorophenyl)acetamido]pyridin-2-yl}-N-(4-fluorophenyl)acetamide;
N-{4-[2-(2-chloro-4,6-difluorophenyl)acetamido]pyridin-2-yl}-N-(4-fluorophenyl)acetamide;
N-(4-fluorophenyl)-N-(4-{2-[2-(trifluoromethoxy)phenyl]acetamido}pyridin-2-yl)acetamide;
N-{4-[2-(2-chlorophenyl)acetamido]pyridin-2-yl}-N-(4-methylphenyl)acetamide;
N-{4-[2-(2-chloro-4-fluorophenyl)acetamido]pyridin-2-yl}-N-(4-methylphenyl)acetamide;
N-{4-[2-(2-chloro-3-fluorophenyl)acetamido]pyridin-2-yl}-N-(4-methylphenyl)acetamide;
N-{4-[2-(2,6-dichlorophenyl)acetamido]pyridin-2-yl}-N-(4-methylphenyl)acetamide;
N-{4-[2-(2-chlorophenyl)acetamido]pyridin-2-yl}-N-[4-(difluoromethoxy)phenyl]acetamide;
N-{4-[2-(2-chloro-3-fluorophenyl)acetamido]pyridin-2-yl}-N-[4-(difluoromethoxy)phenyl]acetamide;
N-{4-[2-(2,6-dichlorophenyl)acetamido]pyridin-2-yl}-N-[4-(difluoromethoxy)phenyl]acetamide;
N-(3-chloro-4-fluorophenyl)-N-{4-[2-(2-chlorophenyl)acetamido]pyridin-2-yl}acetamide;
N-(3-chloro-4-fluorophenyl)-N-{4-[2-(2-chloro-4-fluorophenyl)acetamido]pyridin-2-yl}acetamide;
N-(3-chloro-4-fluorophenyl)-N-{4-[2-(2-chloro-3-fluorophenyl)acetamido]pyridin-2-yl}acetamide;
N-(3-chloro-4-fluorophenyl)-N-{4-[2-(2,6-dichlorophenyl)acetamido]pyridin-2-yl}acetamide;
N-{4-[2-(2-chlorophenyl)acetamido]pyridin-2-yl}-N-(3-fluorophenyl)acetamide;
N-{4-[2-(2-chloro-4-fluorophenyl)acetamido]pyridin-2-yl}-N-(3-fluorophenyl)acetamide;
N-{4-[2-(2,6-dichlorophenyl)acetamido]pyridin-2-yl}-N-(3-fluorophenyl)acetamide;
N-{4-[2-(2,6-dichloro-4-fluorophenyl)acetamido]pyridin-2-yl}-N-(3-fluorophenyl)acetamide;
N-(4-chloro-3-fluorophenyl)-N-{4-[2-(2-chlorophenyl)acetamido]pyridin-2-yl}acetamide;
N-(4-chloro-3-fluorophenyl)-N-{4-[2-(2-chloro-4-fluorophenyl)acetamido]pyridin-2-yl}acetamide;
N-(4-chloro-3-fluorophenyl)-N-{4-[2-(2-chloro-3-fluorophenyl)acetamido]pyridin-2-yl}acetamide;
N-(4-chloro-3-fluorophenyl)-N-{4-[2-(2,6-dichlorophenyl)acetamido]pyridin-2-yl}acetamide;
N-(3,4-difluorophenyl)-N-{4-[2-(2,3-dimethylphenyl)acetamido]pyridin-2-yl}acetamide;
N-(3,4-difluorophenyl)-N-(4-{2-[2-(trifluoromethyl)phenyl]acetamido}pyridin-2-yl)acetamide;
N-{4-[2-(2,4-dichloro-6-methylphenyl)acetamido]pyridin-2-yl}-N-(3,4-difluorophenyl)acetamide;
N-{4-[2-(2-chloro-4,6-dimethylphenyl)acetamido]pyridin-2-yl}-N-(3,4-difluorophenyl)acetamide;
N-(3,4-difluorophenyl)-N-{4-[2-(2,6-dimethylphenyl)acetamido]pyridin-2-yl}acetamide;
N-{4-[2-(2,4-dichlorophenyl)acetamido]pyridin-2-yl}-N-(3,4-difluorophenyl)acetamide;
N-{4-[2-(2-chloro-4-fluorophenyl)acetamido]pyridin-2-yl}-N-(3,4-difluorophenyl)acetamide;
N-{4-[2-(2-chloro-4-nitrophenyl)acetamido]pyridin-2-yl}-N-(3,4-difluorophenyl)acetamide;
N-{4-[2-(2-chloro-4-methoxyphenyl)acetamido]pyridin-2-yl}-N-(3,4-difluorophenyl)acetamide;
N-{4-[2-(2-chloro-3-fluorophenyl)acetamido]pyridin-2-yl}-N-(3,4-difluorophenyl)acetamide;
N-{4-[2-(2,6-dichlorophenyl)acetamido]pyridin-2-yl}-N-(3,4-difluorophenyl)acetamide;
N-{4-[2-(2,6-dichloro-4-fluorophenyl)acetamido]pyridin-2-yl}-N-(3,4-difluorophenyl)acetamide;
N-{4-[2-(2,6-dichloro-4-methylphenyl)acetamido]pyridin-2-yl}-N-(3,4-difluorophenyl)acetamide;
N-{4-[2-(2,6-dichloro-4-ethylphenyl)acetamido]pyridin-2-yl}-N-(3,4-difluorophenyl)acetamide;
N-(3,4-difluorophenyl)-N-(4-{2-[2-(trifluoromethoxy)phenyl]acetamido}pyridin-2-yl)acetamide;
N-{4-[2-(2-chlorophenyl)acetamido]pyridin-2-yl}-N-(3-fluoro-4-methoxyphenyl)acetamide;
N-{4-[2-(2-chloro-4-fluorophenyl)acetamido]pyridin-2-yl}-N-(3-fluoro-4-methoxyphenyl)acetamide;
N-{4-[2-(2-chloro-3-fluorophenyl)acetamido]pyridin-2-yl}-N-(3-fluoro-4-methoxyphenyl)acetamide;
N-{4-[2-(2,6-dichlorophenyl)acetamido]pyridin-2-yl}-N-(3-fluoro-4-methoxyphenyl)acetamide;
N-{4-[2-(2-chloro-3-fluorophenyl)acetamido]pyridin-2-yl}-N-[3-fluoro-4-(methanesulfonyl)phenyl]acetamide;
N-{4-[2-(2,6-dichlorophenyl)acetamido]pyridin-2-yl}-N-[3-fluoro-4-(methanesulfonyl)phenyl]acetamide;
N-(3,5-difluorophenyl)-N-{4-[2-(2,3-dimethylphenyl)acetamido]pyridin-2-yl}acetamide;
N-(3,5-difluorophenyl)-N-(4-{2-[2-(trifluoromethyl)phenyl]acetamido}pyridin-2-yl)acetamide;
N-(3,5-difluorophenyl)-N-{4-[2-(2,6-dimethylphenyl)acetamido]pyridin-2-yl}acetamide;
N-{4-[2-(2-chlorophenyl)acetamido]pyridin-2-yl}-N-(3,5-difluorophenyl)acetamide;
N-{4-[2-(2-chloro-4-fluorophenyl)acetamido]pyridin-2-yl}-N-(3,5-difluorophenyl)acetamide;
N-{4-[2-(2-chloro-3-fluorophenyl)acetamido]pyridin-2-yl}-N-(3,5-difluorophenyl)acetamide;
N-{4-[2-(2,6-dichlorophenyl)acetamido]pyridin-2-yl}-N-(3,5-difluorophenyl)acetamide;
N-{4-[2-(2,6-dichloro-4-fluorophenyl)acetamido]pyridin-2-yl}-N-(3,5-difluorophenyl)acetamide;
N-(3,5-difluorophenyl)-N-(4-{2-[2-(trifluoromethoxy)phenyl]acetamido}pyridin-2-yl)acetamide;
N-{4-[2-(2-chlorophenyl)acetamido]pyridin-2-yl}-N-(3,5-difluoro-4-methylphenyl)acetamide;
N-{4-[2-(2-chloro-4-fluorophenyl)acetamido]pyridin-2-yl}-N-(3,5-difluoro-4-methylphenyl)acetamide;
N-{4-[2-(2-chloro-3-fluorophenyl)acetamido]pyridin-2-yl}-N-(3,5-difluoro-4-methylphenyl)acetamide;
N-{4-[2-(2,6-dichlorophenyl)acetamido]pyridin-2-yl}-N-(3,5-difluoro-4-methylphenyl)acetamide;
N-{4-[2-(2-chlorophenyl)acetamido]pyridin-2-yl}-N-(3,5-difluoro-4-methoxyphenyl)acetamide;
N-{4-[2-(2-chloro-4-fluorophenyl)acetamido]pyridin-2-yl}-N-(3,5-difluoro-4-methoxyphenyl)acetamide;
N-{4-[2-(2-chloro-3-fluorophenyl)acetamido]pyridin-2-yl}-N-(3,5-difluoro-4-methoxyphenyl)acetamide;
N-{4-[2-(2,6-dichlorophenyl)acetamido]pyridin-2-yl}-N-(3,5-difluoro-4-methoxyphenyl)acetamide;
N-{4-[2-(2-chlorophenyl)acetamido]pyridin-2-yl}-N-(3-methoxyphenyl)acetamide;
N-{4-[2-(2-chloro-3-fluorophenyl)acetamido]pyridin-2-yl}-N-(3-methoxyphenyl)acetamide;
N-{4-[2-(2-chlorophenyl)acetamido]pyridin-2-yl}-N-[3-(trifluoromethoxy)phenyl]acetamide;
N-{4-[2-(2-chloro-3-fluorophenyl)acetamido]pyridin-2-yl}-N-(4-fluoro-3-methoxyphenyl)acetamide;
N-{4-[2-(2,6-dichlorophenyl)acetamido]pyridin-2-yl}-N-(4-fluoro-3-methoxyphenyl)acetamide;
N-(2-chlorophenyl)-N-(4-{2-[2-chloro-3-(trifluoromethyl)phenyl]acetamido}pyridin-2-yl)acetamide;
N-(2-chlorophenyl)-N-{4-[2-(2-chlorophenyl)acetamido]pyridin-2-yl}acetamide;
N-{4-[2-(2-chloro-4-fluorophenyl)acetamido]pyridin-2-yl}-N-(2-chlorophenyl)acetamide;
N-{4-[2-(2-chloro-3-fluorophenyl)acetamido]pyridin-2-yl}-N-(2-chlorophenyl)acetamide;
N-(2-chlorophenyl)-N-{4-[2-(2,6-dichlorophenyl)acetamido]pyridin-2-yl}acetamide;
N-(2-chloro-5-fluorophenyl)-N-(4-{2-[2-chloro-3-(trifluoromethyl)phenyl]acetamido}pyridin-2-yl)acetamide;
N-(2-chloro-5-fluorophenyl)-N-{4-[2-(2-chlorophenyl)acetamido]pyridin-2-yl}acetamide;
N-(2-chloro-5-fluorophenyl)-N-{4-[2-(2-chloro-4-fluorophenyl)acetamido]pyridin-2-yl}acetamide;
N-(2-chloro-5-fluorophenyl)-N-{4-[2-(2-chloro-3-fluorophenyl)acetamido]pyridin-2-yl}acetamide;
N-(2-chloro-5-fluorophenyl)-N-{4-[2-(2,6-dichlorophenyl)acetamido]pyridin-2-yl}acetamide;
N-{4-[2-(2-chlorophenyl)acetamido]pyridin-2-yl}-N-(2-fluorophenyl)acetamide;
N-{4-[2-(2-chloro-4-fluorophenyl)acetamido]pyridin-2-yl}-N-(2-fluorophenyl)acetamide;
N-{4-[2-(2-chloro-3-fluorophenyl)acetamido]pyridin-2-yl}-N-(2-fluorophenyl)acetamide;
N-{4-[2-(2,6-dichlorophenyl)acetamido]pyridin-2-yl}-N-(2-fluorophenyl)acetamide;
N-{4-[2-(2-chloro-3-fluorophenyl)acetamido]pyridin-2-yl}-N-[2-fluoro-4-(trifluoromethyl)phenyl]acetamide;
N-{4-[2-(2,6-dichlorophenyl)acetamido]pyridin-2-yl}-N-[2-fluoro-4-(trifluoromethyl)phenyl]acetamide;
N-(4-{2-[2-chloro-3-(trifluoromethyl)phenyl]acetamido}pyridin-2-yl)-N-(2,3-difluorophenyl)acetamide;
N-{4-[2-(2-chlorophenyl)acetamido]pyridin-2-yl}-N-(2,3-difluorophenyl)acetamide;
N-{4-[2-(2-chloro-4-fluorophenyl)acetamido]pyridin-2-yl}-N-(2,3-difluorophenyl)acetamide;
N-{4-[2-(2-chloro-3-fluorophenyl)acetamido]pyridin-2-yl}-N-(2,3-difluorophenyl)acetamide;
N-{4-[2-(2,6-dichlorophenyl)acetamido]pyridin-2-yl}-N-(2,3-difluorophenyl)acetamide;
N-{4-[2-(2-chlorophenyl)acetamido]pyridin-2-yl}-N-(2,4-difluorophenyl)acetamide;
N-{4-[2-(2-chloro-4-fluorophenyl)acetamido]pyridin-2-yl}-N-(2,4-difluorophenyl)acetamide;
N-{4-[2-(2-chloro-3-fluorophenyl)acetamido]pyridin-2-yl}-N-(2,4-difluorophenyl)acetamide;
N-{4-[2-(2,6-dichlorophenyl)acetamido]pyridin-2-yl}-N-(2,4-difluorophenyl)acetamide;
N-{4-[2-(2-chloro-6-fluorophenyl)acetamido]pyridin-2-yl}-N-(2,4-difluorophenyl)acetamide;
N-(3-chlorophenyl)-N-{4-[2-(2-chlorophenyl)acetamido]pyridin-2-yl}acetamide;
N-{4-[2-(2-chloro-4-fluorophenyl)acetamido]pyridin-2-yl}-N-(3-chlorophenyl)acetamide;
N-{4-[2-(2-chloro-3-fluorophenyl)acetamido]pyridin-2-yl}-N-(3-chlorophenyl)acetamide;
N-(3-chlorophenyl)-N-{4-[2-(2,6-dichlorophenyl)acetamido]pyridin-2-yl}acetamide;
N-{4-[2-(2-chloro-6-fluorophenyl)acetamido]pyridin-2-yl}-N-(3-chlorophenyl)acetamide;
N-(3-chloro-5-fluorophenyl)-N-{4-[2-(2-chlorophenyl)acetamido]pyridin-2-yl}acetamide;
N-(3-chloro-5-fluorophenyl)-N-{4-[2-(2-chloro-4-fluorophenyl)acetamido]pyridin-2-yl}acetamide;
N-(3-chloro-5-fluorophenyl)-N-{4-[2-(2-chloro-3-fluorophenyl)acetamido]pyridin-2-yl}acetamide;
N-(3-chloro-5-fluorophenyl)-N-{4-[2-(2,6-dichlorophenyl)acetamido]pyridin-2-yl}acetamide;
N-(3-chloro-5-fluorophenyl)-N-{4-[2-(2-chloro-6-fluorophenyl)acetamido]pyridin-2-yl}acetamide;
N-{4-[2-(2-chloro-4-fluorophenyl)acetamido]pyridin-2-yl}-N-(4-fluorophenyl)butanamide;
N-{4-[2-(2-chloro-6-fluorophenyl)acetamido]pyridin-2-yl}-N-(4-fluorophenyl)butanamide;
N-{4-[2-(2-chlorophenyl)acetamido]pyridin-2-yl}-N-(4-fluorophenyl)butanamide;
N-{4-[2-(2,6-dichlorophenyl)acetamido]pyridin-2-yl}-N-(4-fluorophenyl)butanamide;
N-{4-[2-(2-Chlorphenyl)acetamido]pyridin-2-yl}-N-(3-cyan-5-fluorphenyl)acetamide;
N-(3-Cyan-5-fluorphenyl)-N-{4-[2-(2,6-dichlorphenyl)acetamido]pyridin-2-yl}acetamide;
N-{4-[2-(2-Chlor-3-fluorphenyl)acetamido]pyridin-2-yl}-N-(3-cyan-5-fluorphenyl)acetamide;
N-{4-[2-(2-Chlor-6-fluorphenyl)acetamido]pyridin-2-yl}-N-(3-cyan-5-fluorphenyl)acetamide;
N-{4-[2-(2-Chlor-4-fluorphenyl)acetamido]pyridin-2-yl}-N-(3-cyan-5-fluorphenyl)acetamide;
N-(2-Chlor-4-fluorphenyl)-N-{4-[2-(2-chlorphenyl)acetamido]pyridin-2-yl}acetamide;
N-(2-Chlor-4-fluorphenyl)-N-{4-[2-(2-chlor-4-fluorphenyl)acetamido]pyridin-2-yl}acetamide;
N-(2-Chlor-4-fluorphenyl)-N-{4-[2-(2,6-dichlorphenyl)acetamido]pyridin-2-yl}acetamide;
N-(2-Chlor-4-fluorphenyl)-N-{4-[2-(2-chlor-6-fluorphenyl)acetamido]pyridin-2-yl}acetamide;
N-(2-Chlor-4-fluorphenyl)-N-{4-[2-(2-chlor-3-fluorphenyl)acetamido]pyridin-2-yl}acetamide;
N-[3-Chlor-4-(methylsulfonyl)phenyl]-N-{4-[2-(2,6-dichlorphenyl)acetamido]pyridin-2-yl}acetamide;
N-{4-[2-(2-Chlor-3-fluorphenyl)acetamido]pyridin-2-yl}-N-[3-chlor-4-(methylsulfonyl)phenyl]acetamide;
N-[3-Chlor-4-(methylsulfonyl)phenyl]-N-{4-[2-(2-chlorphenyl)acetamido]pyridin-2-yl}acetamide;
N-{4-[2-(2-Chlor-6-fluorphenyl)acetamido]pyridin-2-yl}-N-[3-chlor-4-(methylsulfonyl)phenyl]acetamide;
N-{4-[2-(2-Chlor-4-fluorphenyl)acetamido]pyridin-2-yl}-N-[3-chlor-4-(methylsulfonyl)phenyl]acetamide;
N-{4-[2-(2-Chlor-3-fluorphenyl)acetamido]pyridin-2-yl}-N-(3-fluor-5-methoxyphenyl)acetamide;
N-{4-[2-(2-Chlorphenyl)acetamido]pyridin-2-yl}-N-(3-fluor-5-methoxyphenyl)acetamide;
N-{4-[2-(2,6-Dichlorphenyl)acetamido]pyridin-2-yl}-N-(3-fluor-5-methoxyphenyl)acetamide;
N-{4-[2-(2-Chlor-4-fluorphenyl)acetamido]pyridin-2-yl}-N-(3-fluor-5-methoxyphenyl)acetamide;
N-{4-[2-(2-Chlor-6-fluorphenyl)acetamido]pyridin-2-yl}-N-(3-fluor-5-methoxyphenyl)acetamide;
N-{4-[2-(2,6-Dichlorphenyl)acetamido]pyridin-2-yl}-N-[2-(difluormethyl)phenyl]acetamide;
N-{4-[2-(2-Chlorphenyl)acetamido]pyridin-2-yl}-N-[2-(difluormethyl)phenyl]acetamide;
N-{4-[2-(2-Chlor-3-fluorphenyl)acetamido]pyridin-2-yl}-N-[2-(difluormethyl)phenyl]acetamide;
N-{4-[2-(2-Chlor-6-fluorphenyl)acetamido]pyridin-2-yl}-N-[2-(difluormethyl)phenyl]acetamide;
N-{4-[2-(2-Chlor-4-fluorphenyl)acetamido]pyridin-2-yl}-N-[2-(difluormethyl)phenyl]acetamide;
N-{4-[2-(2-Chlorphenyl)acetamido]pyridin-2-yl}-N-(2,4-dimethylphenyl)acetamide;
N-{4-[2-(2-Chlor-3-fluorphenyl)acetamido]pyridin-2-yl}-N-(2,4-dimethylphenyl)acetamide;
N-{4-[2-(2-Chlor-6-fluorphenyl)acetamido]pyridin-2-yl}-N-(2,4-dimethylphenyl)acetamide;
N-{4-[2-(2,6-Dichlorphenyl)acetamido]pyridin-2-yl}-N-(2,4-dimethylphenyl)acetamide;
N-{4-[2-(2-Chlor-4-fluorphenyl)acetamido]pyridin-2-yl}-N-(2,4-dimethylphenyl)acetamide;
N-{4-[2-(2-Chlorphenyl)acetamido]pyridin-2-yl}-N-(3-cyan-5-methylphenyl)acetamide;
N-(3-Cyan-5-methylphenyl)-N-{4-[2-(2,6-dichlorphenyl)acetamido]pyridin-2-yl}acetamide;
N-{4-[2-(2-Chlor-3-fluorphenyl)acetamido]pyridin-2-yl}-N-(3-cyan-5-methylphenyl)acetamide;
N-{4-[2-(2-Chlor-4-fluorphenyl)acetamido]pyridin-2-yl}-N-(3-cyan-5-methylphenyl)acetamide;
N-{4-[2-(2-Chlor-6-fluorphenyl)acetamido]pyridin-2-yl}-N-(3-cyan-5-methylphenyl)acetamide;
N-(3-Chlor-4-methylphenyl)-N-{4-[2-(2-chlorphenyl)acetamido]pyridin-2-yl}acetamide;
N-{4-[2-(2-Chlor-3-fluorphenyl)acetamido]pyridin-2-yl}-N-(3-chlor-4-methylphenyl)acetamide;
N-(3-Chlor-4-methylphenyl)-N-{4-[2-(2,6-dichlorphenyl)acetamido]pyridin-2-yl}acetamide;
N-{4-[2-(2-Chlor-6-fluorphenyl)acetamido]pyridin-2-yl}-N-(3-chlor-4-methylphenyl)acetamide;
N-{4-[2-(2-Chlor-6-fluorphenyl)acetamido]pyridin-2-yl}-N-(4-fluor-2,3-dimethylphenyl)acetamide;
N-{5-[2-(2,6-dichlorophenyl)acetamido]pyridazin-3-yl}-N-phenylacetamide;
N-{5-[2-(2-chloro-3-fluorophenyl)acetamido]pyridazin-3-yl}-N-phenylacetamide;
N-{5-[2-(2-chlorophenyl)acetamido]pyridazin-3-yl}-N-phenylacetamide;
N-{5-[2-(2-chloro-4-fluorophenyl)acetamido]pyridazin-3-yl}-N-phenylacetamide;
N-{5-[2-(2-chloro-6-fluorophenyl)acetamido]pyridazin-3-yl}-N-(4-fluorophenyl)acetamide;
N-{5-[2-(2-chloro-4-fluorophenyl)acetamido]pyridazin-3-yl}-N-(4-fluorophenyl)acetamide;
N-{5-[2-(2-chloro-3-fluorophenyl)acetamido]pyridazin-3-yl}-N-(4-fluorophenyl)acetamide;
N-{5-[2-(2-chlorophenyl)acetamido]pyridazin-3-yl}-N-(4-fluorophenyl)acetamide;
N-{5-[2-(2,6-dichlorophenyl)acetamido]pyridazin-3-yl}-N-(4-fluorophenyl)acetamide;
N-{5-[2-(2,6-dichlorophenyl)acetamido]pyridazin-3-yl}-N-(3-fluorophenyl)acetamide;
N-{5-[2-(2-chloro-3-fluorophenyl)acetamido]pyridazin-3-yl}-N-(3-fluorophenyl)acetamide;
N-{5-[2-(2-chlorophenyl)acetamido]pyridazin-3-yl}-N-(3-fluorophenyl)acetamide;
N-{5-[2-(2-chloro-6-fluorophenyl)acetamido]pyridazin-3-yl}-N-(3-fluorophenyl)acetamide;
N-{5-[2-(2-chloro-4-fluorophenyl)acetamido]pyridazin-3-yl}-N-(3-fluorophenyl)acetamide;
N-{5-[2-(2-chlorophenyl)acetamido]pyridazin-3-yl}-N-(3,4-difluorophenyl)acetamide;
N-{5-[2-(2,6-dichlorophenyl)acetamido]pyridazin-3-yl}-N-(3,4-difluorophenyl)acetamide;
N-{5-[2-(2-chloro-3-fluorophenyl)acetamido]pyridazin-3-yl}-N-(3,4-difluorophenyl)acetamide;
N-{5-[2-(2-chloro-4-fluorophenyl)acetamido]pyridazin-3-yl}-N-(3,4-difluorophenyl)acetamide;
N-{5-[2-(2-chloro-6-fluorophenyl)acetamido]pyridazin-3-yl}-N-(3,4-difluorophenyl)acetamide;
N-{5-[2-(2,6-dichlorophenyl)acetamido]pyridazin-3-yl}-N-(3,5-difluorophenyl)acetamide;
N-{5-[2-(2-chlorophenyl)acetamido]pyridazin-3-yl}-N-(3,5-difluorophenyl)acetamide;
N-{5-[2-(2-chloro-3-fluorophenyl)acetamido]pyridazin-3-yl}-N-(3,5-difluorophenyl)acetamide;
N-{5-[2-(2-chloro-4-fluorophenyl)acetamido]pyridazin-3-yl}-N-(3,5-difluorophenyl)acetamide; or
N-{5-[2-(2-chloro-6-fluorophenyl)acetamido]pyridazin-3-yl}-N-(3,5-difluorophenyl)acetamide;
or a stereoisomer, a tautomer, an N-oxide, a hydrate, a solvate, or a salt thereof, or a mixture of same.

11. A compound, wherein the compound is N-{4-[2-(2-chloro-3-fluorophenyl)acetamido]pyridin-2-yl}-N-(4-fluorophenyl)acetamide or a stereoisomer, a tautomer, an N-oxide, a hydrate, a solvate, or a salt thereof, or a mixture of same.

12. A method for the treatment or prophylaxis of a disease in a subject in need thereof, comprising administering a compound of formula (I) according to claim 1 to the subject.

13. A pharmaceutical composition comprising a compound of formula (I) according to claim 1 and one or more pharmaceutically acceptable excipients.

14-15. (canceled)

16. The method of claim 12, wherein the disease is pain, pain syndromes (acute and chronic), inflammatory-induced pain, neuropathic pain, diabetic neuropathic pain, diabetic neuropathy, cancer-associated pain, chemotherapy or intoxication induced pain, pelvic pain, endometriosis-associated pain as well as endometriosis as such, bladder pain syndrome; asthma, bronchiolitis obliterans syndrome, chronic obstructive pulmonary disease (COPD), chronic cough, diseases related to goblet cells and lung fibrosis, liver fibrosis, fatty liver disorders, NASH (Non-Alcoholic Steato-Hepatitis); brain ischemia, ischemic brain injury, ischemic stroke, haemorrhagic stroke, traumatic brain injury, spinal cord injury, aneurysm; chronic itch, pruritus; osteoarthritis, burning mouth syndrome, migraine disorders, irritable bowel disease; urology related syndromes like, overactive urinary bladder, interstitial cystitis, or bladder pain syndrome.

17. A compound of formula (XIV): 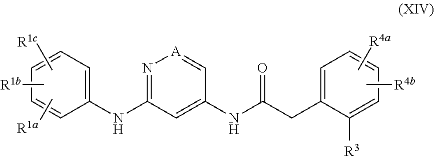
wherein A is CH or N;
R^{1a}, R^{1b}, and R^{1c} are each independently a hydrogen atom, a halogen atom, cyano, (C₁-C₃)-alkyl, (C₁-C₃)-haloalkyl, or (C₁-C₃)-alkoxy;
R³ is a halogen atom, cyano, (C₁-C₃)-alkyl, (C₁-C₃)-haloalkyl, or (C₁-C₃)-alkoxy; and
R^{4a} and R^{4b} are each independently a hydrogen atom, a halogen atom, cyano, (C₁-C₃)-alkyl, (C₁-C₃)-haloalkyl, or (C₁-C₃)-alkoxy;
or a stereoisomer, a tautomer, an N-oxide, a hydrate, a solvate, or a salt thereof, or a mixture of same.

18. The compound of claim 17, wherein the compound is:
N-(6-anilinopyridazin-4-yl)-2-(2,6-dichlorophenyl)acetamide;
N-(6-anilinopyridazin-4-yl)-2-(2-chloro-3-fluorophenyl)acetamide;
N-(6-anilinopyridazin-4-yl)-2-(2-chlorophenyl)acetamide;
N-(6-anilinopyridazin-4-yl)-2-(2-chloro-4-fluorophenyl)acetamide;
2-(2-chloro-6-fluorophenyl)-N-[6-(4-fluoroanilino)pyridazin-4-yl]acetamide;
2-(2-chloro-4-fluorophenyl)-N-[6-(4-fluoroanilino)pyridazin-4-yl]acetamide;
2-(2-chloro-3-fluorophenyl)-N-[6-(4-fluoroanilino)pyridazin-4-yl]acetamide;
2-(2-chlorophenyl)-N-[6-(4-fluoroanilino)pyridazin-4-yl]acetamide;
2-(2,6-dichlorophenyl)-N-[6-(4-fluoroanilino)pyridazin-4-yl]acetamide;
2-(2,6-dichlorophenyl)-N-[6-(3-fluoroanilino)pyridazin-4-yl]acetamide;
2-(2-chloro-3-fluorophenyl)-N-[6-(3-fluoroanilino)pyridazin-4-yl]acetamide;
2-(2-chlorophenyl)-N-[6-(3-fluoroanilino)pyridazin-4-yl]acetamide;
2-(2-chloro-6-fluorophenyl)-N-[6-(3-fluoroanilino)pyridazin-4-yl]acetamide;
2-(2-chloro-4-fluorophenyl)-N-[6-(3-fluoroanilino)pyridazin-4-yl]acetamide;
2-(2-chlorophenyl)-N-[6-(3,4-difluoroanilino)pyridazin-4-yl]acetamide;
2-(2,6-dichlorophenyl)-N-[6-(3,4-difluoroanilino)pyridazin-4-yl]acetamide;
2-(2-chloro-3-fluorophenyl)-N-[6-(3,4-difluoroanilino)pyridazin-4-yl]acetamide;
2-(2-chloro-4-fluorophenyl)-N-[6-(3,4-difluoroanilino)pyridazin-4-yl]acetamide;
2-(2-chloro-6-fluorophenyl)-N-[6-(3,4-difluoroanilino)pyridazin-4-yl]acetamide;
2-(2,6-dichlorophenyl)-N-[6-(3,5-difluoroanilino)pyridazin-4-yl]acetamide;
2-(2-chlorophenyl)-N-[6-(3,5-difluoroanilino)pyridazin-4-yl]acetamide;
2-(2-chloro-3-fluorophenyl)-N-[6-(3,5-difluoroanilino)pyridazin-4-yl]acetamide;
2-(2-chloro-4-fluorophenyl)-N-[6-(3,5-difluoroanilino)pyridazin-4-yl]acetamide;
2-(2-chloro-6-fluorophenyl)-N-[6-(3,5-difluoroanilino)pyridazin-4-yl]acetamide, or 2-(2-chloro-3-fluorophenyl)-N-[2-(4-fluoroanilino)pyridin-4-yl]acetamide,
or a stereoisomer, a tautomer, an N-oxide, a hydrate, a solvate, or a salt thereof, or a mixture of same.

19. A compound of formula (VII), (VIII) or (XIII) 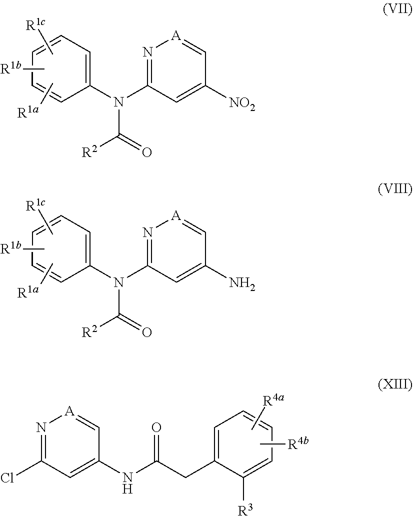
wherein A is CH or N;
R^{1a}, R^{1b}, and R^{1c} are each independently a hydrogen atom, a halogen atom, cyano, (C₁-C₃)-alkyl, (C₁-C₃)-haloalkyl, or (C₁-C₃)-alkoxy;
R² is (C₁-C₃)-alkyl; and
R^{4a}, R^{4b} are each independently a hydrogen atom, a halogen atom, cyano, (C₁-C₃)-alkyl, (C₁-C₃)-haloalkyl, or (C₁-C₃)-alkoxy;
or a stereoisomer, a tautomer, an N-oxide, a hydrate, a solvate, or a salt thereof, or a mixture of same.

20. The compound of claim 19, wherein the compound is:
N-(3,4-difluorophenyl)-N-(4-nitropyridin-2-yl)acetamide;
N-[3-(difluoromethyl)phenyl]-N-(4-nitropyridin-2-yl)acetamide;
N-(4-nitropyridin-2-yl)-N-[3-(trifluoromethyl)phenyl]acetamide;
N-[4-cyano-3-(trifluoromethyl)phenyl]-N-(4-nitropyridin-2-yl)acetamide;
N-(3-cyanophenyl)-N-(4-nitropyridin-2-yl)acetamide;
N-(4-chloro-3-cyanophenyl)-N-(4-nitropyridin-2-yl)acetamide;
N-(3-cyano-4-fluorophenyl)-N-(4-nitropyridin-2-yl)acetamide;
N-(4-nitropyridin-2-yl)-N-phenylacetamide;
N-(4-fluorophenyl)-N-(4-nitropyridin-2-yl)acetamide;
N-(4-methylphenyl)-N-(4-nitropyridin-2-yl)acetamide;
N-[4-(difluoromethoxy)phenyl]-N-(4-nitropyridin-2-yl)acetamide;
N-(3-chloro-4-fluorophenyl)-N-(4-nitropyridin-2-yl)acetamide;
N-(3-fluorophenyl)-N-(4-nitropyridin-2-yl)acetamide;
N-(4-chloro-3-fluorophenyl)-N-(4-nitropyridin-2-yl)acetamide;
N-(3-fluoro-4-methoxyphenyl)-N-(4-nitropyridin-2-yl)acetamide;
N-[3-fluoro-4-(methanesulfonyl)phenyl]-N-(4-nitropyridin-2-yl)acetamide;
N-(3,5-difluorophenyl)-N-(4-nitropyridin-2-yl)acetamide;
N-(3,5-difluoro-4-methylphenyl)-N-(4-nitropyridin-2-yl)acetamide;
N-(3,5-difluoro-4-methoxyphenyl)-N-(4-nitropyridin-2-yl)acetamide;
N-(3-methoxyphenyl)-N-(4-nitropyridin-2-yl)acetamide;
N-(4-nitropyridin-2-yl)-N-[3-(trifluoromethoxy)phenyl]acetamide;
N-(4-fluoro-3-methoxyphenyl)-N-(4-nitropyridin-2-yl)acetamide;
N-(2-chlorophenyl)-N-(4-nitropyridin-2-yl)acetamide;
N-(2-chloro-5-fluorophenyl)-N-(4-nitropyridin-2-yl)acetamide;
N-(2-fluorophenyl)-N-(4-nitropyridin-2-yl)acetamide;
N-[2-fluoro-4-(trifluoromethyl)phenyl]-N-(4-nitropyridin-2-yl)acetamide;
N-(2,3-difluorophenyl)-N-(4-nitropyridin-2-yl)acetamide;
N-(2,4-difluorophenyl)-N-(4-nitropyridin-2-yl)acetamide;
N-(3-chlorophenyl)-N-(4-nitropyridin-2-yl)acetamide;
N-(3-chloro-5-fluorophenyl)-N-(4-nitropyridin-2-yl)acetamide;
N-(3-Cyan-5-fluorphenyl)-N-(4-nitropyridin-2-yl)acetamide;
N-(2-Chlor-4-fluorphenyl)-N-(4-nitropyridin-2-yl)acetamide;
N-[3-Chlor-4-(methylsulfonyl)phenyl]-N-(4-nitropyridin-2-yl)acetamide;
N-(3-Fluor-5-methoxyphenyl)-N-(4-nitropyridin-2-yl)acetamide;
N-[2-(Difluormethyl)phenyl]-N-(4-nitropyridin-2-yl)acetamide;
N-(2,4-Dimethylphenyl)-N-(4-nitropyridin-2-yl)acetamide;
N-(3-Cyan-5-methylphenyl)-N-(4-nitropyridin-2-yl)acetamide;
N-(4-Fluor-2,3-dimethylphenyl)-N-(4-nitropyridin-2-yl)acetamide;
N-(3-Chlor-4-methylphenyl)-N-(4-nitropyridin-2-yl)acetamide;
N-(4-fluorophenyl)-N-(4-nitropyridin-2-yl)butanamide;
N-(4-aminopyridin-2-yl)-N-(3,4-difluorophenyl)acetamide;
N-(4-aminopyridin-2-yl)-N-(4-fluorophenyl)acetamide;
N-(4-aminopyridin-2-yl)-N-[3-(difluoromethyl)phenyl]acetamide;
N-(4-aminopyridin-2-yl)-N-[3-(trifluoromethyl)phenyl]acetamide;
N-(4-aminopyridin-2-yl)-N-[4-cyano-3-(trifluoromethyl)phenyl]acetamide;
N-(4-aminopyridin-2-yl)-N-(3-cyanophenyl)acetamide;
N-(4-aminopyridin-2-yl)-N-(4-chloro-3-cyanophenyl)acetamide;
N-(4-aminopyridin-2-yl)-N-(3-cyano-4-fluorophenyl)acetamide;
N-(4-aminopyridin-2-yl)-N-phenylacetamide;
N-(4-aminopyridin-2-yl)-N-(4-methylphenyl)acetamide;
N-(4-aminopyridin-2-yl)-N-[4-(difluoromethoxy)phenyl]acetamide;
N-(4-aminopyridin-2-yl)-N-(3-chloro-4-fluorophenyl)acetamide;
N-(4-aminopyridin-2-yl)-N-(3-fluorophenyl)acetamide;
N-(4-aminopyridin-2-yl)-N-(4-chloro-3-fluorophenyl)acetamide;
N-(4-aminopyridin-2-yl)-N-(3-fluoro-4-methoxyphenyl)acetamide;
N-(4-aminopyridin-2-yl)-N-[3-fluoro-4-(methanesulfonyl)phenyl]acetamide;
N-(4-aminopyridin-2-yl)-N-(3,5-difluorophenyl)acetamide;
N-(4-aminopyridin-2-yl)-N-(3,5-difluoro-4-methylphenyl)acetamide;
N-(4-aminopyridin-2-yl)-N-(3,5-difluoro-4-methoxyphenyl)acetamide;
N-(4-aminopyridin-2-yl)-N-(3-methoxyphenyl)acetamide;
N-(4-aminopyridin-2-yl)-N-[3-(trifluoromethoxy)phenyl]acetamide;
N-(4-aminopyridin-2-yl)-N-(4-fluoro-3-methoxyphenyl)acetamide;
N-(4-aminopyridin-2-yl)-N-(2-chlorophenyl)acetamide;
N-(4-aminopyridin-2-yl)-N-(2-chloro-5-fluorophenyl)acetamide;
N-(4-aminopyridin-2-yl)-N-(2-fluorophenyl)acetamide;
N-(4-aminopyridin-2-yl)-N-[2-fluoro-4-(trifluoromethyl)phenyl]acetamide;
N-(4-aminopyridin-2-yl)-N-(2,3-difluorophenyl)acetamide;
N-(4-aminopyridin-2-yl)-N-(2,4-difluorophenyl)acetamide;
N-(4-aminopyridin-2-yl)-N-(3-chlorophenyl)acetamide;
N-(4-aminopyridin-2-yl)-N-(3-chloro-5-fluorophenyl)acetamide;
N-(4-Aminopyridin-2-yl)-N-(3-cyan-5-fluorphenyl)acetamide;
N-(4-Aminopyridin-2-yl)-N-(2-chlor-4-fluorphenyl)acetamide;
N-(4-Aminopyridin-2-yl)-N-[3-chlor-4-(methylsulfonyl)phenyl]acetamide;
N-(4-Aminopyridin-2-yl)-N-(3-fluor-5-methoxyphenyl)acetamide;
N-(4-Aminopyridin-2-yl)-N-[2-(difluormethyl)phenyl]acetamide;
N-(4-Aminopyridin-2-yl)-N-(2,4-dimethylphenyl)acetamide;
N-(4-Aminopyridin-2-yl)-N-(3-cyan-5-methylphenyl)acetamide;
N-(4-Aminopyridin-2-yl)-N-(3-chlor-4-methylphenyl)acetamide;
N-(4-aminopyridin-2-yl)-N-(4-fluorophenyl)butanamide;
N-(6-chloropyridazin-4-yl)-2-(2,6-dichlorophenyl)acetamide;
2-(2-chloro-3-fluorophenyl)-N-(6-chloropyridazin-4-yl)acetamide;
2-(2-chlorophenyl)-N-(6-chloropyridazin-4-yl)acetamide;
2-(2-chloro-4-fluorophenyl)-N-(6-chloropyridazin-4-yl)acetamide; or
2-(2-chloro-6-fluorophenyl)-N-(6-chloropyridazin-4-yl)acetamide,
or a stereoisomer, a tautomer, an N-oxide, a hydrate, a solvate, or a salt thereof, or a mixture of same.

21. A method for the treatment or prophylaxis of a disease in a subject in need thereof, comprising administering a compound of formula (I) according to claim 11 to the subject.

22. The method of claim 21, wherein the disease is pain, pain syndromes (acute and chronic), inflammatory-induced pain, neuropathic pain, diabetic neuropathic pain, diabetic neuropathy, cancer-associated pain, chemotherapy or intoxication induced pain, pelvic pain, endometriosis-associated pain as well as endometriosis as such, bladder pain syndrome; asthma, bronchiolitis obliterans syndrome, chronic obstructive pulmonary disease (COPD), chronic cough, diseases related to goblet cells and lung fibrosis, liver fibrosis, fatty liver disorders, NASH (Non-Alcoholic Steato-Hepatitis); brain ischemia, ischemic brain injury, ischemic stroke, haemorrhagic stroke, traumatic brain injury, spinal cord injury, aneurysm; chronic itch, pruritus; osteoarthritis, burning mouth syndrome, migraine disorders, irritable bowel disease; urology related syndromes like, overactive urinary bladder, interstitial cystitis, bladder pain syndrome.

23. A pharmaceutical composition comprising a compound of formula (I) according to claim 11 and one or more pharmaceutically acceptable excipients.
